(19) United States
(12) Patent Application Publication
(10) Pub. No.: US 2025/0066751 A1
(43) Pub. Date: Feb. 27, 2025

(54) Cas9-Cas9 Fusion Proteins

(71) Applicant: University of Massachusetts, Westborough, MA (US)

(72) Inventors: Scott WOLFE, Winchester, MA (US); Mehmet Fatih Bolukbasi, Worchester, MA (US); Ankit Gupta, Worcester, MA (US); Erik J. Sontheimer, Aubumdale, MA (US); Nadia Amrani, Shrewsbury, MA (US)

(21) Appl. No.: 18/899,677

(22) Filed: Sep. 27, 2024

Related U.S. Application Data
(63) Continuation of application No. 17/313,328, filed on May 6, 2021, now abandoned.
Application No. 17/313,328 is a continuation of application No. 16/226,202, filed on Dec. 19, 2018, now Pat. No. 11,028,380.
Application No. 16/226,202 is a continuation of application No. 14/976,196, filed on Dec. 21, 2015, now Pat. No. 10,190,106.
(60) Provisional application No. 62/095,399, filed on Dec. 22, 2014.

(51) Int. Cl. C12N 9/22

(52) U.S. Cl. CPC C12N 9/22; C07K 2319/80; C12Y 301/00

(57) ABSTRACT

The present invention provides a Cas9 platform to facilitate single-site nuclease gene editing precision within a human genome. For example, a Cas9 nuclease/DNA-targeting unit (Cas9-DTU) fusion protein precisely delivers a Cas9/sgRNA complex to a specific target site within the genome for subsequent sgRNA-dependent cleavage of an adjacent target sequence. Alternatively, attenuating Cas9 binding using mutations to the a protospacer adjacent motif (PAM) recognition domain makes Cas9 target site recognition dependent on the associated DTU, all while retaining Cas9's sgRNA-mediated DNA cleavage fidelity. Cas9-DTU fusion proteins have improved target site binding precision, greater nuclease activity, and a broader sequence targeting range than standard Cas9 systems. Existing Cas9 or sgRNA variants (e.g., truncated sgRNAs (tru-gRNAs), nickases and FokI fusions) are compatible with these improvements to further reduce off-target cleavage. A robust, broadly applicable strategy is disclosed to impart Cas9 genome-editing systems with the single-genomic-site accuracy needed for safe, effective clinical application.

### PRIORITY STATEMENT

This application is a continuation of, and claims priority to, co-pending U.S. patent application Ser. No. 17/313,328 filed May 6, 2021, which is a continuation of application Ser. No. 16/226,202 filed Dec. 19, 2018, now U.S. patent Ser. No. 11/028,380, issued Jun. 8, 2021, which is a continuation of U.S. patent application Ser. No. 14/976,196, filed on Dec. 21, 2015, now U.S. patent Ser. No. 10/190,106, issued Jan. 1, 2019, which claims benefit of provisional 62/095,399 filed on Dec. 22, 2014, now expired, each of which is herein incorporated by reference in its entirety.

A Sequence Listing has been submitted in an xml file named “20635.xml” created on Sep. 25, 2024, consisting of 312,251 bytes, the entire content of which is herein incorporated by reference.

### GOVERNMENT SUPPORT

This invention was made with government support under Grant number 1R01AI117839 awarded by National Institutes of Health. The government has certain rights in the invention.

### FIELD OF THE INVENTION

The present invention may be related to the field of genetic engineering. In particular, specific genes or sequences within a genome can be deleted or modified in a directed manner with improved precision when a Cas9 nuclease may be coupled to an independent DNA targeting unit: for example, a programmable DNA-binding domain (pDBD) and/or to an alternate Cas9 isoform. An improvement in the precision of cleavage from this Cas9 nuclease-DNA targeting unit chimera may be realized by attenuating the DNA-binding affinity of the conventional Cas9 nuclease via specific mutations, such that an association of a Cas9 nuclease with its target site may be dependent on the specificity of the associated targeting unit (e.g., for example, either a programmable DNA-binding domain or to an alternate Cas9 isoform). These modifications have an added advantage of increasing the diversity of sequences that can be utilized as a target site, allowing breaks to be positioned more precisely near a specific target of interest. The association of Cas9 and the DNA targeting unit need not be covalent, but can be mediated through drug-dependent dimerization, which affords temporal control over the activity of this chimeric nuclease complex. This chimeric nuclease can be used in conjunction with other variants of Cas9 (e.g., for example, truncated guide RNAs, nickases or FokI fusions) that improve precision to further reduce the chance of cleaving unwanted sites within the treated genome.

### BACKGROUND

Cas9 (clustered regularly interspaced short palindromic repeats; CRISPR-associated system) may be part of a bacterial immune response to foreign nucleic acid introduction. The development of Type II CRISPR/Cas9 systems as programmable nucleases for genome engineering has been beneficial in the biomedical sciences. For example, a Cas9 platform has enabled gene editing in a large variety of biological systems, where both gene knockouts and tailor-made alterations are possible within complex genomes. The CRISPR/Cas9 system has the potential for application to gene therapy approaches for disease treatment, whether for the creation of custom, genome-edited cell-based therapies or for direct correction or ablation of aberrant genomic loci within patients.

The safe application of Cas9 in gene therapy requires exceptionally high precision to ensure that undesired collateral damage to the treated genome may be minimized or, ideally, eliminated. Numerous studies have outlined features of Cas9 that can drive editing promiscuity, and a number of strategies (e.g. truncated single-guide RNAs (sgRNAs), nickases and FokI fusions) have been developed that improve the precision of this system. However all of these systems still suffer from a degree of imprecision (cleavage resulting in lesions at unintended target sites within the genome).

However, what may be needed in the art are further improvements in editing precision to facilitate reliable clinical applications that require simultaneous efficient and accurate editing of multigigabase genomes in billions to trillions of cells, depending on the scope of genetic repair that may be needed for therapeutic efficacy.

### SUMMARY OF THE INVENTION

The present invention may be related to the field of genetic engineering. In particular, specific genes or sequences within a genome can be deleted or modified in a directed manner with improved precision when a Cas9 nuclease may be coupled to an independent DNA targeting unit: for example, a programmable DNA-binding domain and/or to an alternate Cas9 isoform.

An improvement in the precision of cleavage from this Cas9 nuclease-DNA targeting unit chimera may be realized by attenuating the DNA-binding affinity of the conventional Cas9 nuclease via specific mutations, such that an association of a Cas9 nuclease with its target site may be dependent on the specificity of the associated targeting unit (e.g., for example, either a programmable DNA-binding domain or to an alternate Cas9 isoform). These modifications have an added advantage of increasing the diversity of sequences that can be utilized as a target site, allowing breaks to be positioned more precisely near a specific target of interest. In addition, the fusion of a DNA targeting unit to Cas9 can also increase its activity relative to wild-type Cas9. The association of Cas9 and the DNA targeting unit need not be covalent, but can be mediated through drug-dependent or light-dependent dimerization, which afford temporal control over the activity of this chimeric nuclease complex. This chimeric nuclease can be used in conjunction with other variants of Cas9 (e.g., for example, truncated guide RNAs, nickases or FokI fusions) that improve precision to further reduce the chance of cleaving unwanted sites within the treated genome.

In one embodiment, the present invention contemplates a fusion protein comprising a Cas9 nuclease, said nuclease comprising a mutated protospacer adjacent motif recognition domain and a peptide linker, wherein said peptide linker is attached to a DNA binding domain (DBD) protein. In one embodiment, said Cas9 nuclease is selected from the group consisting of SpCas9, SaCas9, NmCas9 and AnCas9. In one embodiment, said mutated protospacer adjacent motif recognition domain is selected from the group consisting of Cas9^{MT1} Cas9^{MT2}, Cas9^{MT3} NmCas9^{SM} and NmCas9^{DM}. In one embodiment, said DBD protein is selected from the group consisting of DBD²⁶⁸, DBD^{TS1}, DBD^{TS2}, DBD^{TS3} and DBD^{TS4}. In one embodiment, said DBD protein is selected from the group consisting of a zinc finger protein and a transcription activator-like effector protein. In one embodiment, said fusion protein further comprises a guide RNA which is attached to a guide sequence element. In one embodiment, said mutated protospacer adjacent motif recognition domain comprises mutated DNA phosphodiester recognition amino acid residues. In one embodiment, said guide RNA is selected from the group consisting of an sgRNA sequence, a crRNA sequence and a tracrRNA sequence. In one embodiment, said guide sequence element is truncated. In one embodiment, said truncated guide sequence element is less than twenty nucleotides.

In one embodiment, the present invention contemplates a fusion protein comprising a Cas9 nuclease, said nuclease comprising a protospacer adjacent motif recognition domain and a truncated peptide linker, wherein said truncated peptide linker is attached to a DNA binding domain (DBD) protein. In one embodiment, said truncated peptide linker is between two and sixty amino acids. In one embodiment, said truncated peptide linker is between twenty-five and sixty amino acids. In one embodiment, said Cas9 nuclease is selected from the group consisting of SpCas9, SaCas9, NmCas9 and AnCas9. In one embodiment, said DBD protein is selected from the group consisting of DBD²⁶⁸, DBD^{TS1}, DBD^{TS2}, DBD^{TS3} and DBD^{TS4}. In one embodiment, said DBD protein is selected from the group consisting of a zinc finger protein and a transcription activator-like effector protein. In one embodiment, said fusion protein further comprises a guide RNA, which contains a guide sequence element. In one embodiment, said guide RNA is selected from the group consisting of an sgRNA sequence, a crRNA sequence and a tracrRNA sequence. In one embodiment, said guide sequence element is truncated. In one embodiment, said truncated guide sequence element is less than twenty nucleotides.

In one embodiment, the present invention contemplates a fusion protein comprising a Cas9 nuclease, said nuclease comprising a mutated protospacer adjacent motif recognition domain and a truncated peptide linker, wherein said truncated peptide linker is attached to a DNA binding domain (DBD) protein. In one embodiment, said truncated peptide linker is between two and sixty amino acids. In one embodiment, said truncated peptide linker is between twenty-five and sixty amino acids. In one embodiment, said mutated protospacer adjacent motif recognition domain is selected from the group consisting of Cas9^{MT1} Cas9^{MT2}, Cas9^{MT3}, NmCas9^{SM} and NmCas9^{DM}. In one embodiment, mutated protospacer adjacent motif recognition domain comprises mutated DNA phosphodiester recognition amino acid residues. In one embodiment, said Cas9 nuclease is selected from the group consisting of SpCas9, SaCas9, NmCas9 and AnCas9. In one embodiment, said DBD protein is selected from the group consisting of DBD²⁶⁸, DBD^{TS1}, DBD^{TS2}, DBD^{TS3} and DBD^{TS4}. In one embodiment, said DBD protein is selected from the group consisting of a zinc finger protein and a transcription activator-like effector protein. In one embodiment, said fusion protein further comprises a guide RNA which contains a guide sequence element. In one embodiment, said guide RNA is selected from the group consisting of an sgRNA sequence, a crRNA sequence and a tracrRNA sequence. In one embodiment, said guide sequence element is truncated. In one embodiment, truncated guide sequence element is less than twenty nucleotides.

In one embodiment, the present invention contemplates a DNA/protein complex comprising a Cas9 nuclease, said nuclease comprises a mutated protospacer adjacent motif recognition domain and a peptide linker, wherein said peptide linker is attached to a DNA binding domain (DBD) protein, wherein said mutated protospacer adjacent motif recognition domain at least partially binds to a DNA protospacer adjacent motif sequence and said DBD protein binds to a DNA target site, where target site cleavage precision has a specificity ratio greater than a Cas9^{WT} nuclease. In one embodiment, the DNA target site is a neighboring DNA target site. In one embodiment, said specificity ratio ranges between a two-fold to a one-hundred and fifty six fold greater than said Cas9^{WT} nuclease. In one embodiment, said Cas9 nuclease is selected from the group consisting of SpCas9, SaCas9, NmCas9 and AnCas9. In one embodiment, the mutated protospacer adjacent motif recognition domain renders target recognition dependent on the DNA-binding domain. In one embodiment, said mutated protospacer adjacent motif recognition domain is selected from the group consisting of Cas9^{MT1} Cas9^{MT2}, Cas9^{MT3}, NmCas9^{SM} and NmCas9^{DM}. In one embodiment, said DBD protein is selected from the group consisting of DBD²⁶⁸, DBD^{TS1}, DBD^{TS2}, DBD^{TS3} and DBD^{TS4}. In one embodiment, said DNA target site is selected from the group consisting of a Zif268 site, a TS1 site, a TS2 site, a TS3 site and a TS4 site. In one embodiment, said DBD protein is selected from the group consisting of a zinc finger protein and a transcription activator-like effector protein. In one embodiment, said complex further comprises a guide RNA attached to a guide sequence element that is complementary to a region of the DNA target site. In one embodiment, said Cas9 nuclease comprises mutated DNA phosphodiester recognition amino acid residues. In one embodiment, said guide RNA is selected from the group consisting of an sgRNA sequence, a crRNA sequence and a tracrRNA sequence. In one embodiment, said guide sequence element is truncated. In one embodiment, said truncated guide sequence element is less than twenty nucleotides.

In one embodiment, the present invention contemplates a DNA/protein complex comprising a Cas9 nuclease, said nuclease comprises a protospacer adjacent motif recognition domain and a truncated peptide linker, wherein said truncated peptide linker is attached to a DNA binding domain (DBD) protein, wherein said protospacer adjacent motif recognition domain binds to a DNA protospacer adjacent motif sequence, and said DBD protein binds to a DNA target site, where target site cleavage precision has a specificity ratio greater than a Cas9^{WT} nuclease. In one embodiment, the DNA target site is a neighboring DNA target site. In one embodiment, said truncated peptide linker is between two and sixty amino acids. In one embodiment, said peptide linker comprises between twenty-five and sixty amino acids. In one embodiment, said specificity ratio ranges between a two-fold to a one-hundred and fifty six fold greater than said Cas9^{WT} nuclease. In one embodiment, said Cas9 nuclease is selected from the group consisting of SpCas9, SaCas9, NmCas9 and AnCas9. In one embodiment, said DNA target site is selected from the group consisting of a Zif268 site, a TS1 site, a TS2 site, a TS3 site and a TS4 site. In one embodiment, said DBD protein is selected from the group consisting of DBD²⁶⁸, DBD^{TS1}, DBD^{TS2}, DBD^{TS3} and DBD^{TS4}. In one embodiment, said DBD protein is selected from the group consisting of a zinc finger protein and a transcription activator-like effector protein. In one embodiment, said complex further comprises a guide RNA attached to a guide sequence element that is complementary to a region of the DNA target site. In one embodiment, said guide RNA is selected from the group consisting of an sgRNA sequence, a crRNA sequence and a tracrRNA sequence. In one embodiment, said guide sequence element is truncated. In one embodiment, said truncated guide sequence element is less than twenty nucleotides.

In one embodiment, the present invention contemplates, a DNA/protein complex comprising a Cas9 nuclease, said nuclease comprises a mutated protospacer adjacent motif recognition domain and a truncated peptide linker, wherein said truncated peptide linker is attached to a DNA binding domain (DBD) protein, wherein said mutated protospacer adjacent motif recognition domain at least partially binds to a DNA protospacer adjacent motif sequence, and said DBD protein binds to a DNA target site, where target site cleavage has a specificity ratio greater than a Cas9^{WT} nuclease. In one embodiment, the DNA target site is a neighboring DNA target site. In one embodiment, said truncated peptide linker comprises between two and sixty amino acids. In one embodiment, said truncated peptide linker comprises between twenty-five and sixty amino acids. In one embodiment, said specificity ratio ranges between a two-fold to a one-hundred and fifty six fold greater than said Cas9^{WT} nuclease. In one embodiment, the mutated protospacer adjacent motif recognition domain renders target recognition dependent on the DNA-binding domain. In one embodiment, said mutated protospacer adjacent motif recognition domain is selected from the group consisting of Cas9^{MT1} Cas9^{MT2}, Cas9^{MT3}, NmCas9^{SM} and NmCas9^{DM}. In one embodiment, said mutated protospacer adjacent motif recognition domain comprises mutated DNA phosphodiester recognition amino acid residues. In one embodiment, said DNA target site is selected from the group consisting of a Zif268 site, a TS1 site, a TS2 site, a TS3 site and a TS4 site. In one embodiment, said DBD protein is selected from the group consisting of DBD²⁶⁸, DBD^{TS1}, DBD^{TS2}, DBD^{TS3} and DBD^{TS4}. In one embodiment, said DBD protein is selected from the group consisting of a zinc finger protein and a transcription activator-like effector protein. In one embodiment, said fusion protein further comprises a guide RNA attached to a guide sequence element that is complementary to a region of the DNA target site. In one embodiment, said guide RNA is selected from the group consisting of an sgRNA sequence, a crRNA sequence and a tracrRNA sequence. In one embodiment, said guide sequence element is truncated. In one embodiment, said truncated guide sequence element is less than twenty nucleotides.

In one embodiment, the present invention contemplates a method for genome editing of DNA, comprising: a) providing: i) a DNA sequence comprising a target site sequence, and a protospacer adjacent motif sequence; and ii) a fusion protein comprising a Cas9 nuclease, said nuclease comprising a mutated protospacer adjacent motif recognition domain and a peptide linker, wherein said peptide linker is attached to a DNA targeting unit (DTU) protein; b) contacting said fusion protein with said target site sequence such that said DTU binds to said target site sequence and said mutated protospacer adjacent motif recognition domain at least partially binds to said protospacer adjacent motif sequence; and c) cleaving said target site with said Cas9 nuclease. In one embodiment, said cleaving is at a single nucleotide target site. In one embodiment, the cleaving performs gene editing. In one embodiment, said DNA sequence is within a cell. In one embodiment, said target site further comprises a sequence complementary to the guide sequence element, a protospacer adjacent motif sequence and a recognition sequence for the DNA targeting unit In one embodiment, said Cas9 nuclease cleaves said target site with a precision that has a specificity ratio between two and one-hundred and fifty six fold greater than a Cas9^{WT} nuclease. In one embodiment, said Cas9 nuclease is selected from the group consisting of SpCas9, SaCas9, NmCas9 and AnCas9. In one embodiment, the mutated protospacer adjacent motif recognition domain renders target recognition dependent on the DNA-binding domain. In one embodiment, said mutated protospacer adjacent motif recognition domain is selected from the group consisting of Cas9^{MT1} Cas9^{MT2}, Cas9^{MT3}, NmCas9^{SM} and NmCas9^{DM}. In one embodiment, said DNA target site is selected from the group consisting of a Zif268 site, a TS1 site, a TS2 site, a TS3 site and a TS4 site. In one embodiment, said DTU is selected from the group consisting of DBD²⁶⁸, DBD^{TS1}, DBD^{TS2}, DBD^{TS3}, DBD^{TS4} and nuclease-dead NmCas9 (NmdCas9). In one embodiment, said DTU is selected from the group consisting of a zinc finger protein, a transcription activator-like effector protein and an alternate Cas9 isoform. In one embodiment, said fusion protein further comprises one guide RNA for each Cas9 isoform incorporated, which contains a guide sequence element that is complementary to a region of the target site sequence. In one embodiment, said mutated protospacer adjacent motif recognition domain comprises mutated DNA phosphodiester recognition amino acid residues. In one embodiment, said guide RNA is selected from the group consisting of an sgRNA sequence, a crRNA sequence and a tracrRNA sequence. In one embodiment, said guide sequence element is truncated. In one embodiment, said truncated guide sequence element is less than twenty nucleotides.

In one embodiment, a method for genome editing of DNA within a cell, comprising: a) providing: i) a DNA sequence comprising a target site sequence, and a protospacer adjacent motif sequence; and ii) a fusion protein comprising a Cas9 nuclease, said nuclease comprising a protospacer adjacent motif recognition domain and a truncated peptide linker, wherein said truncated peptide linker is attached to a DNA targeting unit (DTU); b) contacting said fusion protein with said DNA sequence such that said DTU binds to said target site sequence and said protospacer adjacent motif recognition domain binds to said protospacer adjacent motif sequence; and c) cleaving said target sequence with said Cas9 nuclease. In one embodiment, the cleaving is a single nucleotide target site. In one embodiment, the cleaving performs gene editing. In one embodiment, said target site comprises a sequence complementary to the guide sequence element, a protospacer adjacent motif sequence and a recognition sequence for the DNA targeting unit. In one embodiment, said Cas9 fusion protein cleaves said target site with a precision that has a specificity ratio between two and one-hundred and fifty six fold greater than a Cas9^{WT} nuclease. In one embodiment, said truncated peptide linker is between two and sixty amino acids. In one embodiment, said truncated peptide linker is between twenty-five and sixty amino acids. In one embodiment, said Cas9 nuclease is selected from the group consisting of SpCas9, SaCas9, NmCas9 and AnCas9. In one embodiment, said DNA target site is selected from the group consisting of a Zif268 site, a TS1 site, a TS2 site, a TS3 site and a TS4 site. In one embodiment, said DTU is selected from the group consisting of DBD²⁶⁸, DBD^{TS1}, DBD^{TS2} DBD^{TS3}, DBD^{TS4} and nuclease-dead NmCas9 (NmdCas9). In one embodiment, said DTU is selected from the group consisting of a zinc finger protein, a transcription activator-like effector protein and an alternate Cas9 isoform. In one embodiment, said fusion protein further comprises one guide RNA for each Cas9 isoform incorporated, which contains a guide sequence element that is complementary to a region of the target site sequence. In one embodiment, said guide RNA sequence is selected from the group consisting of an sgRNA sequence, a crRNA sequence and a tracrRNA sequence. In one embodiment, said guide sequence element is truncated. In one embodiment, said truncated guide sequence element is less than twenty nucleotides.

In one embodiment, genome editing of DNA within a cell, comprising: a) providing: i) a DNA sequence comprising a target site sequence, and a protospacer adjacent motif sequence; and ii) a fusion protein comprising a Cas9 nuclease, said nuclease comprising a mutated protospacer adjacent motif recognition domain and a truncated peptide linker, wherein said truncated peptide linker is attached to a DNA targeting unit (DTU); b) contacting said fusion protein with said DNA sequence such that said DTU protein binds within said target site sequence and said protospacer adjacent motif recognition domain binds to said protospacer adjacent motif sequence; and c) cleaving the target site with said Cas9 nuclease. In one embodiment, said target site comprises a sequence complementary to the guide sequence element, a protospacer adjacent motif sequence and a recognition sequence for the DNA targeting unit. In one embodiment, the cleaving is performed at a single nucleotide target site. In one embodiment, the cleaving performs gene editing. In one embodiment, said Cas9 fusion protein cleaves said target site with a precision that has a specificity ratio between two and one-hundred and fifty six fold greater than a Cas9^{WT} nuclease. In one embodiment, said truncated peptide linker is between two and sixty amino acids. In one embodiment, said truncated peptide linker is between twenty-five and sixty amino acids. In one embodiment, the mutated protospacer adjacent motif recognition domain renders target recognition dependent on the DNA-binding domain. In one embodiment, said mutated protospacer adjacent motif recognition domain is selected from the group consisting of Cas9^{MT1} Cas9^{MT2}, Cas9^{MT3}, NmCas9^{SM} and NmCas9^{DM}. In one embodiment, said the mutated protospacer adjacent motif recognition domain comprises mutated DNA phosphodiester recognition amino acid residues. In one embodiment, said Cas9 nuclease is selected from the group consisting of SpCas9, SaCas9, NmCas9 and AnCas9. In one embodiment, said DNA target site is selected from the group consisting of a Zif268 site, a TS1 site, a TS2 site, a TS3 site and a TS4 site. In one embodiment, said DTU is selected from the group consisting of DBD²⁶⁸, DBD^{TS1}, DBD^{TS2}, DBD^{TS3}, DBD^{TS4} and nuclease-dead NmCas9 (NmdCas9). In one embodiment, said DTU is selected from the group consisting of a zinc finger protein, a transcription activator-like effector protein and an alternate Cas9 isoform. In one embodiment, said fusion protein further comprises one guide RNA for each Cas9 isoform incorporated, which contains a guide sequence element that is complementary to a region of the target site sequence. In one embodiment, said guide RNA is selected from the group consisting of an sgRNA sequence, a crRNA sequence and a tracrRNA sequence. In one embodiment, said guide sequence element is truncated. In one embodiment, said trucated guide sequence element is less than twenty nucleotides.

In one embodiment, the present invention contemplates a method of treatment, comprising: a) providing: i) a patient exhibiting at least one symptom of a genetic disease and comprising a cell with a DNA target site, said DNA target site comprising a gene mutation responsible for said genetic disease; ii) a delivery vehicle comprising a Cas9-DNA targeting unit (DTU) fusion protein capable of genome editing selected from the group consisting of: A) a Cas9 nuclease, said nuclease comprising a mutated protospacer adjacent motif recognition domain and a peptide linker, wherein said peptide linker is attached to a DNA targeting unit (DTU); B) a Cas9 nuclease, said nuclease comprising a protospacer adjacent motif recognition domain and a truncated peptide linker, wherein said truncated peptide linker is attached to a DNA targeting unit (DTU); and C) a Cas9 nuclease, said nuclease comprising a mutated protospacer adjacent motif recognition domain and a truncated peptide linker, wherein said truncated peptide linker is attached to a targeting unit (DTU); b) administering said plasmid to said patient such that said plasmid transfects said cell; c) expressing said plasmid within said cell such that said expressed Cas9-DBD fusion protein contacts said DNA target site; d) editing said DNA target site with said Cas9 nuclease; and e) reducing said at least one symptom of said genetic disease in said patient. In one embodiment, said Cas9 fusion protein cleaves said target site with a precision that has a specificity ratio between two and one-hundred and fifty six fold greater than a Cas9wT nuclease. In one embodiment, the delivery vehicle includes, but is not limited to, a plasmid, a vector, a virus or an mRNA. In one embodiment, said truncated peptide linker is between two and sixty amino acids. In one embodiment, said truncated peptide linker is between twenty-five and sixty amino acids. In one embodiment, the mutated protospacer adjacent motif recognition domain renders target recognition dependent on the DNA-binding domain. In one embodiment, said mutated protospacer adjacent motif recognition domain is selected from the group consisting of Cas9^{MT1} Cas9^{MT2}, Cas9^{MT3}, NmCas9^{SM} and NmCas9^{DM}In one embodiment, said mutated protospacer adjacent motif recognition domain comprises mutated DNA phosphodiester recognition amino acid residues. In one embodiment, said Cas9 nuclease is selected from the group consisting of SpCas9, SaCas9, NmCas9 and AnCas9. In one embodiment, said DNA target site is selected from the group consisting of a Zif268 site, a TS1 site, a TS2 site, a TS3 site and a TS4 site. In one embodiment, said DTU is selected from the group consisting of DBD²⁶⁸, DBD^{TS1}, DBD^{TS2}, DBD^{TS3}, DBD^{TS4} and nuclease-dead NmCas9 (NmdCas9). In one embodiment, said DTU is selected from the group consisting of a zinc finger protein, a transcription activator-like effector protein and an alternate Cas9 isoform. In one embodiment, said fusion protein further comprises one guide RNA for each Cas9 isoform incorporated, which contains a guide sequence element that is complementary to a region of the target site sequence. In one embodiment, said guide RNA is selected from the group consisting of an sgRNA sequence, a crRNA sequence and a tracrRNA sequence. In one embodiment, said guide sequence element is truncated. In one embodiment, said truncated guide sequence element is less than twenty nucleotides. In one embodiment, said genetic disease is selected from the group consisting of chronic granulomatous disease, Huntington's disease, myotonic dystrophy, and HIV.

In one embodiment, the present invention contemplates a method of prevention, comprising: a) providing: i) a patient comprising a cell with a DNA target site, said DNA target site comprising a gene mutation responsible for a genetic disease; ii) a plasmid comprising a Cas9-DNA targeting unit (DTU) fusion protein capable of genome editing selected from the group consisting of: A) a Cas9 nuclease, said nuclease comprising a mutated protospacer adjacent motif recognition domain and a peptide linker, wherein said peptide linker is attached to a DNA targeting unit (DTU); B) a Cas9 nuclease, said nuclease comprising a protospacer adjacent motif recognition domain and a truncated peptide linker, wherein said truncated peptide linker is attached to a DNA targeting unit (DTU); and C) a Cas9 nuclease, said nuclease comprising a mutated protospacer adjacent motif recognition domain and a truncated peptide linker, wherein said truncated peptide linker is attached to a DNA targeting unit (DTU); b) administering said plasmid to said patient such that said plasmid transfects said cell; c) expressing said plasmid within said cell such that said expressed Cas9-DTU fusion protein contacts said DNA target site; d) editing said DNA target site with said Cas9 nuclease; and e) preventing the development of said genetic disease in said patient. In one embodiment, said Cas9 fusion protein cleaves said target site with a precision that has a specificity ratio between two and one-hundred and fifty six fold greater than a Cas9wT nuclease. In one embodiment, said truncated peptide linker is between two and sixty amino acids. In one embodiment, said truncated peptide linker is between twenty-five and sixty amino acids. In one embodiment, the mutated protospacer adjacent motif recognition domain renders target recognition dependent on the DNA-binding domain. In one embodiment, said mutated protospacer adjacent motif recognition domain is selected from the group consisting of Cas9^{MT1} Cas9^{MT2}, Cas9^{MT3}, NmCas9^{SM} and NmCas9^{DM}. In one embodiment, said mutated protospacer adjacent motif recognition domain comprises mutated DNA phosphodiester recognition amino acid residues. In one embodiment, said Cas9 nuclease is selected from the group consisting of SpCas9, SaCas9, NmCas9 and AnCas9. In one embodiment, said DNA target site is selected from the group consisting of a Zif268 site, a TS1 site, a TS2 site, a TS3 site and a TS4 site. In one embodiment, said DTU is selected from the group consisting of DBD², DBD^{TS1}, DBD^{TS2}, DBD^{TS3}, DBD^{TS4} and nuclease-dead NmCas9 (NmdCas9). In one embodiment, said DTU is selected from the group consisting of a zinc finger protein, a transcription activator-like effector protein and an alternate Cas9 isoform. In one embodiment, said fusion protein further comprises one guide RNA for each Cas9 isoform incorporated, which contains a guide sequence element that is complementary to a region of the DNA target site. In one embodiment, said guide RNA is selected from the group consisting of an sgRNA sequence, a crRNA sequence and a tracrRNA sequence. In one embodiment, said guide sequence element is truncated. In one embodiment, said trucated guide sequence element is less than twenty nucleotides. In one embodiment, said genetic disease is selected from the group consisting of Chronic granulomatous disease, Huntington's disease, myotonic dystrophy and HIV.

In one embodiment, the present invention contemplates a kit, comprising: a) a first container comprising a Cas9-DTU fusion protein selected from the group consisting of: A) a Cas9 nuclease, said nuclease comprising a mutated protospacer adjacent motif recognition domain and a peptide linker, wherein said peptide linker is attached to a DNA targeting unit (DTU); B) a Cas9 nuclease, said nuclease comprising a protospacer adjacent motif recognition domain and a truncated peptide linker, wherein said truncated peptide linker is attached to a DNA targeting unit (DTU); and C) a Cas9 nuclease, said nuclease comprising a mutated protospacer adjacent motif recognition domain and a truncated peptide linker, wherein said truncated peptide linker is attached to a DNA targeting unit (DTU); b) a second container comprising a single guide RNA complementary to a specific genomic target sequence for each Cas9 isoform present; and c) a set of instructions for genome editing of said specific genomic target. In one embodiment, said Cas9-DTU fusion protein is encoded by a plasmid. In one embodiment, said Cas9 fusion protein cleaves said target site with a precision that has a specificity ratio between two and one-hundred and fifty six fold greater than a Cas9^{WT} nuclease. In one embodiment, said truncated peptide linker is between two and sixty amino acids. In one embodiment, said truncated peptide linker is between twenty-five and sixty amino acids. In one embodiment, the mutated protospacer adjacent motif recognition domain renders target recognition dependent on the DNA-binding domain. In one embodiment, said mutated protospacer adjacent motif recognition domain is selected from the group consisting of Cas9^{MT1} Cas9^{MT2}, Cas9^{MT3}, NmCas9^{SM} and NmCas9^{M}. In one embodiment, said mutated protospacer adjacent motif recognition domain comprises mutated DNA phosphodiester recognition amino acid residues. In one embodiment, said Cas9 nuclease is selected from the group consisting of SpCas9, SaCas9, NmCas9 and AnCas9. In one embodiment, said DNA target site is selected from the group consisting of a Zif268 site, a TS1 site, a TS2 site, a TS3 site and a TS4 site. In one embodiment, said DTU is selected from the group consisting of DBD²⁶⁸, DBD^{TS1}, DBD^{TS2}, DBD^{TS3}, DBD^{TS4} and nuclease-dead NmCas9 (NmdCas9). In one embodiment, said DBD protein is selected from the group consisting of a zinc finger protein, a transcription activator-like effector protein and an alternate Cas9 isoform. In one embodiment, said fusion protein further comprises one guide RNA for each Cas9 isoform incorporated, which contains a guide sequence element that is complementary to a region of the DNA target site. In one embodiment, said guide RNA is selected from the group consisting of an sgRNA sequence, a crRNA sequence and a tracrRNA sequence. In one embodiment, said guide sequence element is truncated. In one embodiment, said trucated guide sequence element is less than twenty nucleotides. In one embodiment, said kit further comprises instructions for treating a genetic disease. In one embodiment, said genetic disease is selected from the group consisting of chronic granulomatous disease, Huntington's disease, myotonic dystrophy and HIV.

In one embodiment, the present invention contemplates a composition comprising a Cas9 nuclease-DNA targeting unit fusion protein. In one embodiment, the DNA targeting unit may be a programmable DNA binding domain such as a zinc finger protein. In one embodiment, the DNA targeting unit may be a programmable DNA binding domain such as a transcription activator-like effector protein. In one embodiment, the DNA targeting unit may be a programmable DNA binding domain such as a homeodomain protein. In one embodiment, the DNA targeting unit may be a different Cas9 isoform that can be independently programmed with a single guide RNA to a neighboring target site. In one embodiment, the Cas9 nuclease comprises a mutated protospacer adjacent motif recognition sequence. In one embodiment, the Cas9 nuclease comprises mutated residues that bind the phosphodiester backbone of the DNA or RNA. In one embodiment, the composition further comprises a single guide RNA which contains a guide sequence element that is complementary to a region of the DNA target site. In one embodiment, the single guide RNA may be truncated. In one embodiment, the composition further comprises a two orthogonal single guide RNAs. In one embodiment, both single guide RNAs are truncated. In one embodiment, the truncated single guide sequence element contains a guide segments that may be less than twenty nucleotides. In one embodiment, the mutated protospacer adjacent motif recognition sequence comprises at least one mutation.

In one embodiment, the present invention contemplates a composition comprising a Cas9 nuclease and a DNA targeting unit. In one embodiment, the DNA targeting unit comprises at least one dimerization domain. In one embodiment, the Cas9 nuclease and said DNA targeting unit are fused at said dimerization domain. In one embodiment, the DNA targeting unit may be a programmable DNA binding domain such as a zinc finger protein. In one embodiment, the DNA targeting unit may be a programmable DNA binding domain such as a transcription activator-like effector protein. In one embodiment, the DNA targeting unit may be a programmable DNA binding domain such as a homeodomain protein. In one embodiment, the DNA targeting unit may be a different Cas9 isoform that can be independently programmed with a single guide RNA to a neighboring target site. In one embodiment, the Cas9 nuclease comprises a mutated protospacer adjacent motif recognition sequence. In one embodiment, the Cas9 nuclease comprises mutated residues that bind the phosphodiester backbone of the DNA or RNA. In one embodiment, the composition further comprises a single guide RNA which contains a guide sequence element that is complementary to a region of the DNA target site. In one embodiment, the single guide RNA may be truncated. In one embodiment, the composition further comprises a two orthogonal single guide RNAs. In one embodiment, both single guide RNAs are truncated within the region complementary to the target site. In one embodiment, the at least one dimerization domain may be heterotypic. In one embodiment, at least one dimerization domain may be fused to an RNA binding protein that recognizes a sequence within the sgRNA. In one embodiment, a complementary RNA binding domain may be fused to a DNA targeting unit (DTU). In one embodiment, at least one dimerization domain may be complementary an RNA segment fused to the sgRNA of an orthogonal Cas9/sgRNA isoform. In one embodiment, the truncated single guide RNA contains a guide segment that may be less than twenty nucleotides. In one embodiment, the mutated protospacer adjacent motif recognition sequence comprises at least one mutation.

In one embodiment, the present invention contemplates a method, comprising: a) providing: i) a cell comprising a specific genomic target, wherein said specific genomic target comprises an on-target binding sequence; ii) a first vector encoding a Cas9 nuclease-DNA targeting unit fusion protein; iii) a second vector comprising a single guide RNA gene capable of expressing a single guide RNA (sgRNA) having complete complementarity to said specific genomic target; and b) expressing said first and second vectors in said cell, wherein a Cas9-DNA targeting unit fusion protein/sgRNA complex may be created; c) binding said Cas9-DNA targeting fusion protein/sgRNA complex to said on-target binding sequence, under conditions such that said specific genomic target may be cleaved. In one embodiment, the Cas9-DNA targeting unit fusion protein further comprises a mutated protospacer adjacent motif recognition sequence. In one embodiment, the mutated protospacer adjacent motif recognition sequence prevents independent binding of the Cas9 to DNA without the prior binding of the DNA-targeting unit. In one embodiment, the mutated protospacer adjacent motif comprises at least one mutation. In one embodiment, the DNA targeting unit may be a programmable DNA binding domain selected from a zinc finger protein, a transcription activator-like effector protein and a homeodomain protein. In one embodiment, the DNA targeting unit may be an alternate Cas9 isoform that may be programmed with an orthogonal sgRNA to the sgRNA that may be used to program the Cas9 nuclease. In one embodiment, the sgRNA sequence may be truncated. In one embodiment, the truncated sgRNA may be complementary to said target site at less than 20 nucleotides. In one embodiment, the specific genomic target may be a gene of interest. In one embodiment, the specific genomic target may be a single allele.

In one embodiment, the present invention contemplates a kit, comprising: a) a first container comprising a Cas9 nuclease-DNA targeting unit fusion protein capable of binding to a specific genomic target; b) a second container comprising a single guide RNA complementary to said specific genomic target or a pair of orthogonal single guide RNAs if the DNA targeting unit may be an alternate Cas9 isoform, and c) a set of instructions for employing these reagents to cleave said specific genomic target. In one embodiment, the DNA targeting unit may be a programmable DNA binding domain selected from the group consisting of a zinc finger protein, a transcription activator-like effector protein and a homeodomain protein. In one embodiment, the Cas9 fusion protein comprises a mutated protospacer adjacent motif recognition sequence. In one embodiment, the single guide RNA may be truncated. In one embodiment, the truncated guide sequence element may be less than twenty nucleotides. In one embodiment, the mutated protospacer adjacent motif recognition sequence comprises at least one mutation.

In one embodiment, the present invention contemplates a kit, comprising: a) a first container comprising a Cas9 nuclease fused to a dimerization domain; b) a second container comprising a DNA targeting unit fused to a complementary dimerization domain capable of binding to a specific sequence neighboring the genomic target site or being programmed to recognize this sequence with an appropriate guide RNA; c) a third container comprising a single guide RNA complementary to said specific genomic target or a pair of orthogonal single guide RNAs if the DNA targeting unit may be an alternate Cas9 isoform, where the second guide RNA recognizes a binding site neighboring the target site, and d) a set of instructions for employing these reagents to cleave said specific genomic target. In one embodiment, the DNA targeting unit may be a programmable DNA binding domain selected from the group consisting of a zinc finger protein, a transcription activator-like effector protein and a homeodomain protein. In one embodiment, the Cas9 fusion protein comprises a mutated protospacer adjacent motif recognition sequence. In one embodiment, the single guide RNA may be truncated. In one embodiment, the truncated guide sequence element may be less than twenty nucleotides. In one embodiment, the mutated protospacer adjacent motif recognition sequence comprises at least one mutation.

In one embodiment, the present invention contemplates a kit, comprising: a) a first container comprising a first vector encoding a Cas9-programmable DNA binding domain fusion protein capable of binding to a specific genomic target; b) a second container comprising a second vector comprising a single guide RNA gene encoding a guide sequence element complementary to said specific genomic target, and c) a set of instructions for deleting said specific genomic target. In one embodiment, the programmable DNA binding domain may be selected from the group consisting of a zinc finger protein and a transcription activator-like effector protein. In one embodiment, the DNA targeting unit may be an alternate Cas9 isoform that may be programmed with an orthogonal sgRNA to the sgRNA that may be used to program the Cas9 nuclease. In one embodiment, the Cas9 fusion protein comprises a mutated protospacer adjacent motif recognition domain sequence. In one embodiment, the Cas9 nuclease comprises mutated residues that bind the phosphodiester backbone of the DNA or RNA. In one embodiment, the guide sequence element may be truncated. In one embodiment, the truncated guide sequence element may be less than twenty nucleotides. In one embodiment, the mutated protospacer adjacent motif recognition domain comprises at least one mutation.

In one embodiment, the present invention contemplates a fusion protein comprising a Cas9 nuclease, said nuclease comprising a mutated protospacer adjacent motif recognition domain and a peptide linker, wherein said peptide linker is attached to a DNA targeting unit (DTU) comprising a second Cas9 nuclease. In one embodiment, the Cas9 nucleases are selected from the group consisting of SpCas9, SaCas9, NmCas9 and AnCas9. In one embodiment, the mutated protospacer adjacent motif recognition domain comprises mutated DNA phosphodiester recognition amino acid residues. In one embodiment, the mutated protospacer adjacent motif recognition domain is selected from the group consisting of SpCas9^{MT1}, SpCas9^{MT2}, SpCas9^{MT3}, NmCas9^{SM} and NmCas9^{DM}. In one embodiment, the DTU includes, but is not limited to, a Cas9 nuclease, a Cas9 nickase or a nuclease-dead Cas9 (dCas9). In one embodiment, the DTU is selected from the group consisting of nuclease-dead NmCas9 (NmdCas9), NmCas9 nuclease, NmCas9 nickase (HNH), and NmCas9 nickase (RuvC). In one embodiment, each Cas9 within the fusion protein has guide RNA attached to a guide sequence element. In one embodiment, said guide RNAs are selected from the group consisting of an sgRNA sequence, a crRNA sequence and a tracrRNA sequence. In one embodiment, the guide sequence element is truncated. In one embodiment, the truncated guide sequence element is less than twenty nucleotides.

In one embodiment, the present invention contemplates a two component Cas9 nuclease DNA targeting unit (DTU) system, wherein said system is inactive until assembled via drug-dependent or light-dependent dimerization thereby improving nuclease precision and activity, said system comprising: a) a fusion protein comprising: i) a Cas9 nuclease, said nuclease comprising a mutated protospacer adjacent motif recognition domain and a first peptide linker, wherein said peptide linker is attached to a first drug-dependent or light-dependent dimerization domain (Cas9 nuclease component); and ii) a DNA targeting unit (DTU) and a second peptide linker, wherein said second peptide linker is attached to a second drug-dependent or light-dependent dimerization domain (DTU component); and b) a DNA target site. In one embodiment, the Cas9 nuclease is selected from the group consisting of SpCas9, SaCas9, NmCas9 and AnCas9. In one embodiment, the mutated protospacer adjacent motif recognition domain renders recognition of said DNA target site by the Cas9 nuclease component dependent on the DTU. In one embodiment, the mutated protospacer adjacent motif recognition domain comprises mutated DNA phosphodiester recognition amino acid residues. In one embodiment, the mutated protospacer adjacent motif recognition domain is selected from the group consisting of SpCas9^{MT1} SpCas9^{MT2}, SpCas9^{MT3}, NmCas9^{SM} and NmCas9^{DM}. In one embodiment, the DTU can be a zinc finger protein, a transcription activator-like effector protein, a Cas9 nuclease, a Cas9 nickase or a nuclease-dead Cas9 (dCas9). In one embodiment, the DTU is selected from the group consisting of nuclease-dead NmCas9 (NmdCas9), NmCas9 nuclease, NmCas9 nickase (HNH), and NmCas9 nickase (RuvC). In one embodiment, the DTU is selected from the group consisting of DBD²⁶⁸, DBD^{TS1}, DBD^{TS2}, DBD^{TS3}, DBD^{TS4} and nuclease-dead NmCas9 (NmdCas9). In one embodiment, the two component Cas9 nuclease DNA targeting unit (DTU) system comprises one guide RNA for each Cas9 protein present, wherein each of said Cas9 proteins contain a guide sequence element that is complementary to a region of the DNA target site. In one embodiment, the guide RNAs are selected from the group consisting of an sgRNA sequence, a crRNA sequence and a tracrRNA sequence. In one embodiment, the guide sequence element is truncated. In one embodiment, the truncated guide sequence element is less than twenty nucleotides. In one embodiment, the drug-dependent dimerization domains within the Cas9 nuclease component are selected from the group consisting of FRB, FRB*, FKBP, ABI and PYL. In one embodiment, the drug-dependent dimerization domains within the DTU component are selected from the group consisting of FRB, FRB*, FKBP, ABI and PYL. In one embodiment, the light-dependent dimerization domains within the DTU component are selected from the group consisting of pMag, nMag, CRY2 and CIB1. In one embodiment, the light-dependent dimerization domains within DTU component are selected from the group consisting of pMag, nMag, CRY2 and CIB1. In one embodiment, the first linker joining the Cas9 nuclease and the drug-dependent or light-dependent dimerization domain is between two and sixty amino acids. In one embodiment, the second linker joining the DTU and the drug-dependent or light-dependent dimerization domain is between two and sixty amino acids. In one embodiment, the two component Cas9 nuclease DNA targeting unit (DTU) system upon addition of the stimulus (drug or light) has improved cleavage activity at the target site relative to the same wild-type Cas9 isoform. In one embodiment, the two component Cas9 nuclease DNA targeting unit (DTU) system upon addition of the stimulus (drug or light) has improved cleavage activity at a target site with suboptimal PAMs relative to the same wild-type Cas9 isoform. In one embodiment, the two component SpCas9 nuclease DNA targeting unit (DTU) system upon addition of the stimulus (drug or light) has improved cleavage activity at a target site with suboptimal PAMs (NAG, NGA or NGC) relative to wild-type SpCas9. In one embodiment, the two component Cas9 nuclease DNA targeting unit (DTU) system upon addition of the stimulus (drug or light) has improved cleavage precision at the target site relative to the same wild-type Cas9 isoform. In one embodiment, the two component SpCas9 nuclease DNA targeting unit (DTU) system upon addition of the stimulus (drug or light) cleaves said target site with a precision that has a specificity ratio between two and one-hundred and fifty six fold greater than a SpCas9^{WT} nuclease.

In one embodiment, the present invention contemplates a two component Split-Cas9 nuclease DNA targeting unit (DTU) system, wherein said system is inactive until assembled via drug-dependent or light-dependent dimerization thereby improving nuclease precision and activity, said system comprising: a) a fusion protein comprising; i) a N-terminal fragment of Cas9 nuclease, said nuclease fragment comprising a first peptide linker, wherein said first peptide linker is attached to a drug-dependent or light-dependent dimerization domain (N-terminal nuclease component); and ii) a C-terminal fragment of Cas9 nuclease, said nuclease fragment comprising a mutated protospacer adjacent motif recognition domain a second peptide linker and a third peptide linker, wherein said second peptide linker is attached to a drug-dependent or light-dependent dimerization domain and said third peptide linker is attached to DNA targeting unit (DTU) (DTU component); and b) a DNA target site. In one embodiment, the Cas9 nuclease are selected from the group consisting of SpCas9, SaCas9, NmCas9 and AnCas9. In one embodiment, the mutated protospacer adjacent motif recognition domain renders target recognition by the Cas9 nuclease dependent on the DTU. In one embodiment, the mutated protospacer adjacent motif recognition domain comprises mutated DNA phosphodiester recognition amino acid residues. In one embodiment, the mutated protospacer adjacent motif recognition domain is selected from the group consisting of SpCas9^{MT1}, SpCas9^{MT2}, SpCas9^{MT3}, NmCas9^{SM} and NmCas9^{DM}. In one embodiment, the DTU can be a zinc finger protein, a transcription activator-like effector protein, a Cas9 nuclease, a Cas9 nickase or a nuclease-dead Cas9 (dCas9). In one embodiment, the DTU is selected from the group consisting of nuclease-dead NmCas9 (NmdCas9), NmCas9 nuclease, NmCas9 nickase (HNH), and NmCas9 nickase (RuvC). In one embodiment, the DTU is selected from the group consisting of DBD²⁶⁸, DBD^{TS1}, DBD^{TS2}, DBD^{TS3}, DBD^{TS4} and nuclease-dead NmCas9 (NmdCas9). In one embodiment, the fusion protein further comprises one guide RNA for each Cas9 present each guide RNA contains a guide sequence element that is complementary to a region of the DNA target site. In one embodiment, the guide RNAs are selected from the group consisting of an sgRNA sequence, a crRNA sequence and a tracrRNA sequence. In one embodiment, the guide sequence element is truncated. In one embodiment, the truncated guide sequence element is less than twenty nucleotides. In one embodiment, the drug-dependent dimerization domain fused to the N-terminal nuclease component are selected from the group consisting of FRB, FRB*, FKBP, ABI and PYL. In one embodiment, the drug-dependent dimerization domain fused to the DTU component are selected from the group consisting of FRB, FRB*, FKBP, ABI and PYL. In one embodiment, the light-dependent dimerization domain fused to the N-terminal nuclease component are selected from the group consisting of pMag, nMag, CRY2 and CIB1. In one embodiment, the light-dependent dimerization domain fused to the DTU component are selected from the group consisting of pMag, nMag, CRY2 and CIB1. In one embodiment, the linker joining the N-terminal Cas9 fragment and the drug-dependent or light-dependent dimerization domain is between two and sixty amino acids. In one embodiment, the linker joining the C-terminal Cas9 fragment and the drug-dependent or light-dependent dimerization domain is between two and sixty amino acids. In one embodiment, the linker joining the C-terminal Cas9 fragment and DTU is between two and sixty amino acids. In one embodiment, the N-terminal Cas9 fragment is composed of residues 2-573 of SpCas9. In one embodiment, the C-terminal Cas9 fragment is composed of residues 574-1368 of SpCas9. In one embodiment, the two component Split-Cas9 nuclease DNA targeting unit (DTU) system upon addition of the stimulus (drug or light) has improved cleavage activity at the target site relative to the same wild-type Cas9 isoform. In one embodiment, the two component Split-Cas9 nuclease DNA targeting unit (DTU) system upon addition of the stimulus (drug or light) has improved cleavage activity at a target site with suboptimal PAMs relative to the same wild-type Cas9 isoform. In one embodiment, the two component Split-SpCas9 nuclease DNA targeting unit (DTU) system upon addition of the stimulus (drug or light) has improved cleavage activity at a target site with suboptimal PAMs (NAG, NGA or NGC) relative to wild-type SpCas9. In one embodiment, the two component Split-Cas9 nuclease DNA targeting unit (DTU) system upon addition of the stimulus (drug or light) has improved cleavage precision at the target site relative to the same wild-type Cas9 isoform. In one embodiment, the two component Split-Cas9 nuclease DNA targeting unit (DTU) system upon addition of the stimulus (drug or light) the resulting nuclease cleaves said target site with a precision that has a specificity ratio between two and one-hundred and fifty six fold greater than a Cas9^{WT} nuclease.

In one embodiment, the present invention contemplates a fusion protein comprising a Cas9 nuclease, said nuclease comprising a peptide linker, wherein said peptide linker is attached to a DNA targeting unit (DTU). In one embodiment, the Cas9 nuclease is selected from the group consisting of SpCas9, SaCas9, NmCas9 and AnCas9. In one embodiment, the DTU can be a zinc finger protein, a transcription activator-like effector protein, a Cas9 nuclease, a Cas9 nickase or a nuclease-dead Cas9 (dCas9). In one embodiment, the DTU is selected from the group consisting of nuclease-dead NmCas9 (NmdCas9), NmCas9 nuclease, NmCas9 nickase (HNH), and NmCas9 nickase (RuvC). In one embodiment, the DTU is selected from the group consisting of DBD²⁶⁸, DBD^{TS1}, DBD^{TS2}, DBD^{TS3}, DBD^{TS4} and nuclease-dead NmCas9 (NmdCas9). In one embodiment, the fusion protein further comprises a guide RNA for each Cas9 module attached to a guide sequence element. In one embodiment, the guide RNA is selected from the group consisting of an sgRNA sequence, a crRNA sequence and a tracrRNA sequence. In one embodiment, the guide sequence element is truncated. In one embodiment, the truncated guide sequence element is less than twenty nucleotides. In one embodiment, the resulting nuclease has improved cleavage activity at the target site relative to the same wild-type Cas9 isoform. In one embodiment, the resulting nuclease has improved cleavage activity at a target site with suboptimal PAMs relative to the same wild-type Cas9 isoform. In one embodiment, the fusion protein comprising SpCas9 as the nuclease has improved cleavage activity at a target site with suboptimal PAMs (NAG, NGA or NGC) relative to wild-type SpCas9.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein may be used to describe specific embodiments of the invention, but their usage does not delimit the invention, except as outlined in the claims.

The term “about” as used herein, in the context of any of any assay measurements refers to +/−5% of a given measurement.

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by the same series in reverse and then by 30 or so base pairs known as “spacer DNA”. The spacers are short segments of DNA from a virus and may serve as a ‘memory’ of past exposures to facilitate an adaptive defense against future invasions (PMID 25430774).

As used herein, the term “Cas” or “CRISPR-associated (cas)” refers to genes often associated with CRISPR repeat-spacer arrays (PMID 25430774).

As used herein, the term “Cas9” refers to a nuclease from Type II CRISPR systems, an enzyme specialized for generating double-strand breaks in DNA, with two active cutting sites (the HNH and RuvC domains), one for each strand of the double helix. Jinek combined tracrRNA and spacer RNA into a “single-guide RNA” (sgRNA) molecule that, mixed with Cas9, could find and cleave DNA targets through Watson-Crick pairing between the guide sequence within the sgRNA and the target DNA sequence (PMID 22745249).

As used herein, the term “nuclease deficient Cas9”, “nuclease dead Cas9” or “dCas9” refers to a modified Cas9 nuclease wherein the nuclease activity has been disabled by mutating residues in the RuvC and HNH catalytic domains. Disabling of both cleavage domains can convert Cas9 from a RNA-programmable nuclease into an RNA-programmable DNA recognition complex to deliver effector domains to specific target sequences (Qi, et al. 2013 (PMID 23452860) and Gilbert, et al. 2013 PMID 23849981) or to deliver an independent nuclease domain such as FokI. A nuclease dead Cas9 can bind to DNA via its PAM recognition sequence and guide RNA, but will not cleave the DNA.

The term “nuclease dead Cas9 FokI fusion” or “FokI-dCas9” as used herein, refers to a nuclease dead Cas9 that may be fused to the cleavage domain of FokI, such that DNA recognition may be mediated by dCas9 and the incorporated guide RNA, but that DNA cleavage may be mediated by the FokI domain (Tsai, et al. 2014 (PMID 24770325) and Guilinger, et al. (PMID 24770324)). FokI normally requires dimerization in order to cleave the DNA, and as a consequence two FokI-dCas9 complexes must bind in proximity in order to cleave the DNA. FokI can be engineer such that it functions as an obligate heterodimer.

As used herein, the term “catalytically active Cas9” refers to an unmodified Cas9 nuclease comprising full nuclease activity.

The term “nickase” as used herein, refers to a nuclease that cleaves only a single DNA strand, either due to its natural function or because it has been engineered to cleave only a single DNA strand. Cas9 nickase variants that have either the RuvC or the HNH domain mutated provide control over which DNA strand is cleaved and which remains intact (Jinek, et al. 2012 (PMID 22745249) and Cong, et al. 2013 (PMID 23287718)).

The term “DNA targeting unit”, “DTU” as used herein, refers to any type of system that can be programmed to recognize a specific DNA sequence of interest. Such DNA targeting units can include, but are not limited to a “programmable DNA binding domain” (either called a pDBD or simply a DBD), as defined below, and/or a CRISPR/Cas9 or CRISPR/Cpf1 system that may be programmed by a RNA guide (either a single guide RNA or a crRNA and tracrRNA combination) to recognize a particular target site.

The term, “trans-activating crRNA”, “tracrRNA” as used herein, refers to a small trans-encoded RNA. For example, CRISPR/Cas (clustered, regularly interspaced short palindromic repeats/CRISPR-associated proteins) constitutes an RNA-mediated defense system, which protects against viruses and plasmids. This defensive pathway has three steps. First a copy of the invading nucleic acid is integrated into the CRISPR locus. Next, CRISPR RNAs (crRNAs) are transcribed from this CRISPR locus. The crRNAs are then incorporated into effector complexes, where the crRNA guides the complex to the invading nucleic acid and the Cas proteins degrade this nucleic acid. There are several pathways of CRISPR activation, one of which requires a tracrRNA, which plays a role in the maturation of crRNA. TracrRNA is complementary to base pairs with a pre-crRNA forming an RNA duplex. This is cleaved by RNase III, an RNA-specific ribonuclease, to form a crRNA/tracrRNA hybrid. This hybrid acts as a guide for the endonuclease Cas9, which cleaves the invading nucleic acid.

The term “programmable DNA binding domain” as used herein, refers to any protein comprising a pre-determined sequence of amino acids that bind to a specific nucleotide sequence. Such binding domains can include, but are not limited to, a zinc finger protein, a homeodomain and/or a transcription activator-like effector protein.

The term “protospacer adjacent motif” (or PAM) as used herein, refers to a DNA sequence that may be required for a Cas9/sgRNA to form an R-loop to interrogate a specific DNA sequence through Watson-Crick pairing of its guide RNA with the genome. The PAM specificity may be a function of the DNA-binding specificity of the Cas9 protein (e.g., a “protospacer adjacent motif recognition domain” at the C-terminus of Cas9).

As used herein, the term “sgRNA” refers to single guide RNA used in conjunction with CRISPR associated systems (Cas). sgRNAs are a fusion of crRNA and tracrRNA and contain nucleotides of sequence complementary to the desired target site (Jinek, et al. 2012 (PMID 22745249)). Watson-Crick pairing of the sgRNA with the target site permits R-loop formation, which in conjunction with a functional PAM permits DNA cleavage or in the case of nuclease-deficient Cas9 allows binds to the DNA at that locus.

As used herein, the term “orthogonal” refers targets that are non-overlapping, uncorrelated, or independent. For example, if two orthogonal Cas9 isoforms were utilized, they would employ orthogonal sgRNAs that only program one of the Cas9 isoforms for DNA recognition and cleavage (Esvelt, et al. 2013 (PMID 24076762)). For example, this would allow one Cas9 isoform (e.g. S. pyogenes Cas9 or spCas9) to function as a nuclease programmed by a sgRNA that may be specific to it, and another Cas9 isoform (e.g. N. meningitidis Cas9 or nmCas9) to operate as a nuclease dead Cas9 that provides DNA targeting to a binding site through its PAM specificity and orthogonal sgRNA. Other Cas9s include S. aureus Cas9 or SaCas9 and A. naeslundii Cas9 or AnCas9.

The term “truncated” as used herein, when used in reference to either a polynucleotide sequence or an amino acid sequence means that at least a portion of the wild type sequence may be absent. In some cases truncated guide sequences within the sgRNA or crRNA may improve the editing precision of Cas9 (Fu, et al. 2014 (PMID 24463574)).

The term “dimerization domain” as used herein, refers to a domain, either protein, polynucleotide that allows the associate of two different molecules. A dimerization domain can allow homotypic and/or heterotypic interactions. Dimerization domains can also be drug-dependent (i.e. depending on the presence of a small molecule in order to function) (Liang, et al. (PMID 21406691) and Ho, et al. 1996 (PMID 8752278)).

The term “base pairs” as used herein, refer to specific nucleobases (also termed nitrogenous bases), that are the building blocks of nucleotide sequences that form a primary structure of both DNA and RNA. Double stranded DNA may be characterized by specific hydrogen bonding patterns, base pairs may include, but are not limited to, guanine-cytosine and adenine-thymine) base pairs.

The term “specific genomic target” as used herein, refers to any pre-determined nucleotide sequence capable of binding to a Cas9 protein contemplated herein. The target may include, but may be not limited to, a nucleotide sequence complementary to a programmable DNA binding domain or an orthogonal Cas9 protein programmed with its own guide RNA, a nucleotide sequence complementary to a single guide RNA, a protospacer adjacent motif recognition sequence, an on-target binding sequence and an off-target binding sequence.

The term “on-target binding sequence” as used herein, refers to a subsequence of a specific genomic target that may be completely complementary to a programmable DNA binding domain and/or a single guide RNA sequence.

The term “off-target binding sequence” as used herein, refers to a subsequence of a specific genomic target that may be partially complementary to a programmable DNA binding domain and/or a single guide RNA sequence.

The term “fails to bind” as used herein, refers to any nucleotide-nucleotide interaction or a nucleotide-amino acid interaction that exhibits partial complementarity, but has insufficient complementarity for recognition to trigger the cleavage of the target site by the Cas9 nuclease. Such binding failure may result in weak or partial binding of two molecules such that an expected biological function (e.g., nuclease activity) fails.

The term “cleavage” as used herein, may be defined as the generation of a break in the DNA. This could be either a single-stranded break or a double-stranded break depending on the type of nuclease that may be employed.

As used herein, the term “edit” “editing” or “edited” refers to a method of altering a nucleic acid sequence of a polynucleotide (e.g., for example, a wild type naturally occurring nucleic acid sequence or a mutated naturally occurring sequence) by selective deletion of a specific genomic target or the specific inclusion of new sequence through the use of an exogenously supplied DNA template. Such a specific genomic target includes, but may be not limited to, a chromosomal region, mitochondrial DNA, a gene, a promoter, an open reading frame or any nucleic acid sequence.

The term “delete”, “deleted”, “deleting” or “deletion” as used herein, may be defined as a change in either nucleotide or amino acid sequence in which one or more nucleotides or amino acid residues, respectively, are, or become, absent.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” may be complementary to the sequence “A-C-T-G.” Complementarity can be “partial” or “total.” “Partial” complementarity may be where one or more nucleic acid bases may be not matched according to the base pairing rules. “Total” or “complete” complementarity between nucleic acids may be where each and every nucleic acid base may be matched with another base under the base pairing rules. The degree of complementarity between nucleic acid strands has significant effects on the efficiency and strength of hybridization between nucleic acid strands. This may be of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which may be partially complementary, i.e., “substantially homologous,” to a nucleic acid sequence may be one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This may be not to say that conditions of low stringency are such that non-specific binding may be permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be detected in a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which may be a “homolog” may be defined herein as an oligonucleotide sequence which exhibits greater than or equal to 50% identity to a sequence, when sequences having a length of 100 bp or larger are compared.

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “gene of interest” as used herein, refers to any pre-determined gene for which deletion may be desired.

The term “allele” as used herein, refers to any one of a number of alternative forms of the same gene or same genetic locus.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide”, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and may be, in a preferred embodiment, free of other genomic nucleic acid).

The terms “amino acid sequence” and “polypeptide sequence” as used herein, are interchangeable and to refer to a sequence of amino acids.

As used herein the term “portion” when in reference to a protein (as in “a portion of a given protein”) refers to fragments of that protein. The fragments may range in size from four amino acid residues to the entire amino acid sequence minus one amino acid.

The term “portion” when used in reference to a nucleotide sequence refers to fragments of that nucleotide sequence. The fragments may range in size from 5 nucleotide residues to the entire nucleotide sequence minus one nucleic acid residue.

As used herein, the term “hybridization” may be used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) may be impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ” may be used in reference to the “melting temperature.” The melting temperature may be the temperature at which a population of double-stranded nucleic acid molecules becomes half dissociated into single strands. As indicated by standard references, a simple estimate of the Tₘ value may be calculated by the equation: Tₘ=81.5+0.41 (% G+C), when a nucleic acid may be in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tₘ.

As used herein the term “stringency” may be used in reference to the conditions of temperature, ionic strength, and the presence of other compounds such as organic solvents, under which nucleic acid hybridizations are conducted. “Stringency” typically occurs in a range from about Tₘ to about 20° C. to 25° C. below Tₘ. A “stringent hybridization” can be used to identify or detect identical polynucleotide sequences or to identify or detect similar or related polynucleotide sequences. For example, when fragments are employed in hybridization reactions under stringent conditions the hybridization of fragments which contain unique sequences (i.e., regions which are either non-homologous to or which contain less than about 50% homology or complementarity) are favored. Alternatively, when conditions of “weak” or “low” stringency are used hybridization may occur with nucleic acids that are derived from organisms that are genetically diverse (i.e., for example, the frequency of complementary sequences may be usually low between such organisms).

As used herein, the term “amplifiable nucleic acid” may be used in reference to nucleic acids which may be amplified by any amplification method. It may be contemplated that “amplifiable nucleic acid” will usually comprise “sample template.”

As used herein, the term “sample template” refers to nucleic acid originating from a sample which may be analyzed for the presence of a target sequence of interest. In contrast, “background template” may be used in reference to nucleic acid other than sample template which may or may not be present in a sample. Background template may be most often inadvertent. It may be the result of carryover, or it may be due to the presence of nucleic acid contaminants sought to be purified away from the sample. For example, nucleic acids from organisms other than those to be detected may be present as background in a test sample.

“Amplification” may be defined as the production of additional copies of a nucleic acid sequence and may be generally carried out using polymerase chain reaction. Dieffenbach C. W. and G. S. Dveksler (1995) In: PCR Primer, a Laboratory Manual, Cold Spring Harbor Press, Plainview, N.Y.

As used herein, the terms “restriction endonucleases” and “restriction enzymes” refer to bacterial enzymes, each of which cut double-stranded DNA at or near a specific nucleotide sequence.

DNA molecules are said to have “5′ ends” and “3′ ends” because mononucleotides are reacted to make oligonucleotides in a manner such that the 5′ phosphate of one mononucleotide pentose ring may be attached to the 3′ oxygen of its neighbor in one direction via a phosphodiester linkage. Therefore, an end of an oligonucleotide may be referred to as the “5′ end” if its 5′ phosphate may be not linked to the 3′ oxygen of a mononucleotide pentose ring. An end of an oligonucleotide may be referred to as the “3′ end” if its 3′ oxygen may be not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “bind”, “binding”, or “bound” as used herein, includes any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measuring. The “binding” interaction may be brief as in the situation where binding causes a chemical reaction to occur. That may be typical when the binding component may be an enzyme and the analyte may be a substrate for the enzyme. Reactions resulting from contact between the binding agent and the analyte are also within the definition of binding for the purposes of the present invention.

### BRIEF DESCRIPTION OF THE FIGURES

FIG. 1 presents a schematic overview of an exemplary CRISPR/Cas9 system. For example, SpCas9 may recognize a target sequence through Watson-Crick pairing of approximately 20 bases of the sgRNA and recognition of a neighboring PAM sequence (e.g., for example, NGG). Upon binding to its target, Cas9 generates a double stranded break (DSB) via cleavage of each strand (arrowheads).

FIG. 2A and FIG. 2B present schematic overviews of an exemplary CRISPR/Cas9 system fused to a DNA-targeting unit (in this case a programmable DNA-binding domain; DBD). For example, Cas9 recognizes a target sequence through Watson-Crick pairing of approximately 20 bases of an sgRNA (sequence) with one strand of the target DNA sequence and recognition of the neighboring PAM sequence (NGG—letters) by the PAM-Interacting domain of the protein. Upon binding a target sequence, Cas9 generates a double stranded break (DSB) by cleaving each strand (arrowheads). The DBD can be fused to the N- or C-terminus (or perhaps both, where “N-” and “-C” indicate the N-terminus and C-terminus, respectively) with a linker molecule and programmed to recognize a neighboring sequence (pDBD binding site) to enhance specificity or increase the range of target sequences that can be cleaved by Cas9.

FIG. 2C presents a schematic showing that a Cas9-pDBD can be conferred with drug-dependent activity by inserting drug-dependent dimerization domains into a linker.

FIG. 3 the top panel presents an illustrative schematic of orientation and spacing parameters for the presently disclosed chimeric Cas9-pDBD constructs. The position and 5′ to 3′ orientation of the DBD binding site may be represented by an arrow relative to the PAM element of the Cas9 binding site. The bottom panel displays the activity profile of Cas9 on an NGG or NAG PAM), Cas9-Zif268 (also referred to as Cas9-DBD²⁶⁸) (NAG PAM) or Cas9-TAL268 (NAG PAM) on a common sgRNA target site. DBD site orientation may be either Watson (W) or Crick (C), and spacing may be 5, 8, 11 or 14 bp from the PAM (see schematic). While no activity for Cas9 was detected above background on an AG PAM (relative to the no guide control), on an NAG PAM, Cas9-Zif268 displayed activity on all AG PAM target sites. A TALE domain programmed to recognize the same target site (TAL268) may be also functional on a subset of spacings and orientations of the DBD binding site. Data are from three independent biological replicates performed on different days, where HEK293T cells transfected with 50 ng Cas9/Cas9-Zif268/Cas9-TAL268 plasmid, 50 ng sgRNA plasmid, 150 ng GFP reporter with target site and 100 ng mCherry control plasmid. Error bars indicate standard error of the mean.

FIG. 4 presents one embodiment of a structural model for SpCas9-Zif²⁶⁸ (also referred as SpCas9-DBD²⁶⁸). A B-DNA model containing Zif268 binding site (Watson-11bp) may be constructed using 3DNA (PMID 18600227) and appended 3′ to the PAM in the SpCas9 structure. In parallel with the spacing parameters in accordance with FIG. 3, a W-11bp configuration Zif268 does not generate steric clashes within the model to SpCas9.

FIG. 5 presents exemplary data showing an activity profile of SpCas9 or SpCas9-Zif268 on a common target site with different PAM sequences and a neighboring Zif268 site (Watson-5 bp). SpCas9 may be active only on the NGG PAM, whereas SpCas9-Zif268 may be active on NGG, NAG, NGA and NGC PAMs. Data are from three independent biological replicates performed on different days, where HEK293T cells transfected with 50 ng Cas9/Cas9-Zif268 plasmid, 50 ng sgRNA plasmid, 150 ng GFP reporter with target site and 100 ng mCherry control plasmid.

FIG. 6 presents exemplary data showing an activity profile of SpCas9 or SpCas9-Zif268 (also referred to as SpCas9-DBD²⁶⁸) with sgRNAs of different length (truncated) on a common target site with a NGG PAM sequences and a neighboring Zif268 site 5 base pairs away in Watson orientation. Cas9-Zif268 display higher activity between 15 and 20 nt of length in the guide sequence. Data are from three independent biological replicates performed on different days, where HEK293T cells transfected with 50 ng Cas9/Cas9-Zif268 plasmid, 50 ng sgRNA plasmid, 150 ng GFP reporter with target site and 100 ng mCherry control plasmid.

FIG. 7 presents exemplary data showing an activity profile of SpCas9 and SpCas9-TAL268 in the GFP reporter assay with sgRNAs of 20 nt vs 16 nt lengths on NGG, NAG, NGA, NGC PAM target sites with a neighboring Zif268 site (Watson-5 bp). SpCas9 displays robust activity only on the NGG PAM, whereas SpCas9-TAL268 may be active on NGG, NAG, NGA and NGC PAMs. Data are from three independent biological replicates performed on different days, where HEK293T cells transfected with 50 ng Cas9/Cas9-TAL268 plasmid, 50 ng sgRNA plasmid, 150 ng GFP reporter with target site and 100 ng mCherry control plasmid.

FIG. 8 presents exemplary data showing a quantification of lesion frequencies from three independent biological replicates performed on different days in HEK293T cells. Error bars indicate standard error of the mean. Genomic activity profiles of SpCas9 and SpCas9-Zif268 programmed independently with 4 different sgRNAs targeting 4 different genomic sites with neighboring Zif⁶⁸ binding sites (Watson-11bp). SpCas9 cuts efficiently only the GG PAM, but SpCas9-Zif²⁶⁸ also cuts efficiently at AG, GA or GC PAMs. Genomic regions were PCR-amplified, and lesions (i.e., for example, cleavages and mutagenic NHEJ's) were detected by T7 Endonuclease I (T7EI) assay. Top panel may be the exemplary agarose gel image displaying DNA lesion profile after T7EI treatment. The bottom panel may be the quantification of lesion frequencies data from three independent biological replicates performed on different days, where HEK293T cells transfected with 50 ng Cas9/Cas9-Zif268 plasmid, 50 ng sgRNA plasmid, and 100 ng mCherry control plasmid.

FIG. 9 illustrates one embodiment of PAM-interacting amino acid residues neighboring a NGG PAM in the structure of SpCas9 (PMID 25079318; Top panel). The bottom panel presents an activity profile of SpCas9 or SpCas9-Zif268 bearing mutations at positions 1333 or 1335 in the PAM recognition sequence in comparison to wild-type (WT) SpCas9. SpCas9 bearing these mutations may be inactive on its own, but when fused to Zif268 (also referred to as DBD²⁶⁸), activity may be restored due to a nearby a Zif268 binding site (Watson-5 bp). MT3 (R1335K) appears to have more stringent specificity (NAG PAM may be not functional). Data are from three independent biological replicates performed on different days, where HEK293T cells transfected with 50 ng Cas9^{MT}/Cas9^{MT}-Zif268 plasmid, 50 ng sgRNA plasmid, 150 ng GFP reporter with target site and 100 ng mCherry control plasmid.

FIG. 10 presents one embodiment of a T7EI assay on PCR products spanning a genomic target site with an NGG PAM and neighboring Zif268 site (Watson-11bp) for various SpCas9 or SpCas9-Zif268 mutants (MT #). For SpCas9^{MT2} & SpCas9^{MT3}, strong activity may be observed when Zif268 is fused. Top Panel: Agarose gel images used showing three independent replicates (R1, R2, R3) for each nuclease platform. Cleaved bands indicating nuclease activity at each locus are indicated by dots. Bottom Panel: The quantification of lesion frequencies data from three independent biological replicates performed on different days, where HEK293T cells transfected with 50 ng Cas9/Cas9-Zif268 plasmid, 50 ng sgRNA plasmid, and 100 ng mCherry control plasmid.

FIG. 11A&B present exemplary data of an analysis of the activity of SpCas9 mutants (e.g., MT1, MT2 & MT3) on different PAM-containing target sites with a neighboring Zif268 site (Watson-5 bp) in a GFP reporter assay.

FIG. 11A: Local sequences of the PAM interacting domain mutants at positions 1333 or 1335 of SpCas9.

FIG. 11B: Analysis of SpCas9 mutant activity on different nGn or nnG PAM-containing target sites in the GFP reporter assay. Mutations that alter the interaction of R1333 with its guanine contact (nGn) reveal modest activity at nnG PAMs. Correspondingly, mutations that alter the interaction of R1335 with its guanine contact (nnG) reveal modest activity at nGn PAMs. Data are from three independent biological replicates performed on different days in HEK293T cells. Residues above each panel are positions 1333 through 1335 in SpCas9. Data are from three independent biological replicates performed on different days, where HEK293T cells transfected with 50 ng Cas9^{MT}/Cas9^{MT}-Zif268 plasmid, 50 ng sgRNA plasmid, 150 ng GFP reporter with target site and 100 ng mCherry control plasmid. Error bars indicate standard error of the mean.

FIG. 12 presents exemplary data of T7EI assays on PCR products spanning target site TS3 or off-target site 2 (OT3-2) in nuclease treated (or control) HEK293T cells (PMID 24463574). An sgRNA for TS3 (sgRNA-TS3) was used to program cleavage of SpCas9^{WT}, SpCas9-ZFP^{TS3} SpCas9^{MT3} or SpCas9^{MT3}-ZFP^{TS3}, where the ZFP was assembled from an archive of zinc fingers of defined specificity. Zhu et al., Using defined finger-finger interfaces as units of assembly for constructing zinc-finger nucleases. Nucleic Acids Research. 2013 Feb. 1; 41(4):2455-65; and Gupta et al., An optimized two-finger archive for ZFN-mediated gene targeting. Nature Methods. 2012 Apr. 29; 9(6):588-90. Top Panel: An exemplary agarose gel image displaying DNA lesion profile after T7EI treatment. Cleaved bands indicating nuclease activity at each locus are indicated by dots. SpCas9^{MT3}-ZFP^{TS3} shows no apparent activity at OT3-2, whereas it cleaves the target site efficiently. Bottom Panel: Quantification of lesion frequencies data from three independent biological replicates, where HEK293T cells transfected with 50 ng Cas9/Cas9^{MT3}/Cas9-ZFP^{TS3}/Cas9^{MT3}-ZFP^{TS3} plasmid, 50 ng sgRNA plasmid, and 100 ng mCherry control plasmid.

FIG. 13 presents exemplary data of T7EI assays on PCR products spanning TS3 or off-target site 2 (OT3-2) in nuclease treated (or control) HEK293T cells. An sgRNA for TS3 (sgRNA-TS3) was used to program cleavage of SpCas9^{WT}, SpCas9^{MT3} or SpCas9^{MT3}-ZFP^{TS3} where the ZFP was assembled from an archive of zinc fingers of defined specificity. Zhu et al., Using defined finger-finger interfaces as units of assembly for constructing zinc-finger nucleases. Nucleic Acids Research. 2013 Feb. 1; 41(4):2455-65; and Gupta et al., An optimized two-finger archive for ZFN-mediated gene targeting. Nature Methods. 2012 Apr. 29; 9(6):588-90. Cleaved bands indicating nuclease activity at each locus are indicated by dots. SpCas9^{MT3}-ZFP^{TS3} may be programmed with a non-cognate sgRNA-TS4, or SpCas9^{MT3} may be fused to a ZFP recognizing a different binding site (SpCas9^{MT3}-ZFP^{TS4}) such that no activity may be observed at TS3.

FIG. 14 presents exemplary data of T7EI assays on PCR products spanning TS2 or off-target site 2 (OT2-1) in nuclease treated (or control) HEK293T cells (PMID 24463574). An sgRNA for TS2 (sgRNA-TS2) was used to program cleavage of SpCas9^{WT}, SpCas9-ZFP^{TS2}, SpCas9^{MT3} or SpCas9^{MT3}-ZFP^{TS2}, where the ZFP was assembled from an archive of zinc fingers of defined specificity. Zhu et al., Using defined finger-finger interfaces as units of assembly for constructing zinc-finger nucleases. Nucleic Acids Research. 2013 Feb. 1; 41(4):2455-65; and Gupta et al., An optimized two-finger archive for ZFN-mediated gene targeting. Nature Methods. 2012 Apr. 29; 9(6):588-90. Cleaved bands indicating nuclease activity at each locus are indicated by dots. SpCas9^{MT3}-ZFP^{TS2} shows no apparent activity at OT2-1, whereas it cleaves the target site efficiently.

FIG. 15 presents exemplary data of T7EI assays on PCR products spanning TS4 in nuclease treated (or control) HEK293T cells (PMID 24463574). An sgRNA for TS4 (sgRNA-TS4) was used to program cleavage of SpCas9^{WT}, SpCas9^{MT3} or SpCas9^{MT3}-ZFP^{TS4}, where the ZFP was assembled from an archive of zinc fingers of defined specificity. Zhu et al., Using defined finger-finger interfaces as units of assembly for constructing zinc-finger nucleases. Nucleic Acids Research. 2013 Feb. 1; 41(4):2455-65; and Gupta et al., An optimized two-finger archive for ZFN-mediated gene targeting. Nature Methods. 2012 Apr. 29; 9(6):588-90. Cleaved bands indicating nuclease activity at each locus are indicated by dots.

FIG. 16 presents illustrative schematic of the orientation and spacing parameters examined in these assays. Top Panel: Position and 5′ to 3′ orientation of the DTU binding site may be represented by an arrow relative to the PAM element of the Cas9 binding site. Bottom Panel: Displays an exemplary activity profile of Cas9 (on an NGG or NAG PAM) or Cas9-SL_Zif268 (also referred to as Cas9^{SL}-ZFP268) (NAG PAM), where SL stands for shortened peptide linker between Cas9 and the DTU, on a common sgRNA target site. DTU site orientation may be either Watson (W) or Crick (C), and spacing may be 5, 8, 11 or 14 bp from the PAM (see schematic). No activity was detected for Cas9 above background on an NAG PAM, a Cas9-SL_Zif268 construct displayed activity on all AG PAM target sites in various levels. These data suggest that the linker length can be utilized as a parameter to adjust increased specificity on a desired target. Datum may be from single replicate, where HEK293T cells transfected with 50 ng Cas9/Cas9-Zif268/Cas9-TAL268 plasmid, 50 ng sgRNA plasmid, 150 ng GFP reporter with target site and 100 ng mCherry control plasmid.

FIG. 17 presents a schematic of the orientation and spacing parameters examined in these assays (Top Panel). The position and 5′ to 3′ orientation of the DTU binding site may be represented by an arrow relative to the position of 5′ nucleotide of the sgRNA of the Cas9 binding site. The bottom panel displays the activity profile of Cas9 (on an NGG or NAG PAM), N-Zif268-Cas9 (NAG PAM) or N-TAL268-Cas9 (NAG PAM) on a common sgRNA target site. DTU site orientation may be either Watson (W) or Crick (C), and spacing may be 6, 8, 10, 12, 14 or 16 bp from the 5′ of sgRNA (see schematic). Enhanced nuclease activity was not detected for either Cas9 or N-Zif268-Cas9 nor N-TAL268-Cas9 above the background on an AG PAM. Data are from three independent biological replicates on performed on different days, where HEK293T cells transfected with 50 ng Cas9/N-Zif268-Cas9/N-TAL268-Cas9 plasmid, 50 ng sgRNA plasmid, 150 ng GFP reporter with target site and 100 ng mCherry control plasmid.

FIG. 18 presents a schematic of the orientation and spacing parameters examined in these assays (top panel). The position and 5′ to 3′ orientation of the DTU binding site may be represented by an arrow relative to the position of 5′ nucleotide of the sgRNA of the Cas9 binding site. The bottom panel displays the activity profile of Cas9 (on an NGG or NAG PAM), Cas9-Zif268 (NAG PAM) or Cas9-TAL268 (NAG PAM) on a common sgRNA target site. DTU site orientation may be either Watson (W) or Crick (C), and spacing may be 6, 8, 10, 12, 14 or 16 bp from the 5′ of sgRNA (see schematic). While no activity was detected for Cas9 and for Cas9-TAL268 above background on an NAG PAM, Cas9-Zif268 displayed modest activity on most of the NAG PAM target sites above the background on an NAG PAM. Datum may be from single replicates, where HEK293T cells transfected with 50 ng Cas9/Cas9-Zif268/Cas9-TAL268 plasmid, 50 ng sgRNA plasmid, 150 ng GFP reporter with target site and 100 ng mCherry control plasmid.

FIG. 19 presents a schematic of the orientation and spacing parameters examined in these assays (top panel). The position and 5′ to 3′ orientation of the DTU binding site may be represented by an arrow relative to the PAM element of the Cas9 binding site (Watson 11bp). No activity was detected for Cas9 and for N-TAL²⁶⁸-Cas9 above background on an NAG PAM. However, Cas9-Zif268, N-Zif268-Cas9, and Cas9-TAL²⁶⁸ all displayed activity on this NAG PAM target site above the background on an NAG PAM. Datum may be from single replicates, where HEK293T cells transfected with 50 ng Cas9/N-Zif268-Cas9/Cas9-Zif268/N-TAL²⁶⁸-Cas9/Cas9-TAL²⁶⁸ plasmid, 50 ng sgRNA plasmid, 150 ng GFP reporter with target site and 100 ng mCherry control plasmid.

FIG. 20 presents a schematic of SpCas9^{MT} and Nm-dCas9 fusions. (Top) SpCas9^{MT}-Nm-dCas9 may be linked through a dimerization domain. (Bottom) SpCas9^{MT}-Nm-dCas9 may be fused through peptide linker.

FIG. 21 presents a schematic of coupling SpCas9 to a programmable DBD via dimerization domain.

FIG. 22 illustrates a schematic of chimeric SpCas9-DTU in the context of existing SpCas9 variants (e.g., for example, truncated sgRNAs, nickases, and FokI-dCas9). These platforms can be combined with a Cas9 nuclease-DTU to use mutant versions of Cas9 that are attentuated (star) to maintain activity dependence on the DTU.

FIG. 23A presents a schematic overview of a B2H system where interaction domains on RNA polymerase and dSpCas9-DBD facilitate recruitment of polymerase and promoter activation upon target site recognition within a reporter vector. The selection of an optimal linker from a randomized library that promotes efficient binding by dSpCas9-DBD should be possible in this framework; and

FIG. 23B presents an initial test of a dSpCas9 system on an NGG PAM target site (e.g., no DBD). Right: a 10× dilution series on non-selective media; Left: the same series but on selective media with 2 mM 3-AT and no histidine. The dCas9/sgRNA-programmed cells with a complementary target site in the reporter survive the selection. Further optimization of the expression construct may yield cells that grow at a rate even closer to that of the positive control.

FIG. 24 presents one embodiment of a structural model of potential DNA phosphate contacts in SpCas9. A B-DNA model was constructed using (PMID 18600227) and appended 3′ to the PAM in a SpCas9 structure. Lysines in proximity to the DNA backbone are shown (no arginines are nearby with the exception of the PAM recognition residues (shown)). These phosphate contacting residues are examples of potential mutagenesis positions to attenuate the independent DNA binding affinity of Cas9 to increase its dependence on the attached DTU.

FIG. 25 illustrates one embodiment of a domain organization of NmCas9 based on a structure of a related Type II-C Cas9. Jinek et al., Structures of Cas9 endonucleases reveal RNA mediated conformational activation. Science. 2014 Mar. 14; 343(6176):1247997. The PAM-interacting residues are likely to be found in the Topo or CTD regions based in part on comparison to SpCas9. Anders et al., Structural basis of PAM-dependent target DNA recognition by the Cas9 endonuclease. Nature. 2014 Sep. 25; 513(7519):569-73. Sequence alignment of NmCas9 with 9 related Type II-C orthologs showing conservation of Arg1025 (circle) and Lys1013 (circle) residues that are candidates for mutagenesis.

FIG. 26 presents a DNA sequence alignment of a short region nearby PAM interacting residues (highlighted residues 1333 to 1335) of wild type SpCas9 and mutants described here.

FIG. 27 presents one embodiment of a plasmid expressing SpCas9-Zif268 fusion protein. SpCas9 sequence may be underlined where PAM interacting residues (AGAAAGCGG) are highlighted and the long Zif268 is also highlighted.

FIG. 28 presents one embodiment of a plasmid expressing Zif268-SpCas9 fusion protein. SpCas9 sequence may be underlined where the short PAM interacting residues (AGAAAGCGG) are highlighted and the long Zif268 is also highlighted.

FIG. 29 presents one embodiment of a plasmid expressing SpCas9-TAL268 fusion protein. SpCas9 sequence may be underlined where the short PAM interacting residues (AGAAAGCGG) are highlighted and the long TAL268 is highlighted.

FIG. 30 presents one embodiment of a plasmid expressing TAL268-SpCas9 fusion protein. SpCas9 sequence may be underlined where the short PAM interacting residues (AGAAAGCGG) are highlighted and the long TAL268 is highlighted.

FIG. 31 presents one embodiment of a plasmid expressing SpCas9-ZFP^{TS2} fusion protein. SpCas9 sequence may be underlined where the short PAM interacting residues (AGAAAGCGG) are highlighted and the long ZFP^{TS2} is highlighted.

FIG. 32 presents one embodiment of a plasmid expressing SpCas9^{MT3}-ZFP^{TS2} fusion protein. SpCas9 sequence may be underlined where the short PAM interacting residues (AGAAAGAAG) are highlighted and the long ZFP^{TS2} is highlighted.

FIG. 33 presents one embodiment of a plasmid expressing SpCas9-ZF^{TS3} fusion protein. SpCas9 sequence may be underlined where the short PAM interacting residues (AGAAAGCGG) are highlighted and the long ZFP^{TS3} is highlighted.

FIG. 34 presents one embodiment of a plasmid expressing SpCas9^{MT3}-ZFP^{TS3} fusion protein. SpCas9 sequence may be underlined where the short PAM interacting residues (AGAAAGAAG) are highlighted and the long ZFP^{TS3} is highlighted.

FIG. 35 presents one embodiment of a plasmid expressing SpCas9-ZFP^{TS4} fusion protein. SpCas9 sequence may be underlined where the short PAM interacting residues (AGAAAGCGG) are highlighted and the long ZFP^{TS4} is highlighted.

FIG. 36 presents one embodiment of a plasmid expressing SpCas9^{MT3}-ZFP^{TS4} fusion protein. SpCas9 sequence may be underlined where the short PAM interacting residues (AGAAAGAAG) are highlighted and the long ZFP^{TS4} is highlighted.

FIG. 37 presents one embodiment of a sequence of Cas9-Zif268 (also referred to as DBD²⁶⁸) fusion protein.

FIG. 38 presents one embodiment of a sequence of Zif268-Cas9 fusion protein.

FIG. 39 presents one embodiment of a sequence of Cas9-TAL268 fusion protein.

FIG. 40 presents one embodiment of a sequence of TAL268-Cas9 fusion protein.

FIG. 41 presents one embodiment of a sequence of Cas9-ZFP^{TS4} fusion protein.

FIG. 42 presents one embodiment of a sequence of Cas9-ZFP^{TS2*} fusion protein.

FIG. 43 presents one embodiment of a sequence of Cas9-ZFP^{TS3} fusion protein.

FIG. 44A-D presents exemplary data showing that SpCas9^{MT}-ZFP chimeras have improved precision.

FIG. 44A: Sequences of Target Site 2 (TS2), TS3 and TS4^{20,26} with the 12 bp ZFP binding sites highlighted, respectively, with the arrow indicating the bound DNA strand.

FIG. 44B: Lesion rates determined by T7EI assay^{77,78} for SpCas9, SpCas9^{MT3} and SpCas9^{MT3}-ZFP at TS2, TS3 and TS4. Data are from three independent biological replicates performed in HEK293T cells. Error bars indicate s.e.m.

FIG. 44C: Deep sequencing analysis of SpCas9^{MT3}-ZFP precision. Lesion rates for target sites and off-target sites with significant activity assayed by sequencing PCR products spanning each genomic locus for SpCas9, SpCas9^{MT3}-ZFP and Neg control. Error bars indicate s.e.m. Asterisks indicate OT sites where the cleavage rate for SpCas9^{MT3}-ZFP is significantly above the NegCT.

FIG. 44D: Example GUIDE-seq peaks for Cas9^{WT} (top) and SpCas9^{MT3}-ZFP^{TS2} (bottom). Both have strong peaks at TS2 target site, but only Cas9^{WT} has signal at OT2-1. The position of each site is indicated above the peak.

FIG. 45 presents exemplary data comparing lesion rates at TS2 and OT2-2 as determined by T7EI assay for SpCas9^{WT} and SpCas9^{MT3}-ZFP^{TS2} variants that alter the number of ZFPs or change them completely (TS2*). The binding site for the TS2*-ZFP is highlighted. Removing finger 1 (F2-4) or 4 (F1-3) from the four-finger TS2 ZFP (F1-4) modestly impacts target site activity, but dramatically improves precision. Data are from three independent biological replicates from different days in HEK293T cells. Error bars indicate s.e.m.

FIG. 46 presents exemplary data showing an activity profile of SpCas9 (gray) and SpCas9^{MT3}-ZFP^{TS3} (white) at TS3 target site with guides containing single-base mismatches at the 20 positions (M1-M20) across the target site. Both nucleases have similar activity at the TS3 target site with a fully cognate guide (leftmost bars), but SpCas9^{MT3}-ZFP^{TS3} has dramatically enhanced sensitivity to mismatches between the guide and target site. Data are from T7EI assays on amplicons spanning the genomic target site from three independent biological replicates performed on different days in HEK293T cells. Error bars indicate s.e.m.

FIG. 47 presents exemplary data showing precise rapamycin-dependent cleavage by Cas9^{MT}-FRB and or FKBP-TALE^{TS3} nucleases. T7EI assay on PCR products spanning the TS3 target site (Top) or OT3-2 off-target site (Bottom) genomic loci. Cas9^{WT}, Cas9^{MT}TALE^{TS3} and Cas9^{MT}-FRB FKBP-TALE^{TS3} (+ or −) of 20 nM rapamycin (Rap). Cas9^{MT}-FRB FKBP-TALE^{TS3} activity at the target site is Rap-dependent (arrows). At OT3-2, only Cas9^{WT} produces lesions (weak T7EI activity in in all lanes).

FIG. 48 presents exemplary data showing T7E1 analysis reveals efficient NmCas9 editing of a site adjacent to a GATT PAM within the Vegfa/TS3 amplicon. 24-nt NmCas9 guides with either one (G23) or two (GG22) G residues at the 5′ end edit the target as efficiently as the canonical SpCas9/TS3 sgRNA combination.

FIG. 49A&B present exemplary data showing that attenuated NmCas9-PAM interactions can be rescued by a fused DBD. Data are from three independent replicates on different days in HEK293 cells. Error bars indicate s.e.m.

FIG. 49A: Activity profile of NmCas9 and Zif268-NmCas9 on a common target site with different PAM sequences and a neighboring Zif268 site.

FIG. 49B: Activity profile of NmCas9, NmCas9^{DM} (attenuated by K1013A and R1025A), and Zif268-NmCas9^{DM} on a common target site, with different spacings between the GATT PAM and a Zif268 site.

FIG. 50 presents an illustrative overview of a gene-correction strategy via minigene knock-in into an early intron of CYBB. For example, Cas9-pDBDs can be programmed to cleave intron 1 (or 2) in the context of a repair cassette that contains exons 2-13 (or 3-13) flanked by a strong splice acceptor (SA, human 0-globin) and a polyA sequence (BGH polyA). These elements may be flanked by homology arms to facilitate HDR-based insertion of the repair cassette.

FIG. 51 presents exemplary data showing T7EI activity of SpCas9/sgRNAs in intron 2 of CYBB in HEK293T and PLB985 cells (top panel), and a PCR assay showing CYBB minigene cassette insertion by NHEJ mediated ligation (bottom panel).

FIG. 52A-C present an illustrative overview of the distribution of potential SpCas9 off-target sites within the human genome.

FIG. 52A: Schematic of the SpCas9/sgRNA system and the two sequential stages of licensing required for cleavage: Stage 1-PAM recognition (nGG is highly preferred) and Stage 2-complementary R-loop formation between the 20 nucleotide guide RNA and the interrogated DNA sequence.

FIG. 52B: Genome-wide analysis using CRISPRseek²¹ of the potential off target sites for a representative set of 124,793 guide RNAs targeting human exons sequences. Guides were binned based on the predicted off-target site with the smallest number of mismatches to the guide sequence. A perfect match indicates the presence of an off-target site with a perfect guide match (wedge). Only 1.6% of these guide sequences do not have an off-target site with 3 or fewer mismatches to the guide sequence (wedge). This subset would be the best candidates for precise genome editing. The vast majority of guides typically have many potential off-target sequences with 3 or fewer mismatches.

FIG. 52C: Genome-wide analysis of the minimum number of mismatches in off-target sites for a representative set of 55,687 guide RNAs targeting human promoter regions (binned as describe above). Only 1% of these guide sequences do not have an off-target site with 3 or fewer mismatches to the guide sequence (wedge).

FIG. 53A-D present an illustrative overview of the distribution of potential SpCas9 offtarget sites within a human genome.

FIG. 53A: Genome-wide analysis of the sum of off-target scores determined by CRISPRseek²⁸ for the top 10 off-target sites for a representative set of 124,793 guide RNAs targeting gene exons. These were binned into five different categories where a lower score is better. An off-target site is scored as 100 if it is a perfect match to the guide sequence.

FIG. 53B: Genome-wide analysis of the sum of the off-target scores determined by CRISPRseek²⁸ for the top 10 off-target sites for a representative set of 55,687 guide RNAs targeting gene promoter regions.

FIG. 53C: Guide RNAs targeting gene exons with no predicted off-targets with <=3 mismatches (wedge) are analyzed for off-target sites with potential bulges in the sgRNA:DNA heteroduplex²⁹. Wedges indicate the fraction of guides that have one or more off-target sites that have perfect complementarity with the exception of a single bulge.

FIG. 53D: Guide RNAs targeting gene promoters with no predicted off-targets with <=3 mismatches (wedge) are analyzed for off-target sites with potential bulges in the sgRNA:DNA duplex. Wedges indicate the fraction of guides that have one or more off-target sites that have perfect complementarity with the exception of a single bulge.

FIG. 54 presents exemplary data showing a protein expression analysis of SpCas9 and SpCas9-Zif268 and SpCas9-TAL268 platforms. HEK293T cells are transfected with the indicated Cas9 plasmid which has triple HA-tag. Top Panel: Full length protein is probed with anti-HA antibody. Bottom Panel: Alpha-tubulin is used as loading control.

FIG. 55 presents exemplary data showing SpCas9 or SpCas9-Zif268 programmed independently with four different sgRNAs targeting four different genomic sites with neighboring Zif268 binding sites (highlighted)(Top Panel), and that SpCas9 cuts efficiently only at the target site with a nGG PAM, but SpCas9-Zif²⁶⁸ cuts efficiently at additional target sites with nAG, nGA or nGC PAMs (Bottom Panel). Genomic regions were PCR-amplified, and lesions (e.g., insertions or deletions within a local sequence) were detected by T7 Endonuclease I (T7EI) assay.

FIG. 56A presents exemplary data showing T7 Endonuclease I (T7EI) assays on PCR products spanning a genomic target site (underlined) with an NGG PAM and neighboring Zif268 site (highlight) for SpCas9 or SpCas9 mutants with or without a Zif268 fusion. For SpCas9^{MT2} & SpCas9^{MT3}, robust nuclease activity is only observed when Zif268 is fused to the C-terminus. The gel image is representative of T7EI assays at this genomic target site, where cleaved products are noted by arrowheads.

FIG. 56B presents exemplary data showing quantification of average T7EI-based lesion rates at the PLXNB2 locus from three independent biological replicates performed on different days in HEK293T cells. Error bars indicate standard error of the mean.

FIG. 57 presents exemplary data showing an analysis of the genomic activity profile of SpCas9 mutants (MT1, MT2, MT3 & MT4) independently and as SpCas9-Zif268 fusions at the PLXNB2 locus at a target site with an nGG PAM and a Zif268 binding site 11 bp away on the Watson strand. T7EI assay data from PCR products spanning the target site in three independent biological replicates (Rep1, Rep2, Rep3) performed on different days in HEK293T cells. Cleaved products are indicated by arrowheads.

FIG. 58 presents exemplary data showing an analysis of the genomic activity profile of SpCas9^{MT1} at TS2, TS3 and TS4 sites. T7EI assay data from PCR products spanning the target site in three independent biological replicates (Rep1, Rep2, Rep3) performed on different days in HEK293T cells. Cleaved products are indicated by arrowheads.

FIG. 59 presents exemplary data of an analysis of a genomic activity profile of SpCas9^{MT3}-ZFP^{DCLK2} and SpCas9^{MT3}-ZFP^{F9} at DNAJC6 and PLXDC2 sites respectively. These sequences have compatible binding sites for the DCLK2⁷ and Factor IX¹ ZFPs. T7EI assay data from PCR products spanning the target site from single experiment done in HEK293T cells. Cleaved products are indicated by arrowheads. Similar analysis of SpCas9^{MT3}-ZFP^{HEBP2} (targeting a compatible binding site for the HEBP2 ZFP6) at GPRC5B did not detect any lesions for this SpCas9^{MT3}-ZFP fusion (data not shown).

FIG. 60A-D present exemplary data demonstrating improved precision of SpCas9^{MT}-ZFP chimeras.

FIG. 60A: Sequences of Target Site 2 (TS2), Target Site 3 (TS3) and Target Site 4 (TS4) for the SpCas9/sgRNAs described by Joung and colleagues^{14,25}. The 12 bp ZFP binding sites for TS2, TS3 and TS4 are highlighted, respectively, with the arrow indicating the strand that is bound.

FIG. 60B: Lesion rates determined by T7EI assay for SpCas9, SpCas9^{MT3} and SpCas9^{MT3}-ZFP at TS2, TS3 and TS4. Data are from three independent biological replicates performed on different days in HEK293T cells. Error bars indicate standard error of the mean.

FIG. 60C: Representative T7EI assay comparing lesion rates at TS3 and off-target site 2 (OT3-2)²⁵ for various SpCas9-chimera/sgRNA combinations. The activity at the target site for SpCas9^{MT3}-ZFP is dependent on the cognate sgRNA and ZFP, where SpCas9^{MT3}-ZFP^{TS3} can discriminate between TS3 and OT3-2.

FIG. 60D: Genomic target site cleavage activity by SpCas9, SpCas9^{WT}-ZFP^{TS3} and SpCas9^{MT3}-ZFP^{TS3} in response to dinucleotide mismatches placed at different positions within the guide sequence targeting the TS3 site. (Top Panel) T7EI assay data from PCR products spanning TS3 site in three independent biological replicates performed on different days in HEK293T cells. Error bars indicate standard error of the mean. (Bottom Panel) Schematic indicating the position of the dinucleotide mismatches across the guide sequence. SpCas9^{MT3}-ZFP^{TS3} displays superior discrimination to SpCas9 for dinucleotide mismatches in the sgRNA recognition sequence.

FIG. 61 presents exemplary data of a T7EI activity profile of SpCas9^{MT3}-ZFP^{TS3} at the TS3 genomic locus as a function of the number of incorporated fingers. Both Cas9^{WT} and SpCas9^{MT3}-ZFP^{TS3} with four fingers (F1-4) achieve efficient target cleavage. Removing a single finger from either end of the zinc finger array (F1-3 or F2-4) dramatically reduces the activity of the SpCas9^{MT3}-ZFP chimera. Cleaved products are indicated by arrowheads. The bar graph displays the mean lesion rate in three independent biological replicates (Rep1, Rep2, Rep3) performed on different days in HEK293T cells. Error bars indicate standard error of the mean.

FIG. 62 presents exemplary data showing an analysis of a genomic activity profile of SpCas9^{MT3}-TALE^{TS3} and SpCas9^{MT3}-TALE^{TS4} at the TS3 and TS4 sites, respectively. An arrow indicates the strand (Watson) of the highlighted sequence that is bound by the TALE. Two different TALE repeat lengths (9.5 and 15.5) were examined at each target site. T7EI assay data from PCR products spanning the target site in three independent biological replicates (Rep1, Rep2, Rep3) performed on different days in HEK293T cells. Cleaved products are indicated by arrowheads.

FIG. 63A-C present exemplary data showing an activity profile of SpCas9^{MT3}-ZFP^{TS3/TS4} with truncated sgRNAs (tru-gRNA)³⁴.

FIG. 63A: Nuclease activity based on T7EI assay for SpCas9^{WT} and SpCas9^{MT3}-ZFP^{TS3} with a 17 nucleotide truncated guide at the TS3 target site.

FIG. 63B: Nuclease activity based on T7EI assay for SpCas9^{WT} and SpCas9^{MT3}-ZFP^{TS4} with an 18 nucleotide truncated guide at the TS4 target site. Cleaved products are indicated by arrowheads.

FIG. 63C: Target sites for the TS3 and TS4 tru-gRNAs and graph showing the average activity at each target site in three independent biological replicates performed on different days in HEK293T cells. Error bars indicate standard error of the mean. For both TS3 and TS4, the SpCas9^{MT3}-ZFP chimera is more sensitive to the truncation of the guide sequence, which is consistent with the greater sensitivity of this system to guide mismatches.

FIG. 64A-C present exemplary data showing a deep sequencing analysis of SpCas9^{MT3}-ZFP chimera precision.

FIG. 64A: Lesion rates for target sites and off-target sites with significant activity assayed by deep sequencing PCR products spanning each genomic locus for SpCas9, SpCas9^{MT3}, SpCas9^{WT}-ZFP and SpCas9^{MT3}-ZFP and untreated (NegCT). Error bars indicate standard error of the mean.

FIG. 64B: Improvement in precision of SpCas9^{MT3}-ZFP relative to SpCas9^{WT} as measured by the relative Specificity Ratio of target site lesion rate relative to each off-target lesion rate (Specificity Ratio=Target site lesion rate/Off-target lesion rate).

FIG. 64C: Comparison of average lesion rates at TS2 and OT2-2 determined by T7EI assay for SpCas9^{WT} and SpCas9^{MT3}-ZFP^{TS2} variants that alter the number of zinc fingers or change them completely (TS2*). The binding site for the ZFP^{TS2*} is indicated. Removing finger 1 (F2-4) or finger 4 (F1-3) from the four finger TS2 ZFP array (F1-4) at most modestly impacts target site activity, but it dramatically improves precision. Data are from three independent biological replicates performed on different days in HEK293T cells. Error bars indicate standard error of the mean.

FIG. 65 presents an exemplary OT2-2 genomic sequence. The sequence complementary to the guide is underlined with the two mismatched positions in bold. The nGG PAM is shown and the potential ZFP^{TS2} binding site highlighted. Below the genomic sequence is predicted consensus recognition motif and sequence logo for ZFP^{TS2} based on a Random Forest model of ZFP recognition³⁵. The predicted recognition motif only differs substantially at one position in the finger 4 binding site (C versus A).

FIG. 66A&B present exemplary data showing a T7EI activity profile of SpCas9^{MT3}-ZFP^{TS2} at the TS2 genomic locus and OT2-2 as a function of the number of incorporated fingers.

FIG. 66A: Both Cas9^{WT} and SpCas9^{MT3}-ZFP^{TS2} with four fingers (F1-4) result in efficient cleavage at the TS2 target site (arrowheads indicate cleaved products). Removing a single finger from either end of the zinc finger array (F1-3 or F2-4) at most modestly reduces activity of the SpCas9^{MT3}-ZFP chimera. Removing a both terminal fingers from the zinc finger array (F2-3) dramatically reduces activity of the SpCas9^{MT3}-ZFP chimera. Construction of an alternate ZFP (TS2*) that recognizes an overlapping target site can also promote target cleavage.

FIG. 66B: Both Cas9^{WT} and SpCas9^{MT3}-ZFP^{TS2} with four fingers (F1-4) result in efficient cleavage at the OT2-2 off-target site (arrowheads indicate cleaved products). Removing a single finger from either end of the zinc finger array (F1-3 or F2-4) dramatically reduces activity of the SpCas9^{MT3}-ZFP chimera. As does the utilization of an alternate ZFP (TS2*) that recognizes a different target site. Data from three independent biological replicates (Rep1, Rep2, Rep3) performed on different days in HEK293T cells.

FIG. 67A&B present exemplary data for a genome-wide off-target analysis of SpCas9^{WT3}-ZFPs by GUIDE-seq¹⁷.

FIG. 67A: Number of off-target sites with nuclease activity detected for SpCas9^{WT} and SpCas9^{MT3}-ZFP with TS2, TS3 and TS4 guides.

FIG. 67B-67D: Number of unique reads captured by GUIDE-seq for nuclease active sites within the genome (TS2/TS3/TS4 target site [bold] and off-target sites). Previously defined off-target sites are colored black^{14,17} and potential new off-target sites that were identified in this analysis are SpCas9^{WT} or SpCas9^{MT3}-ZFP. Some sites (e.g. OGT2-10 & OGT2-20) contain only reads from a single library for SpCas9MT3-ZFP and are not binned as off-target sites.

FIG. 68 provides an illustrative model of the three stages of target site licensing that may play a role in the ability of SpCas9^{MT3}-pDBD to cleave DNA. Due to the attenuation of SpCas9 DNA-binding affinity (mutation indicated by star), the efficient engagement of a sequence for PAM recognition or guide RNA complementarity requires the presence of a neighboring DNA sequence that can be bound by the attached pDBD. This requirement for pDBD binding adds a third stage of target site licensing for efficient cleavage.

FIG. 69 presents exemplary data for the impact of a single point mutation of a conserved arginine (Arg1025) residue in the PAM interaction domain on NmCas9 activity in a GFP reporter assay. HEK293 cells in 24-well plates were transfected with 100 ng split-GFP reporter (Wilson, K. A., Chateau, M. L. & Porteus, M. H. Design and Development of Artificial Zinc Finger Transcription Factors and Zinc Finger Nucleases to the hTERT Locus. Mol Ther Nucleic Acids 2, e87 (2013)), into which we had cloned a protospacer (with its NNNNGATT PAM) (SEQ ID NO: 1) targeted by the natural N. meningitidis 8013 CRISPR spacer 9. Variants carrying PAM mutations (as indicated) were also used. Also included in the transfections were ng of an mCherry-expressing plasmid (as a transfection marker), and 290 ng of a plasmid expressing wt NmCas9 or mutants that change a candidate PAM recognition residue (Arg1025) to Ala, Lys, or Ser, respectively. The NmCas9-expressing plasmid also encoded the spacer 9-containing sgRNA. Three identical transfections were done on different days. In each case, after 48 hours post-transfection, cells were harvested and analyzed by flow cytometry to identify the fraction of mCherry-positive cells that were also GFP-positive.

FIG. 70 presents exemplary data for the impact of a double mutation of a conserved arginine (Arg1025) and lysine (Lys1013) residue in the PAM interaction domain on NmCas9 activity in a GFP reporter assay. HEK293 cells in 24-well plates were transfected with 100 ng split-GFP reporter, into which we had cloned a protospacer (with its NNNNGATT PAM) (SEQ ID NO:1) targeted by the natural N. meningitidis 8013 CRISPR spacer 9. Variants carrying PAM mutations (as indicated) were also used. Also included in the transfections were 10 ng of an mCherry-expressing plasmid (as a transfection marker), and 290 ng of a plasmid expressing wt NmeCas9 or mutants that change a candidate PAM recognition residue (Arg1025) to Ala, Lys, or Ser, respectively, each in combination with a second mutation changing Lys1013 to Ser. The NmeCas9-expressing plasmid also encoded the spacer 9-containing sgRNA. Three identical transfections were done on different days. In each case, after 48 hours post-transfection, cells were harvested and analyzed by flow cytometry to identify the fraction of mCherry-positive cells that were also GFP-positive.

FIG. 71 presents exemplary data examining the ability of an N-terminal or C-terminal fusion of Zif268 to NmCas9 to be able to rescue the cleavage activity of attenuated NmCas9 mutants. The split-GFP reporter system was modified to include a Zif268 binding site, either upstream (US) (i.e. on the opposite side of the protospacer to the NNNNGATT PAM) (SEQ ID NO:1) or downstream (DS) (i.e., on the same side as the protospacer as the PAM). In all cases, the Zif268 binding site started 5 bp away from the protospacer (US) or PAM (DS), and was either in the Watson (W) or Crick (C) orientation. The NmCas9-expressing plasmid encoded either WT NmCas9, the R1025A single-mutant NmCas9 (SM), or the K1013A/R1025A double-mutant NmCas9 (DM). In addition, the NmCas9 was fused to no additional domains, N-terminal Zif268 (Nter-Zif268), or C-terminal Zif268 (Zif268-Cter). HEK293 cells in 24-well plates were transfected with 100 ng split-GFP reporter. Also included in the transfections were 10 ng of an mCherry-expressing plasmid (as a transfection marker), and 290 ng of the plasmid expressing NmeCas9 and the spacer 9-containing sgRNA. Three identical transfections were done on different days. In each case, after 48 hours post-transfection, cells were harvested and analyzed by flow cytometry to identify the fraction of mCherry-positive cells that were also GFP-positive.

FIG. 72 presents exemplary data examining the impact of spacing of a downstream Zif268 binding site relative to the PAM on the activity of NmCas9^{SM} or NmCas9^{DM} fused to Zif268 in the split-GFP reporter assay. The spacing between the PAM and the downstream Zif268 binding site (W orientation) was varied as indicated. In addition, only the fusion with Zif268 at the N-terminus of NmeCas9 was used, and NmeCas9 included the 1025A single mutation alone (top) or the K1013A/R1025A double mutation (bottom).

FIG. 73 presents exemplary data demonstrating the ability of a ZFP fusion (Zif268) to enhance the activity of NmCas9 at a preferred GATT PAM genomic target with a 5 bp spacing and Crick (C) orientation of the ZFP binding site. T7E1 assay to detect NmCas9-catalyzed genome editing of a chromosomal target site (N-TS5) that has a GATT PAM adjacent to a naturally occurring Zif268-binding DNA sequence. HEK293 cells in 24-well format were transfected with 300 ng of a plasmid expressing NmCas9, or of NmCas9 derivatives as indicated. The plasmid also expressed an NmCas9 sgRNA with a guide sequence complementary to the chromosomal target site. 72 hours after transfection, genomic DNA was prepared from the cells and subjected to T7E1 analysis according to standard protocols. The percent editing for Cas9 and Cas9-Zif268 is given underneath the corresponding lanes. The right-most two lanes are negative controls with NmCas9-Zif268, in which the sgRNA construct included no cloned spacer (“Cas9-Zif268-No spacer”) or a non-cognate spacer (Cas9-Zif268-Sg-N-TS9). The results show that a ZFP domain fusion to the C-terminus of NmeCas9 can improve editing efficiency of a chromosomal target site that has a GATT PAM.

FIG. 74 presents exemplary data demonstrating that a ZFP fusion can restore activity of the single and double NmCas9 mutants at a chromosomal target site that has a GATT PAM with a 9 bp spacing and Crick (C) orientation of the ZFP binding site. Experiment performed as in FIG. 73, except that a different chromosomal target (N-TS7) site was tested.

FIG. 75 presents exemplary data demonstrating that a ZFP fusion can restore activity of the single NmCas9 mutant at a chromosomal target site that has a GATT PAM with an 11 bp spacing and Crick (C) orientation of the ZFP binding site. Experiment performed as in FIG. 73, except that a different chromosomal target (N-TS9) site was tested. There is weak background T7EI cleavage activity in all of the lanes including the controls.

FIG. 76 presents exemplary data demonstrating that a ZFP fusion can restore activity of the double NmCas9 mutant at a chromosomal target site that has a GATT PAM with an 9 bp spacing and Watson (W) orientation of the ZFP binding site. Experiment performed as in FIG. 73, except that a different chromosomal target (N-TS8) site was tested.

FIG. 77 presents exemplary data demonstrating that a ZFP fusion can enhance activity of NmCas9 at a chromosomal target site that has a GATT PAM with an 12 bp spacing and Watson (W) orientation of the ZFP binding site. Experiment performed as in FIG. 73, except that a different chromosomal target (N-TS10) site was tested. There is weak background T7EI cleavage activity in all of the lanes including the controls.

FIG. 78 presents exemplary data demonstrating that a ZFP fusion can restore activity of the single NmCas9 mutant at a chromosomal target site that has a GATT PAM with an 14 bp spacing and Watson (W) orientation of the ZFP binding site. Experiment performed as in FIG. 73, except that a different chromosomal target (N-TS11) site was tested.

FIG. 79 presents exemplary data demonstrating that a ZFP fusion can restore activity of the single or double NmCas9 mutants at a number of alternate PAMs in the split-GFP reporter assay. These reporters include a Zif268 binding site (Watson orientation), 5 base pairs downstream of the PAM. The PAM was either wild-type (GATT), or mutated as indicated. The NmCas9-expressing plasmid encoded either WT NmeCas9, the R1025A single-mutant NmCas9 (SM, top panel), or the K1013A/R1025A double-mutant NmCas9 (DM, lower panel). In addition, NmCas9 was fused to no additional domains [wild-type NmCas9; SM NmCas9 (top panel) or DM NmCas9 (bottom panel)], or to N-terminal Zif268. HEK293 cells in 24-well plates were transfected with 100 ng split-GFP reporter. Also included in the transfections were 10 ng of an mCherry-expressing plasmid (as a transfection marker), and 290 ng of the plasmid expressing NmCas9 and the spacer 9-containing sgRNA. Three identical transfections were done on different days. In each case, after 48 hours post-transfection, cells were harvested and analyzed by flow cytometry to identify the fraction of mCherry-positive cells that were also GFP-positive.

FIG. 80 presents exemplary data demonstrating that a ZFP fusion can enhance activity of NmCas9 at a chromosomal target site that has a GTTT PAM (left panel) or GTCT PAM (right panel) with a 5 bp spacing and Watson (W) orientation of the ZFP binding site. Experiment performed as in FIG. 73, except that a different chromosomal targets (N-TS20, left panel; N-TS21, right panel) sites were tested.

FIG. 81 presents exemplary data demonstrating that a ZFP fusion can enhance activity of NmCas9 at a chromosomal target site that has a GTTT PAM (left panel) or GCTT PAM (right panel) with a 5 bp spacing and Watson (W) orientation of the ZFP binding site. Experiment performed as in FIG. 73, except that a different chromosomal targets (N-TS20, left panel; N-TS22, right panel) sites were tested.

FIG. 82 presents exemplary data demonstrating that a ZFP fusion can permit activity of NmCas9 at a chromosomal target site that has a GACA PAM (left panel) or restore activity of a single or double mutant NmCas9 at GATA PAM (right panel) with a 5 bp spacing and Watson (W) orientation of the ZFP binding site. Experiment performed as in FIG. 73, except that different chromosomal targets (N-TS24, left panel; N-TS25, right panel) sites were tested.

FIG. 83 presents exemplary data demonstrating that a ZFP fusion can restore activity of a single or double mutant NmCas9 at GATT PAM (right panel) with a 5 bp spacing and Watson (W) orientation of the ZFP binding site. Experiment performed as in FIG. 73, except that a different chromosomal target (N-TS3) site was tested at different concentrations of transfected DNA.

FIG. 84 presents one embodiment of a sequence of NmCas9.

FIG. 85 presents one embodiment of a sequence of NmCas9 R1025A (also referred to as NmCas9^{SM}) fusion protein.

FIG. 86 presents one embodiment of a sequence of NmCas9 K1013A/R1025A (also referred to as NmCas9^{DM}) fusion protein.

FIG. 87 presents one embodiment of a sequence of Zif268-NmCas9 fusion protein.

FIG. 88 presents one embodiment of a sequence of NmCas9-Zif268 fusion protein.

FIG. 89 presents one embodiment of a sequence of Zif268-NmCas9^{SM} fusion protein.

FIG. 90 presents one embodiment of a sequence of NmCas9^{SM}-Zif268 fusion protein.

FIG. 91 presents one embodiment of a sequence of Zif268-NmCas9^{DM} fusion protein.

FIG. 92 presents one embodiment of a sequence of NmCas9^{DM}-Zif268 fusion protein.

FIG. 93 presents exemplary data demonstrating that SpCas9^{MT3}-NmdCas9 nucleases programmed with orthogonal guides for neighboring target sites can function as a cohesive unit to cleave DNA. Four different combinations (D1 through D4) of SpCas9 (underlined target sequence neighboring TGG PAM) and NmCas9 (underlined target sequence neighboring GATT or AATC PAM (SEQ ID NO:2) depending on DNA strand that is bound). These different target site orientations are separated by 6 to 30 bp of DNA (6 bp “gctagc” spacer shown in each sequence) in the Split-GFP reporter assay. The bar graph represents the mean activities of SpCas9 (SpWT), NmCas9 (NmWT), SpCas9^{MT3} (SpMT3) or SpCas9^{MT3}-NmdCas9 (SpMT3-Nmd—purple bars) for biological triplicate experiments. Error bars represent standard error of the mean. SpCas9^{MT3}-NmdCas9 displays good activity on D1 and D2 oriented sites at most spacings. Note—SpCas9^{MT3} has only background activity on site D1-6 bp.

FIG. 94 presents exemplary data demonstrating that NmdCas9-SpCas9^{MT3} nucleases programmed with orthogonal guides for neighboring target sites can function as a cohesive unit to cleave DNA. Four different combinations (D1 through D4) of SpCas9 (underlined target sequence neighboring TGG PAM) and NmCas9 (underlined target sequence neighboring GATT or AATC PAM depending on DNA strand that is bound). These different target site orientations are separated by 6 to 30 bp of DNA (6 bp “gctagc” spacer shown in each sequence) in the Split-GFP reporter assay. The bar graph represents the mean activities of SpCas9 (SpWT), SpCas9^{MT3} (SpMT3) or NmdCas9-SpCas9^{MT3} (Nmd-SpMT3) for biological triplicate experiments. Error bars represent standard error of the mean. NmdCas9-SpCas9^{MT3} displays good activity on some D1 and D2 oriented sites depending on the spacing between the domains. Note—SpCas9^{MT3} has only background activity on site D1-6 bp.

FIG. 95 presents exemplary data demonstrating that the SpCas9^{MT3}-NmdCas9 nucleases programmed with orthogonal guides for neighboring target sites can target genomic sequences. T7 Endonuclease I (T7EI) assay showing cleavage activity of SpCas9^{MT3}-dNmCas9 fusions at a genomic target site. (Top) Organization of the target site where the binding sites of SpCas9^{MT3} and dNmCas9 are oriented with the PAMs between the protospacers. (SpCas9 NGG PAM on Watson strand and dNmCas9 NNNNGATT PAM (SEQ ID NO:1) on the Crick Strand, where 20 and 24 bp represent the sgRNA complementary regions for SpCas9 and NmCas9, respectively.) (Bottom) T7EI nuclease assay on PCR products of genomic regions spanning the SpCas9^{MT3}-dNmCas9 target site. Different combinations of fused or unfused SpCas9 and NmCas9 or SpCas9^{MT3}-dNmCas9 are examined with different combinations of sgRNAs. Both the wildtype (WT) SpCas9 (arrowheads, lane 3) and NmCas9 nuclease can cleave their respective target sites. However, SpCas9^{MT3} is ineffective, but can be rescued by dNmCas9 fused to the C-terminus, which substitutes as the DTU (lane 6).

FIG. 96 presents exemplary data demonstrating that the SpCas9^{MT3}-NmdCas9 nucleases programmed with orthogonal guides for neighboring target sites can efficiently cleave the TS3 target site. (Top) Organization of the target site where the binding sites of SpCas9^{MT3} and dNmCas9 are oriented with the PAMs on the same strand. (SpCas9 NGG PAM—red and dNmCas9 NNNNGATT PAM (SEQ ID NO:1), where underlined 20 and 24 bp represent the sgRNA complementary regions for SpCas9 and NmCas9, respectively.). (Bottom) T7EI nuclease assay on PCR products of genomic regions spanning the SpCas9^{MT3}-NmdCas9 target site (TS3). Wild-type SpCas9 (SpCas9wT) and wild-type NmCas9 (NmCas9^{WT}) programmed with their sgRNAs can cleave the target site. Attenuated SpCas9 (SpCas9^{MT3}) cannot cleave the target unless tethered to NmdCas9 (SpCas9^{MT3}-NmdCas9).

FIG. 97 presents exemplary data demonstrating that the SpCas9^{MT3}-NmdCas9 nucleases programmed with orthogonal guides for neighboring target sites have greatly improved precision relative to wild-type SpCas9 (SpCas9^{WT}). Genomic DNA treated with the constructs from FIG. 95 (i.e. programmed to target the TS3 genomic site) were analyzed to examine the off-target activity at site OT3-2, which is the most active off-target site for wild-type SpCas9. T7EI nuclease assay on PCR products of genomic regions spanning the OT3-2 off-target site for cells treated with each nuclease programmed with sgRNAs for the TS3 locus. Wild-type SpCas9 efficiently cleaves this off-target site whereas SpCas9^{MT3}-NmdCas9 displays no activity. This demonstrates a dramatic improvement in the precision of our attenuated dual Cas9 fusion protein.

FIG. 98 presents exemplary data demonstrating that SpCas9^{MT3}-NmCas9 fusions can generate local deletions within the genome. Cells were treated with the corresponding nuclease and their complementary sgRNAs. The local genomic sequence was PCR amplified from the genomic DNA of treated cells and run on an agarose gel. There is evidence of a deletion (smaller amplified product) at the genomic locus in the SpCas9^{MT3}-NmCas9 dual nuclease treated cells.

FIG. 99 presents exemplary data demonstrating that the fusion partners of the FRB and FKBP domains influences activity of the nuclease and that the incorporation of a Nuclear localization sequence (NLS) and nuclear export sequence (NES) on different components are critical for improving activity and reducing the background cleavage rate in the absence of the dimerizing drug (Rapamycin). Improvements in the substituents and the order of the localization and dimerization domains on the SpCas9 and pDBD scaffold plays an important role in improving function. Top: Fusion of Cas9 to FKBP and a C-terminal fusion of destabilized FRB (FRB*) to ZF was found to be superior for achieving maximum activity in presence of drug (Rapamycin: Rap) and lowest background in absence of drug. (arrowheads denote bands indicating nuclease activity) Bottom: Removing NLS from Cas9 and adding 2×NES and 2×NLS to the DBD component reduces background and increases drug-dependent activity at the target site.

FIG. 100 presents exemplary data demonstrating that fusion of Zif268 to Split-SpCas9 broadens the targeting range of this system. Top: schematic of drug-inducible split-cas9-DBD fusion. Bottom: Fusion of DBD to the C-terminal component of drug-inducible split Cas9 results in high activity at the target site containing a suboptimal NAG PAM and a neighboring zinc finger binding site only in the presence of the drug (Rapamycin). Lesions in the genomic DNA are detected by T7EI assay, where the bands indicative of nuclease modification of the genome are indicated by arrows.

FIG. 101 presents exemplary data demonstrating the analysis of various combinations of NES and NLS sequences on Split-Cas9 and Split-Cas9-ZFP activity at a target site containing and AG PAM at the Pmpca locus. The N-terminal domain of the Split-Cas9 contains FKBP and the C-terminal domain contains FRB. Activity should be realized by the presence of Rapamycin, where this is assessed via a T7EI assay. Modulating the presence and number of the NLS and NES domains on each component can dramatically change the drug-dependent activity and background cleavage rates.

FIG. 102 presents exemplary data demonstrating the improved specificity of out engineered drug dependent systems programmed with the sgRNA and ZFP targeting the TS2 target site based on GUIDE-seq. (Top) comparison of the sequence of the TS2 target site and a highly active off-target site (OTG2-1), where the PAM is bold and the non-canonical bases in OTG2-1. The FIG. shows a pileup of sequence reads around each locus for wild-type SpCas9 (spCas9-WT), Split-SpCas9 from the Zhang lab (Split-SpCas9), our drug-dependent SpCas9-FKBP/ZFP-FRB and our drug-dependent Split-SpCas9^{MT3}-pDBD, where the latter three are all in the presence of rapamycin. All constructs have high activity at the target site, but off-target activity is limited to the spCas9-WT and Split-SpCas9 from the Zhang lab.

FIG. 103 presents one embodiment of a sequence of the 2×NLS-Cas9^{MT3}-NLS-FKBP fusion protein. NLS (bold), SpCas9^{MT3} (italics), FKBP (underlined).

FIG. 104 presents one embodiment of a sequence of the 2×NLS-3× Flag-2×NES-TS2^{zF}-FRB* fusion protein. NLS (bold), NES (underlined), ZFP^{TS2} (italics), FRB* (highlighted).

FIG. 105 presents one embodiment of a sequence of the NLS-Split-NCas9-NLS-FRB fusion protein. NLS (bold), N-terminal SpCas9 fragment (italics), FRB (highlighted).

FIG. 106 presents one embodiment of a sequence of the FKBP-Split-CCas9^{MT3}-NLS-3×HA-NLS-TS2^{ZF}-3×FLAG-2×NLS fusion protein. FKBP (underlined), NLS (bold), C-terminal SpCas9^{MT3} (italics, mutant R1335K bold), ZFP^{TS2} (highlight).

FIG. 107 presents exemplary data comparing the lesion rates at target sites T5, T6 & Z1 for SpCas9 and three different SpCas9^{MT3}-ZFPs by T7EI assay. Nuclease constructs and sgRNA expression vectors were transfected into a Jurkat cell line with an integrated HIV provirus (J-Lat line) and after 72 hours the lesion rates within the 5′ LTR of HIV were analyzed by T7EI assay. The T5 nuclease in particular displays good activity (arrowheads indicate the bands indicative of target site lesions and the values below each column indicate the calculated lesion rate). Each target site is listed above the gel where the sgRNA target site is underlined, the NGG PAM is in italics and the ZFP binding site is in highlights.

FIG. 108 presents exemplary data comparing the off-target lesion rates for SpCas9 and three different SpCas9^{MT3}-ZFP^{T5} programmed with the T5 sgRNA by T7EI assay. Nuclease constructs and sgRNA expression vectors were transfected into a Jurkat cell line with an integrated HIV provirus (J-Lat line) and after 72 hours the lesion rates were analyzed by T7EI assay. Comparison of lesion rates at one computationally predicted off-target sites for the T5 sgRNAs with either wild-type SpCas9 or SpCas9^{MT3}-ZFP^{T5} by T7EI assay. Lesions are evident for SpCas9 at this off-target site (arrowheads) but these are absent for SpCas9^{MT3}-ZFP^{T5}.

FIG. 109 presents one embodiment of a sequence of the Cas9^{MT3}-NLS-3×HA-NLS-ZFP^{T5} that targets the T5 site in the HIV LTR.

FIG. 110 presents one embodiment of a sequence of the Cas9^{MT3}-NLS-3×HA-NLS-ZFP^{T6} that targets the T6 site in the HIV LTR.

FIG. 111 presents one embodiment of a sequence of the Cas9^{MT3}-NLS-3×HA-NLS-ZFP^{Z}i that targets the Z1 site in the HIV LTR.

FIG. 112 representative sgRNA sequences for various loci for SpCas9 and NmCas9. The guide sequence element is indicated in underline.

FIG. 113 presents one embodiment of a sequence of the NmdCas9-SpCas9^{MT3}, where NmCas9 is nuclease dead.

FIG. 114 presents one embodiment of a sequence of the SpCas9^{MT3}-NmdCas9, where NmCas9 is nuclease dead.

FIG. 115 presents one embodiment of a sequence of the SpCas9^{MT3}-NmCas9n^{RuvC}, where NmCas9 is a nickase via inactivation of the HNH domain.

FIG. 116 presents one embodiment of a sequence of the SpCas9^{MT3}-NmCas9n^{HNH}, where NmCas9 is a nickase via inactivation of the RuvC domain.

FIG. 117 presents one embodiment of a sequence of the SpCas9^{MT3}-NmCas9^{WT} dual nuclease system.

### DETAILED DESCRIPTION OF THE INVENTION

The present invention may be related to the field of genetic engineering. In particular, specific genes can be cleaved, edited or deleted using Cas9 nucleases with improved precision when coupled to DNA targeting units, which can be either programmable DNA-binding domains or an alternate isoform of Cas9 that are programmed to recognize a site neighboring the sequence targeted by the Cas9 nuclease.

The CRISPR/Cas9 system is commonly employed in biomedical research; however, the precision of Cas9 is sub-optimal for gene therapy applications that involve editing a large population of cells. Variations on a standard Cas9 system have yielded improvements in the precision of targeted DNA cleavage, but often restrict the range of targetable sequences. It remains unclear whether these variations can limit lesions to a single site within the human genome over a large cohort of treated cells. In some embodiments, the present invention contemplates that fusing a programmable DNA-binding domain (pDBD) to Cas9 combined with an attenuation of Cas9's inherent DNA binding affinity produces a Cas9-pDBD chimera with dramatically improved precision and increased targeting range. Because the specificity and affinity of this framework is easily tuned, Cas9-pDBDs provide a flexible system that can be tailored to achieve extremely precise genome editing at nearly any genomic locus—characteristics that are ideal for gene therapy applications.

Conventional CRISPR technology has been used to effect genome editing with Cas9 nuclease activity in combination with specific guide RNAs (sgRNAs) to place the enzyme on specific genomic DNA sequence where a double-stranded break is generated. Target location by Cas9 nuclease is typically a two step process. First, the PAM specificity of Cas9 acts as a first sieve by defining a subset of sequences that are bound for a sufficient length of time to be interrogated by the incorporate guide RNA. This step may be a kinetic selection for functional target sequences. Sequences with sufficient homology to a PAM specificity of the Cas9 nuclease are interrogated by the incorporate guide RNA through R-loop formation that allows Watson-Crick pairing between the guide RNA and the bound DNA target site. If there may be sufficient complementarity in this interaction the nuclease domains within Cas9 (the RuvC and HNH domains) will generate a double-stranded break in the DNA. Szczelkun et al., Direct observation of R-loop formation by single RNA-guided Cas9 and Cascade effector complexes. Proc Natl Acad Sci USA. 2014 Jul. 8; 111(27):9798-803.

The precision of a Cas9 nuclease-DNA targeting unit chimera may be improved by attenuating an independent recognition of target sites by a Cas9 nuclease, which can be achieved by altering its PAM recognition sequence and/or its affinity for a phosphodiester backbone by mutating residues that are involved in contacting the RNA or DNA. Further attenuation can be achieved by using a truncated single guide RNA to program a Cas9 nuclease. By attenuating the affinity of Cas9 for the DNA, the ability of a Cas9 nuclease to achieve a kinetic selection of a target sequence may be abrogated. Consequently, a Cas9 nuclease may be completely dependent on a coupled DNA targeting unit to achieve sufficient residence time on the DNA to allow R-loop formation with the incorporated guide RNA. Complementarity between a PAM specificity of a Cas9 nuclease and a target site may be still required for R-loop formation, but it may be no longer sufficient for initiating this event. This creates a system where the cleavage of a target site may be dependent on at least three features of the Cas9 nuclease-DNA targeting unit chimera: 1) recognition of the sequence by the DNA-targeting unit, 2) complementarity between the Cas9 nuclease PAM specificity and the sequence, and 3) complementarity between the guide RNA and the target site. An added advantage of the Cas9 nuclease-DNA targeting unit fusion may be that it expands the targeting range of the Cas9 nuclease by allowing a broader range of PAM sequences to be utilized, as normally low affinity PAM sequences can be utilized.

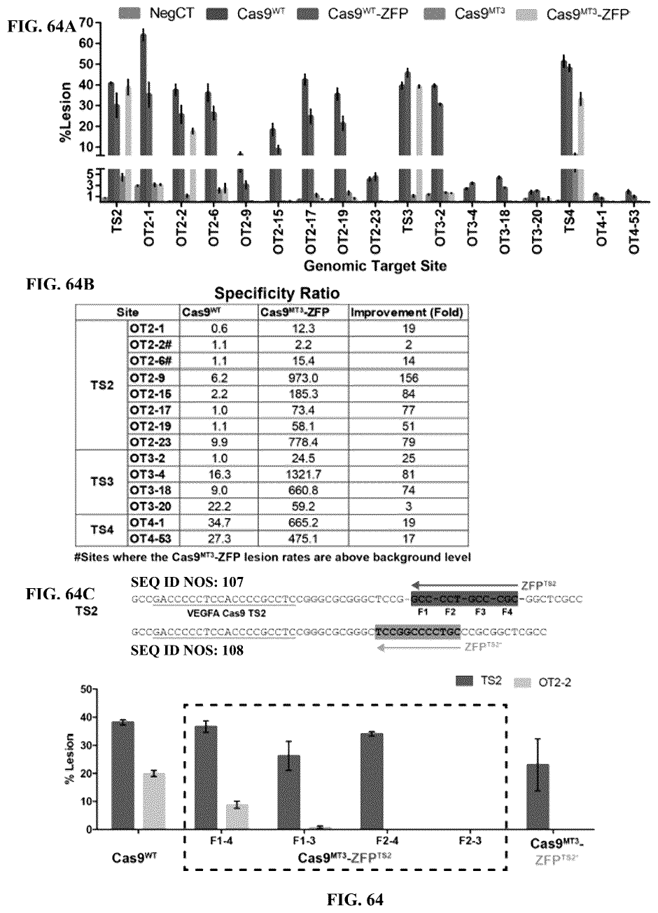

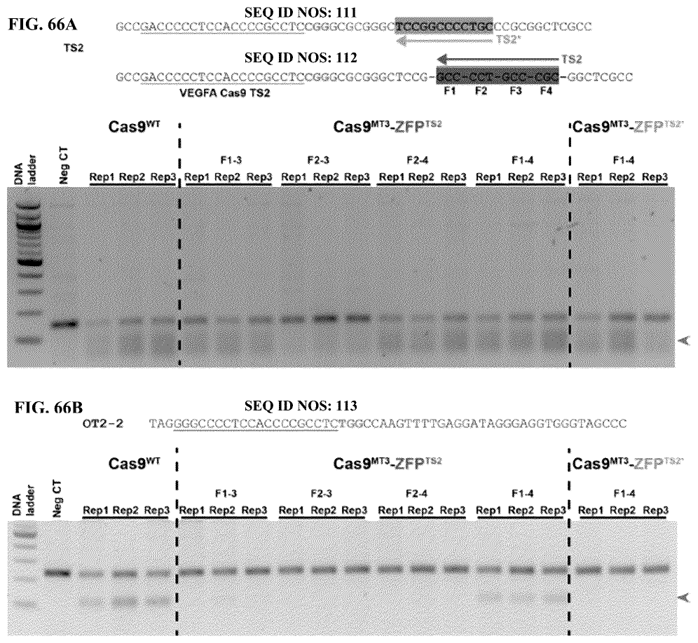

One potential advantage of a SpCas9-pDBD system over other Cas9 platforms is an ability to rapidly tune affinity and specificity of an attached pDBD to further improve its precision. Consequently, improved precision of SpCas9^{MT3}-ZFP^{TS2} was obtained by truncating a zinc finger protein (commonly abbreviated as ZFP, ZnF or ZF) to reduce its affinity for target site OT2-2. Constructs with a truncation of any of the terminal zinc fingers may display high activity at a target site. However, these truncations also reduced or eliminated off-target activity at OT2-2, reflecting a profound improvement in the precision of SpCas9^{MT3}-ZFP^{TS2}, FIG. 64C and FIG. 66A&B.

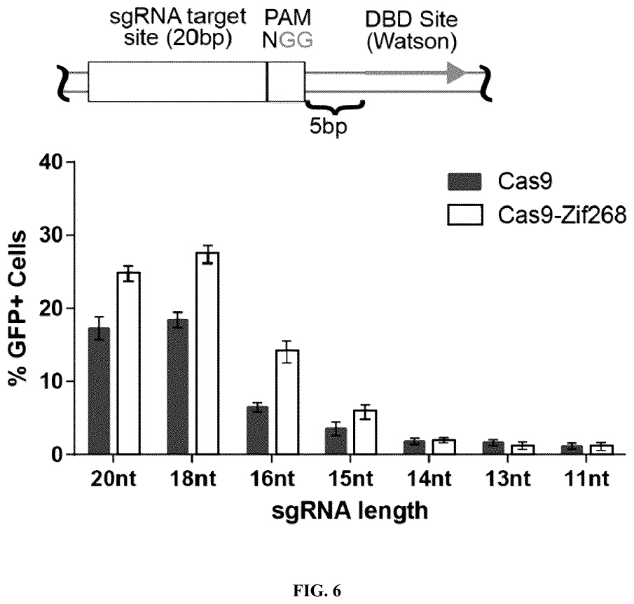

Similarly, utilization of a ZFP (e.g., TS2*) that recognizes an alternate sequence neighboring an TS2 guide target site also abolishes off-target activity at OT2-2, confirming that cleavage by SpCas9^{MT3}-ZFP^{TS2} at this off-target site is dependent on an ZFP. FIG. 64C & FIG. 6. Given the improvements in precision realized by these simple adjustments in the composition of a ZFP, it should be possible to achieve even greater enhancements in precision via more focused modification of a ZFP composition and a linker connecting a ZFP to a SpCas9 protein.

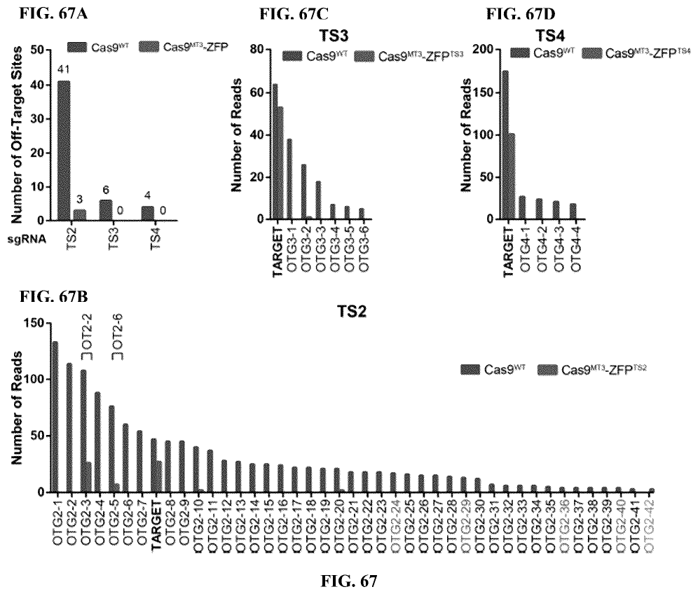

GUIDE-seq¹⁷ was employed to provide an unbiased assessment of the propensity for SpCas9^{MT3}-ZFP chimeras to cleave at alternate off-target sites within a genome. Using a modified protocol with a customized bioinformatics analysis of peaks within a genome, genome-wide DSB induction by SpCas9 and SpCas9^{MT3}-ZFP^{TS2/TS3/TS4} were assessed. This analysis reveals a dramatic enhancement of the precision of the SpCas9^{MT3}-ZFPs at all three of the target sites. FIG. 67A&B. For SpCas9^{MT3}-ZFP^{TS3} and SpCas9^{MT3}-ZFP^{TS4} nuclease dependent-oligonucleotide capture did not detect at any cleavage site besides the target site. For SpCas9^{MT3}-ZFP^{TS2}, which retains three active off-target sites that overlap with SpCas9, there is a dramatic reduction in cleavage activity at all of these alternate sequences. In addition, there is one new weak off-target site (OTG2-42) for SpCas9MT3-ZFP^{TS2}. Thus, these data demonstrate that the presence of the ZFP fusion does not generate a new category of ZFP-mediated highly active off-target sequences for SpCas9^{MT3}.

In some embodiments, the present invention contemplates compositions and methods that improve Cas9 effector systems. In some embodiments, Cas9 fusion proteins are contemplated comprising a DNA targeting unit that may be a DNA binding domain (DBD). In some embodiments, Cas9 fusion proteins are contemplated comprising a DNA targeting unit that may be another Cas9 isoform (e.g. SpCas9-NmCas9) programmed with an orthogonal sgRNA. In some embodiments a Cas9 nuclease would be directly fused to the DNA-targeting unit. In some embodiments, a Cas9 nuclease would be associated with the DNA-targeting unit via a dimerization domain. In some embodiments, the dimerization domain would be a heterotypic dimerization domain, which would allow control over component association. In some embodiments, the dimerization domain would be drug-dependent, which would provide temporal control over the activity of the nuclease based on the presence of the small molecule dimerizer within the cell.

Improvements in targeting precision have been achieved through the use of truncated sgRNAs (e.g., for example, less than 20 complementary bases). Fu et al., Improving CRISPR-Cas nuclease specificity using truncated guide RNAs. Nature Biotechnology (2014). Previous studies on truncated sgRNA have suggested that sgRNAs for spCas9 with less than 17 base pairs of complementarity to the target sequence have not been shown to be active in a genomic context. Improvements in precision have also been achieved by using pairs of Cas9 nickases to generate a double strand break. Mali et al., CAS9 transcriptional activators for target specificity screening and paired nickases for cooperative genome engineering. Nature Biotechnology 31, 833-838 (2013); and Cho et al., Analysis of off-target effects of CRISPR/Cas-derived RNA-guided endonucleases and nickases. Genome Research 24, 132-141 (2014). In addition, nuclease dead Cas9 (dCas9) variants have been fused to the FokI nuclease domain to generate programmable nucleases where dCas9 serves as the DNA-targeting unit and FokI may be the cleavage domain. Tsai et al., Dimeric CRISPR RNA-guided FokI nucleases for highly specific genome editing. Nature Biotechnology. 2014 June; 32(6):569-76; and Guilinger et al., Fusion of catalytically inactive Cas9 to FokI nuclease improves the specificity of genome modification. Nature Biotechnology. 2014 June; 32(6):577-82.

The proposed strategies described herein provide improved and more efficient Cas9-pDBD platforms that facilitate the construction of compact Cas9 orthologs. These compact orthologs permit alternate delivery methods (e.g. adeno-associated virus or AAV) broadening the clinical therapeutic modalities available for diseases including, but not limited to CGD. These strategies are also applicable to the treatment of a wide range of other monogenic disorders.

### I. Conventional Cas9 Protein Modifications

Recently, an RNA-guided adaptive immune system that may be widespread in bacteria and archaea has been adapted for achieving targeted DNA cleavage or gene regulation in prokaryotic and eukaryotic genomes. Clustered Regularly Interspaced Short Palindromic Repeat (CRISPR) RNA sequences and CRISPR-associated (Cas) genes form catalytic protein-RNA complexes that utilize the incorporated RNA to generate sequence-specific double strand breaks at a complementary DNA sequence. This nuclease platform has displayed remarkable robustness for targeted gene inactivation or tailor-made genome editing. Sander et al., CRISPR-Cas systems for editing, regulating and targeting genomes. Nature Biotechnology 32, 347-355 (2014); Mali et al. RNA-guided human genome engineering via Cas9. Science 339, 823-826 (2013); Ran et al. Double Nicking by RNA-Guided CRISPR Cas9 for Enhanced Genome Editing Specificity. Cell 154, 1380-1389 (2013); Fu et al., Improving CRISPR-Cas nuclease specificity using truncated guide RNAs. Nature Biotechnology 32, 279-284 (2014); and Wang et al. One-step generation of mice carrying mutations in multiple genes by CRISPR/Cas-mediated genome engineering. Cell 153, 910-918 (2013).

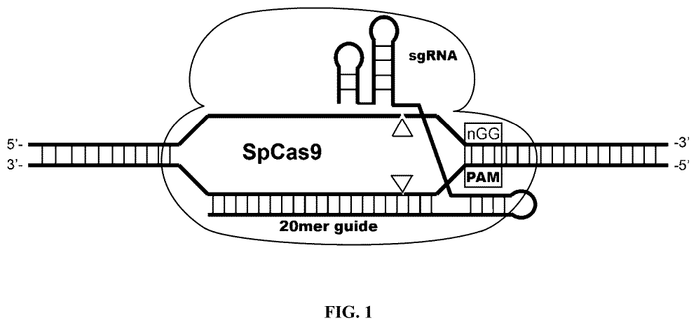

The CRISPR/Cas9 genome engineering system is revolutionizing biological sciences due to its simplicity and efficacy¹⁻³. The most commonly studied Cas9 nuclease originates from Streptococcus pyogenes (SpCas9)⁴. SpCas9 and its associated guide RNA license a DNA sequence for cleavage based on at least two stages of sequence interrogation⁴⁻⁸: i) compatibility of a PAM element with the specificity of the PAM-interacting domain, and ii) complementarity of a guide RNA sequence with the target site. Because it is straightforward to program Cas9 to cleave a desired target site through incorporation of a complementary single guide RNA (sgRNA)⁴, a primary constraint on Cas9 targeting is the presence of a compatible PAM element^{4,9,10}. For example, a PAM-interacting domain of wild-type SpCas9 (SpCas9^{WT}) preferentially recognizes a nGG element⁴, although it can inefficiently utilize other PAM sequences (e.g. nAG, nGA)^{9,11}. The simplicity of a SpCas9/sgRNA system allows facile editing of genomes in a variety of organisms and cell lines¹⁻³. Target specificity may be a function of recognition by both the guide RNA (through Watson-Crick base pairing) and an inherent specificity of Cas9 through recognition of a neighboring motif (e.g., for example, a protospacer adjacent motif (PAM)). FIG. 1.

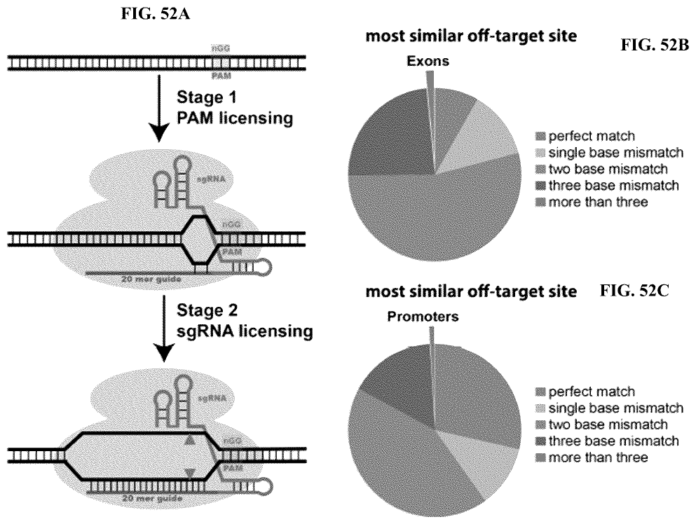

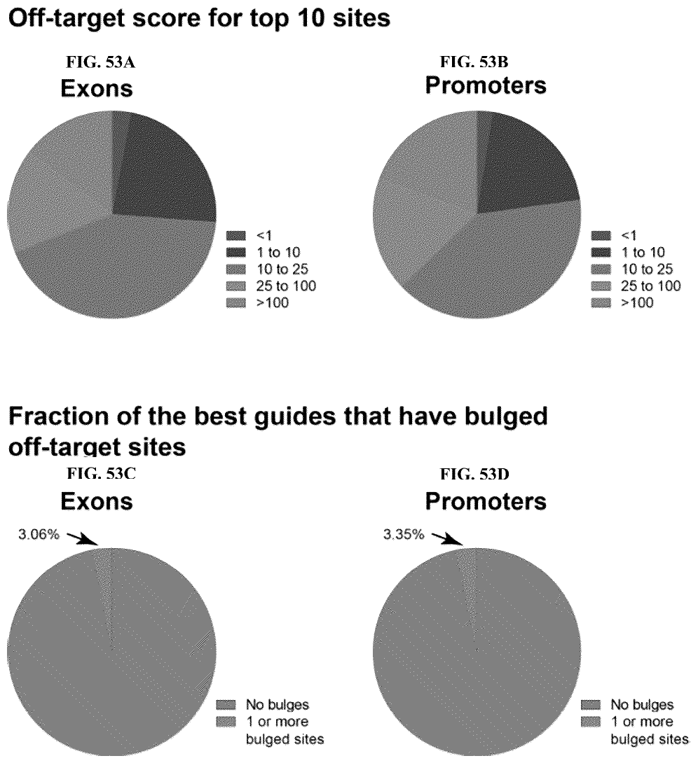

SpCas9 targeting precision is sub-optimal for most gene therapy applications involving editing of a large population of cells^{12,13}. Numerous studies have demonstrated that SpCas9 can cleave a genome at unintended sites^{9,14-20} with some guides acting at more than 100 off-target sites¹⁷. Recent genome-wide analyses of SpCas9 precision indicate that a majority of genomic loci that differ from a guide RNA sequence at 2 nucleotides, and a subset of genomic loci that differ at 3 nucleotides are cleaved with moderate activity¹⁷⁻²⁰. For some guides, off-target sites that differ by up to 6 nucleotides can be inefficiently cleaved¹⁷⁻²⁰. In addition, at some off-target sites bulges can be accommodated within the sgRNA:DNA heteroduplex to allow cleavage¹⁵. In this light, a global analysis was performed of potential SpCas9 target sites in exons or promoter regions using CRISPRseek^{21,22} to assess the general frequency of potential off-target sites with three or fewer mismatches for guide RNAs falling in two categories of sequence elements: exon regions or promoter regions. A vast majority of guides (˜98% in exons and ˜99% in promoters) was found to have one or more off-target sites with 3 or fewer mismatches and thus are likely to have some level of off-target activity, FIG. 52 A-C and FIG. 53A-D. Because off-target breaks have the potential to cause both local mutagenesis and genomic rearrangements (e.g., segmental deletions, inversions and translocations)^{17,18,23,24}, the resulting collateral damage for SpCas9 could have adverse consequences in therapeutic applications.

Reduced off-target cleavage rates have been reported with several modifications to the structure or delivery of a CRISPR/Cas9 system. Examples include, but are not limited to: changing guide sequence length and composition^{25,26}; employing pairs of Cas9 nickases²⁶⁻²⁸. dimeric FokI-dCas9 nucleases^{10,29}; inducible assembly of split Cas9³⁰⁻³³; Cas9 PAM variants with enhanced specificity³⁴; and delivery of Cas9/sgRNA ribonucleoprotein complexes³⁵⁻³⁷. However, it remains uncertain whether these variations can restrict cleavage to a single site within the human genome over a large cohort of treated cells^{12,38}. In addition, some of the most promising approaches (e.g., paired nickases or dimeric FokI-dCas9) restrict a targetable sequence space by requiring the proximity of two sequences compatible with Cas9 recognition.

Cas9 isoforms derived from different species can display different PAM specificities. Esvelt et al., Orthogonal Cas9 proteins for RNA-guided gene regulation and editing. Nature Methods 10, 1116-1121 (2013); Zhang et al., Processing-independent CRISPR RNAs limit natural transformation in Neisseria meningitidis. Molecular Cell 50, 488-503 (2013); Hou et al., Efficient genome engineering in human pluripotent stem cells using Cas9 from Neisseria meningitides (NmCas9). Proceedings of the National Academy of Sciences (2013); and Fonfara et al., Phylogeny of Cas9 determines functional exchangeability of dual-RNA and Cas9 among orthologous type II CRISPR-Cas systems. Nucleic Acids Research 42, 2577-2590 (2014). The Cas9 nuclease from Streptococcus pyogenes (hereafter, Cas9, or SpCas9 or catalytically active Cas9) can be guided to specific sites in a genome through base-pair complementation between a nucleotide guide region of an engineered RNA (sgRNA) and a genomic target sequence. Cho et al., Targeted genome engineering in human cells with the Cas9 RNA-guided endonuclease. Nature Biotechnology 31, 230-232 (2013); Cong et al., Multiplex genome engineering using CRISPR/Cas systems. Science 339, 819-823 (2013); Jinek et al., RNA-programmed genome editing in human cells. eLife 2, e00471 (2013); and Sternberg et al., DNA interrogation by the CRISPR RNA-guided endonuclease Cas9. Nature 507, 7490 (2014).

Structural information may be also available on Cas9 and Cas9-sgRNA-DNA complexes. Jinek et al., Structures of Cas9 Endonucleases Reveal RNA-Mediated Conformational Activation. Science (2014); Nishimasu et al., Crystal Structure of Cas9 in Complex with Guide RNA and Target DNA. Cell 1-23 (2014); and Anders et al., Structural basis of PAM-dependent target DNA recognition by the Cas9 endonuclease. Nature, 2014 Sep. 25; 513(7519):569-73. Various other studies have reported on Cas9 precision (e.g., activity at its target site relative to off-target sequences) within a genome. Studies on Cas9 nuclease have demonstrated that off-target cleavage can occur at both NGG and NAG PAMs, where there can be up to 5 mismatches within the guide recognition sequence. Fu et al., High-frequency off-target mutagenesis induced by CRISPR-Cas nucleases in human cells. Nature Biotechnology 31, 822-826 (2013); Pattanayak et al., High-throughput profiling of off-target DNA cleavage reveals RNA-programmed Cas9 nuclease specificity. Nature Biotechnology 31, 839-843 (2013); and Hsu et al., DNA targeting specificity of RNA-guided Cas9 nucleases. Nature Biotechnology 31, 827-832 (2013).

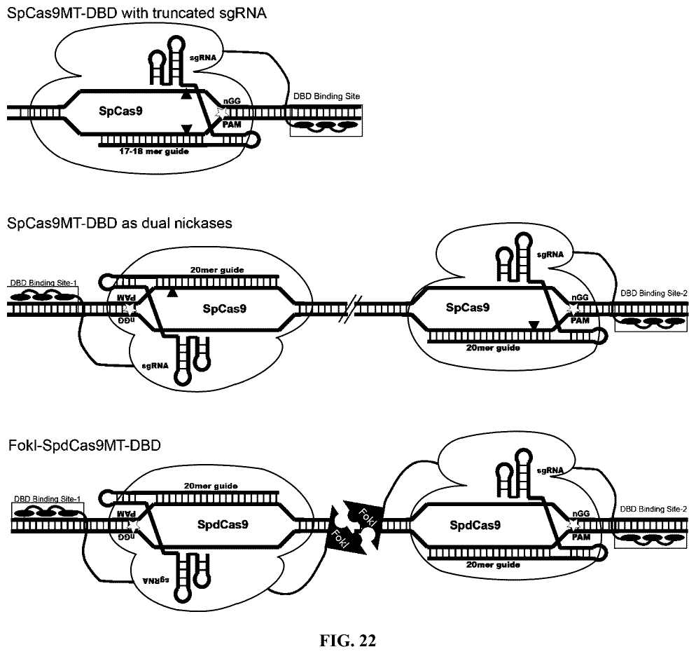

Other Cas9 variants for improving specificity have also been investigated. For example, double-strand breaks may be generated through the nicks generated in each strand by RuvC and HNH nuclease domains of Cas9. Jinek et al., A programmable dual-RNA-guided DNA endonuclease in adaptive bacterial immunity. Science 337, 816-821 (2012); and FIG. 22. Inactivation of one of the two nuclease sites within Cas9 (e.g., for example, a D10A mutation in the RuvC domain) generates a nickase that cleaves only a single strand. Alternatively, a pair of nickases that cut opposite strands in close proximity can generate a double-strand break and thereby improve precision since cleavage by a single nickase at a target site should not be as mutagenic as a double-strand break. Mali et al., CAS9 transcriptional activators for target specificity screening and paired nickases for cooperative genome engineering. Nature Biotechnology 31, 833-838 (2013); and Cho et al., Analysis of off-target effects of CRISPR/Cas-derived RNA-guided endonucleases and nickases. Genome Research 24, 132-141 (2014).

However, it has recently been shown that single nickases can be mutagenic with lesion rates >1% depending on the target site. Tsai et al., Dimeric CRISPR RNA-guided FokI nucleases for highly specific genome editing. Nature Biotechnology (2014); and Guilinger et al., Fusion of catalytically inactive Cas9 to FokI nuclease improves the specificity of genome modification. Nature Biotechnology 32, 577-582 (2014). Alternately, a catalytically-inactive, programmable, RNA-dependent DNA-binding protein (dCas9) can be generated by mutating both endonuclease domains within Cas9. Larson et al., CRISPR interference (CRISPRi) for sequence-specific control of gene expression. Nat Protoc 8, 2180-2196 (2013); and Qi et al., Repurposing CRISPR as an RNA-guided platform for sequence-specific control of gene expression. Cell. 2013 Feb. 28; 152(5):1173-83. When fused to a FokI endonuclease domain this construct can be used like zinc fingers or TALE domains to create the above dimeric nucleases, which display improved precision over a standard Cas9. FIG. 22. However, recently reported ChIP-seq datasets on dCas-sgRNA complexes reveal much more permissive binding (e.g., off-target binding) than cleavage, such that many sites are bound by Cas9 but not cut. Kuscu et al., Genome-wide analysis reveals characteristics of off-target sites bound by the Cas9 endonuclease. Nature Biotechnology 32, 677-683 (2014). This type of permissive binding may be a problem for FokI-dCas9 nucleases, leading to a greater number of off-target sites that are cleaved. Thus, there may be a need for an improved Cas9 platform having greater binding precision that may provide a platform for future gene therapy applications.

Type II CRISPR/Cas9 systems have been used for targeted genome editing in complex genomes, Barrangou et al., CRISPR-Cas systems: Prokaryotes upgrade to adaptive immunity. Molecular Cell. 2014 Apr. 24; 54(2):234-44; Hsu et al., Development and Applications of CRISPR-Cas9 for Genome Engineering. Cell. 2014 Jun. 5; 157(6):1262-78; and Sander et al., CRISPR-Cas systems for editing, regulating and targeting genomes. Nature Biotechnology. 2014 April; 32(4):347-55. Editing sites can be selected based primarily on two features: complementarity to a single-guide RNA (sgRNA), and proximity to a short (2-5 base pair) sequence called a protospacer adjacent motif (PAM). Subsequent DNA cleavage and repair enables gene inactivation by non-homologous end joining (NHEJ), or sequence correction and/or insertion by homology-directed repair (HDR). This technology has relevance to the construction of animal and cell models and gene therapy. Hu et al., RNA-directed gene editing specifically eradicates latent and prevents new HIV-1 infection. Proceedings of the National Academy of Sciences. 2014 Aug. 5; 111(31):11461-6; and Yin et al., Genome editing with Cas9 in adult mice corrects a disease mutation and phenotype. Nature Biotechnology. 2014 June; 32(6):551-3.

Despite these advantages, clinical genome editing may require even greater precision. Numerous reports have described promiscuity of standard Cas9, which leads to collateral damage at unintended sites. Fu et al., High-frequency off-target mutagenesis induced by CRISPR-Cas nucleases in human cells. Nature Biotechnology. 2013 September; 31(9):822-6; Pattanayak et al., High-throughput profiling of off-target DNA cleavage reveals RNA-programmed Cas9 nuclease specificity. Nature Biotechnology. 2013 September; 31(9):839-43; Hsu et al., DNA targeting specificity of RNA-guided Cas9 nucleases. Nature Biotechnology. 2013 September; 31(9):827-32; and Lin et al., CRISPR/Cas9 systems have off-target activity with insertions or deletions between target DNA and guide RNA sequences. Nucleic Acids Research. 2014; 42(11):7473-85.

Cas9/sgRNA variations that can improve precision but do not eliminate off-target activity include, but are not limited to: i) dual nickases (Mali et al., CAS9 transcriptional activators for target specificity screening and paired nickases for cooperative genome engineering. Nature Biotechnology. 2013 September; 31(9):833-8; and Ran et al., Double Nicking by RNA-Guided CRISPR Cas9 for Enhanced Genome Editing Specificity. Cell. 2013 Sep. 12; 154(6):1380-9.); ii) truncated sgRNAs (tru-sgRNAs; FIG. 22; Fu et al., Improving CRISPR-Cas nuclease specificity using truncated guide RNAs. Nature Biotechnology. 2014 March; 32(3):279-84); and iii) FokI fusions to dCas9 (Tsai et al., Dimeric CRISPR RNA-guided FokI nucleases for highly specific genome editing. Nature Biotechnology. 2014 June; 32(6):569-76; and Guilinger et al., Fusion of catalytically inactive Cas9 to FokI nuclease improves the specificity of genome modification. Nature Biotechnology. 2014 June; 32(6):577-82.). Consequently, there may be an unmet need for a Cas9-based system that can be cut at only a single site within a genome.

The PAM interaction residues for SpCas9 have been described (Anders et al., (2014) Structural Basis of PAM-Dependent Target DNA Recognition by the Cas9 Endonuclease, Nature 513(7519), 569-573.), but this study does not provide information on how to generate an improved Cas9 fusion protein with a DNA targeting unit or truncated sgRNA sequences.

It has been reported that PAM recognition sequences may play a role to efficiently engage Cas9 nucleolytic activity, thereby providing an explanation for low off-target editing rates. While describing Cas9 modification of DNA, this reference does not describe fusion proteins combining the elements, nor does it discuss modification of the Cas9 PAM site or other modifications beyond the targeting RNA. Cencic et al., (2014) Protospacer Adjacent Motif (PAM)-Distal Sequences Engage CRISPR Cas9 DNA Target Cleavage, PLoS. ONE 9(10), e109213.

An X-ray crystal structural analysis of Cas9 in a complex with guide RNA and target DNA has been reported. Nishimasu et al., (2014) Crystal Structure of Cas9 in Complex with Guide RNA and Target DNA, Cell 156(5), 935-949. E-published Feb. 13, 2014. This structural analysis provides insight into the identity of a Cas9 protospacer adjacent motif recognition domain and other sequence recognition features. While describing the orientation and features of the Cas9 in complex with and sgRNA and DNA, this reference does not describe the type of Cas9 modifications, fusion proteins, or mutations needed to make an attenuated Cas9.

A fusion protein using catalytically inactive Cas9 and FokI nuclease (FokI-dCas9) has been reported. Guilinger et al. (2014) Fusion of Catalytically Inactive Cas9 to FokI Nuclease Improves the Specificity of Genome Modification, Nat Biotech 32(6), 577-582. Cleavage of the sequence requires the combination of two of these FokI-dCas9 monomers where the targeting was greater than 140 fold higher specificity than wild type Cas9 with the same efficiency. While describing a Cas9 fusion protein complex that increases targeting, this reference does not describe a fusion protein with specific DNA binding proteins, modification of the PAM site, or truncated targeting sequences.

The fusion of both zinc fingers and TAL effectors as programmable DNA binding protein with non-Cas9 proteins has been reported to produce various effects upon targeted DNA sequences. Strauβ et al., (2013) Zinc Fingers, Tal Effectors, or Cas9-Based DNA Binding Proteins: What's Best for Targeting Desired Genome Loci?, Mol. Plant 6(5), 1384-1387. While describing zinc fingers, TAL effectors, and Cas9, this reference does not describe fusion proteins combining these elements, nor does it discuss any modification of the Cas9 protein (e.g., for example, specific mutations), beyond the targeting RNA.

dCas9 or TALE proteins have been fused with effector constructs (e.g., activation or repression domains) to modulate the expression of the Oct4 genes. Hu et al., (2014) Direct Activation of Human and Mouse Oct4 Genes Using Engineered TALE and Cas9 Transcription Factors, Nucleic Acids Res. 42(7), 4375-4390. While describing zinc fingers, TAL effectors, and Cas9, this reference does not describe fusion proteins combining these elements, nor does it discuss modification of the Cas9 (e.g., for example, specific mutations), beyond the targeting RNA.

CRISPR/Cas systems has been reported to be generally useful for genomic modification and gene modulation. Wu, F. “CRISPR/Cas Systems for Genomic Modification and Gene Modulation,” United States Patent Application Publication Number US 2014-0273226 (herein incorporated by reference). While describing Cas9 modification of DNA, this reference does not describe fusion proteins combining these elements, nor does it discuss modification of the Cas9 (e.g., for example, specific mutations), beyond the targeting RNA.

A single Cas enzyme has been programmed by a short RNA molecule to recognize a specific DNA target, in other words, the reported Cas enzyme can be recruited to a specific DNA target using said short RNA molecule. Cong et al., “CRISPR-Cas Component Systems, Methods and Compositions for Sequence Manipulation,” United States Patent Application Publication Number US 2014/0273231 (herein incorporated by reference). The reference describes a vector system that delivers the elements of the Cas system to affect changes to the DNA target. The reference also describes the importance of the PAM sequences into target DNA. While describing Cas9 modification of DNA, this reference does not describe fusion proteins combining these elements, nor does it discuss modification of the Cas9 PAM recognition domain (e.g., for example, specific mutations) or other modifications beyond the targeting RNA.

Non-Cas9/TALE fusion proteins have been reported where the TALEs are engineered, programmable DNA-binding domains which bind specifically to a preselected target sequence. Joung et al., “Transcription Activator-Like Effector (TALE)-Lysine-Specific Demethylase 1 (Lsdl) Fusion Proteins,” WO/2014/059255. This reference does not describe a fusion protein with Cas9 systems, nor does it discuss modification of the Cas9 PAM recognition domain (e.g., for example, specific mutations) or other modifications.

It has been reported that a mutation within an active site of an enzyme results in a change in DNA binding affinity. Shroyer et al., (1999) Mutation of an Active Site Residue in Escherichia coli Uracil-DNA Glycosylase: Effect on DNA Binding, Uracil Inhibition and Catalysis, Biochemistry 38(15), 4834-4845. This reference does not describe Cas9 fusion proteins, nor does it discuss modification of the Cas9 PAM recognition domain (e.g., for example, specific mutations) or other modifications beyond the targeting RNA.

### II. Cas9 Nuclease-DNA Targeting Unit Fusion Proteins

In some embodiments, the present invention contemplates a Cas9 nuclease-DNA Targeting Unit (Cas9-DTU) fusion protein that cleaves a single site within a genome. In one embodiment, the Cas9-DTU fusion protein may be compatible with previously reported specificity-enhancing variations of Cas9. In some embodiments, the present invention contemplates Cas9-DTU fusion proteins using a wide variety of Cas9 orthologs including, but not limited to, SpCas9 (e.g., Type II-A) and NmCas9 (e.g., Type II-C), both of which are validated as genome-editing platforms. Jinek et al., A programmable dual-RNA-guided DNA endonuclease in adaptive bacterial immunity. Science. 2012 Aug. 7; 337(6096):816-21; Hou et al., Efficient genome engineering in human pluripotent stem cells using Cas9 from Neisseria meningitidis. Proceedings of the National Academy of Sciences. 2013 Sep. 24; 110(39):15644-9; Jinek et al., RNA-programmed genome editing in human cells. eLife. 2013; 2:e00471; Mali et al., RNA-guided human genome engineering via Cas9. Science. 2013 Feb. 15; 339(6121):823-6; and Cong et al., Multiplex genome engineering using CRISPR/Cas systems. Science. 2013 Feb. 15; 339(6121):819-23. Because >90% of known Cas9 orthologs are either Type II-A or Type II-C (Fonfara I, Le Rhun A, Chylinski K, Makarova K S, Lécrivain A-L, Bzdrenga J, Koonin E V, Charpentier E. Phylogeny of Cas9 determines functional exchangeability of dual-RNA and Cas9 among orthologous type II CRISPR-Cas systems. Nucleic Acids Research. 2014 Feb. 1; 42(4):2577-90. PMCID: PMC3936727), the present invention facilitates embodiments to nearly any desired Type II Cas9 system.

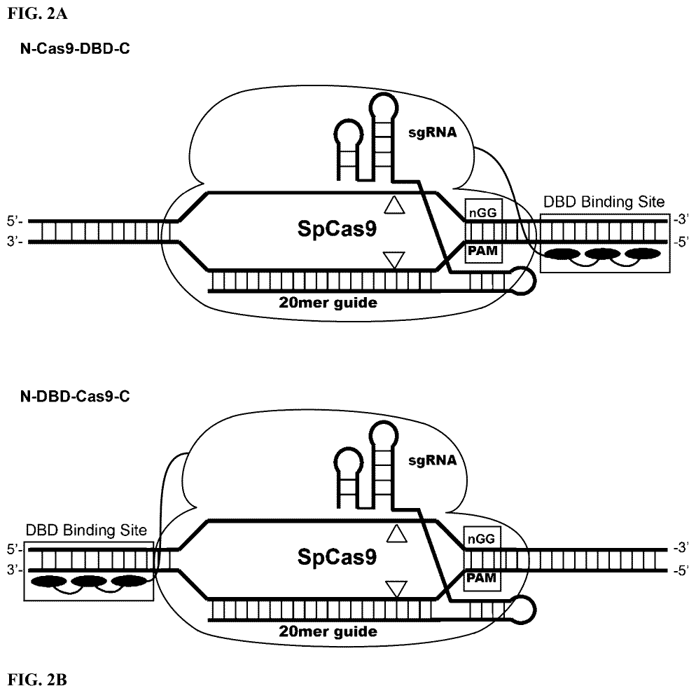

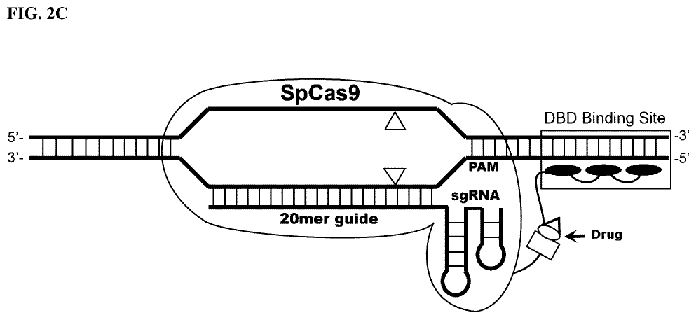

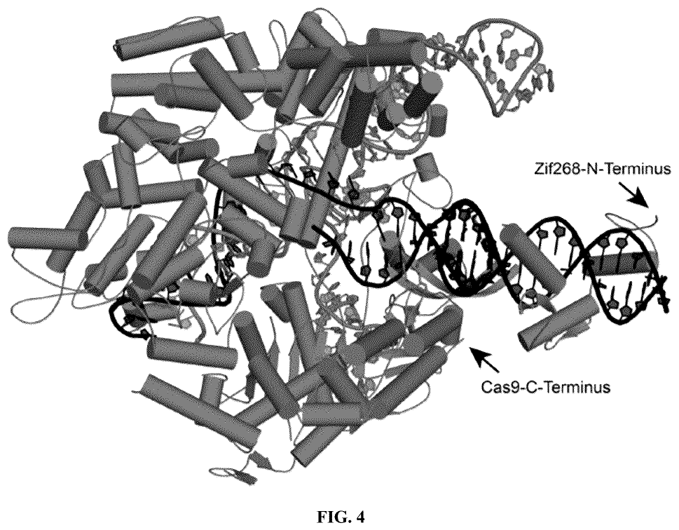

In one embodiment, the present invention contemplates an improved Cas9 platform, where target recognition precision is improved by incorporation of a programmable DNA-binding domain (pDBD), such as Cys2-His2 zinc finger protein (ZFPs)³⁹ or transcription-activator like effectors (TALEs)⁴⁰. FIGS. 2A and 4. Both of these pDBD platforms can be programmed to recognize nearly any sequence within the genome³⁹⁻⁴². Indeed, pDBDs have been employed with great success as targeting domains for programmable nucleases by incorporating non-specific FokI nuclease domain (ZFNs³⁹ and TALENs⁴⁰) or sequence-specific nuclease domains (e.g. megaTAL⁴³).

One favorable characteristic of the presently disclosed pDBDs is their inherent modularity whereby specificity and affinity can be rationally tuned by adjusting the number and composition of incorporated modules and the linkage between modules^{44,45}. In one embodiment, the present invention contemplates that a fusion of a pDBD to a mutant SpCas9 with an attenuated DNA-binding affinity generates a chimeric nuclease fusion protein comprising a broad sequence targeting range and dramatically improved precision (as compared to conventional Cas9 platforms). Although it is not necessary to understand the mechanism of an invention, it is believed that the present disclosed SpCas9-pDBD platforms have favorable properties for genome engineering applications. In addition, it is shown herein that these SpCas9-pDBD chimeras provide new insights into the barriers involved in licensing target site cleavage by a SpCas9/sgRNA complex.

Innovations to achieve an ultimate goal of precisely editing a single site within a genome comprise two general strategies that have applicability to all Cas9 systems. First, a DTU could be a programmable DBD fusion protein comprising either a ZFP (Urnov et al., Genome editing with engineered zinc finger nucleases. Nat Rev Genet. 2010 Sep. 1; 11(9):636-46) or a TALE protein (Joung J K, Sander J D. TALENs: a widely applicable technology for targeted genome editing. Nat. Rev. Mol. Cell Biol. 2013 January; 14(1):49-55). These DTU fusion proteins can precisely deliver a Cas9/sgRNA complex to a specific site within a genome and thereby facilitate sgRNA-dependent cleavage of an adjacent target sequence. Alternately, a DTU could be an orthogonal Cas9 isoform (e.g. nmCas9) that through the use of an orthogonal sgRNA targets the Cas9 nuclease to a specific site in the genome. In some embodiments, an orthogonal Cas9 DTU would be a nuclease-dead Cas9, so that it merely functions as a DNA recognition domain. In some embodiments, an orthogonal Cas9 DTU would be an active nuclease (either a nickase or nuclease), so that it can also break the DNA. In some embodiments, an orthogonal Cas9 DTU could also have attenuated DNA-binding affinity (NmCas9^{DM}, FIG. 49A&B), such that both attenuated Cas9s bind cooperatively. Second, attenuation of an initial binding of a Cas9 nuclease to a PAM or to DNA in general though mutations in contact to the phosphodiester backbone makes target site acquisition reliant on an accessory DTU, all the while retaining Cas9's RNA-guided cleavage fidelity.

In one embodiment, the present invention contemplates a coupled DNA cleavage system including at least three levels of licensing: 1) recognition of a neighboring site by an attached DTU, 2) PAM recognition, and 3) sgRNA complementarity. The data presented herein indicate that PAM specificity of a Cas9 can be tuned, which provides an opportunity to alter and/or refine the sequence preference of Cas9 to a high levels of precision, and may also allow allele-specific targeting using SNPs as discriminators—e.g., for inactivation of dominant disease alleles. In some embodiments, a combined DTU fusion protein and altered PAM recognition strategy may be also compatible with all prior variants of Cas9 (e.g., dual nickases, tru-sgRNAs, or FokI fusions) further extending the precision of these constructs. In some embodiments, a Cas nuclease-DTU will extend the number of target sites that are functional sequences, allowing the efficient discrimination of alleles based on SNPs that distinguish these alleles, where these SNPs if present in the PAM recognition sequence would be the discriminators between active and inactive target sites. Although it may be not necessary to understand the mechanism of an invention, it is believed that the presently disclosed Cas9-DTU fusion proteins yield constructs that provide a single site precision sufficient for targeted genome editing, thereby facilitating gene therapy applications.

In one embodiment, the present invention contemplates a flexible, highly precise Cas9-based nuclease platform that cleaves only a single site within a multigigabase genome. This level of precision facilitates Cas9-based in vivo gene corrections, which may require precise genome editing of billions to trillions of cells. Currently achievable levels of genome editing specificity with conventional platforms must be increased to circumvent the hazards of unintended, difficult-to-predict off-target mutations. Although it may be not necessary to understand the mechanism of an invention, it is believed that the specificity and activity of Cas9 gene editing can be dramatically improved through an incorporation of an appended, programmable DNA-binding domain (pDBD). It is also believed that such improvements in editing specificity may result from a Cas9 platform that comprises: i) PAM recognition by Cas9; ii) DNA recognition by an sgRNA; and iii) flanking sequence recognition by a DBD. The data herein demonstrate the improvement in precision with SpCas9 systems (Type II-A) and functionality with NmCas9 systems (Type II-C), but one of skill in the art would appreciate that the disclosed strategy is applicable to all Cas9 based systems, such as Staphylococcus aureus (SaCas9) systems (Type II-A). Ran, F. A. et al. In vivo genome editing using Staphylococcus aureus Cas9. Nature 520, 186-191 (2015).

The development of Cas9-DBDs in the context of these two most prevalent subtypes (with their distinct domain arrangements) facilitates application of the present invention to nearly any Cas9-based genome editing system. Jinek et al., Structures of Cas9 endonucleases reveal RNA mediated conformational activation. Science. 2014 Mar. 14; 343(6176):1247997. In addition, the presently disclosed Cas9-DBD framework should also be compatible with existing variants (e.g. dual nickases, tru-sgRNAs and/or FokI fusions) that have been reported to increase nuclease precision thereby enhancing precision. Fu et al., Improving CRISPR-Cas nuclease specificity using truncated guide RNAs. Nature Biotechnology. 2014 March; 32(3):279-84; Tsai et al., Dimeric CRISPR RNA-guided FokI nucleases for highly specific genome editing. Nature Biotechnology. 2014 June; 32(6):569-76; and Guilinger et al., Fusion of catalytically inactive Cas9 to FokI nuclease improves the specificity of genome modification. Nature Biotechnology. 2014 June; 32(6):577-82.

In some embodiments, the present invention contemplates a method for improving precision in genome editing using a Cas9-DBD fusion protein by engineering two representative Cas9 orthologs: S. pyogenes Cas9 (SpCas9; Type IIA) and N. meningitidis Cas9 (NmCas9; Type II-C, almost 300 aa smaller than SpCas9). These orthologs are validated genome-editing platforms, and the Type II-A and II-C families together encompass >90% of all Cas9 sequences. Modifications are presented that permit fused DBDs to increase precision and activity of both of these Cas9 orthologs as well as refine their inherent targeting range. One of skill in the art recognizes that the embodiments presented herein may be extended to other Cas9 systems or related CRISPR nuclease effectors (e.g. Cpf1; Zetsche, B. et al. Cpf1 Is a Single RNA-Guided Endonuclease of a Class 2 CRISPR-Cas System. Cell (2015). doi:10.1016/j.cell.2015.09.038), since it may be possible that alternative Cas9 variants within these classes or other CRISPR nuclease effectors may have equivalent or superior properties for clinical applications.

Based on reported structures of Cas9, some embodiments of the present invention contemplate fusions between any Cas9 protein and programmable DNA-binding domains (e.g., for example, Cys2His2 zinc fingers (ZFP), homeodomains or TALE domains). Both ZFPs, homeodomains and TALEs can be easily programmed to recognize a variety of DNA sequences, and have been employed with FokI nuclease to generate dimeric nucleases. Urnov et al., Genome editing with engineered zinc finger nucleases. Nat Rev Genet 11, 636-646 (2010); and Joung et al., TALENs: a widely applicable technology for targeted genome editing. Nat. Rev. Mol. Cell Biol. 14, 49-55 (2013); PMID 22539651. Although it may not be necessary to understand the mechanism of an invention, it is believed that by fusing a Cas9 to a DNA-binding domain (DBD), a hybrid nuclease may be created where the activity of the Cas9 component may be defined, in part, by an associated DNA-binding domain, FIGS. 2A and 2B.

The genome editing precision of available nuclease platforms may be improved to circumvent the hazards of unintended, difficult-to-predict off-target mutations¹, which can alter gene function through direct mutagenesis or translocations. Although it is not necessary to understand the mechanism of an invention, it is believed that the present method improves the specificity of Cas9 through an attachment of a pDBD to Cas9 with attenuated DNA-binding affinity, thereby establishing a system where Cas9 target site cleavage is dependent on sequence recognition by a pDBD. In addition, the present invention contemplates regulatable Cas9-pDBD prototypes where, for example, drug-dependent dimerization domains control the association of Cas9 and a pDBD, FIG. 2C.

In some embodiment of the present invention an association of a Cas9-nuclease and the DTU may be mediated by dimerization domains. These dimerization domains could be, but are not limited to homotypic dimerization domains, heterotypic dimerization domains, light mediated dimerization domains and/or drug-dependent dimerization domains. These dimerization domains could be, but are not limited to protein or RNA.

In one embodiment, the present invention contemplates a Cas9-pDBDs chimeric protein for target recognition and cleavage purposes by using a variety of Cas9 orthologs. In one embodiment, the method optimizes a SpCas9-pDBD system. In one embodiment, the method extends an approach to NmCas9 (Type II-C) and SaCas9¹⁶, which are more amenable to viral delivery. Although it is not necessary to understand the mechanism of an invention, it is believed that the development of Cas9-pDBDs in the context of the two most prevalent subtypes facilitates application of some of the present embodiments into future Cas9-based genome editing system. In one embodiment, the present invention provides a Cas9 editing platform that establishes efficient and precise gene correction. For example, by applying this approach in HSPCs an avenue for the ex vivo generation of a cell-based therapy can be established. Once established, this approach should be applicable to other HSPC-based monogenic disorders.

Preliminary data were collected using a Cas9-ZFP fusion protein (e.g., Zif268), where a ZFP was bound to both a Cas9 N-terminus (Zif268-Cas9) and/or a Cas9 C-terminus (Cas9-Zif268) via a long linker to provide flexibility in binding. The Zif268 sequence recognizes a nucleic acid target sequence of 5′-GCGTGGGCG-3′ (SEQ ID NO:3). C-terminal Cas9-ZFP/sgRNA complex activity was demonstrated using a GFP reporter assay, where the reporter construct may be inactive until a double-strand break was created within a target sequence (e.g., demonstrating a gain of function readout). The data demonstrated that both N-terminal Zif268-Cas9 and C-terminal Cas9-Zif268 were active, but that C-terminal Cas9-Zif268 showed the greatest activity.

### A. Development And Validation

Based on SpCas9 structures, a fusion protein was designed between SpCas9 and a programmable DBD, wherein a DBD comprised either ZFP or TALE domains (e.g. FIG. 4). Urnov et al. Genome editing with engineered zinc finger nucleases. Nat Rev Genet. 2010 Sep. 1; 11(9):636-46; Joung et al., TALENs: a widely applicable technology for targeted genome editing. Nat. Rev. Mol. Cell Biol. 2013 January; 14(1):49-55; Jinek et al., Structures of Cas9 endonucleases reveal RNA mediated conformational activation. Science. 2014 Mar. 14; 343(6176):1247997; and Nishimasu et al., Crystal structure of Cas9 in complex with guide RNA and target DNA. Cell. 2014 Feb. 27; 156(5):935-49.

Both ZFPs and TALEs can be programmed to recognize nearly any sequence within a genome, where their affinities and specificities can be tuned based on the number of modules incorporated. Rebar et al., Heritable targeted gene disruption in zebrafish using designed zinc-finger nucleases. Nature Biotechnology. 2008 Jun. 25; 26(6):702-8; Bhakta et al., Highly active zinc-finger nucleases by extended modular assembly. Genome Research. 2013 March; 23(3):530-8; Zhu et al., Using defined finger-finger interfaces as units of assembly for constructing zinc-finger nucleases. Nucleic Acids Research. 2013 Feb. 1; 41(4):2455-65; Kim et al., Preassembled zinc-finger arrays for rapid construction of ZFNs. Nature Methods. 2011; 8(1):7; Meckler et al., Quantitative analysis of TALE-DNA interactions suggests polarity effects. Nucleic Acids Research. 2013 April; 41(7):4118-28; and Reyon et al., FLASH assembly of TALENs for high-throughput genome editing. Nature Biotechnology. 2012 May; 30(5):460-5. Preliminary experiments discussed herein resulted in the fusion of Cas9 with Zif268 or a TALE domain programmed to recognize the same sequence (TAL268), to the N-terminus (e.g. Zif268-SpCas9) or C-terminus (e.g. SpCas9-Zif268) of SpCas9 via a long linker. Cermak et al., Efficient design and assembly of custom TALEN and other TAL effector-based constructs for DNA targeting. Nucleic Acids Research. 2011 July; 39(12):e82-2; and Meng et al., Counter-selectable marker for bacterial-based interaction trap systems. Biotechniques. 2006 February; 40(2):179-84. Although it may be not necessary to understand the mechanism of an invention, it is believed that a DBD, by recruiting Cas9 to a target site, would allow suboptimal PAM sequences to be cleaved efficiently, since there may be a kinetic barrier to R-loop formation by Cas9 at suboptimal PAM sequences. Szczelkun et al., Direct observation of R-loop formation by single RNA-guided Cas9 and Cascade effector complexes. Proc Natl Acad Sci USA. 2014 May 27.

SpCas9 may be believed to have a strong sequence preference for NGG over NAG and NGA PAMs and may be essentially inactive at other NXX PAM trinucleotides. Hsu et al., DNA targeting specificity of RNA-guided Cas9 nucleases. Nature Biotechnology. 2013 September; 31(9):827-32; Jiang et al., RNA-guided editing of bacterial genomes using CRISPR-Cas systems. Nature Biotechnology. 2013 March; 31(3):233-9; and Zhang et al., Comparison of non-canonical PAMs for CRISPR/Cas9-mediated DNA cleavage in human cells. Sci Rep. 2014; 4:5405. It has been reported that an SpCas9 target site with an NAG PAM shows increased activity mediated by an appended DBD. A co-transfected plasmid GFP reporter system assay in Human Embryonic Kidney (HEK 293T) cells may be used to measure targeted DSB activity. Wilson et al., Expanding the Repertoire of Target Sites for Zinc Finger Nuclease-mediated Genome Modification. Mol Ther Nucleic Acids. 2013 April; 2(4):e88.

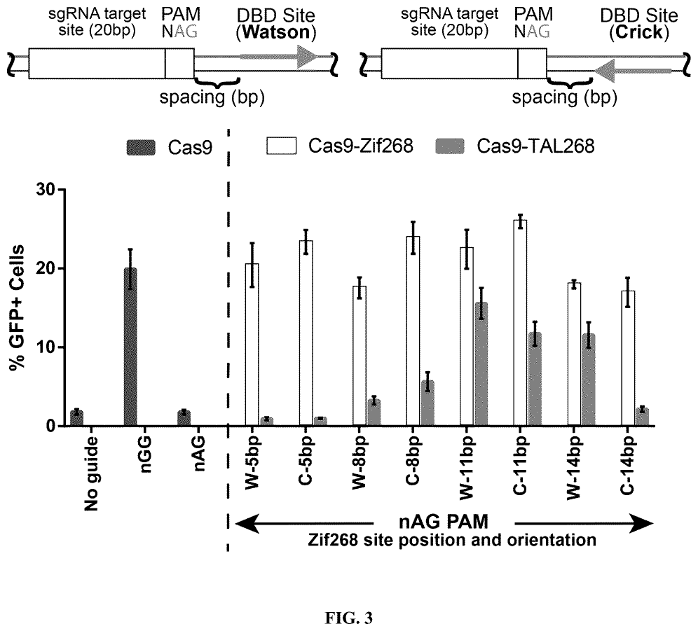

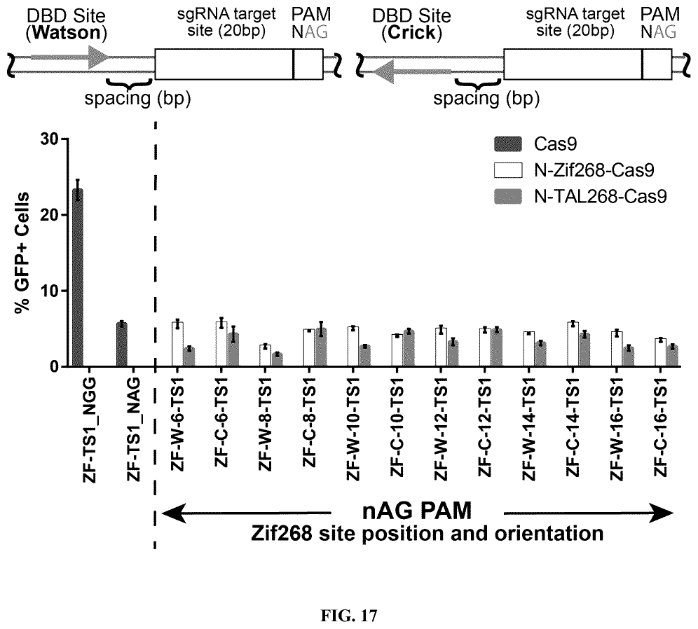

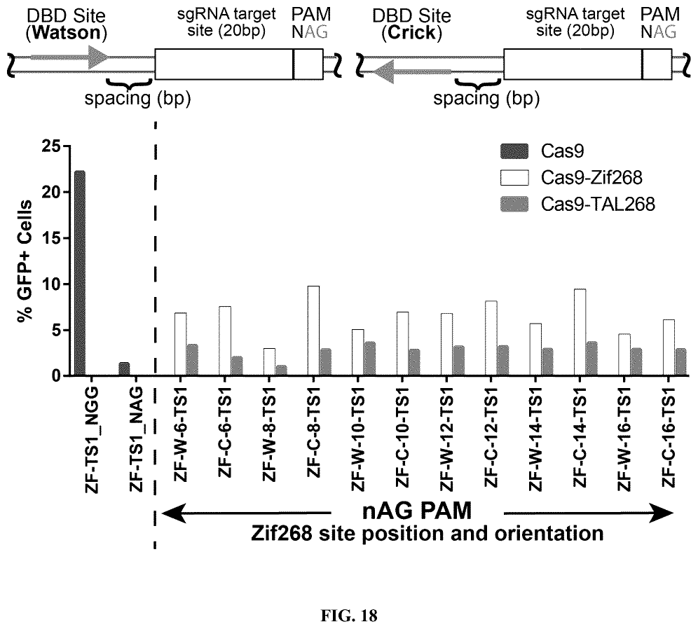

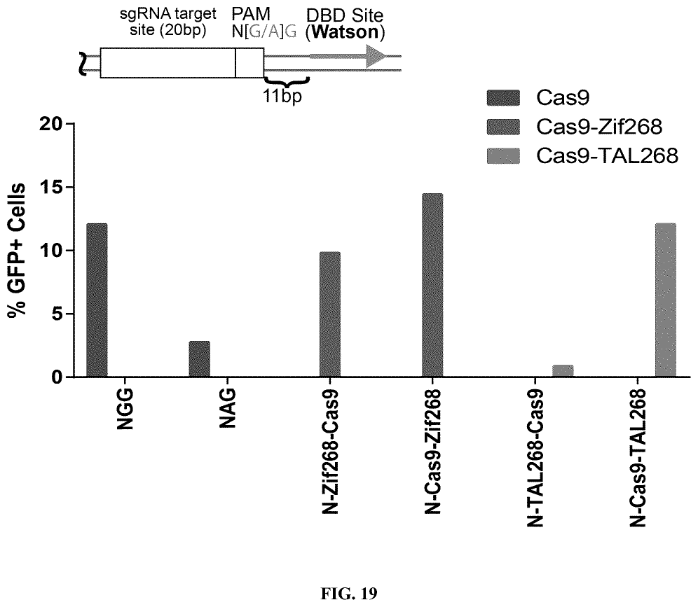

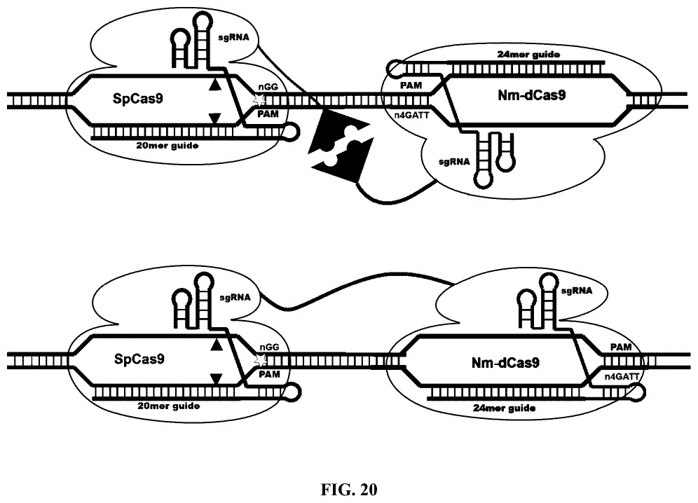

It was observed that C-terminal DBD fusions (e.g., SpCas9-Zif268) display superior activity to N-terminal fusions (FIG. 17, FIG. 18, FIG. 19). Consequently, the activity of SpCas9-Zif268 and SpCas9-TAL268 were further examined on a number of different arrangements of their binding sites to define optimal spacing and orientation for DBD recognition sites relative to a Cas9 target sequence for cleavage. A pDBD fusion (ZFP or TALE) to an SpCas9 may enhance nuclease activity when a pDBD binding sites are located at different positions and orientations relative to the Cas9 target site, FIG. 3. In preliminary experiments, the most robust activity was observed when using a C-terminal fusion of a ZFP or a TALE to SpCas9 and the pDBD binding sites were positioned 3′ to the PAM element (data not shown).

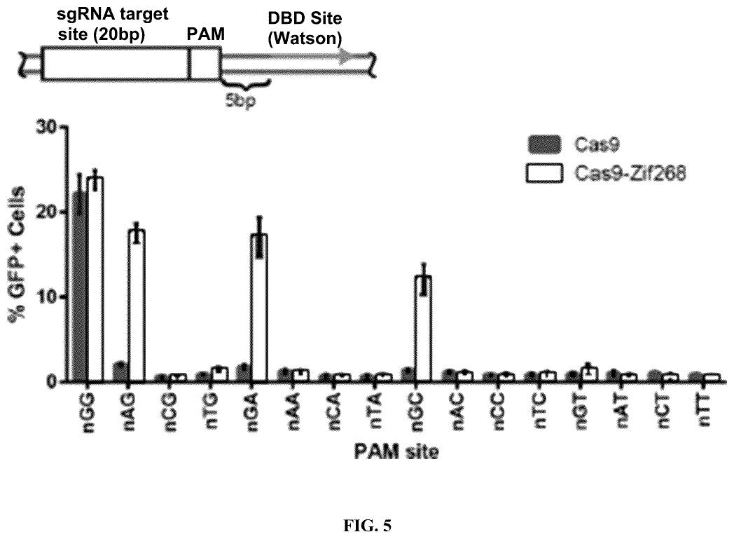

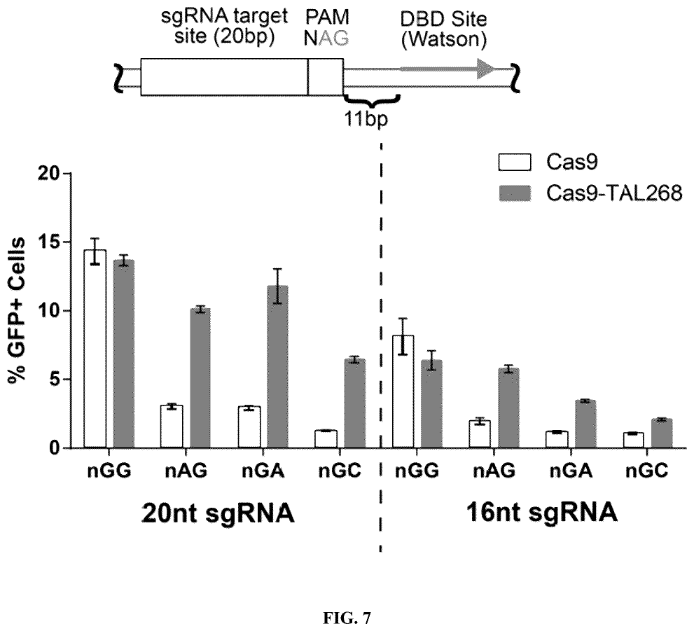

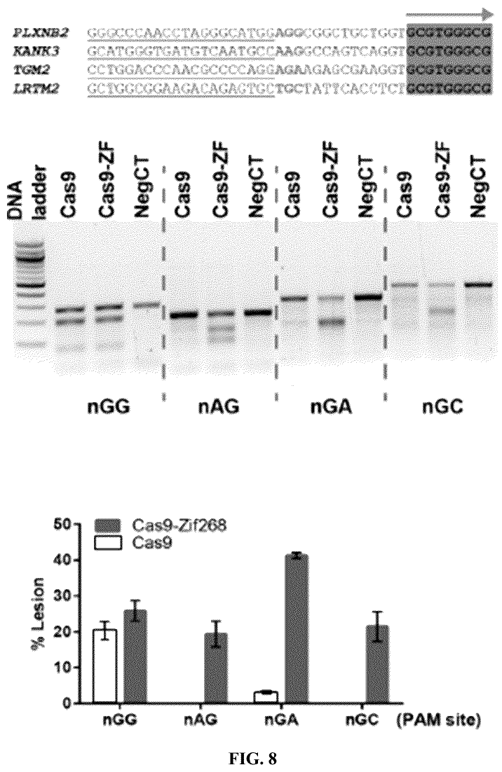

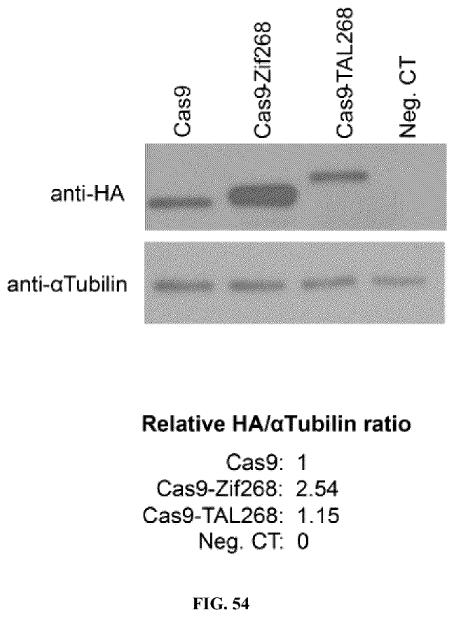

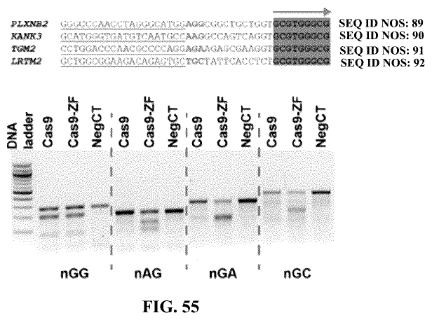

Both SpCas9-ZFP and SpCas9-TALE proteins can dramatically enhance nuclease activity on a nAG PAM target to a level comparable to wild-type SpCas9 (SpCas9wT) activity on a nGG PAM while being expressed at similar levels, FIG. 54. SpCas9-pDBD nuclease activity remains dependent on the length of the guide sequence, confirming that a chimera retains a guide-dependent licensing stage for sequence cleavage, FIG. 6. To define the functional PAM elements utilized by a SpCas9-pDBD, activity at each of the 16 possible sequence combinations was examined. In contrast to wild type SpCas9, SpCas9-pDBD displayed high activity for nAG, nGA, nGC as well as the standard nGG PAM, FIG. 5 and FIG. 7. Importantly, a more flexible PAM recognition of SpCas9-pDBDs was also observed at genomic target sites, FIG. 55 and FIG. 8. Accounting for reverse complements of the functional PAM elements, SpCas9-pDBD chimeras can recognize 7 of the 16 possible dinucleotide sequence combinations, which markedly increases the number of accessible target sites. Because of a smaller size of SpCas9-ZFPs relative to SpCas9-TALEs, ZFP chimeras have advantages for delivery by certain viral delivery systems⁴⁷. Consequently, SpCas9-ZFP are preferred chimeras for many embodiments disclosed herein.

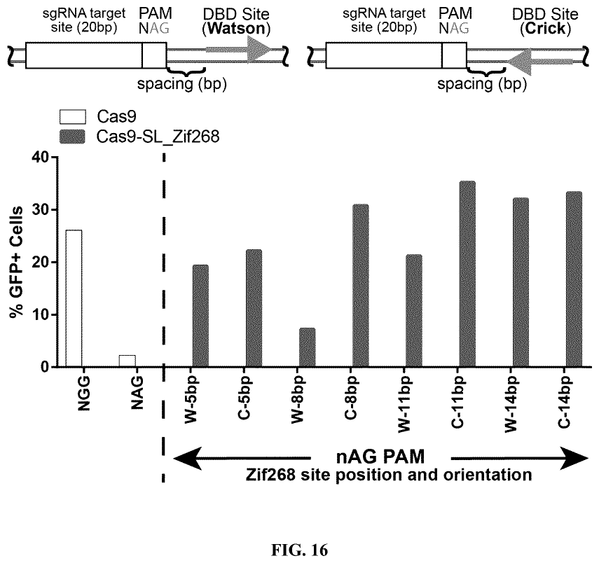

In one embodiment, a linker between a Cas9 nuclease and a DBD contains a plurality of amino acids (e.g., for example, approximately fifty-eight (58) amino acids) thereby providing good flexibility between the nuclease and the DBD. The data show that a standard SpCas9/sgRNA may be only functional with an NGG PAM, but not on an NAG PAM. SpCas9-Zif268 may be active on all spacings and orientations of the tested binding sites. SpCas9-TAL268 has a much more restricted spacing and orientation, but strong activity can nonetheless be observed. Shorter linkers (e.g., for example, approximately twenty-five (25) amino acids) between a Cas9 nuclease and Zif268 have also been evaluated which provide a more restricted spacing between the nuclease and the DBD (FIG. 16). When fused to a DTU (e.g., for example, Zif268) Cas9 nuclease activity may be still dependent on the sgRNA, as demonstrated by determining Cas9 activity in a GFP reporter assay using truncated sgRNAs (FIG. 6). The activity profile of Cas9 and Cas9-Zif268 on a target site with a neighboring Zif268 binding site and an NGG PAM may be similar. Interestingly, Cas9-DTU fusions may be able to tolerate truncations of the sgRNA to 16 nucleotides within the guide segment, which has not been demonstrated previously. This may allow further improvements in precision with a truncated guide RNAs. Fu et al., Improving CRISPR-Cas nuclease specificity using truncated guide RNAs. Nat Biotechnol. 2014 March; 32(3):279-84.

SpCas9 and SpCas9-Zif268 were tested on all sixteen (16) possible NXX PAM combinations to define the breadth of sequences that can be targeted. It was found that NGG, NGA, NAG, and NGC PAMs have very similar activity for SpCas9-Zif268 in the presence of a neighboring Zif268 target site, whereas SpCas9 only cleaved NGG PAM efficiently, FIG. 5. This extension in the activity of alternate PAM specificities was also observed for SpCas9-TAL268 (FIG. 7). This extended activity profile was recapitulated for endogenous (i.e., not reporter) genomic target sites with suboptimal PAMs. FIG. 8. These data demonstrate that SpCas9-DBD fusions have increased activity and broader targeting range than a standard Cas9 system. Given that Cas9 fusion proteins contemplated by the present invention can target 7 of the 16 potential NXX or XXN permutations (e.g., CCN, TCN & CTN by targeting the opposite strand), and that TALEs can be programmed to recognize any sequence, SpCas9-TALEs can be programmed for cleavage of sites that occur roughly every other base pair within the genome. Lamb et al., Directed evolution of the TALE N-terminal domain for recognition of all 5′ bases. Nucleic Acids Research. 2013 November; 41(21):9779-85.

### B. Attenuated Cas9 Platforms

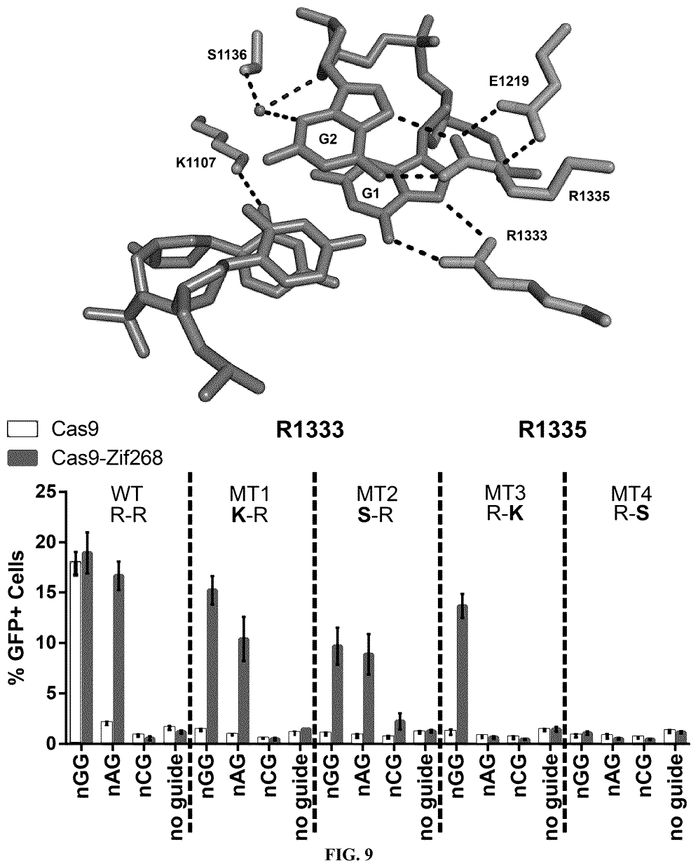

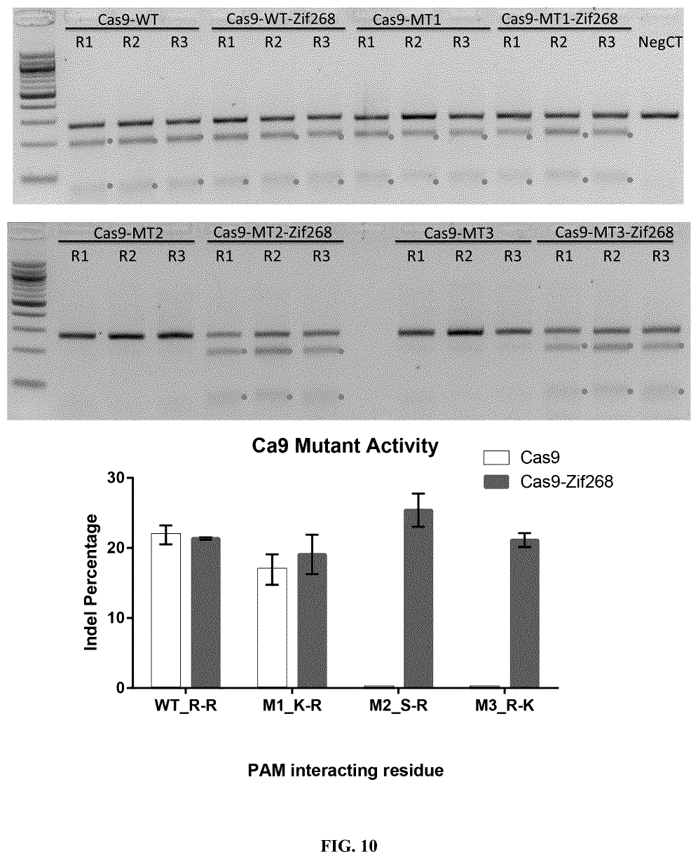

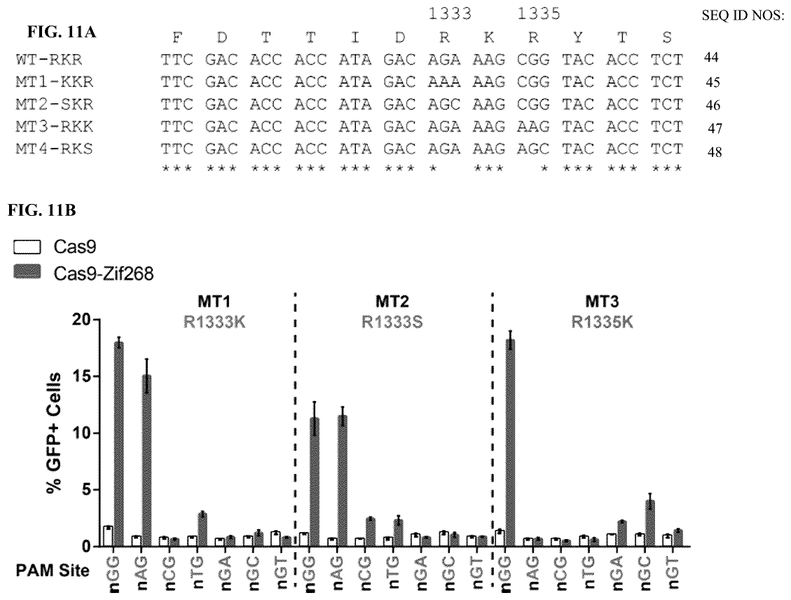

In one embodiment, the present invention contemplates an attenuated SpCas9 comprising a mutated PAM recognition sequence, wherein an SpCas9 has a reduced affinity for a specific target sequence (Cas9^{MT} protein). Based on the structure of a SpCas9/sgRNA/target complex and conservation in phylogenetically neighboring Cas9 orthologs, two arginines involved in PAM recognition (R¹³³³ and R¹³³⁵) were identified as mutation targets (FIG. 9). Nishimasu et al., Crystal structure of Cas9 in complex with guide RNA and target DNA. Cell. 2014 Feb. 27; 156(5):935-49; and Anders et al., Structural basis of PAM-dependent target DNA recognition by the Cas9 endonuclease. Nature. 2014 Sep. 25; 513(7519):569-73. The impact of two different mutations at each site (R mutated to K or S) was examined using an SpCas9 and SpCas9-DBD GFP reporter activity assay. The data show that each mutation dramatically attenuates SpCas9 activity. Surprisingly, however, three of the four mutations regained functionality when incorporated into a SpCas9-DBD fusion protein. FIG. 9. On endogenous targets in HEK293T cells, two of these mutations appear to drastically inactivate SpCas9, whereas they remain fully functional as SpCas9-Zif268 fusion proteins (FIG. 10). Notably, mutations to SpCas9 PAM recognition residues can also yield SpCas9^{MT}-DBD nucleases with altered PAM preferences. The R1335K mutant (MT3) displays a strong preference for GG over AG at this target, unlike the original SpCas9 (FIG. 11). These results suggest that SpCas9 PAM specificity can be refined or potentially even altered, but that a fused DBD may be necessary to unmask this behavior.

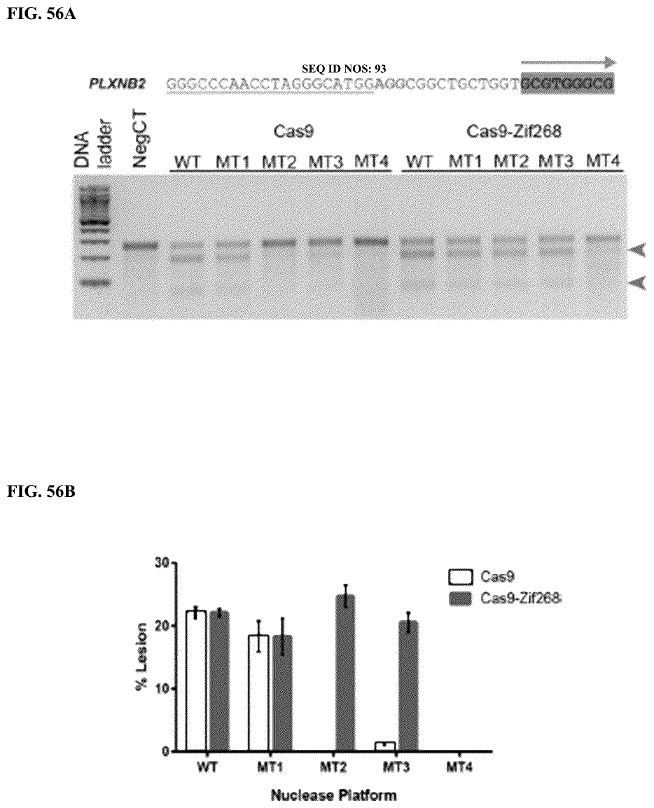

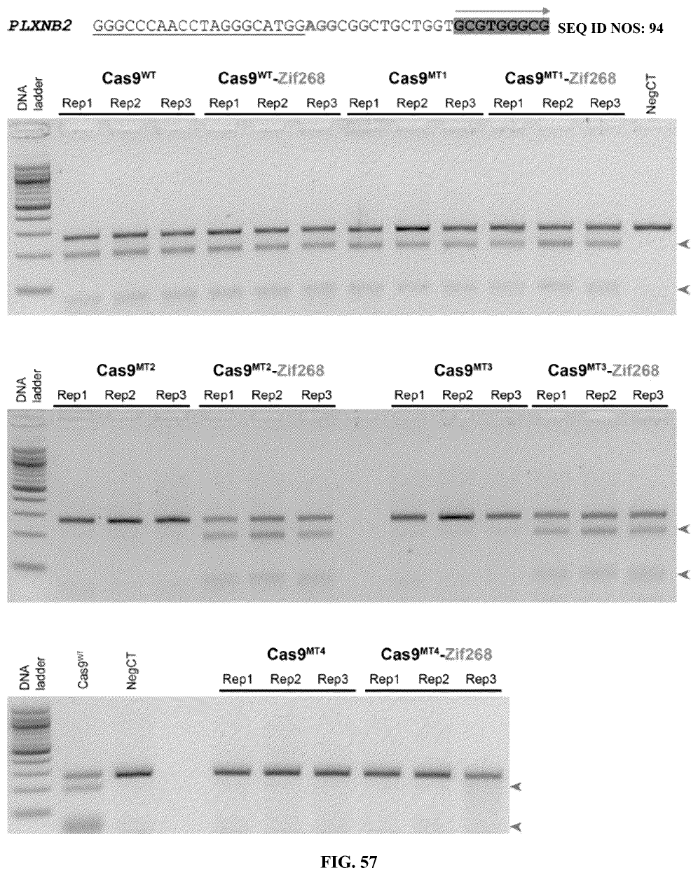

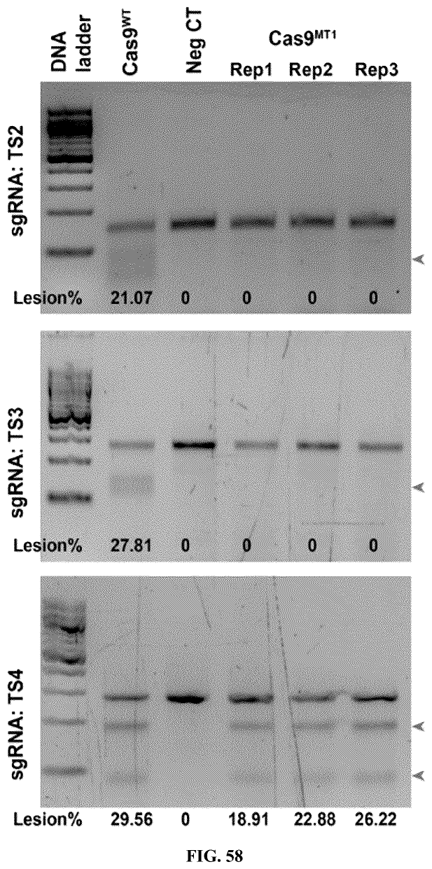

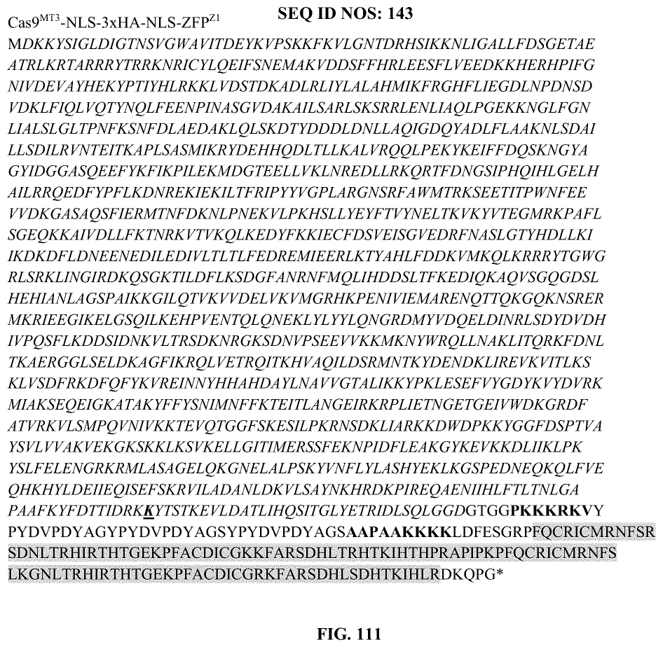

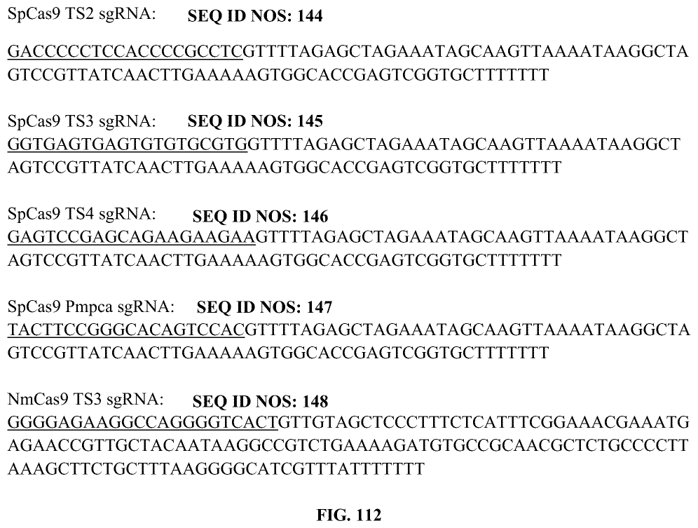

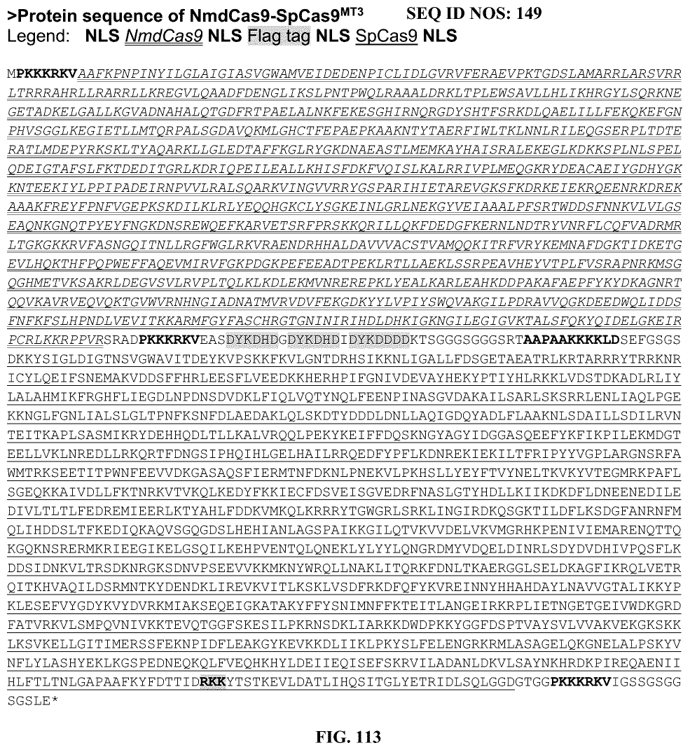

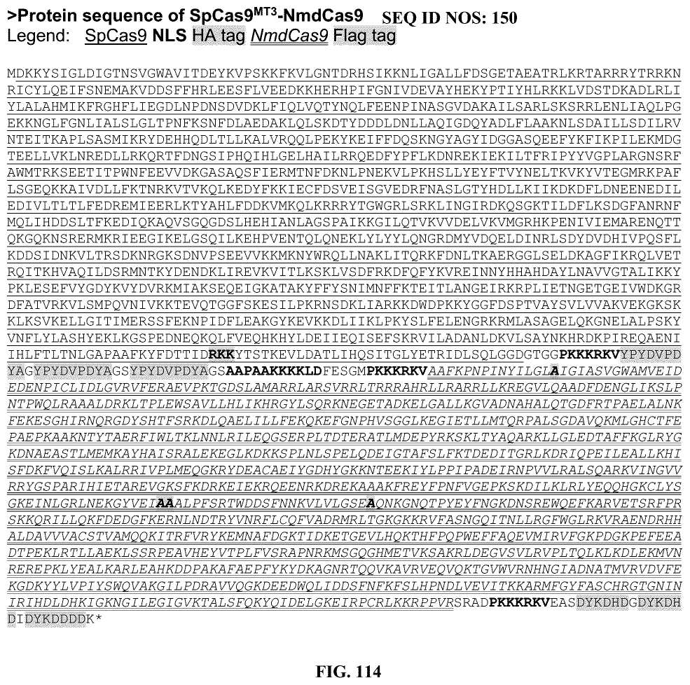

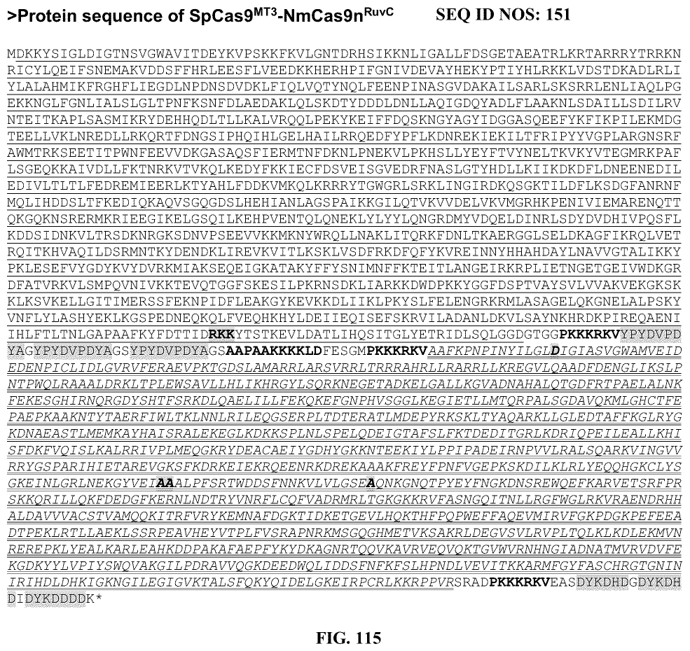

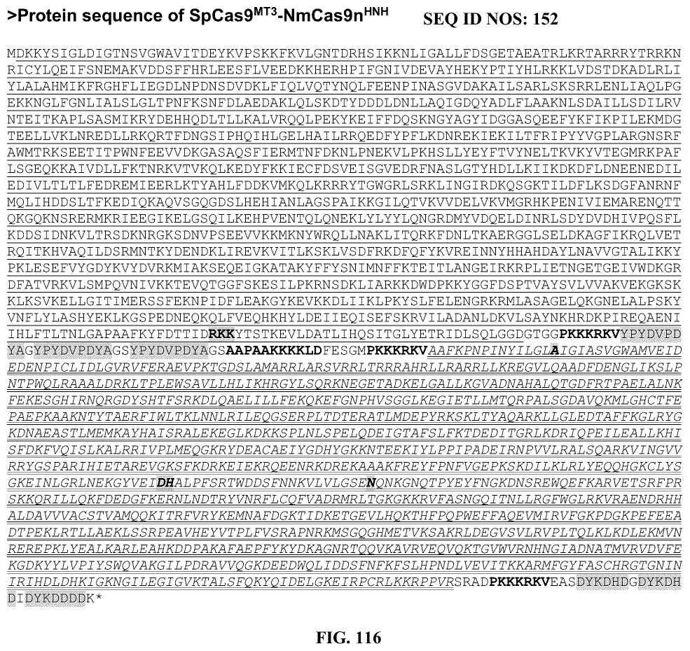

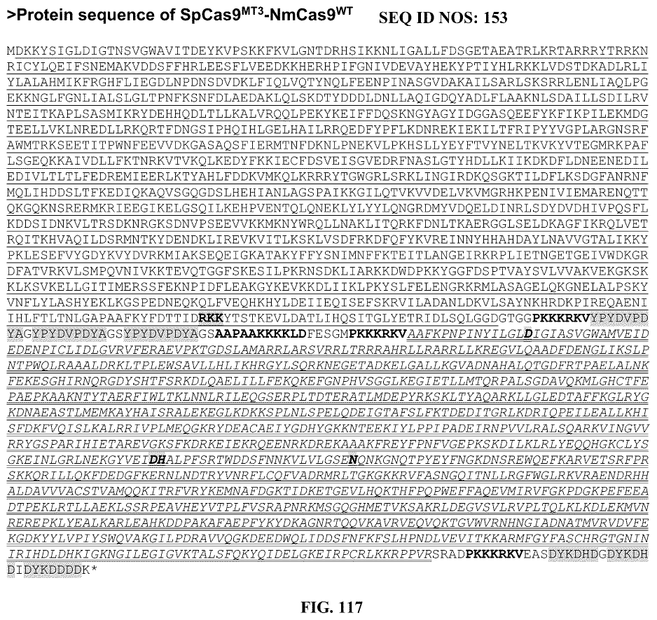

The fusion of a pDBD to SpCas9 should increase nuclease precision if target cleavage is dependent on DNA recognition by the pDBD. To achieve this, DNA-binding affinity of SpCas9 was attenuated by independently mutating the key PAM recognition residues (Arg1333 and Arg1335)⁷ to either Lysine or Serine, FIG. 9A and FIG. 11A. In a plasmid reporter assay, all four of these mutations reduced the nuclease activity of wild type SpCas9 to background levels; a fused ZFP domain and complementary target site restored nuclease activity in all mutants except R1335S (SpCas9^{MT4}), FIG. 9B. It was found that R1335K (SpCas9^{MT3}) was not functional with the nAG PAM even as a SpCas9-ZFP fusion. A broader evaluation of PAM specificity of the three active SpCas9-ZFP mutants determined a weak activity at alternate PAMs that retained an unaltered arginine-guanine interaction⁷ (i.e. R1333 mutants prefer nnG PAMs, whereas the R1335K mutant prefers nGn PAMs), FIG. 111B. Activity of each SpCas9 mutant was also determined independently, or as a ZFP fusion, on a compatible genomic target site with an nGG PAM, FIG. 56A&B, FIG. 57 and FIG. 58.

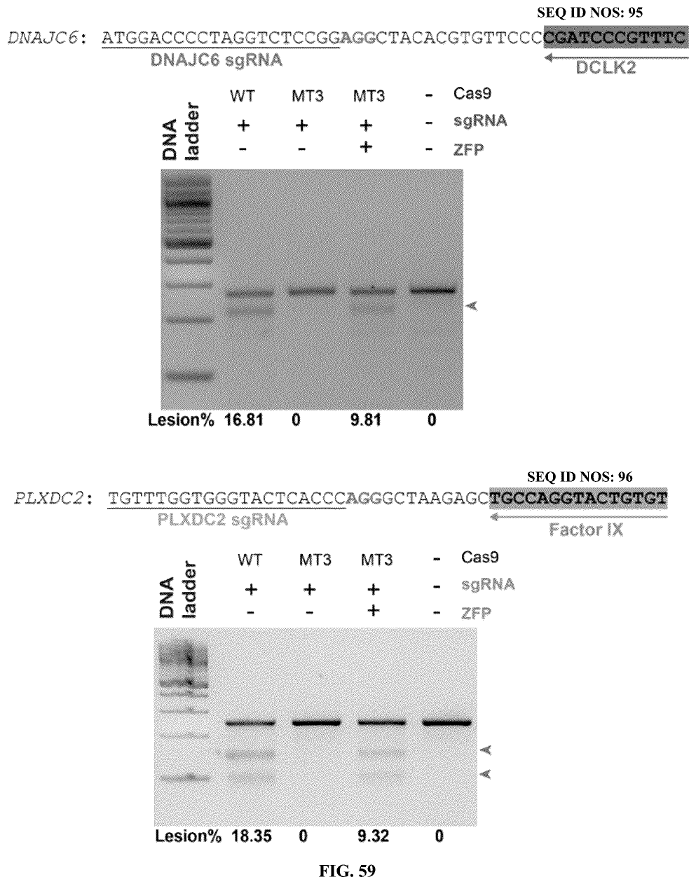

R1333K (SpCas9^{MT1}) retained independent activity on a subset of target sequences, whereas R1333S (SpCas9^{MT2}) and R1335K (SpCas9^{MT3}) display only background activity, which could be restored to wild type levels in the presence of a ZFP fusion. To confirm that the ZFP-dependent restoration of activity is general, the nuclease activity of three additional SpCas9^{MT3}-ZFP fusions were assessed, two of which restore nuclease function, FIG. 59 and Table 1.

TABLE 1
Summary of SpCas9^{MT3}-pDBD nuclease activities (T7EI)

| SEQ ID | | | | | Activity |
|---|---|---|---|---|---|
| NO: | pDBD Name | Type | Target Sequence | sgRNA | (%Lesion) |
| 4 | ZFP^{TS2} | 4 Finger ZGP | GCGGGCAGGGGC | TS2 | 36.64 |
| 5 | ZFP^{TS2} | 4 Finger ZGP | GCAGGGGCCGGA | TS2 | 23.04 |
| 6 | ZFP^{TS3} | 4 Finger ZGP | GGCGTTGGAGCG | TS3 | 26.75 |
| 7 | ZFP^{TS4} | 4 Finger ZGP | CCGGTTGATGTG | TS4 | 12.86 |
| 8 | Zif268 | 3 Finger ZGP | GCGTGGGCG | PLXNB2 | 26.81 |
| 9 | ZFP | 4 Finger ZGP | GAAACGGGATCG | DNAJC6 | 9.32 |
| 10 | ZFP^{FactorIX} | 5 Finger ZGP | ACACAGTACCTGGCA | PLXDC2 | 9.90 |
| 11 | ZFP^{HEBP2} | 4 Finger ZGP | GAAAAGTATCAA | GPRC5B | N.D |
| 12 | TAL268 | 8.5 Module Tale | TGCGTGGGCG | PLXNB2 | N.D |
| 13 | TALE^{TS3-S} | 9.6 Module Tale | TTGGAGCGGGG | TS3 | 8.00 |
| 14 | TALE^{TS3-L} | 16.6 Module Tale | TTGGAGCGGGGAGAAGG | TS3 | 16.26 |
| 15 | TALE^{TS4-S} | 9.5 Module Tale | TCAACCGGTGG | TS4 | 2.01** |
| 16 | TALE^{TS4-L} | 19.6 Module Tale | TCAACCGGTGGCGCATT | TS4 | 1.82** |

N.D: Not Detected
**: Not above background independent activity for SpCas9^{MT3}.

Thus, altering an affinity of Cas9 PAM recognition domains through mutation generates SpCas9 variants that are dependent on an attached pDBD for efficient function. This dependence on an attached pDBD establishes a third stage of target site licensing for the presently disclosed SpCas9MT3-pDBDs, which are observed to increase their precision.

To evaluate precision of an SpCas9MT-DBD fusion protein, validated SpCas9 target sites were tested (e.g., TS2, TS3 & TS4; all with NGG PAMs). Fu et al., Improving CRISPR-Cas nuclease specificity using truncated guide RNAs. Nature Biotechnology. 2014 March; 32(3):279-84; Fu, Y., Foden, J. A., Khayter, C., Maeder, M. L., Reyon, D., Joung, J. K., & Sander, J. D. (2013). High-frequency off-target mutagenesis induced by CRISPR-Cas nucleases in human cells. Nature Biotechnology, 31(9), 822-826. doi:10.1038/nbt.2623. SgRNAs that recognize these sites have well-defined on- and off-target activities, and thus provide a benchmark to rapidly assess improvements in precision by evaluating activity at high-efficiency off-target sites.

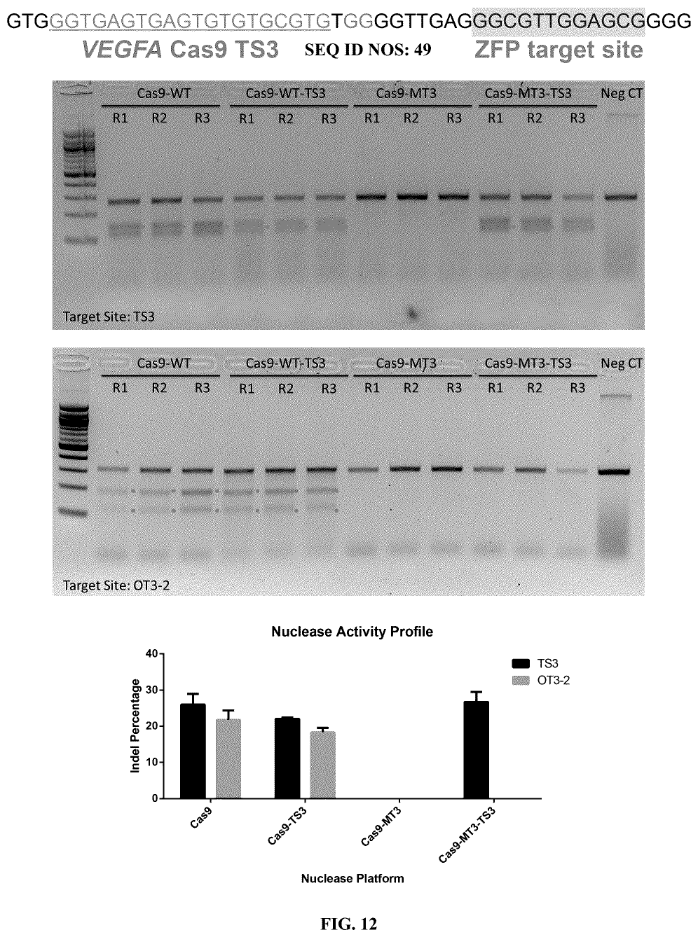

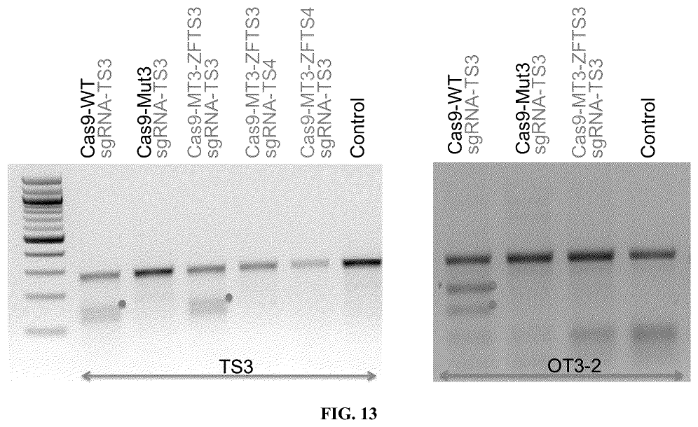

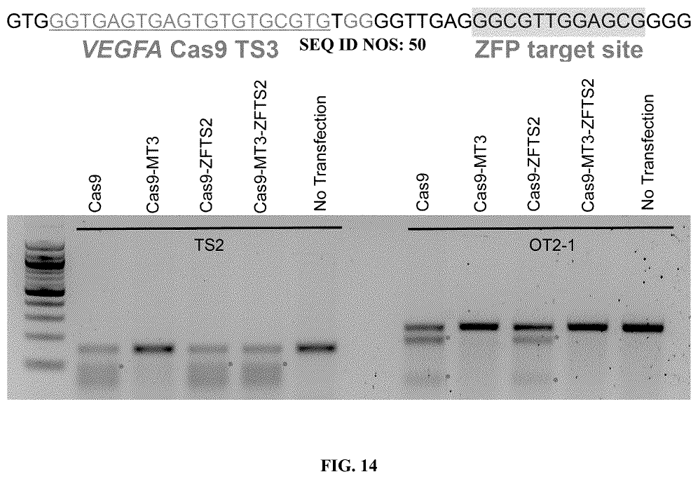

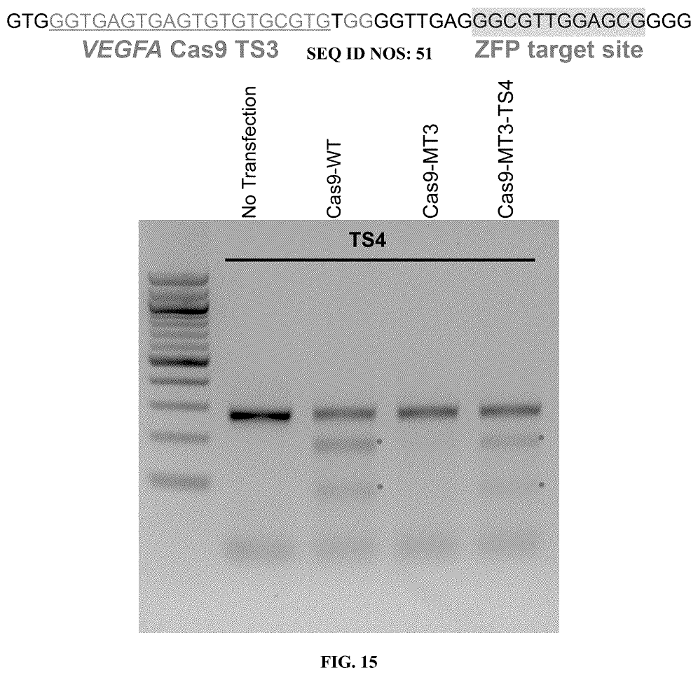

A ZFP DBD (i.e., for example, ZFP^{TS3}) was designed to recognize a sequence near a TS3 target site (FIG. 12) and the editing activities were compared of TS3 sgRNA-programmed SpCas9, SpCas9^{MT3} adSC9^{MT3}-ZFP^{TS3} at the target site and a highly active off-target site (OT3-2), FIG. 10. The data confirms that a standard SpCas9 was highly active at both sites, and that SpCas9^{MT3} was inactive at both sites. Remarkably, SpCas9^{MT3}-ZFP^{TS3} displays high activity only at the target site (TS3), and appears inactive at OT3-2. Cleavage of a target site may be still sgRNA-dependent, as a non-cognate guide (sgRNA-TS4) fails to drive TS3 cleavage (FIG. 13). Likewise, a non-cognate ZFP (i.e., for example, ZFP^{TS4}) fails to target TS3 when fused to SpCas9^{MT3} loaded with a TS3-targeting sgRNA (FIG. 13). These data are in comparison with other data showing OT3-2 cleavage with standard SpCas9 even with a specificity-enhancing tru-sgRNA. Fu et al., Improving CRISPR-Cas nuclease specificity using truncated guide RNAs. Nature Biotechnology. 2014 March; 32(3):279-84. Thus, SpCas9^{MT} editing activity may be abrogated, but can be regained in a highly specificity-enhanced fashion when constructed as a fusion protein with a programmable DBD, as contemplated by the invention herein. Similar ability to selectively target Cas9^{MT3} to function at TS2 (FIG. 14) and TS4 (FIG. 15) using an attached zinc finger protein has been achieved.

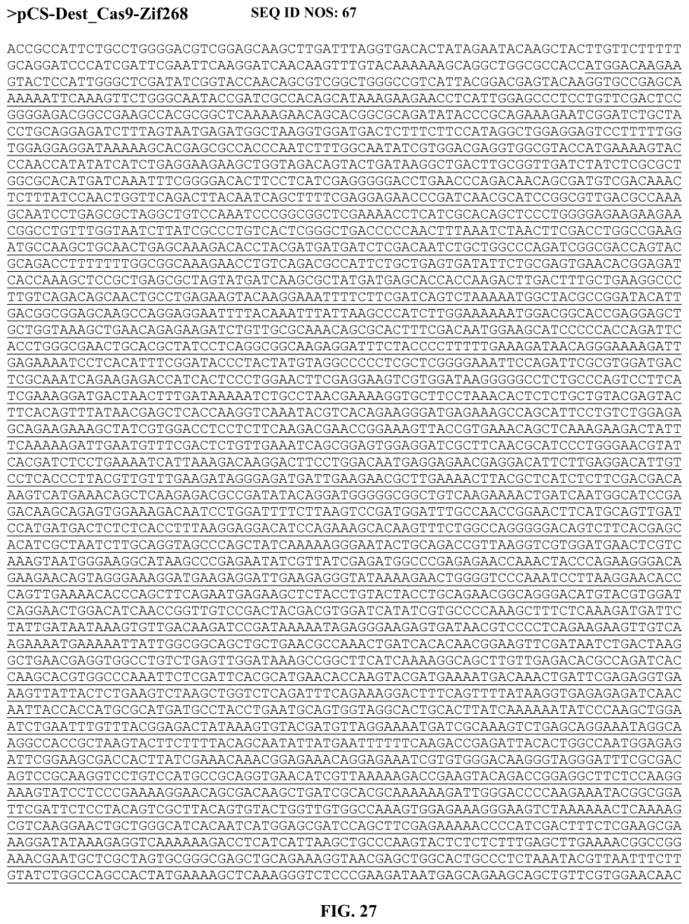

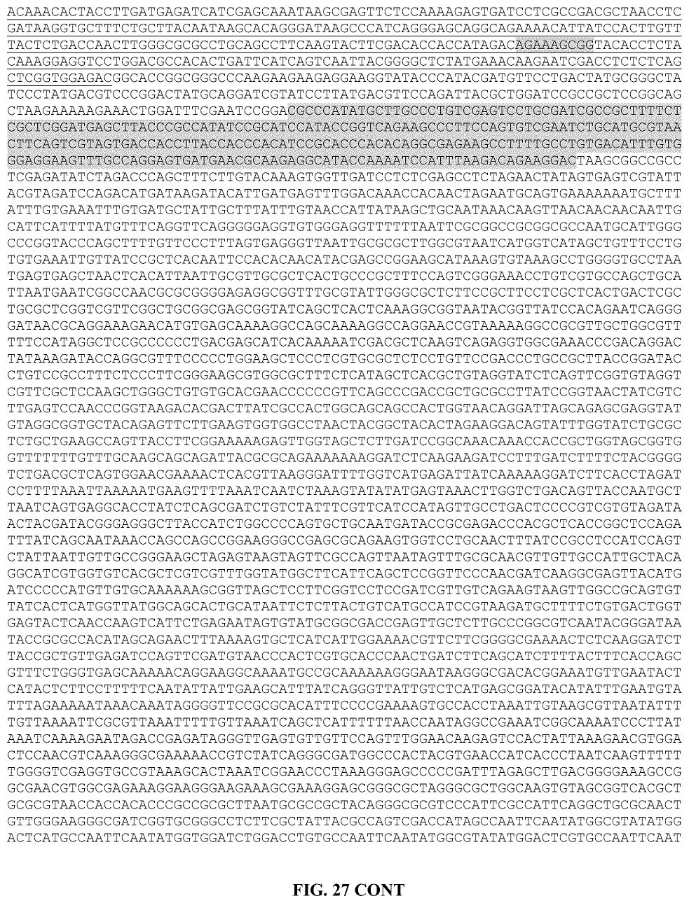

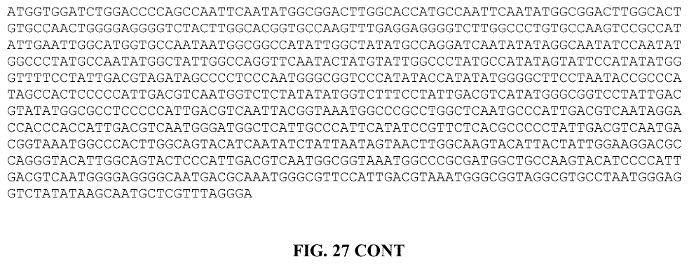

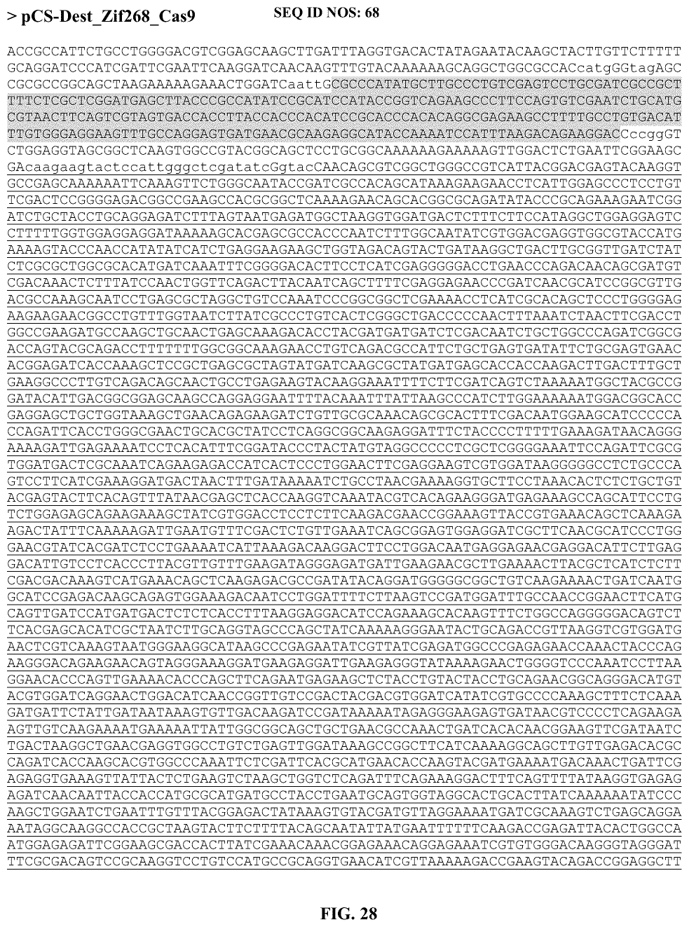

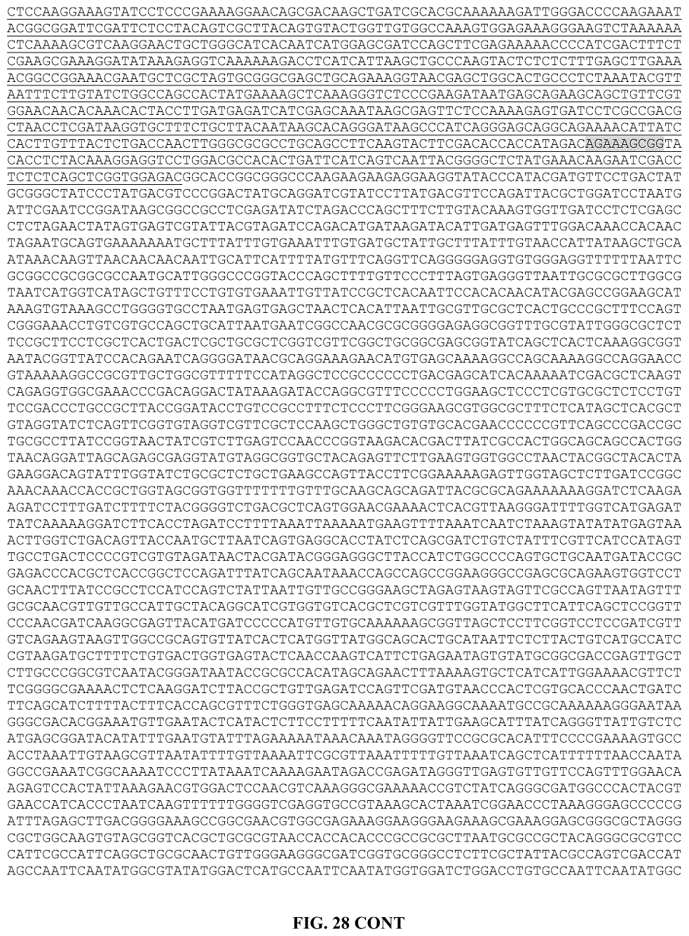

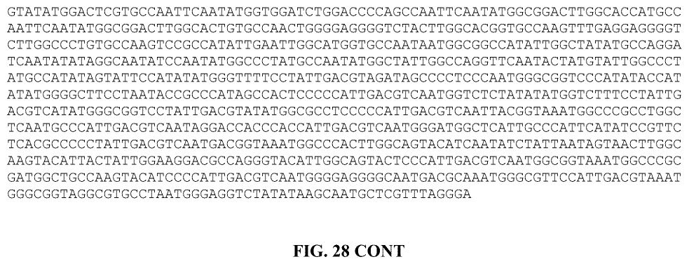

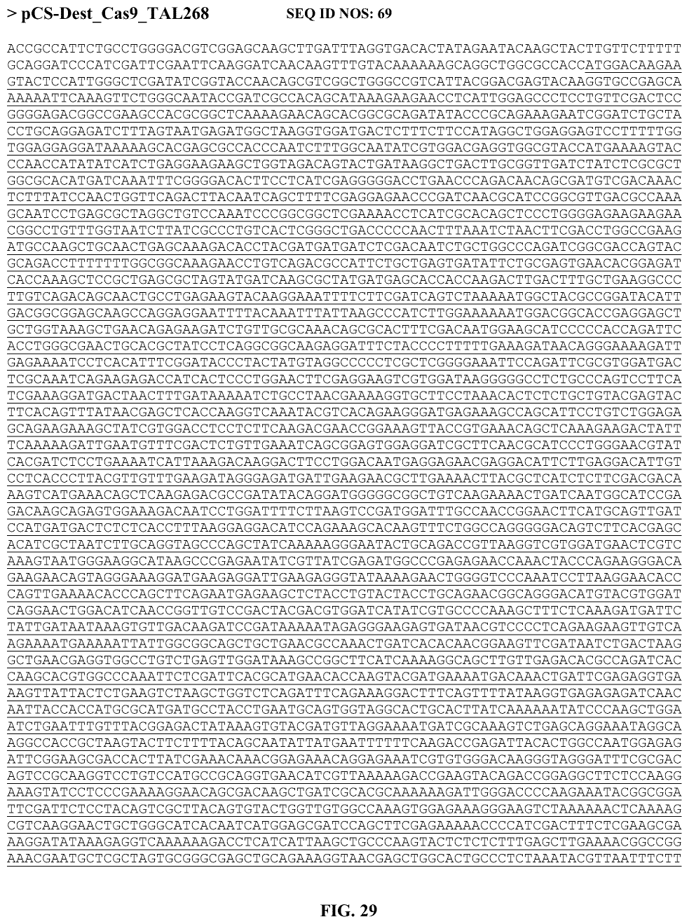

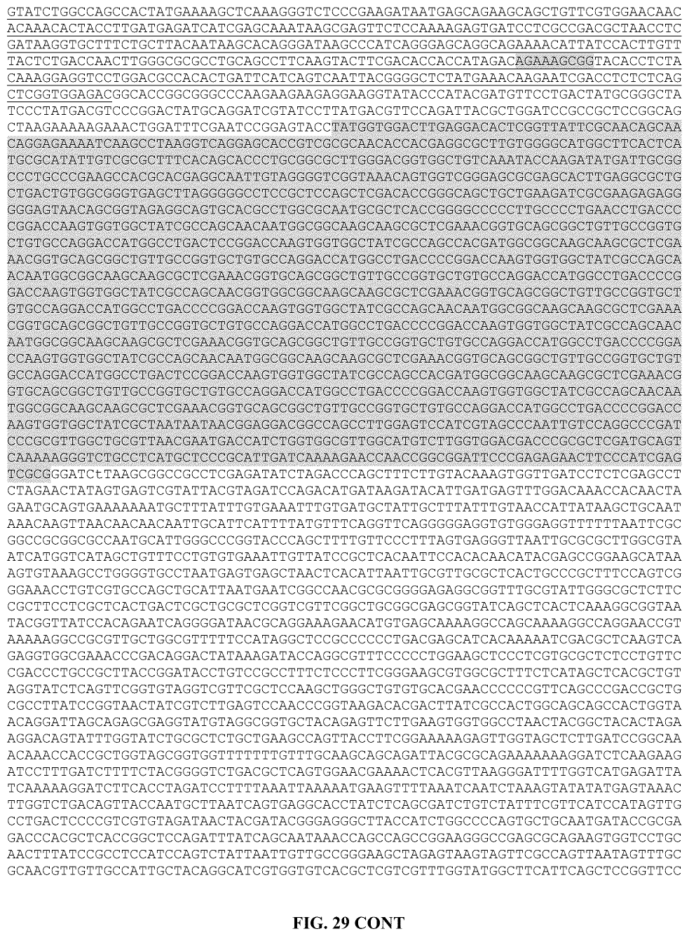

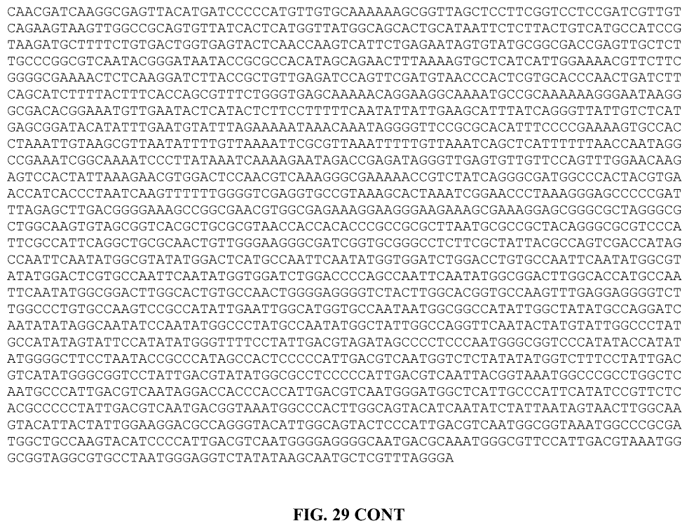

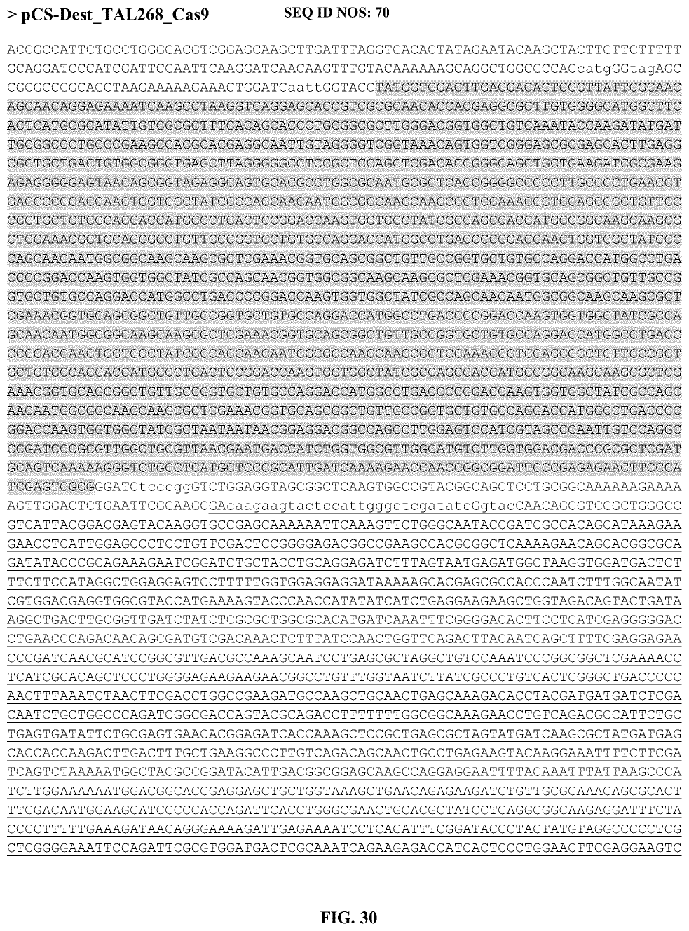

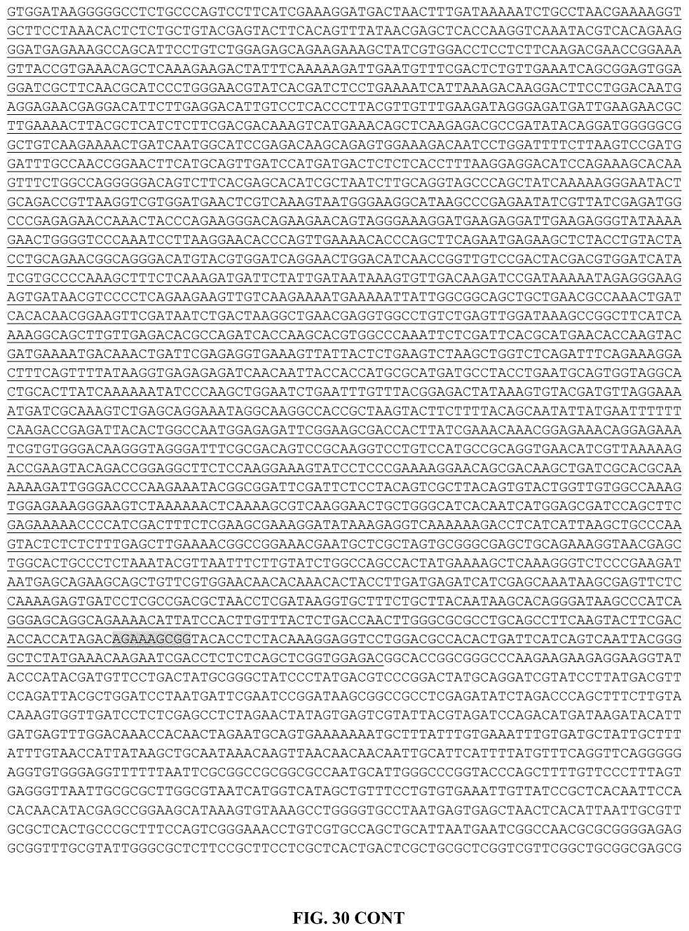

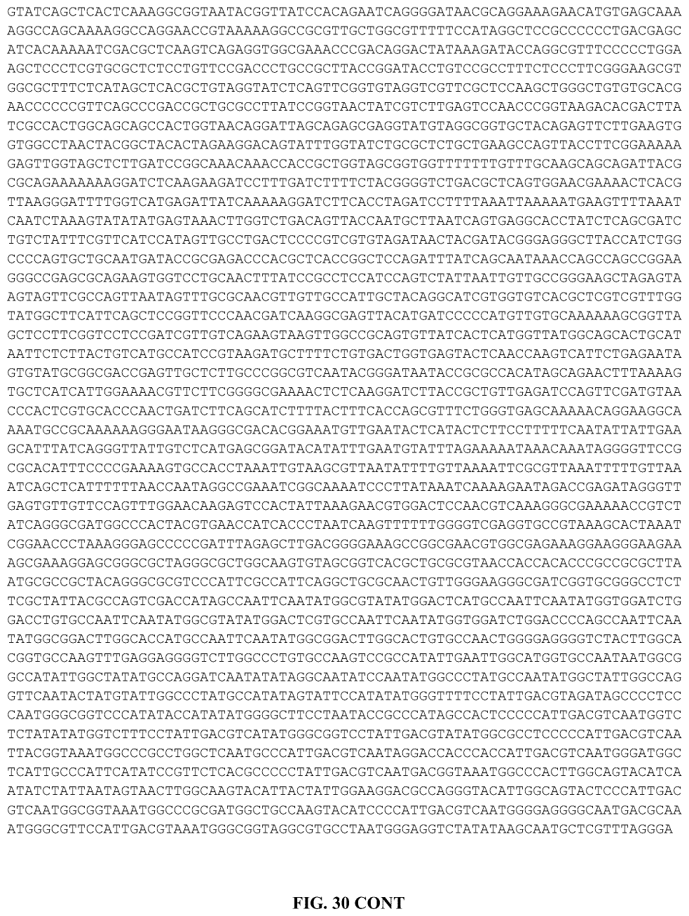

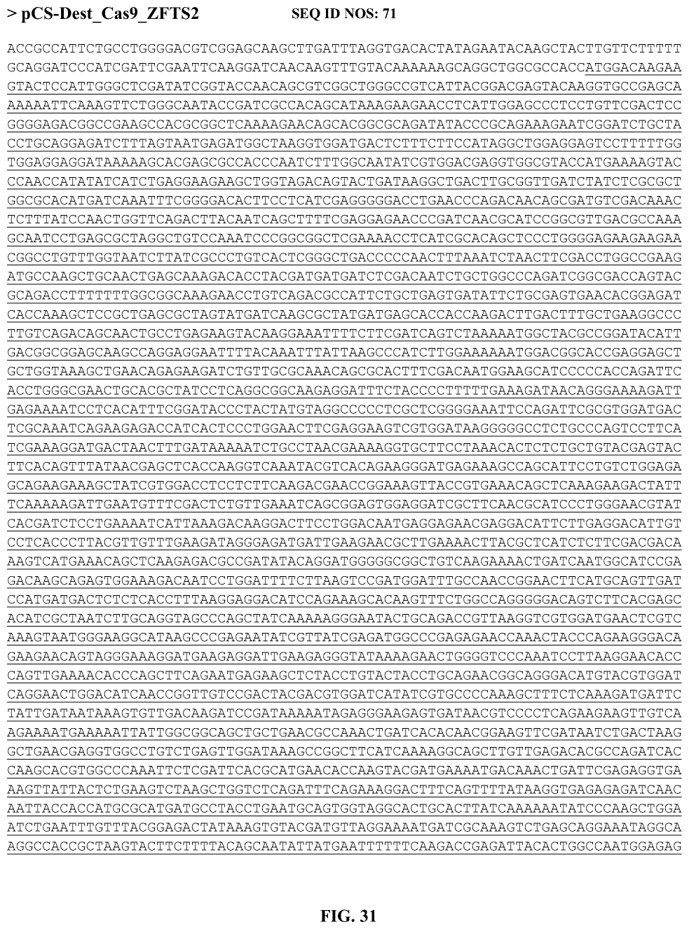

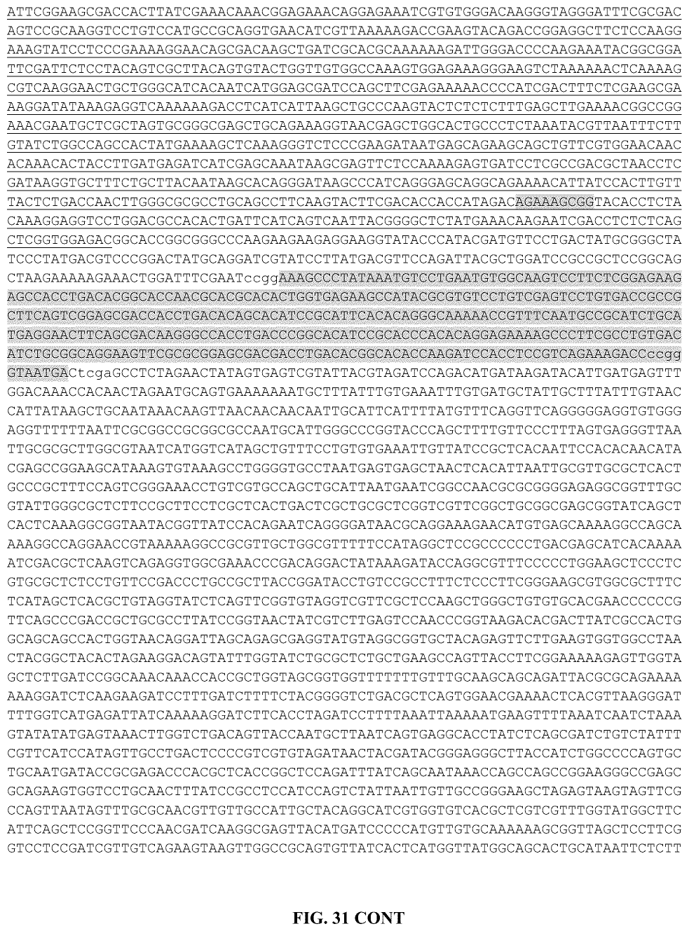

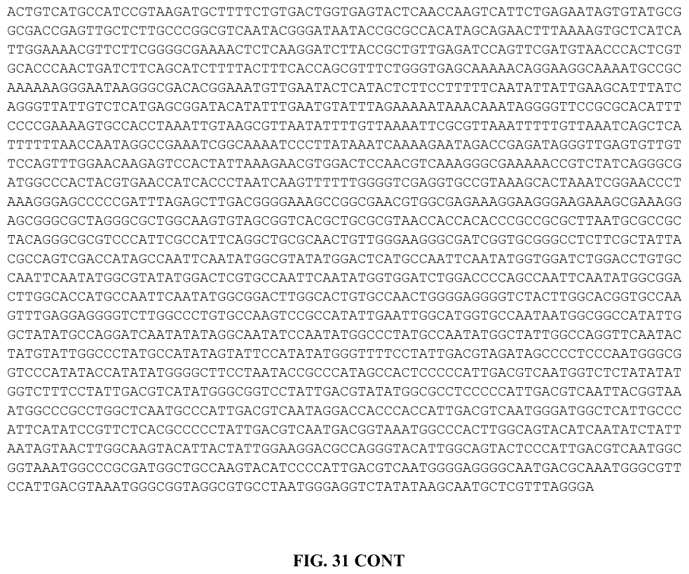

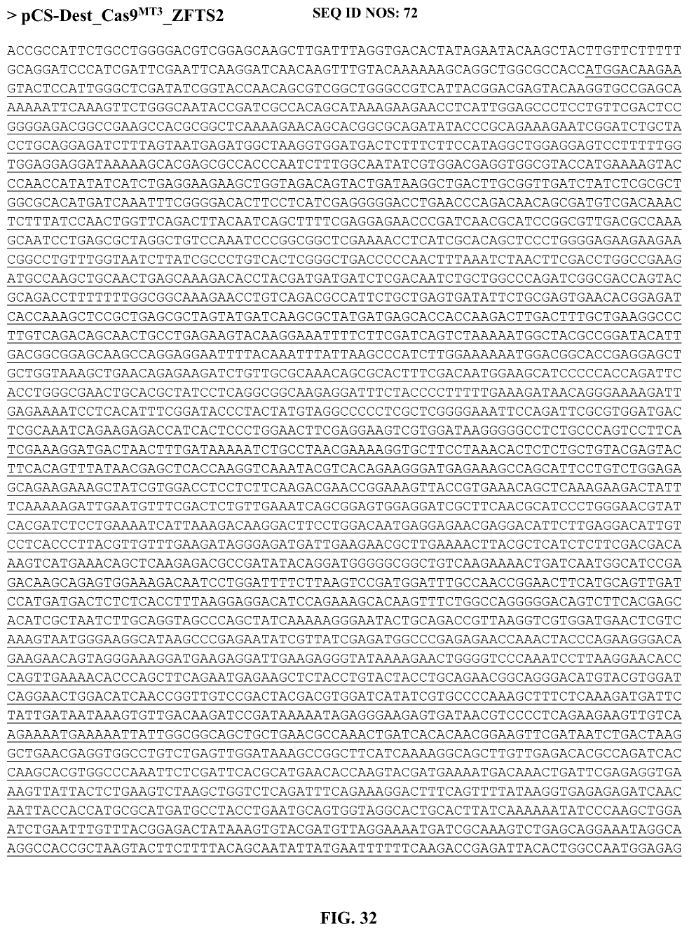

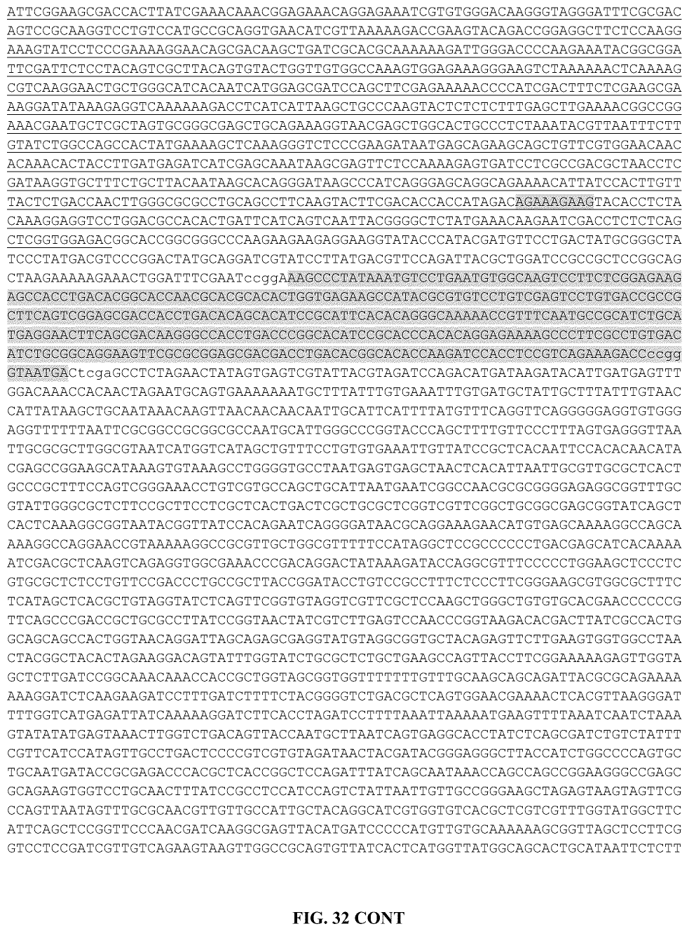

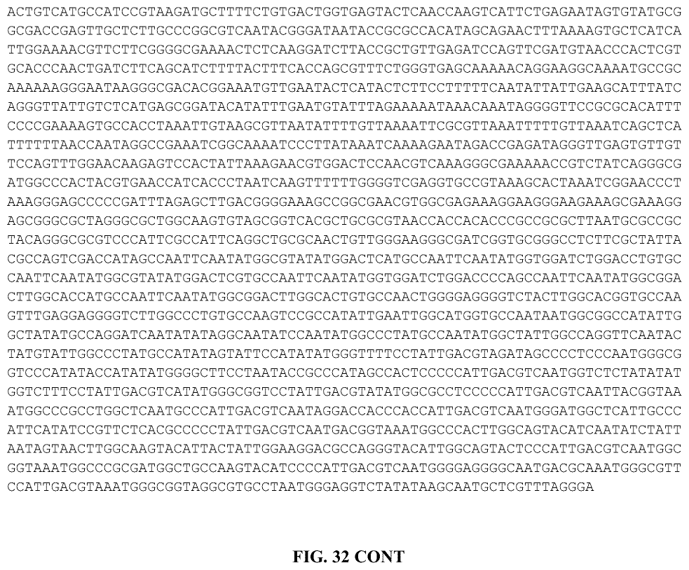

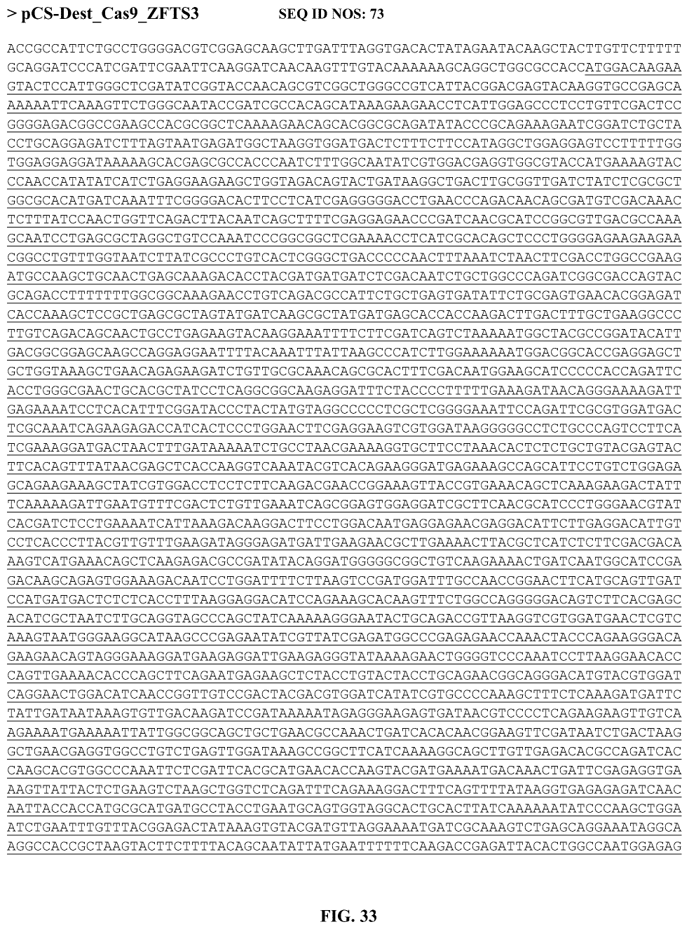

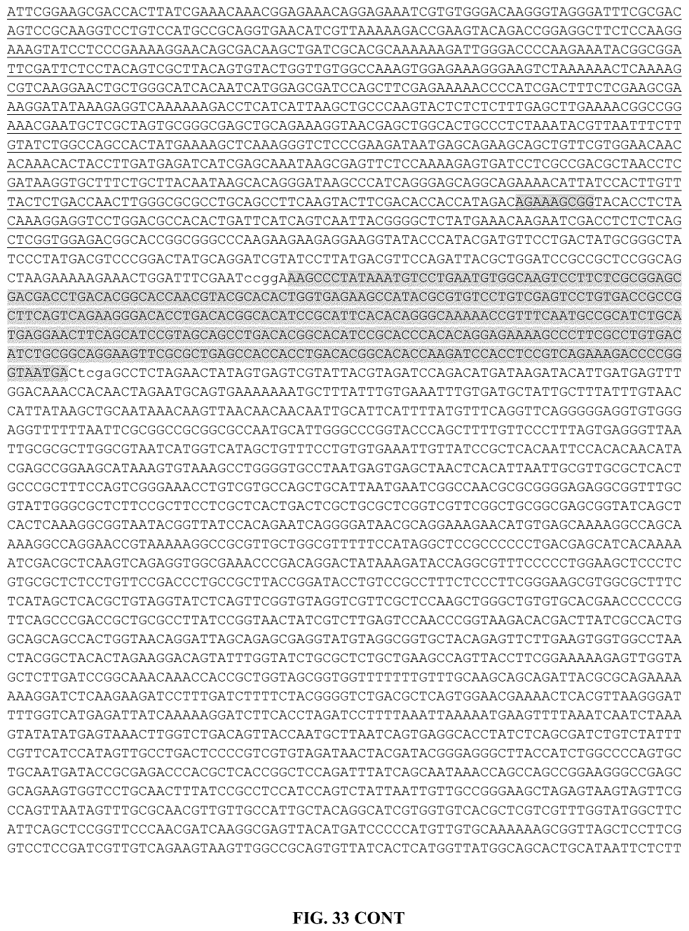

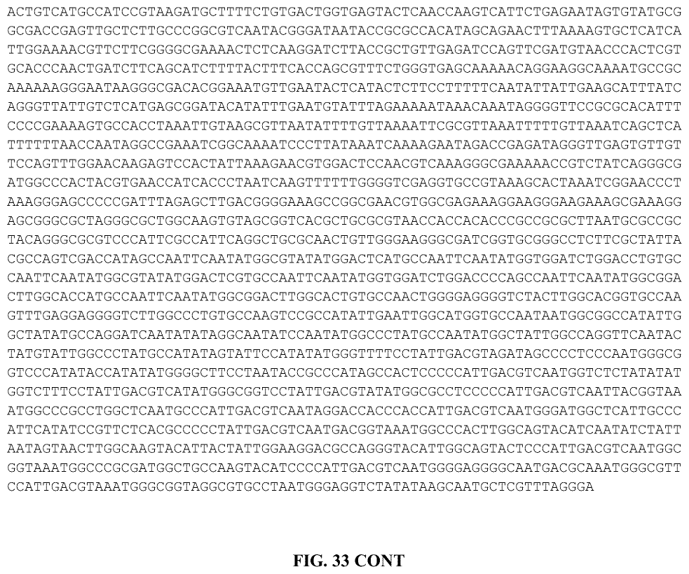

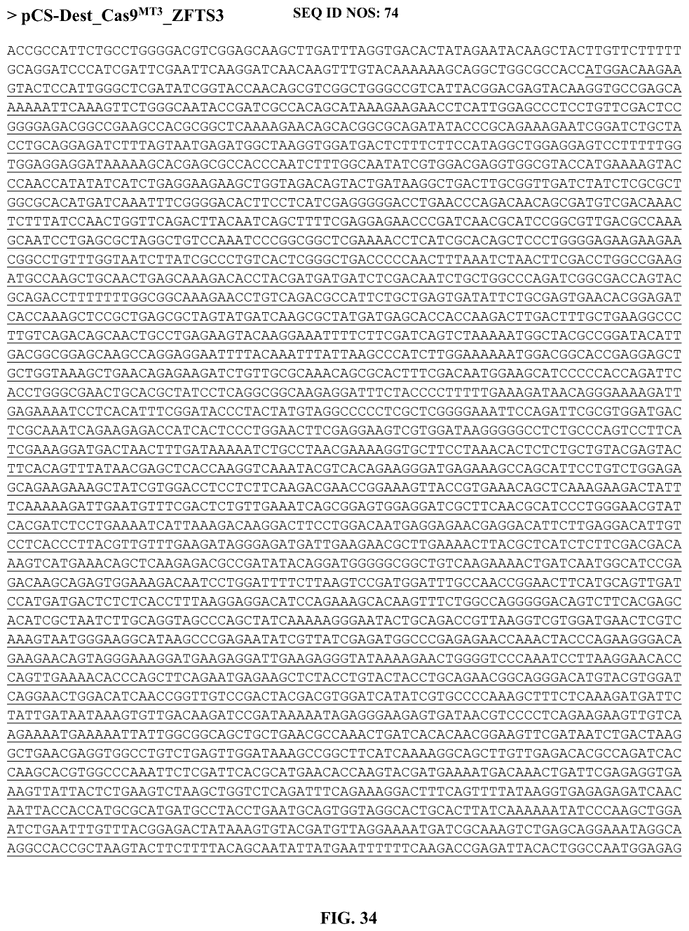

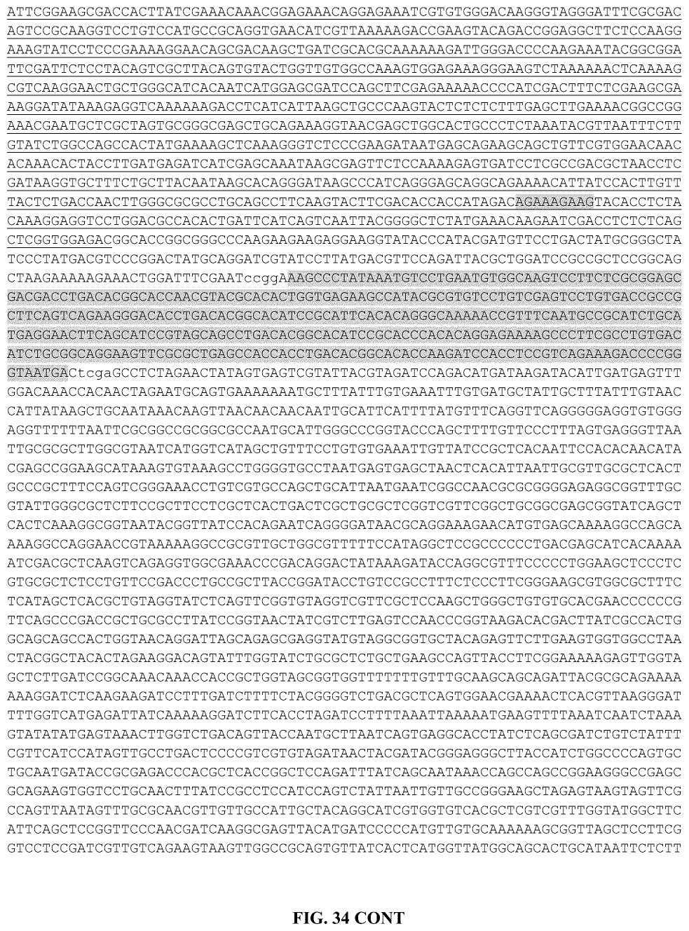

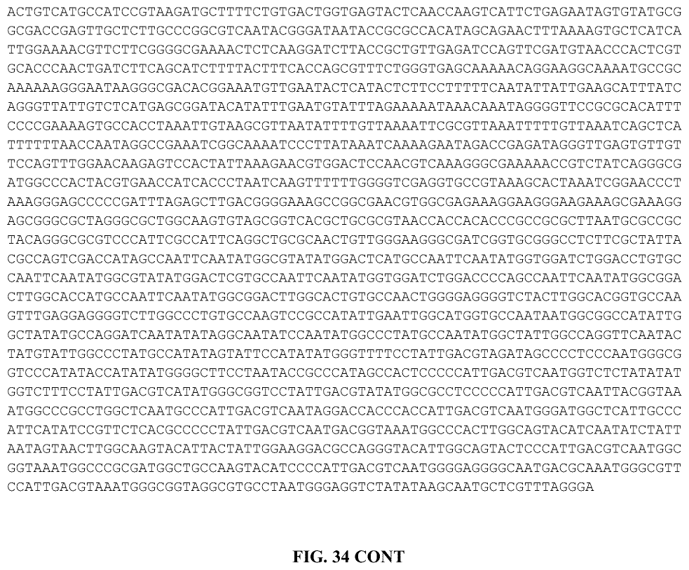

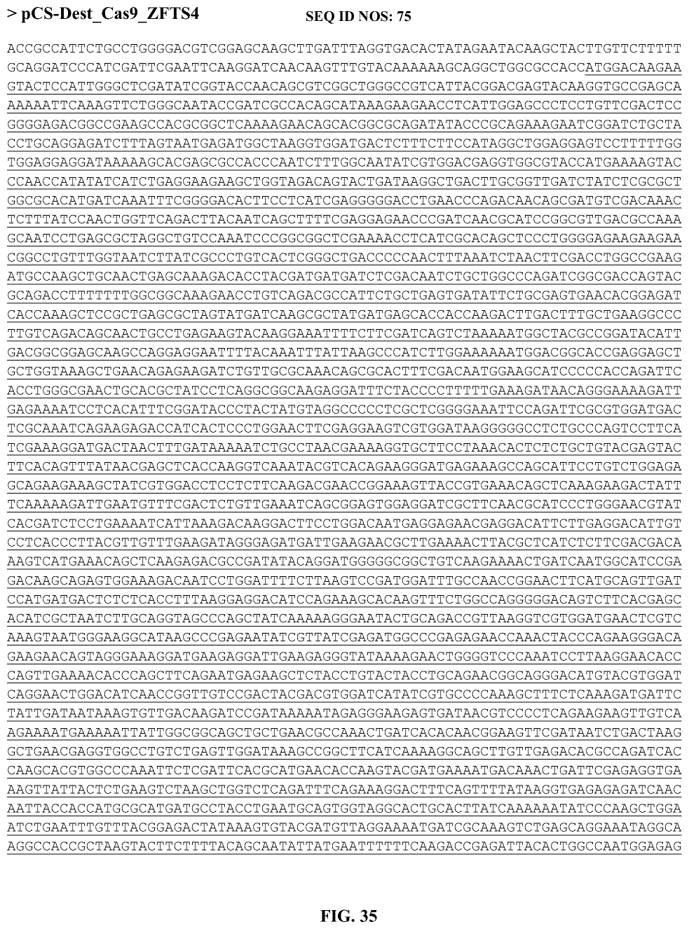

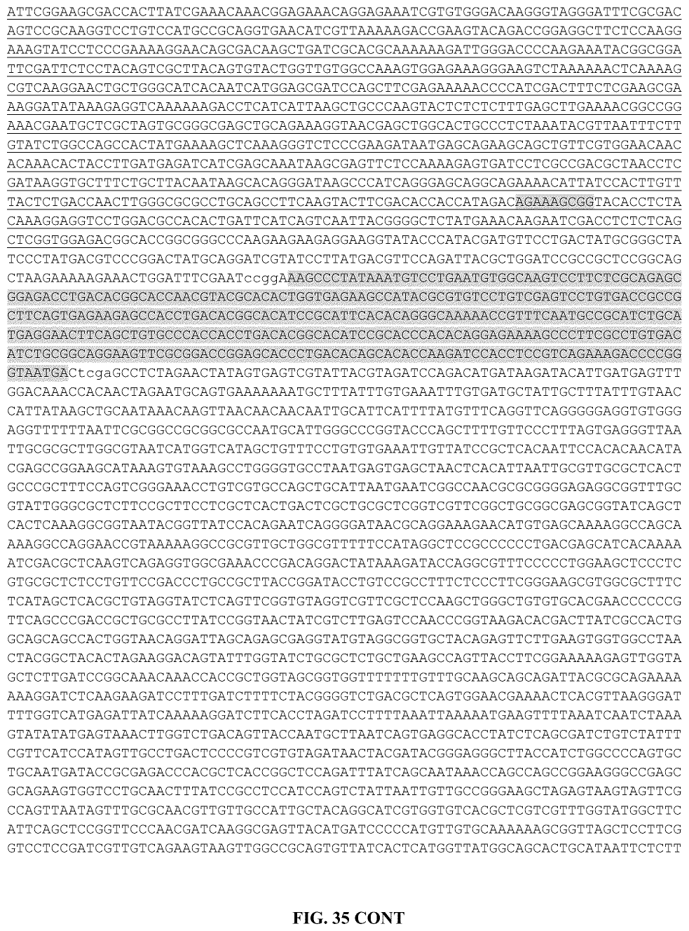

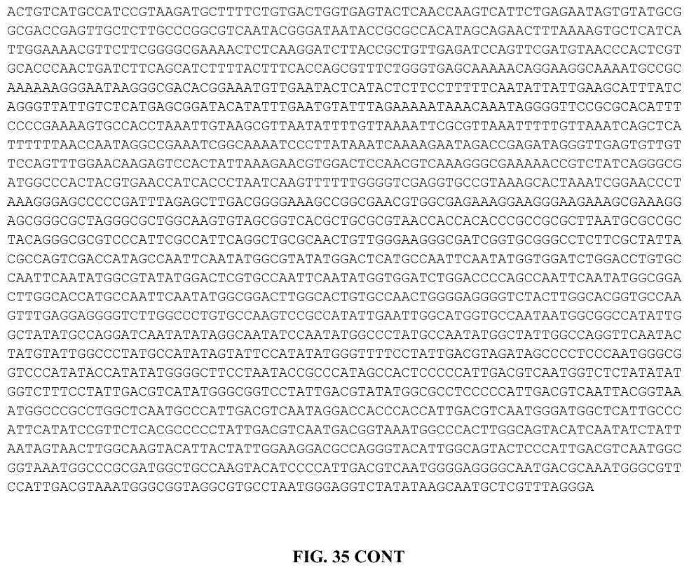

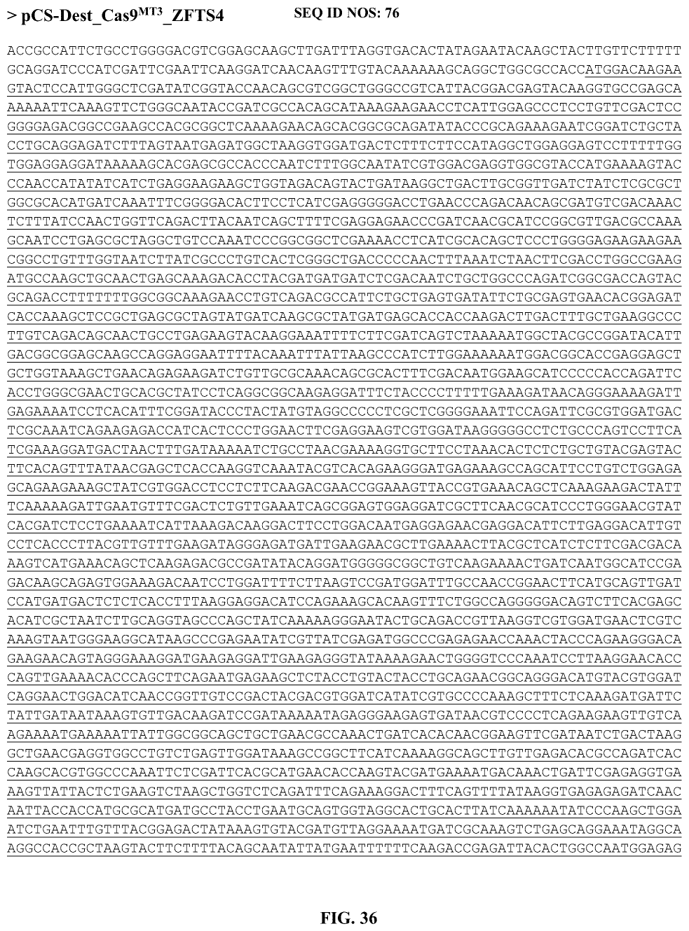

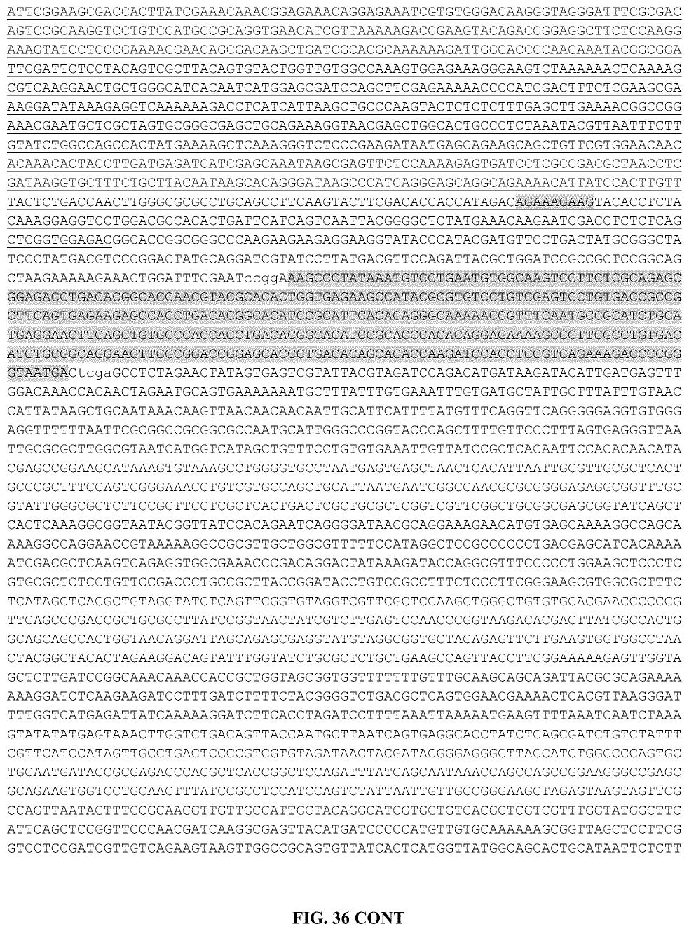

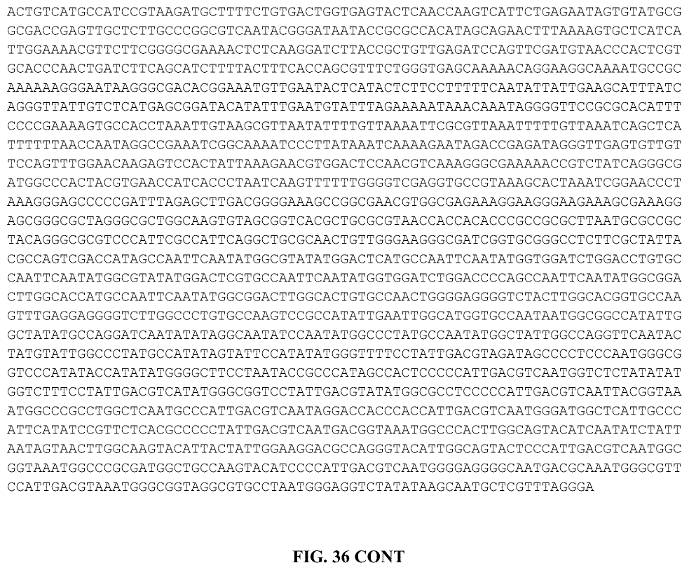

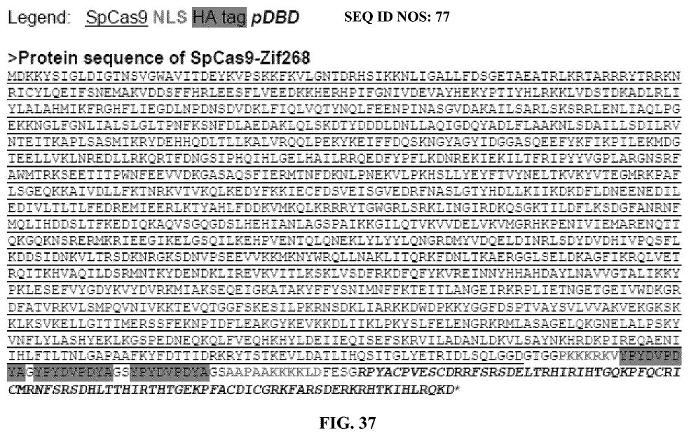

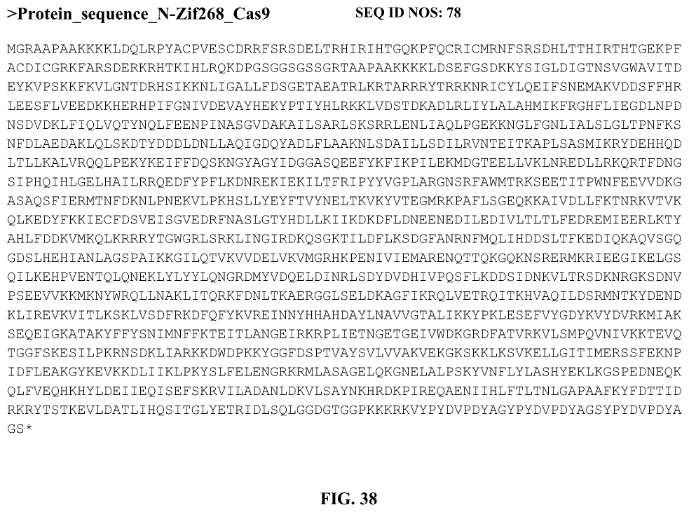

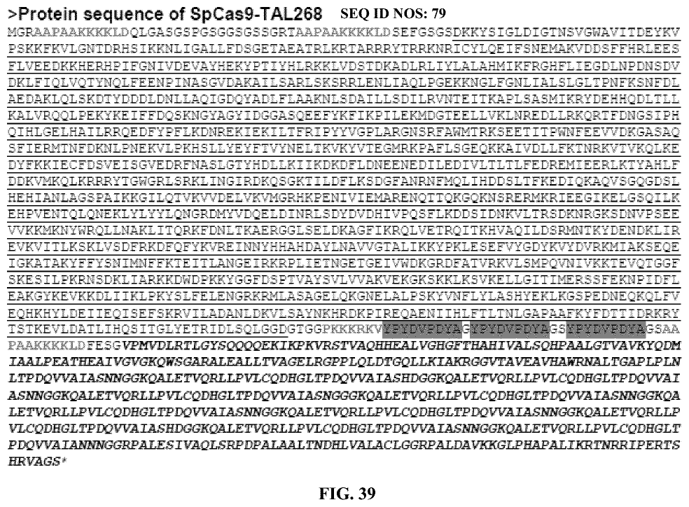

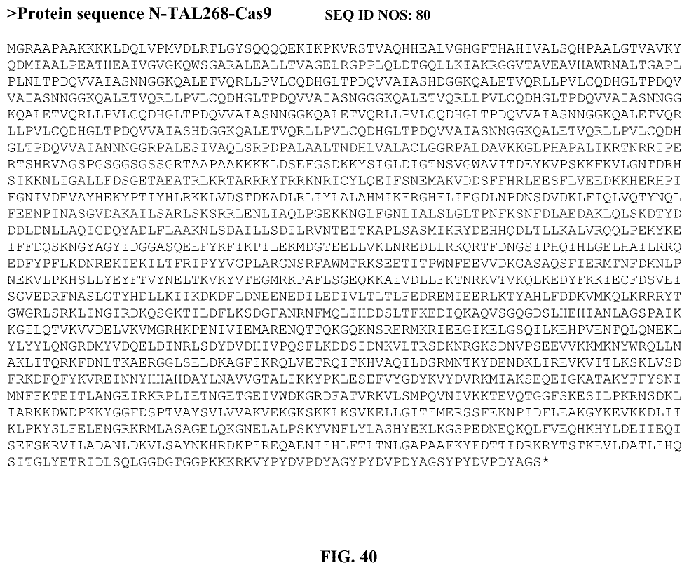

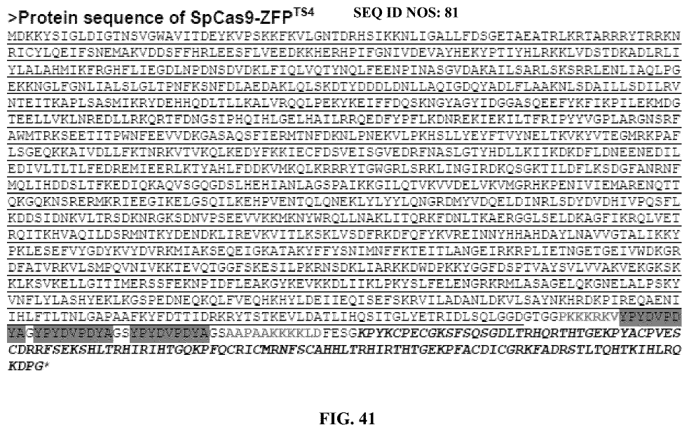

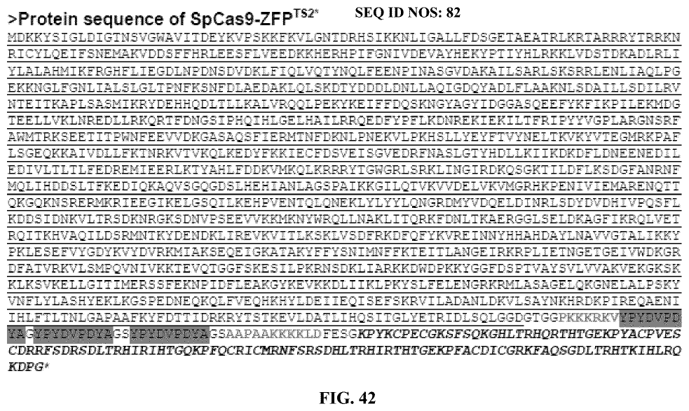

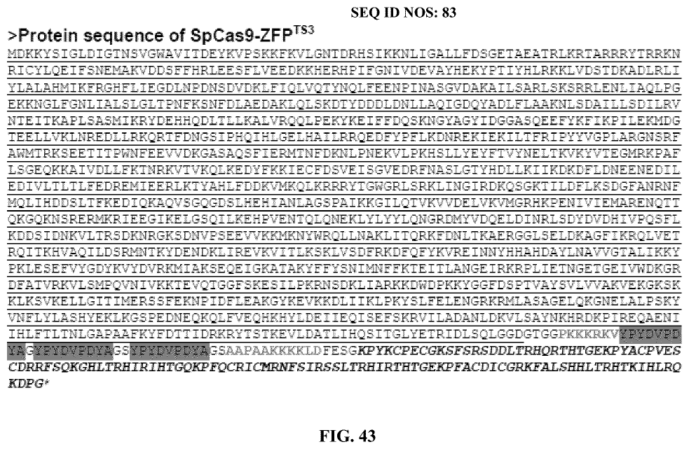

Sequences of a number of the Cas9-DTU fusions used in these preliminary studies are presented in FIG. 27-36. Number of amino acid sequences of Cas9-DTU fusions used in these studies are presented in FIG. 37-43

### C. NmCas9 Gene Editing Platform

Cas9 is believed to be a Type II CRISPR/Cas system and may be further subdivided into three subtypes: i) II-A (including the 1368-aa SpCas9); ii) II-B; and iii) II-C. Barrangou et al., CRISPR-Cas systems: Prokaryotes upgrade to adaptive immunity. Molecular Cell. 2014 Apr. 24; 54(2):234-44. Type II-C Cas9s are believed to be compact and more prevalent than the other two subtypes; (e.g., for example, ˜55% II-C; ˜38% (II-A); ˜7% (II-B)). Further, Type II-C Cas9s may serve to extend the potential targeting specificity via their range of PAM recognition requirements. Fonfara et al., Phylogeny of Cas9 determines functional exchangeability of dual-RNA and Cas9 among orthologous type II CRISPR-Cas systems. Nucleic Acids Research. 2014 Feb. 1; 42(4):2577-90. The shorter length of some Type II-C Cas9s (as small as ˜970-1100 aa) may facilitate delivery, as viral payload limitations make the larger SpCas9 suboptimal for some clinical applications (e.g., adeno-associated viruses). Daya et al., Gene therapy using adeno-associated virus vectors. Clin. Microbiol. Rev. 2008 October; 21(4):583-93.

An in-depth analysis of a Neisseria meningitidis Type II-C system (NmCas9), including a definition of its apparent PAM (5′-NNNNGATT-3′) (SEQ ID NO: 1), has been reported. Zhang et al., Processing-independent CRISPR RNAs limit natural transformation in Neisseria meningitidis. Molecular Cell. 2013 May 23; 50(4):488-503. Further, a 1082-aa NmCas9 has been validated as an efficient genome-editing platform in human cells. Hou et al., Efficient genome engineering in human pluripotent stem cells using Cas9 from Neisseria meningitidis. Proceedings of the National Academy of Sciences. 2013 Sep. 24; 110(39):15644-9; and Esvelt et al., Orthogonal Cas9 proteins for RNAguided gene regulation and editing. Nature Methods. 2013 November; 10(11):1116-21. The structure of a different Type II-C Cas9 from Actinomyces naeslundii (AnCas9) may be known, revealing a distinct arrangement of peripheral domains (in comparison with SpCas9) around a similarly structured nuclease core, though AnCas9's PAM specificity and genome editing efficacy have not been reported. Jinek et al., Structures of Cas9 endonucleases reveal RNA-mediated conformational activation. Science. 2014 Mar. 14; 343(6176):1247997.

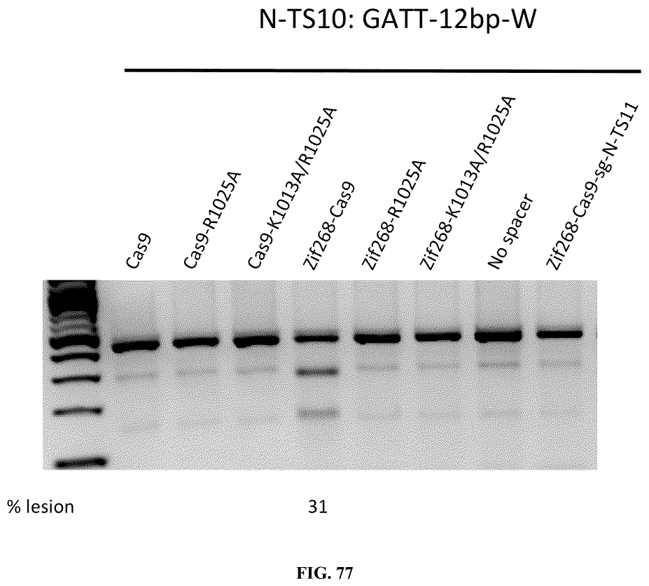

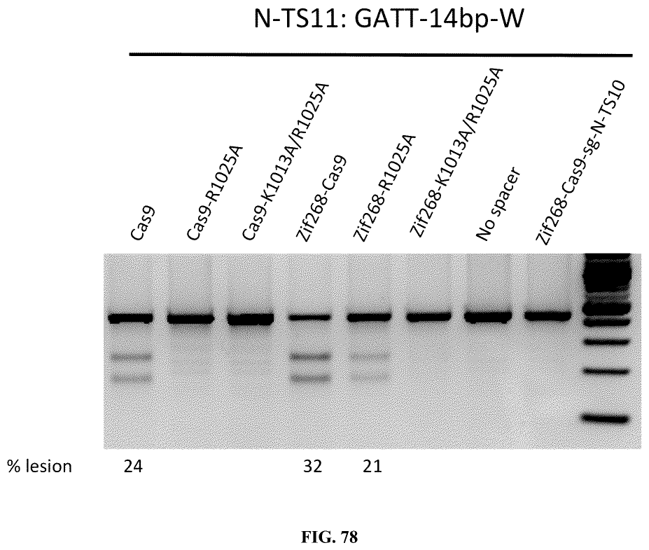

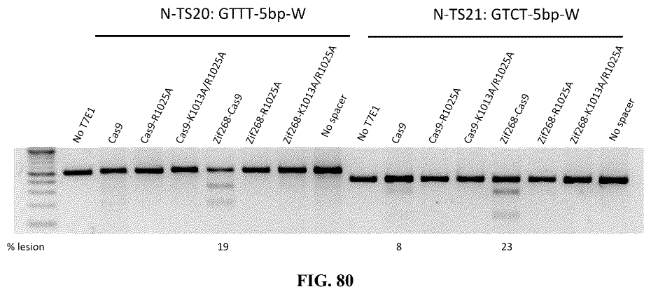

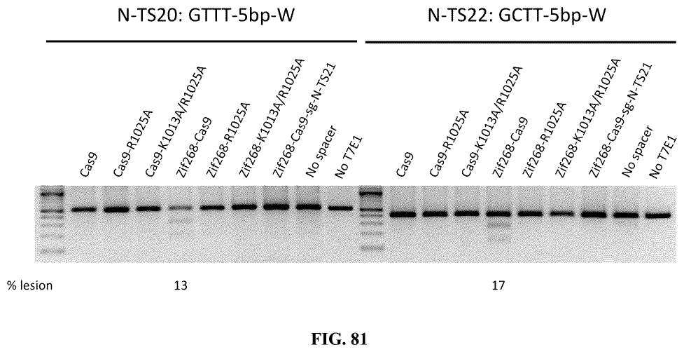

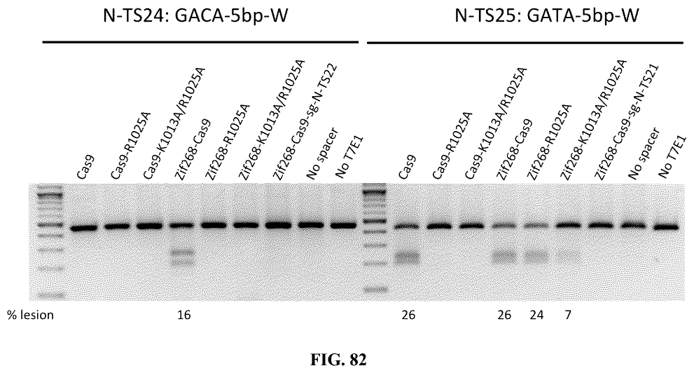

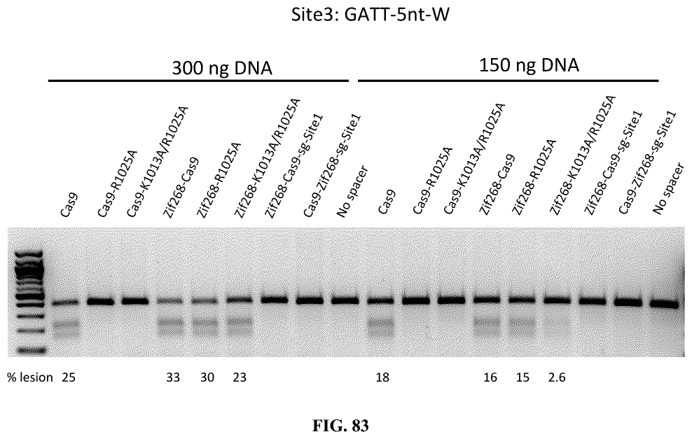

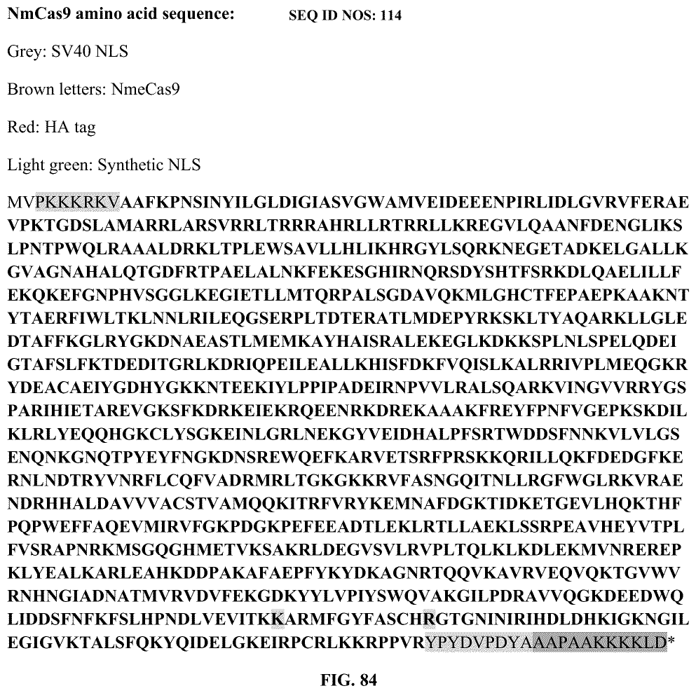

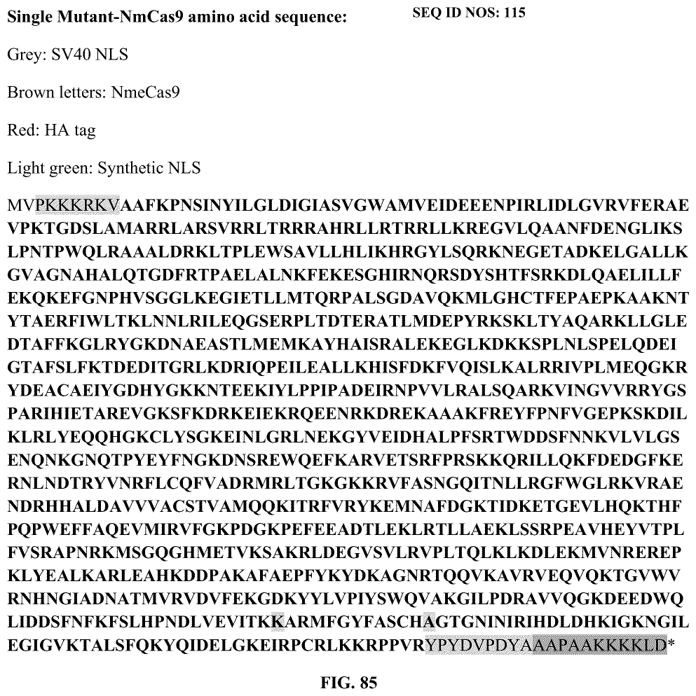

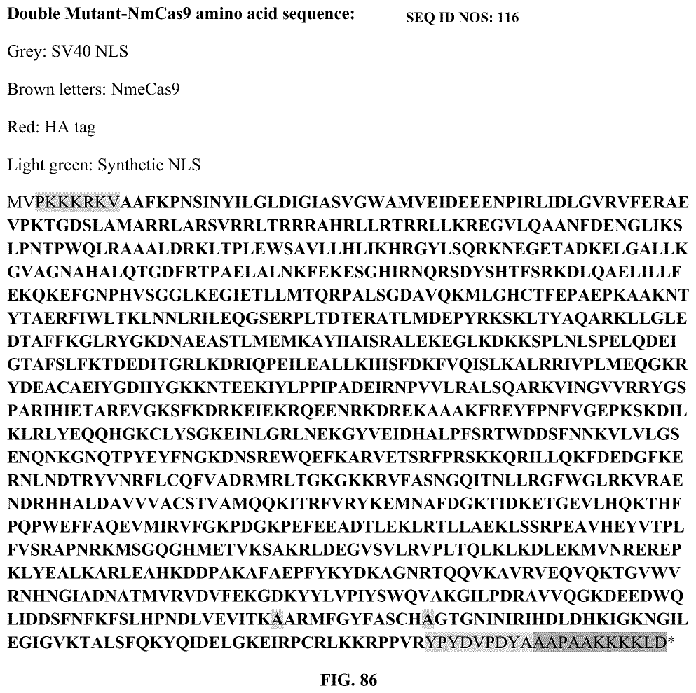

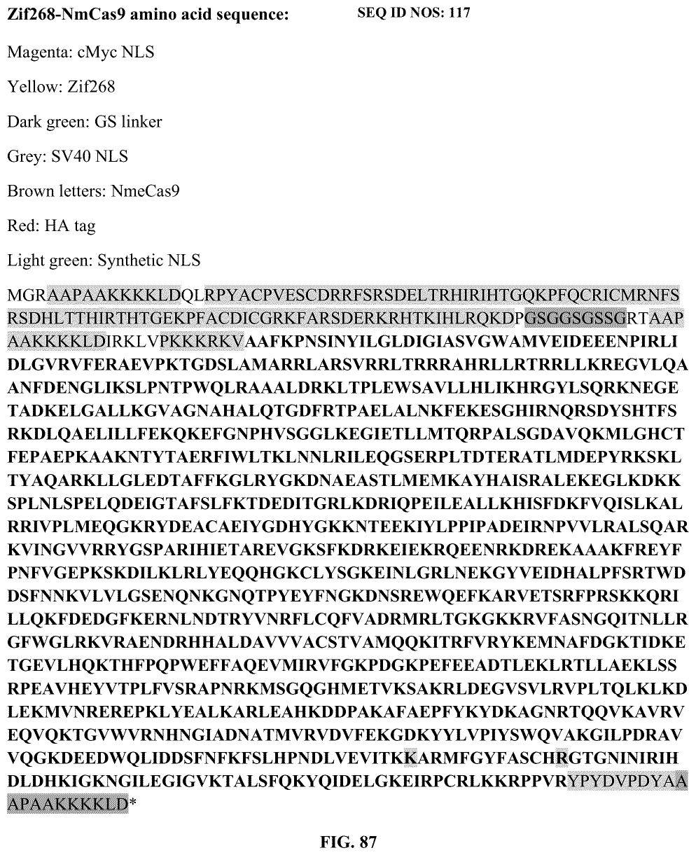

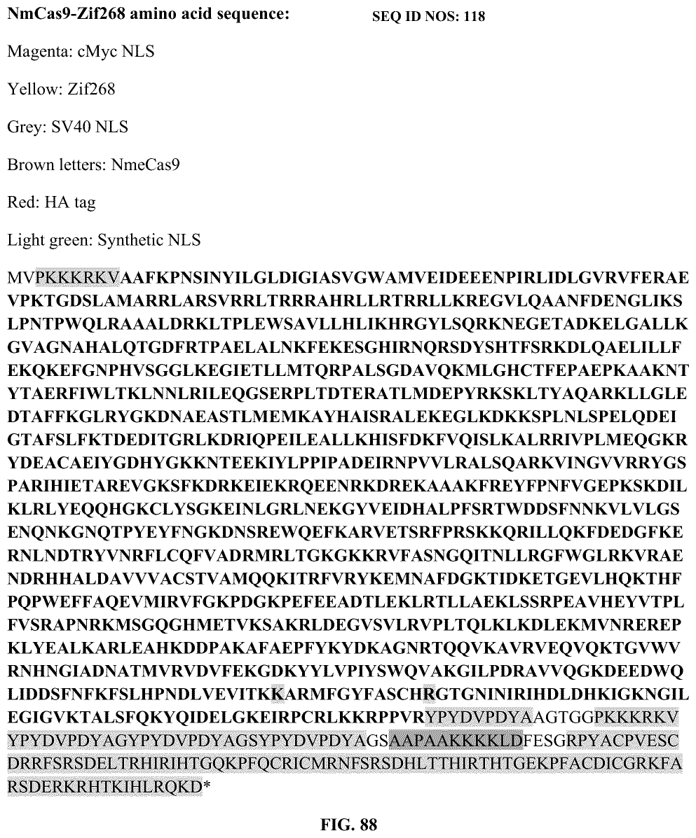

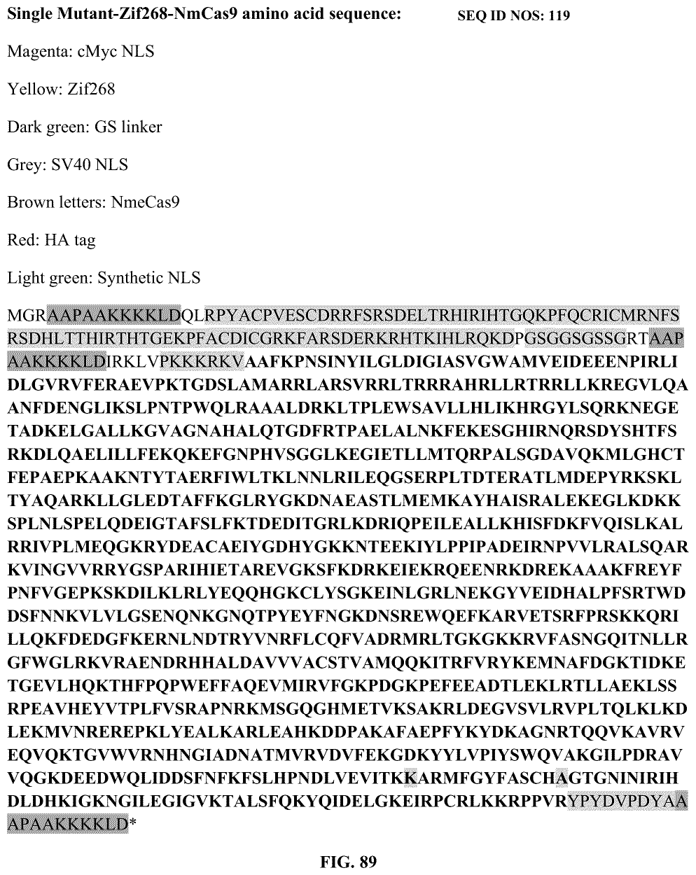

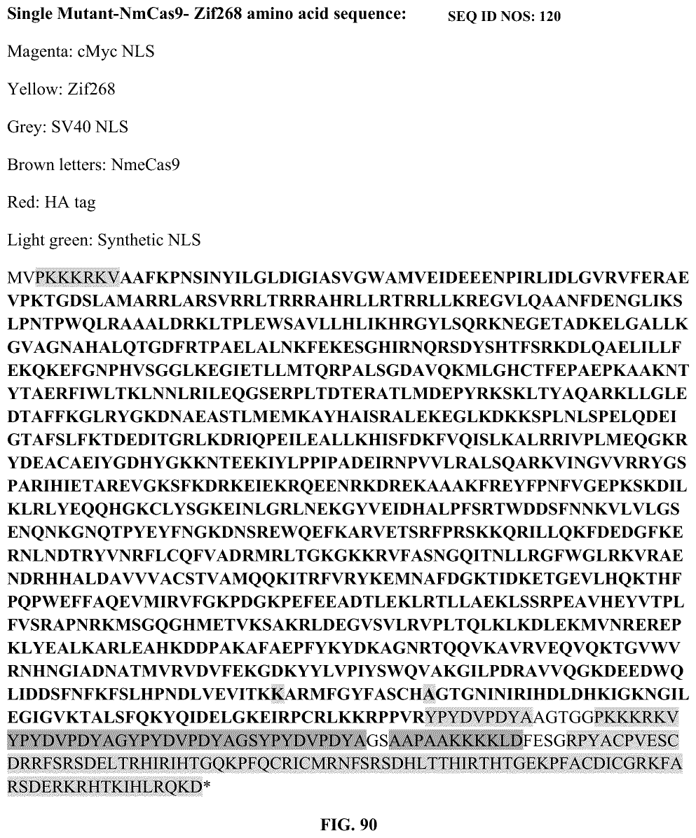

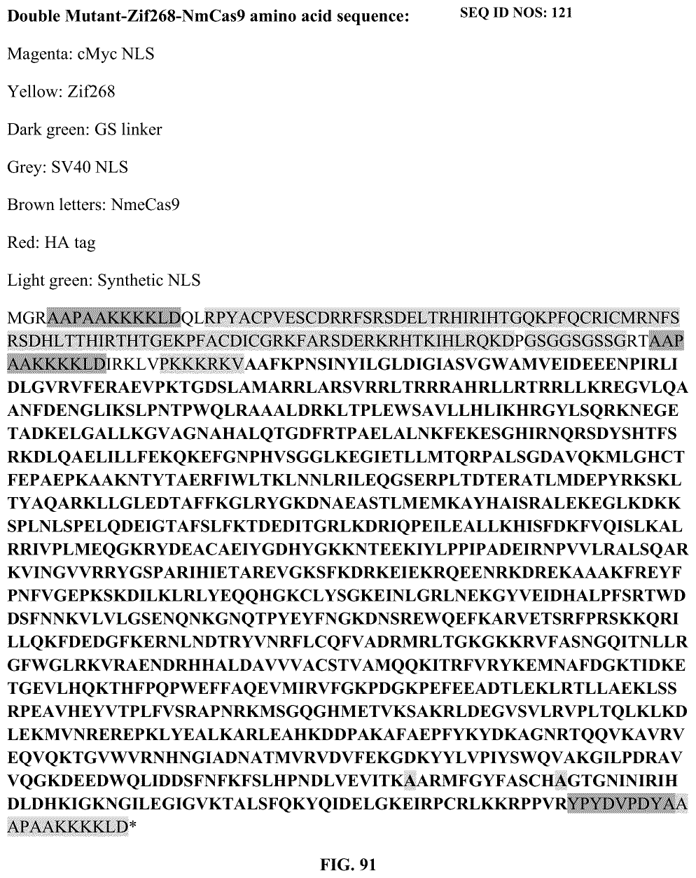

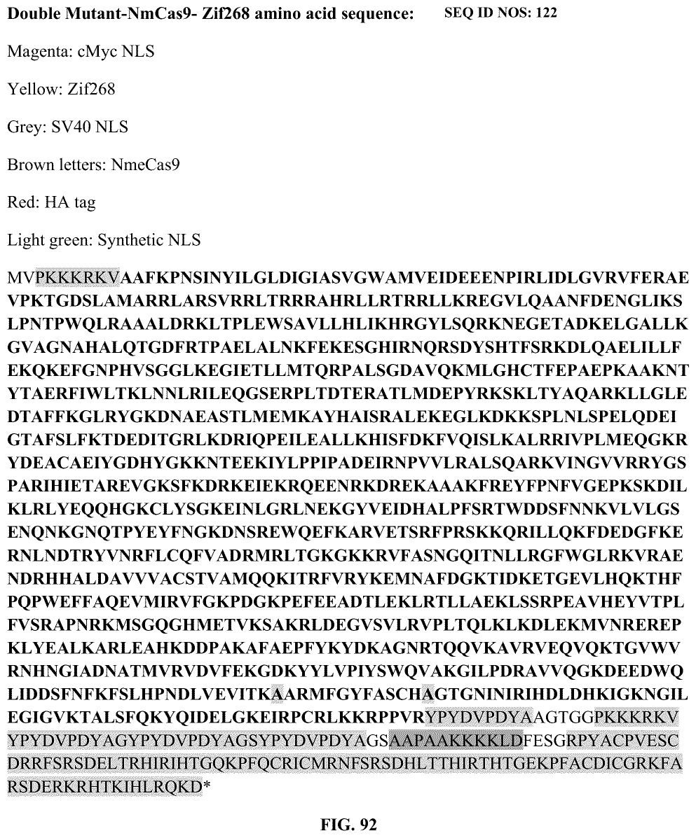

In mammalian cells, PAM requirements efficient editing by nmCas9 has been observed with NNNNG(A/C/T)TT PAMs (SEQ ID NO: 17). Hou et al., Efficient genome engineering in human pluripotent stem cells using Cas9 from Neisseria meningitidis. Proceedings of the National Academy of Sciences. 2013 Sep. 24; 110(39):15644-9; and Esvelt et al., Orthogonal Cas9 proteins for RNA guided gene regulation and editing. Nature Methods. 2013 November; 10(11):1116-21. An ability of a pDBD fusion to extend the range of targetable PAMs has been examined for NmCas9 as previously shown with SpCas9. On genomic target sites with a ZFP (Zif268) fused to the N-terminus or the C-terminus and where a Zif268 binding site is downstream of the PAM an extension of the range of targetable sequences is observed. These data demonstrate that while wild-type NmCas9 is inactive at these genomic loci, the Zif268 fusion permits robust cleavage (FIG. 80, and FIG. 82; e.g. NNNNGTCT (SEQ ID NO: 18), NNNNGACA (SEQ ID NO: 19)). In addition, at some canonical PAM domains (NNNNGATT, FIG. 75, FIG. 77, FIG. 78; NNNNGCTT (SEQ ID NO: 20), FIG. 81; NNNNGTTT (SEQ ID NO: 21), FIG. 80) the ZFP fusion to NmCas9 enhances the activity of the nuclease, in many cases providing activity where unfused NmCas9 is inactive. Thus, a pDBD fusion may provide a method to fully activate Cas9 nucleases even at canonical PAM sites for which they have poor or no activity.

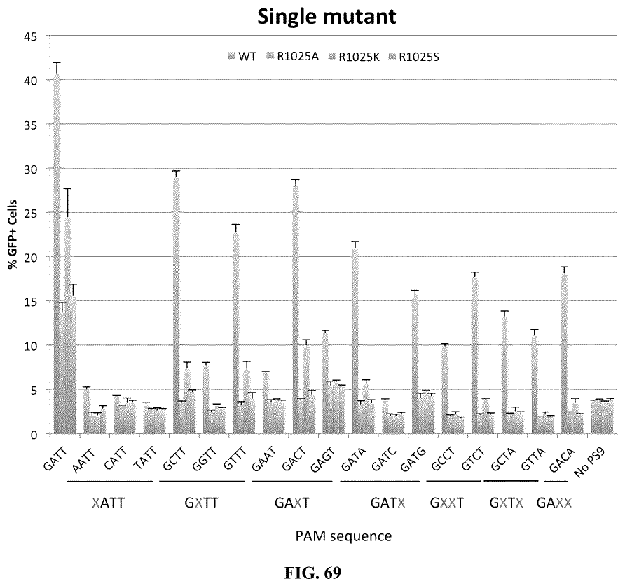

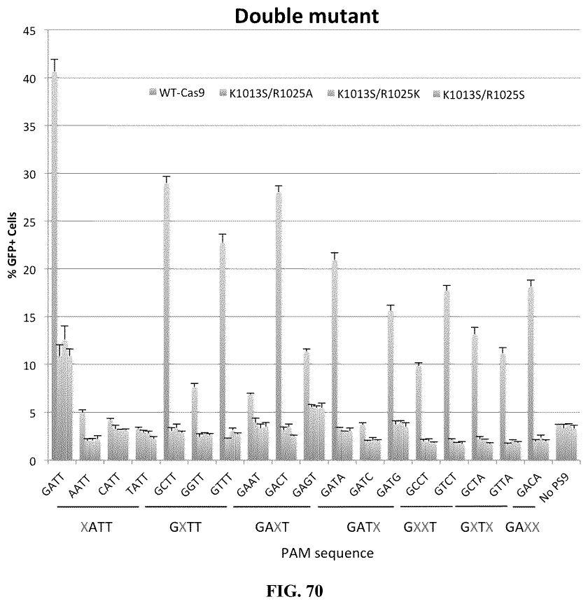

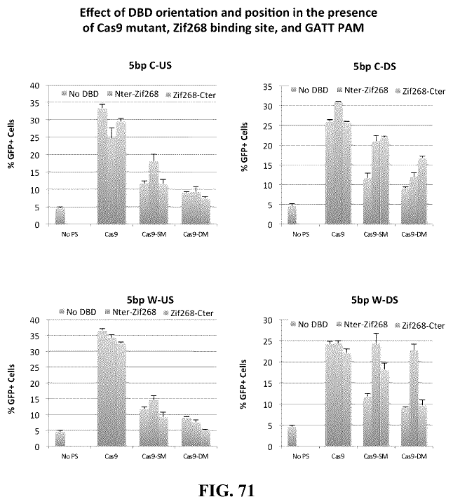

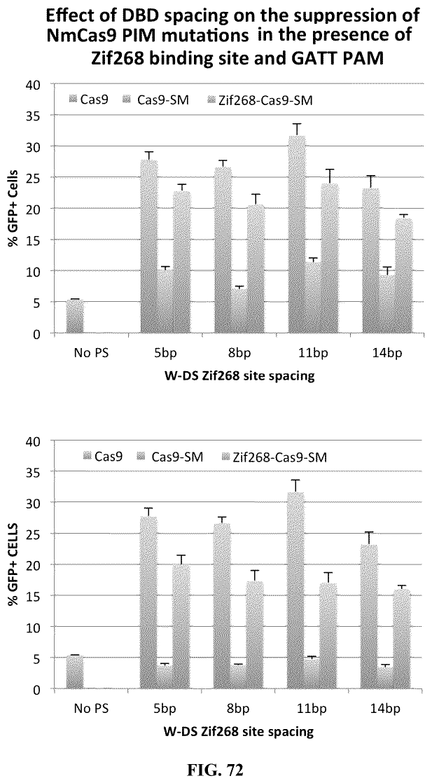

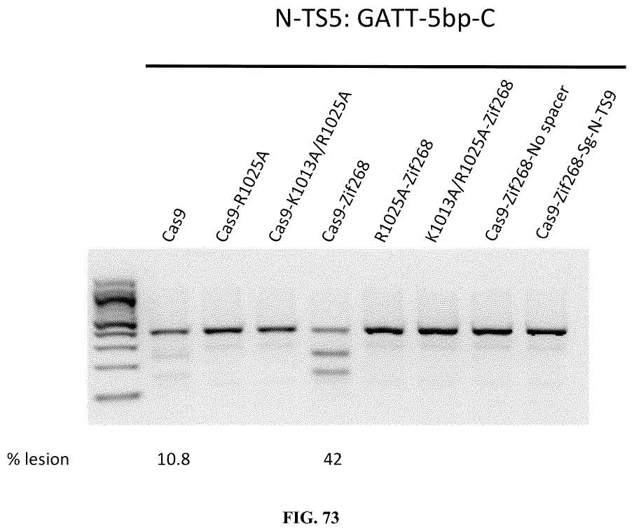

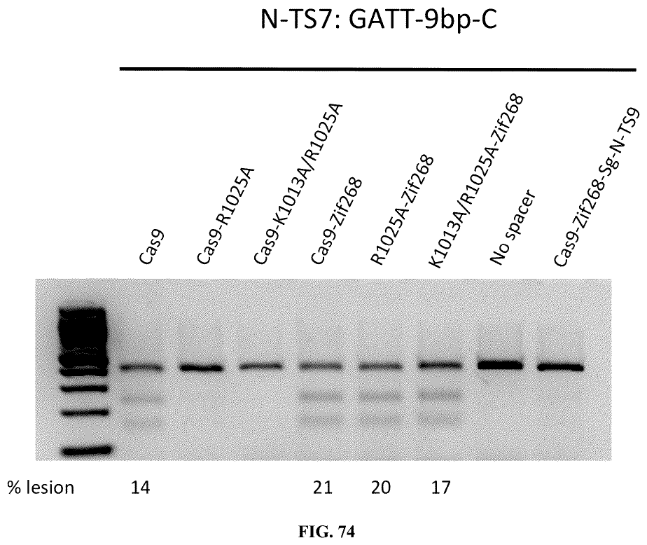

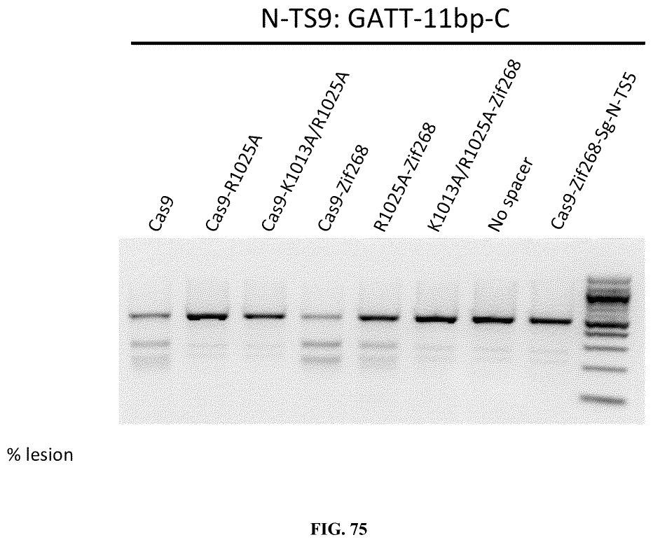

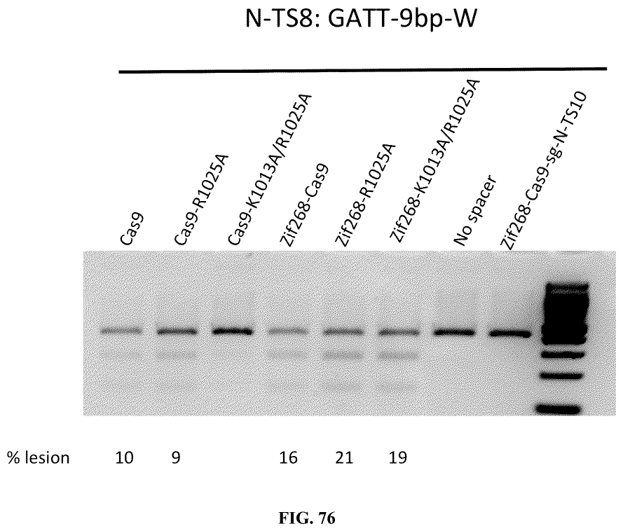

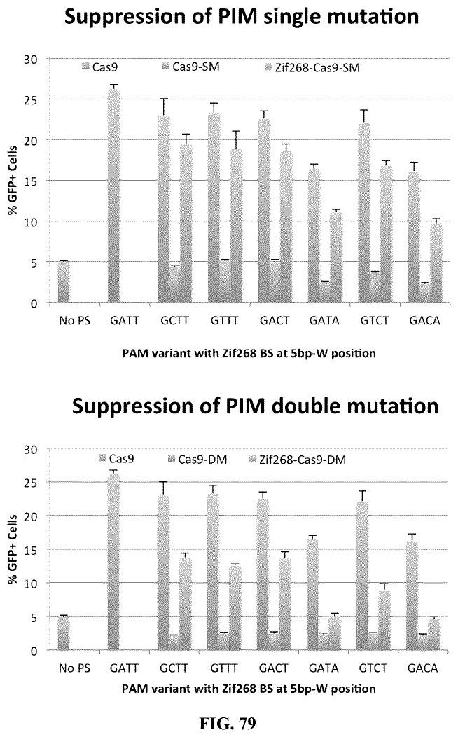

Although the molecular structure of NmCas9 is not known, we have utilized sequence homology with other Type IIC Cas9s from related species to identify residues that are likely involved in PAM recognition or DNA phosphodiester backbone contacts (e.g. K1013 and R1025; FIG. 25). Mutation of arginine 1025 to alanine, serine or lysine dramatically reduces activity at targets sites containing a broad range of functional PAMs (FIG. 69). Mutation of arginine 1025 to alanine, serine or lysine in combination with a mutation of lysine 1013 to serine eliminates any activity above background (FIG. 70). The activity of the R1025A single-mutant NmCas9 (SM), or the K1013A/R1025A double-mutant NmCas9 (DM) can be rescued in GFP reporter assays by the fusion of a ZFP to the N-terminus or C-terminus when the binding site for the ZFP is downstream of the PAM (FIG. 71). ZFP-NmCas9^{SM} or ZFP-NmCas9^{DM} constructs are functional with ZFP binding sites at a number of positions relative to the PAM (FIG. 72) or with a number of different PAM variants (FIG. 79). ZFP fusions to NmCas9^{SM} or NmCas9^{DM} constructs can also restore activity at genomic loci based on T7EI analysis (FIGS. 74, 75, 76, 78 and 82). Thus, we have generated a Type IIC Cas9 platform that is attenuated similar to the SpCas9 Type IIA system using the same principles (summary of activity presented in Table 2).

Although it may be not necessary to understand the mechanism of an invention, it is believed that the above improvements in activity and precision realized by a fusion of a DBD to SpCas9 and NmCas9 and the corresponding attenuating mutations are broadly applicable to other Cas9s. Common design principles between Type II-A and Type II-C Cas9-DBD fusions that achieve excellent precision and improvements in activity demonstrate the applicability of the present invention to all Cas9 platforms and all specific genomic targets. These design principles may be applicable to other CRISPR-based single protein nuclease effector systems (e.g. Type V Cpf1).

TABLE 2
wild-type, attenuated and ZFP fused NmCaz9 editing efficiency at various genomic target sites

| | Editing Efficiency | | | | | |
|---|---|---|---|---|---|---|
| | | Cas9- | Cas9-K1019A/- | | Zif268- | Z11268-K1013A/ |
| SITE NAME | Cas9 | R1025A | R1025A | Zif268-Cas9 | R1025A | R1025A |
| N-TS3(GATT-5bp-W) | 25, 39, 33, 20 | 0 | 0 | 33, 34 | 30, 40 | 23, 16 |
| N-TS5(GATT-5bp-C) | 11, 9, 20 | 0 | 0 | 42 | 0 | 0 |
| N-TS7(GATT-9bp-C) | 14, 24, 33 | 0 | 0 | 21 | 20 | 17 |
| N-TS8(GATT-9bp-W) | 10, 19, 34 | 9 | 1 | 16 | 21 | 19 |
| N-TS9(GATT-11bp-C) | 20, 27 | | | | | |
| N-TS10(GATT-12bp-W) | 0 | 0 | 0 | 31 | 0 | 0 |
| N-TS11(GATT-14bp-W) | 24, 13, 13 | 0 | 0 | 32 | 21 | 0 |
| N-TS20(GATT-5bp-W) | 0 | 0 | 0 | 19, 18, 12, 15, 13 | 0 | 0 |
| N-TS21(GTCT-5bp-W) | 8, 13, 3, 4, 8 | 0 | 0 | 23, 23, 11, 14, 23 | 0 | 0 |
| N-TS22(GCTT-5bp-W) | 0 | 0 | 0 | 18, 16, 12, 10, 17 | 0 | 0 |
| N-TS24(GACA-5bp-W) | 0 | 0 | 0 | 18, 14, 43, 19, 16 | 0 | 0 |
| N-TS25(GACA-5bp-W) | 22, 32, 28, 26 | 0 | 0 | 26, 30, 33, 31, 26 | 25, 25, 35, 33, 24 | 6, 8, 23, 20, 7 |

### D. Broadened Range of Cas9 Specific Target Sequences

In one embodiment, the present invention contemplates a method comprising differentially controlling functional recognition of a target site and subsequent cleavage by sequence elements within a Cas9 protein. One of the current limitations of Cas9 may be that, although target site recognition sequence can be programmed with a sgRNA, the ability to bind and cleave the target site sequence may be also dictated by a Cas9 PAM recognition sequence. In some Cas9 isoforms, a PAM sequence of NGG may be highly preferred both for binding and for cleavage. Hsu et al., DNA targeting specificity of RNA-guided Cas9 nucleases. Nature Biotechnology 31, 827-832 (2013); Wu et al., Genome-wide binding of the CRISPR endonuclease Cas9 in mammalian cells. Nature Biotechnology (2014); and Kuscu et al., Genome-wide analysis reveals characteristics of off-target sites bound by the Cas9 endonuclease. Nature biotechnology (2014). Lower cleavage activity was observed for NAG PAMs, whereas other PAMs have no activity.

The data presented herein shows the activity of SpCas9 or SpCas9-Zif268 with a common sgRNA on target sites that have each of the 16 different PAM sequences with a flanking Zif268 site 5 base pairs away. Remarkably, a SpCas9-Zif268 construct may be highly active at multiple PAMs (i.e., for example, NGG, NAG, NGC and NGA) with a common sgRNA recognition sequence, equivalent activity at non-NGG PAMs has not been previously described. FIG. 5. Although it may be not necessary to understand the mechanism of an invention, it is believed that an ability to expand the targeting capabilities of Cas9 would be particularly valuable for targeting genomic sequences that lack a canonical PAM within a local region of interest (mutation requiring correction in gene therapy applications) or for allele-specific targeting taking advantage of SNPs that distinguish the alleles that represent an active and an inactive PAM, allowing one of the two sequences to be cleaved specifically. This could be a powerful approach for the inactivation of dominant-negative disease causing alleles, such as that Huntington's disease or Myotonic Dystrophy.

Conventional SpCas9 sgRNAs (e.g., for example, TS2, TS3 & TS4; all NGG PAMs) are known to have well-defined off-target sites. Fu et al., Improving CRISPR-Cas nuclease specificity using truncated guide RNAs. Nature Biotechnology. 2014 March; 32(3):279-84; Fu, Y., Foden, J. A., Khayter, C., Maeder, M. L., Reyon, D., Joung, J. K., & Sander, J. D. (2013). High-frequency off-target mutagenesis induced by CRISPR-Cas nucleases in human cells. Nature Biotechnology, 31(9), 822-826. doi:10.1038/nbt.2623. On- and off-target cleavage efficiencies at these sites may be evaluated for SpCas9-DBD constructs, where an attached DBD recognizes a sequence near each target site. Further, improved linkers may be combined with improved SpCas9 PAM recognition domain mutants to construct a Cas9 fusion protein most likely to eliminate off-target activity at the previously identified sequences.

Initial assessment of SpCas9-DBD precision may be done via T7EI assays on PCR amplicons from target and predicted off-target sites. For promising constructs, deep-sequencing of these amplicons will be used quantify lesion rates at each site. Gupta et al., Zinc finger protein-dependent and -independent contributions to the in vivo off-target activity of zinc finger nucleases. Nucleic Acids Research. 2011 Jan. 1; 39(1):381-92. To assess nuclease activity at sites throughout a genome, GUIDE-seq analysis can be performed (Tsai, S. Q. et al. GUIDE-seq enables genome-wide profiling of off-target cleavage by CRISPR-Cas nucleases. Nature biotechnology 33, 187-197 (2015).). Regions exhibiting significant GUIDE-seq oligonucleotide incorporation may be characterized for off-target cleavage rates in the nuclease-treated cells using the same PCR-based deep sequencing approach described above. Given preliminary results, it may be anticipated that the precision of Cas9^{mut}-DBD has vastly improved and superior activity as compared to Cas9.

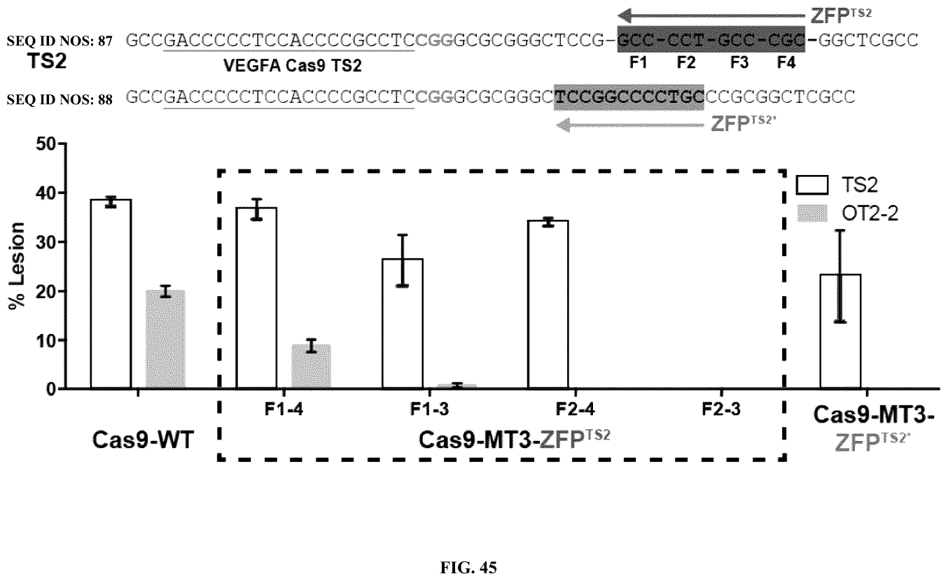

TALE or ZFP binding site length may also be varied to provide optimal binding precision. For example, binding site size and affinity of TALEs or ZFPs can be tuned by changing the number of recognition modules that are incorporated into the Cas9 fusion protein (FIG. 45). Bhakta et al., Highly active zinc-finger nucleases by extended modular assembly. Genome Research. 2013 March; 23(3):530-8; Meckler et al., Quantitative analysis of TALE-DNA interactions suggests polarity effects. Nucleic Acids Research. 2013 April; 41(7):4118-28; and Reyon et al., FLASH assembly of TALENs for high-throughput genome editing. Nature Biotechnology. 2012 May; 30(5):460-5. On-target versus off-target cleavage activity may then be evaluated for different length TALE or ZFP variants to understand how this affects precision. Likewise if a orthogonal Cas9 isoform may be used as the DNA targeting unit the affinity for its target site can potentially be tuned to optimize the on-target versus off-target cleavage rate by truncating its guide RNA (Fu et al., Improving CRISPR-Cas nuclease specificity using truncated guide RNAs. Nature Biotechnology. 2014 March; 32(3):279-84). The association between the two orthogonal Cas9/sgRNAs could be via a direct linkage or a dimerization domain. (FIG. 20)

### E. ZFP Or TALE Cas9 Fusion Proteins

In one embodiment, the present invention contemplates a method comprising binding a Cas9 fusion protein comprising a ZFP or TALE to a non-standard PAM target site. In one embodiment, a non-standard PAM target site comprises a NAG PAM sequence. Although it may be not necessary to understand the mechanism of an invention, it is believed that a NAG PAM sequence may be weakly cleaved by the standard SpCas9 (e.g., a sub-optimal PAM sequence).

The data presented herein examines spacing and orientation requirements between a DBD target site and a neighboring PAM sequence. For this analysis, a TALE protein was generated that recognized a Zif268 binding site (TAL268). This provided the advantage that the same reporter system to examine the activity of SpCas9, SpCas9-Zif268 and SpCas9-TAL268. The data show that a standard SpCas9/sgRNA may be only functional with a NGG PAM, but not on an NAG PAM. However, SpCas9-Zif268 may be active at an NAG PAM on all spacings and orientations of its binding site. A similar broadening of targeting range is observed with ZFP fusions to NmCas9 (Table 2, above). SpCas9-TAL268 has a much more restricted spacing and orientation for favorable activity, FIG. 3. Given that TALEs can be programmed to recognize nearly any sequence within the genome (Lamb et al., Directed evolution of the TALE N-terminal domain for recognition of all 5′ bases. Nucleic Acids Research. 2013 November; 41(21):9779-85), it should be possible to generate a DBD that may be complementary to almost any desired region of the genome to recruit Cas9 to a desired target site.

### F. Cas9-Cas9 Fusion Proteins

In one embodiment, the present invention contemplates a method comprising binding of a Cas9-Cas9 fusion protein (dual Cas9 system) to a composite binding site. This could involve one Cas9 component serving as the nuclease and the other nuclease-dead Cas9 (dCas9) component serving as the targeting domain (analogous to the ZFP or TALE component of the Cas9-ZFP/TALE fusions; FIG. 20). Other embodiments envision the construction of Cas9-Cas9 fusion proteins where both components contain active nuclease domains, where these could be combinations of the fully active nucleases, nuclease-nickase or nickase-nickase combinations, where the nickases could be generated either by inactivation the HNH or the RuvC domains.

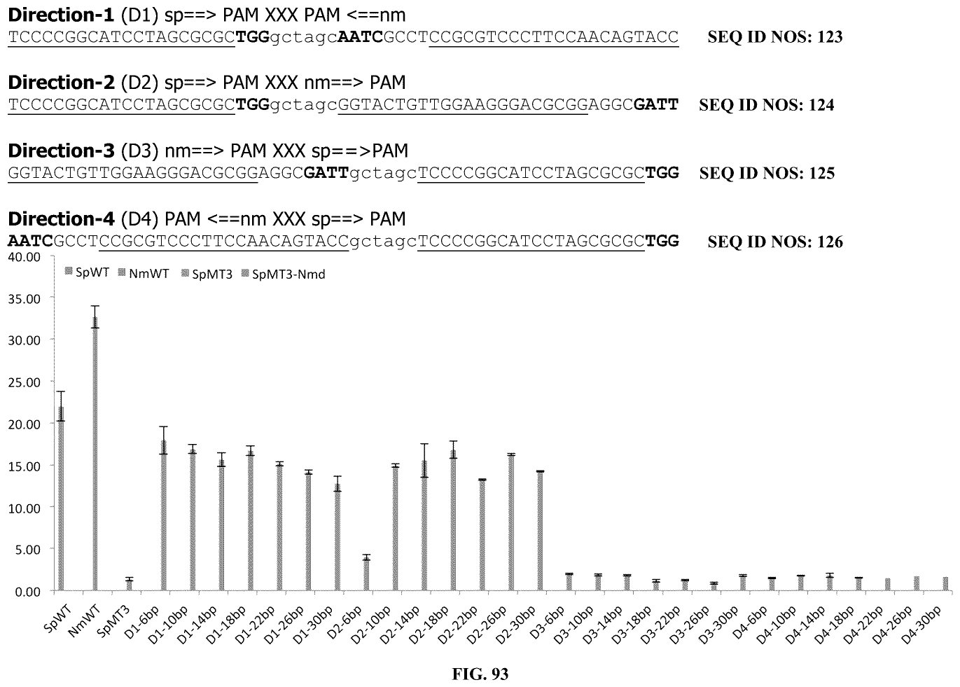

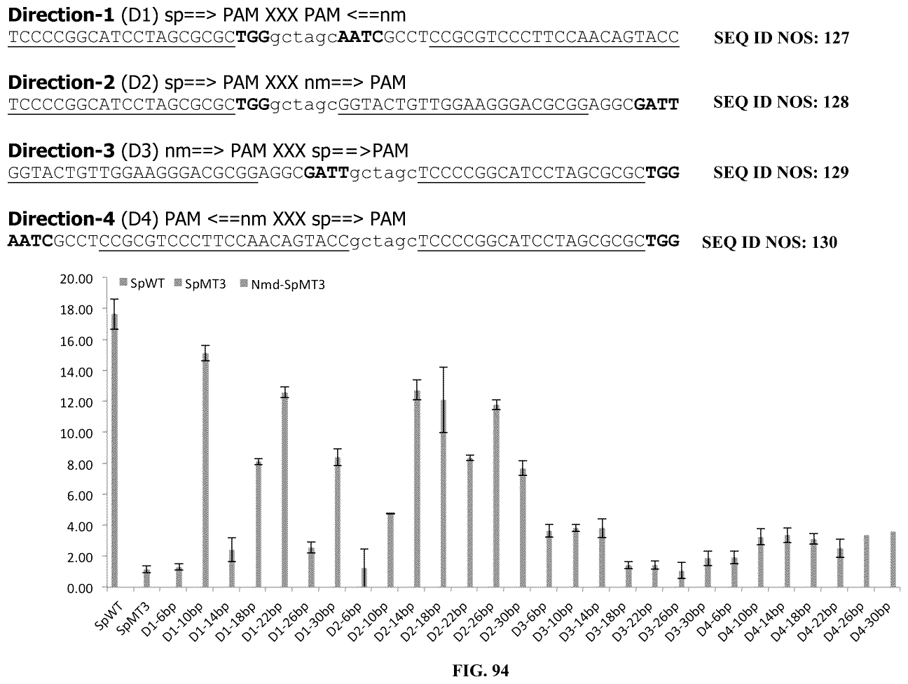

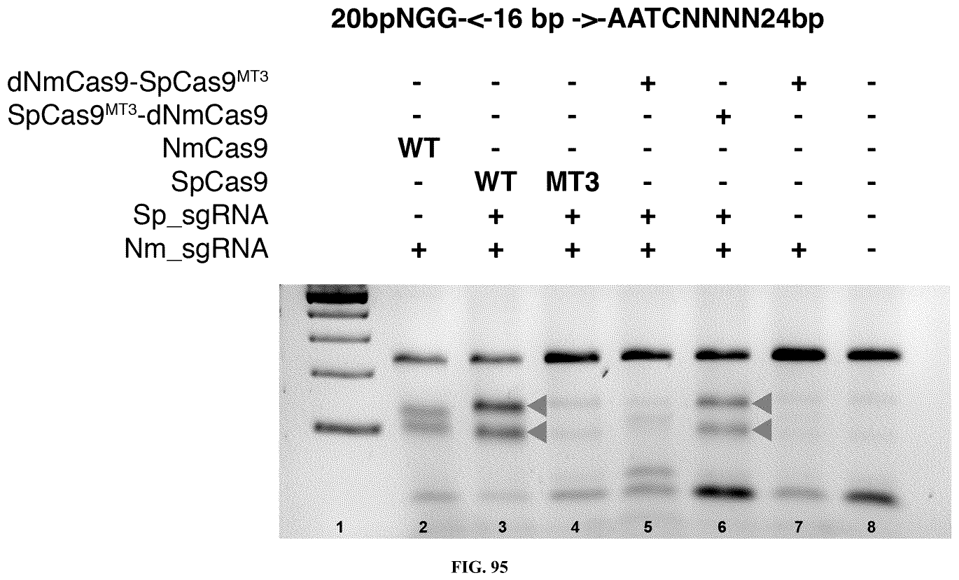

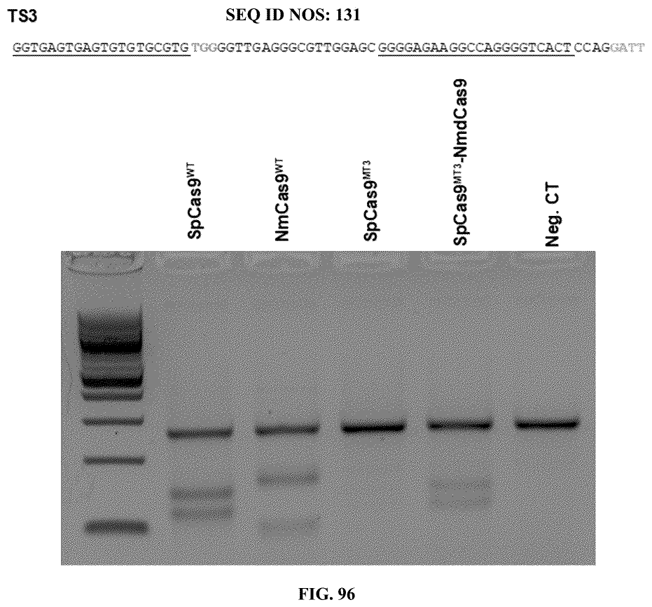

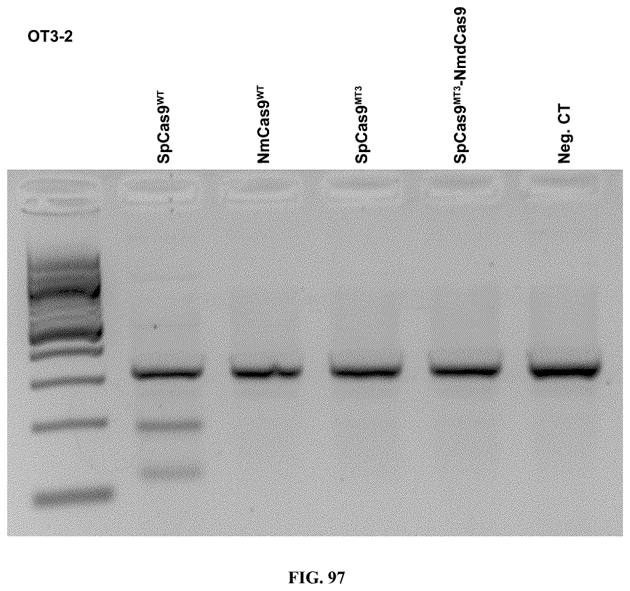

A split-GFP reporter assay was employed to demonstrate that SpCas9^{MT3}-NmdCas9 and NmdCas9-SpCas9^{MT3} can generate target cleavage with certain arrangements of target sites for NmdCas9 (nuclease-dead) and SpCas9^{MT3} (attenuated), FIG. 93 and FIG. 94. In these constructs the nuclease-dead NmCas9 targets the attenuated SpCas9^{MT3} to the desired target site facilitating cleavage. The SpCas9^{MT3}-NmdCas9 construct is capable of targeting genomic sequences, where it efficiently induces lesions (FIG. 95). The presence of some kind of DNA targeting unit is absolutely required, as SpCas9^{MT3} on its own lacks detectable activity. Like SpCas9^{MT3}-pDBDs, SpCas9^{MT3}-NmdCas9 constructs dramatically increase the precision of SpCas9. SpCas9^{MT3}-NmdCas9 was programmed to recognize the TS3 target site that was the focus of some of our SpCas9^{MT3}-ZFP studies. The dual Cas9 system efficiently generates lesions at the TS3 target site (FIG. 96). However, at the most active off-target site for wild-type Cas9 (OT3-2) programmed with the TS3 sgRNA, which has lesion rates that are similar to the TS3 target site, the dual Cas9 platform (SpCas9^{MT3}-NmdCas9) has no apparent activity (FIG. 97). Thus, like the SpCas9^{MT3}-pDBD fusions, the dual Cas9 platform has greatly improved precision. In addition, since it can be programmed simply through the use of orthogonal sgRNAs for Sp and NmCas9, the programming of this nuclease is straightforward.

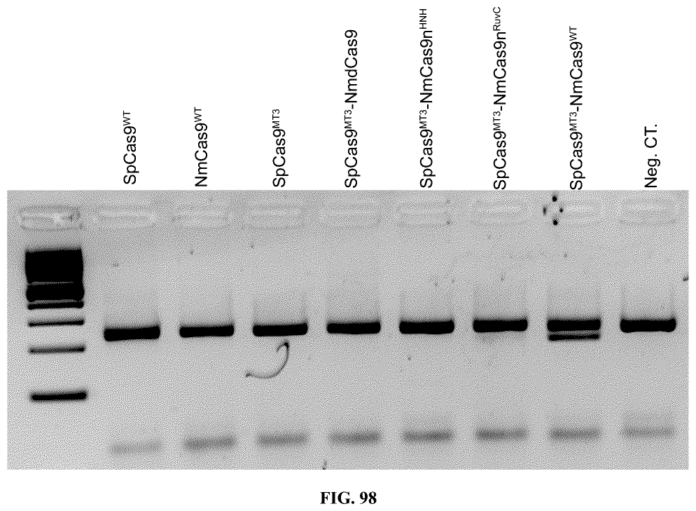

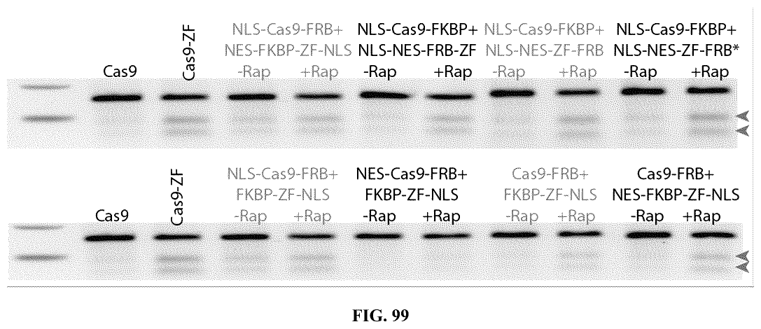

One of the advantages of the dual Cas9 system over the Cas9-pDBD system is the ability to utilize both nuclease domains to achieve coordinated cleavage at two neighboring positions within the genome. For example, attenuated SpCas9 can be coupled to NmCas9 that is either a nickase or a double-strand nuclease to allow the formation of a single-strand nick neighboring a break or two double-strand breaks together. If a NmCas9 nickase is utilized, the strand that is cleaved can be controlled by the nuclease domain (either HNH or RuvC) that is inactivated. This can in principle be utilized to create extended 5′ or 3′ overhangs neighboring the blunt double-strand break that is generated by attenuate SpCas9, which are likely to have improved properties for certain types of DNA repair (alternate non-homologous end joining or homology directed repair from an exogenous template). These combinations of dual nuclease-nickase or dual nucleases are functional, and in the case of the dual nucleases provide clear deletions of the intervening sequence (FIG. 98).

G. Drug-Dependent Cas9 pDBD Systems

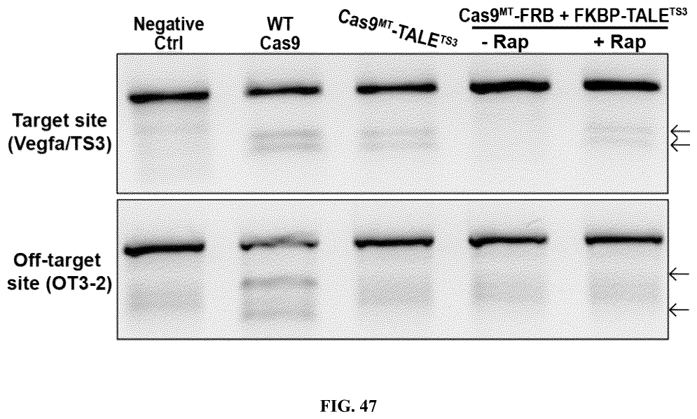

In one embodiment, the present invention contemplates a method comprising binding of a drug-dependent nuclease system where the attenuated Cas9 and the pDBD (or alternate DTU such as an different Cas9 isoform) where the temporal activity of the nuclease can be controlled by the presence of a small molecule. Small molecule-(Yoshimi K, et. al. Nature Communications. 2014; 5:4240; Spencer D M, et. al. Science. 1993 Nov. 12; 262(5136):1019-24; Hathaway N A, et. al. Cell. Elsevier Inc; 2012 Jun. 22; 149(7):1447-60; Liang F-S, et al. Science Signaling. 2011; 4(164):rs2-rs2.) or light-dependent (Konermann S, et al. Nature. 2013 Aug. 22; 500(7463):472-6) dimerization systems have been developed that permit the control of activity of a two-component system. Since SpCas9/sgRNA off-target activity is dose dependent, these systems have been adapted to regulate the association of two fragments of Cas9 (Split-Cas9; Nihongaki Y, et. al. Nature biotechnology. 2015 July; 33(7):755-60; Wright A V, et al. Proceedings of the National Academy of Sciences. 2015 Mar. 10; 112(10):2984-9; Zetsche B, et. al. Nature biotechnology. 2015 February; 33(2):139-42; Davis K M, et. al. Nat Chem Biol. 2015 May; 11(5):316-8.). However, this framework may not be ideal, as drug-dependent Split-SpCas9 displays reduced target activity and retains modest off-target activity (Zetsche B, et. al. Nature biotechnology. 2015 February; 33(2):139-42.). SpCas9-pDBD systems are amenable to the incorporation of a drug- or light-dependent dimerization system that regulates the association of SpCas9 and the pDBD by replacing the covalent linker with a conditional dimerization system (drug or light dependent) (FIG. 2C). A working Rapamycin-dependent prototype was developed for SpCas9-FRB/FKBP-ZFPs and SpCas9-FRB/FKBP-TALEs (FIG. 47). For example, the target activity (with drug) is similar to wild-type SpCas9 without sacrificing the enhanced precision of the SpCas9-pDBD system: wild-type SpCas9 displays activity at the off-target sequence OT3-2, whereas no activity is observed for the drug-dependent system.

Activity and drug-responsiveness of this system has been improved through a number of additional modifications. To increase the turnover of the pDBD in the absence of drug, which can potentially compete with SpCas9-FKBP/FRB-ZFPs complexes if in excess, a destabilized FRB domain has been incorporated (i.e., for example, a PLF triple mutant-FRB*; Stankunas K, et. al. Chembiochem. 2007 Jul. 9; 8(10):1162-9.) on the pDBD component. The cellular localization sequences on Cas9 and the pDBD has also been improved. An absence of a nuclear import (NLS) or export (NES) sequence on Cas9 was found to provide the lowest background levels of cleavage while providing the largest drug-dependent activity. For the pDBD the presence of a combination of 2×NLS and 2×NES, which is believed to cause constant cycling between the nucleus and cytoplasm, thereby resulting in improved activity (FIG. 99). The organization of these domains (e.g. FRB* on the N- or C-terminus) also influences activity. These modifications of the system play a role in the generation of the highest levels of performance. This type of regulation should be possible with other small molecule- or light-dependent dimerization systems, and thereby should provide tighter control over activity for gene therapy based uses (gene correction, gene replacement or cell-based therapeutics).

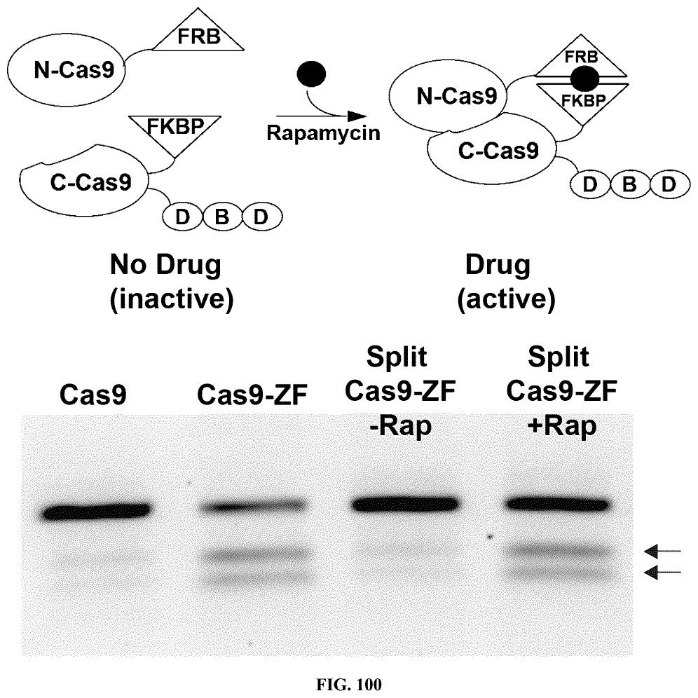

Regulated nuclease activity can be obtained by breaking the Cas9 protein into two independent components (e.g., termed herein “split-Cas9”), where assembly can be controlled. Switching into an active state can be driven through the delivery of a small molecule (Zetsche B, et. al. Nature biotechnology. 2015 February; 33(2):139-42. Davis K M, et. al. Nat Chem Biol. 2015 May; 11(5):316-8.) or light of a suitable wavelength (Nihongaki Y, et al. Nature biotechnology. 2015 July; 33(7):755-60.). Most of these platforms display lower activity at the target site and off-target sites when compared with standard Cas9. Fusion of a pDBD to one of the Split-SpCas9 components has been demonstrated that dramatically increases its activity at alternate PAM sequences (e.g NAG, FIG. 100).

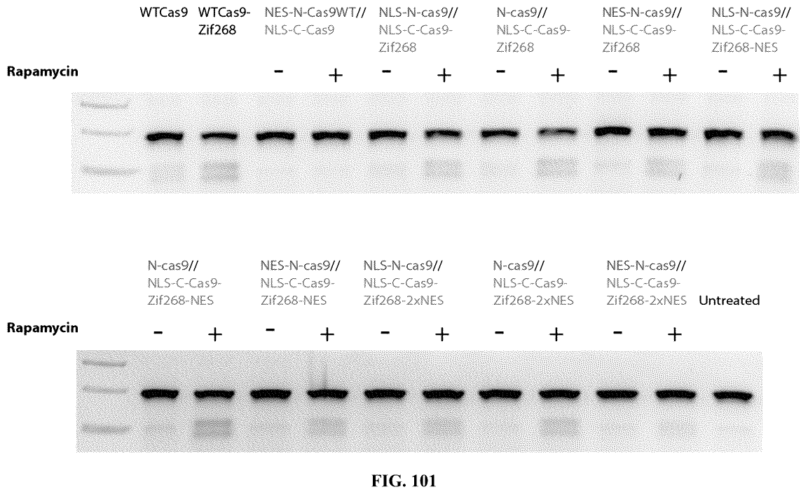

Activity and drug-responsiveness of the Split-Cas9-ZFP system has also been improved through a number of additional modifications. Using, for example, the cellular localization sequences on the N-terminal and C-terminal components of Split-Cas9. Inclusion or absence of a nuclear import (NLS) or export (NES) sequence on these segments was found to influence the background and drug-dependent cleavage rates of these constructs (FIG. 101).

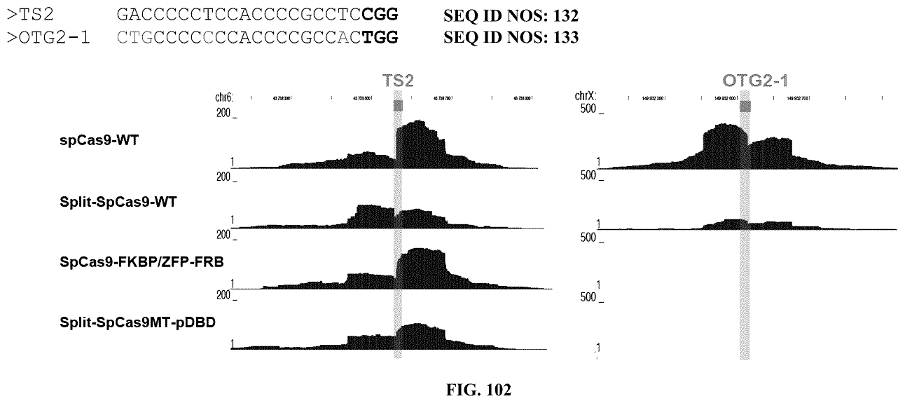

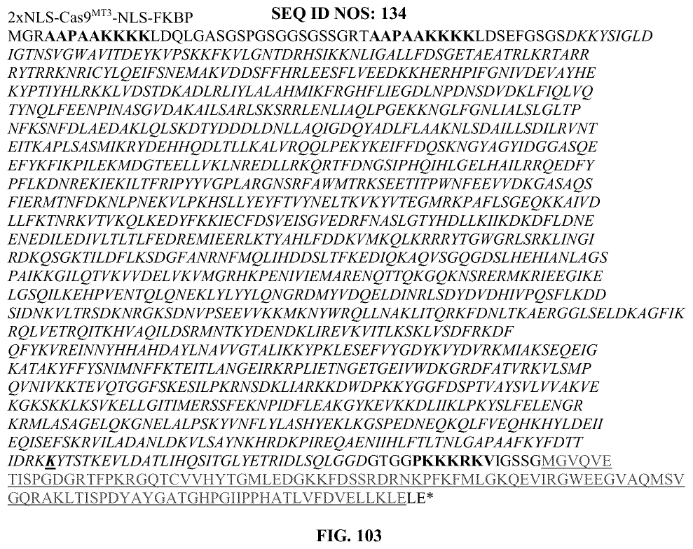

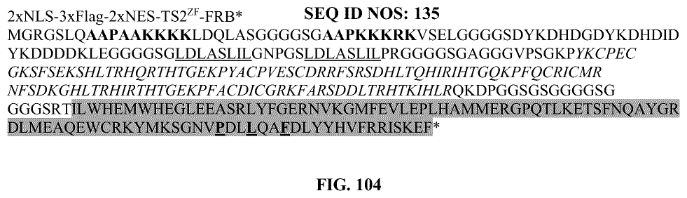

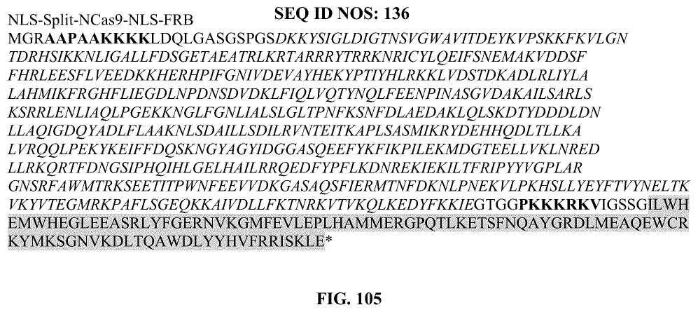

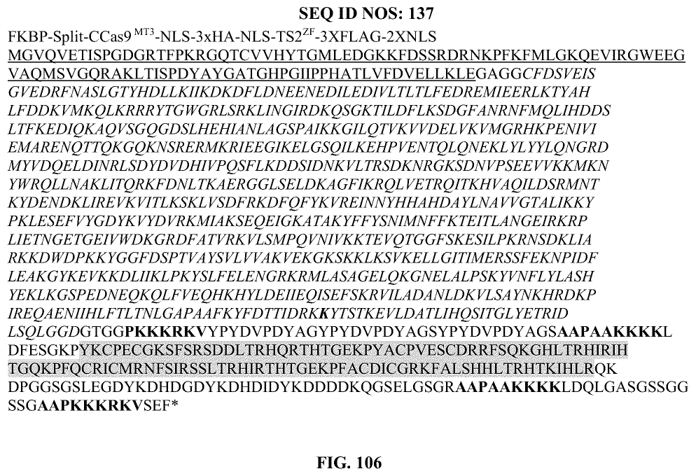

To generate a more precise system, MT3 attenuating mutations were introduced into the split-SpCas9 system. Using this system tethered to a ZFP that recognizes a neighboring sequence within the TS2 genomic region (split-SpCas9^{MT3}-ZFP^{TS2} and a TS3 sgRNA) drug-dependent cleavage of the TS2 target site was achieved. To demonstrate the improvements in precision achieved through drug-dependent systems GUIDE-seq was employed (Tsai, S. Q. et al. Nature biotechnology 33, 187-197 (2015).) For this analysis, the precision of wild-type Cas9 was compared to ae drug-dependent Split-Cas9 system (Zetsche B, et. al. Nature biotechnology. 2015 February; 33(2):139-42); a drug-dependent SpCas9-FKBP/ZFP-FRB* and a drug-dependent split-SpCas9^{MT3}-ZFP^{TS2} through Illumina sequencing of genomic regions that have incorporated GUIDE-seq oligonucleotides. The number of reads that are associated with a locus are indicative of the nuclease cleavage activity. When the nuclease activity of these constructs are assayed with a sgRNA (and ZFP) programmed to recognize the TS2 locus, all of these constructs have high activity at the TS2 target site (FIG. 102). The precision of these constructs is quite different as assessed at one of the most active off-target site (OTG2-1). Here both wild-type Cas9 and the Zhang Split-Cas9 display robust activity, whereas an attenuated SpCas9-FKBP/ZFP-FRB* and a drug-dependent split-SpCas9^{MT3}-ZFP^{TS3} display no apparent activity. Thus nuclease attenuated drug-dependent systems as disclosed herein display dramatic improvements in precision.

H. Increased sgRNA Activity

Truncated sgRNAs (i.e. less than 20 bases of complementarity) have been utilized to increase precision of Cas9/sgRNA complexes by reducing the degree of potential complementarity with off-target sequences. Fu et al., Improving CRISPR-Cas nuclease specificity using truncated guide RNAs. Nature Biotechnology (2014). Cleavage activity of truncated sgRNA was compared between SpCas9 and SpCas9-Zif268. The data demonstrate that SpCas9-Zif268 displays a higher cleavage activity than SpCas9 where both comprise an identical sgRNA, whether the sgRNA may be a full length sequence or a truncated sequence. FIG. 6. This phenomenon was also observed for NmCas9 fusions to ZFPs at endogenous targets for some canonical PAM domains (NNNNGATT (SEQ ID NO: 1), FIG. 75, FIG. 77, FIG. 78; NNNNGCTT (SEQ ID NO: 20), FIG. 81; NNNNGTTT (SEQ ID NO: 21), FIG. 80) the ZFP fusion to NmCas9 enhances the activity of the nuclease, in many cases providing activity where unfused NmCas9 is inactive. Thus, a pDBD fusion may provide a method to fully activate Cas9 nucleases even at canonical PAM sites for which they have poor or no activity. Although it may be not necessary to understand the mechanism of an invention, it is believed that this improved activity represents an additional advantage over the standard nuclease frameworks when using genomic targets.

### I. Cas9 PAM Recognition Sequence Mutations

The PAM interaction domain (PI) has been defined based on structural information on the Cas9/sgRNA/target complex and domain substitution studies. Jinek et al., Structures of Cas9 Endonucleases Reveal RNA-Mediated Conformational Activation. Science (2014); Nishimasu et al., Crystal Structure of Cas9 in Complex with Guide RNA and Target DNA. Cell 1-23 (2014); PMID 25079318. Based on the reported crystal structures, there is evidence that for a conservation of residues within the PI domain between Cas9 isoforms from different species share a common PAM recognition sequence. Fonfara et al., Phylogeny of Cas9 determines functional exchangeability of dual-RNA and Cas9 among orthologous type II CRISPR-Cas systems. Nucleic Acids Research 42, 2577-2590 (2014).

In some embodiments, the present invention contemplates a SpCas9 protein comprising two arginine residues at positions 1333 and 1335 (i.e., a RKR motif) that may be a NGG PAM recognition domain. In one embodiment, the present invention contemplates a mutated Cas9 protein (Cas9MT^{#}) comprising an ¹³³³R→¹³³³K mutation or an ¹³³⁵R→S335S mutation. The activity of a Cas9^{MT#} or a Cas9^{MT#}-Zif268 were tested using a target site that contains NGG, NAG or NCG PAMs with a neighboring Zif268 site. The data show that Cas9^{MT#} may be inactivated by a single mutation, only modestly effect Cas9^{MT#}-Zif268 activity, with the exception of the ¹³³⁵R→¹³³⁵S mutation (#4) where activity may be abrogated. The ¹³³³R→¹³³³K mutant (#1) displays similar activity to the wild type (WT) Cas9-Zif268 fusion. FIG. 9. These data suggest that a mutant version of Cas9 that may not be competent for cleavage on its own, but requires an associated DNA-binding domain for function, while still retaining the specificity of the sgRNA and PAM recognition site sequence. This coordination should dramatically improve Cas9 nuclease function precision. It may also be possible to select alternate residues at these positions that allow the PAM specificity of Cas9 to be reprogrammed.

As disclosed herein, a SpCas9-DBD fusion protein displays an improved activity and precision, especially when combined with a mutated PAM recognition sequence that attenuates intrinsic DNA binding affinity. While the presently disclosed mutations weaken native cleavage activity, it may be likely that further attenuation of the DNA-binding affinity of SpCas9 may increase absolute DBD dependence. For example, mutagenizing at least two regions of Cas9 may be expected to reduce its intrinsic activity: 1) the PAM recognition residues, and 2) apparent phosphate-contacting residues near the PAM binding site.

In one embodiment, the present invention contemplates mutations to the PAM recognition residues comprising arginines (e.g., R¹³³³ & R¹³³⁵) that participate in base-specific binding. In one embodiment, the mutation may be a substitution. In one embodiment, the mutation may be a combination mutation (e.g., a R1333K and a R1335K). In one embodiment, the mutations that abrogate the independent binding of the Cas9 nuclease to its target site are in phosphodiester backbone contacting residues that reduce the affinity of Cas9 for the DNA. A GFP reporter assay may be used with the array of 16 PAM target sites to monitor nuclease activity of each mutant with and without the DBD.

In one embodiment, the present invention contemplates mutations to Cas9 comprising arginine or lysine residues that participate in DNA phosphate binding. Neutralization of phosphate contacts within DBDs may be a demonstrated method to modulate their binding affinities. Khalil et al., A synthetic biology framework for programming eukaryotic transcription functions. Cell. 2012 Aug. 3; 150(3):647-58. Lysines that are well-positioned to make non-specific contacts with the DNA downstream of the PAM contacting residues. Anders et al., Structural basis of PAM-dependent target DNA recognition by the Cas9 endonuclease. Nature. 2014 Sep. 25; 513(7519):569-73; and FIG. 22. These lysine residues are distal from an sgRNA-DNA interaction site, and so it would not be expected to affect the efficiency of R-loop formation or the precision of DNA cleavage. Szczelkun et al., Direct observation of R-loop formation by single RNA-guided Cas9 and Cascade effector complexes. Proc Natl Acad Sci USA. 2014 May 27; and Sternberg et al., DNA interrogation by the CRISPR RNAguided endonuclease Cas9. Nature. 2014 Mar. 6; 507(7490):62-7. These lysines may be mutated to alanine individually, or in combination, and then nuclease activity of these mutants are compared with or without an attached DBD on genomic targets by using, for example, a T7EI assay. Promising lysine mutants may be combined with PAM mutants identified above to further attenuate Cas9 DNA-binding affinity.

Mutations may be identified in a PAM interaction domain and non-specific phosphate contacts that completely inactivate Cas9 activity independent of an attached DBD. Further characterization of promising constructs may be performed using PCR amplification of a genomic target and deep sequencing to quantify SpCas9^{MT} activity with and without the DBD. PAM recognition domains serve not only as an initial DNA-binding toehold for Cas9, but the binding energy may be also used to provide local DNA unwinding in preparation for (or coupled to) R-loop nucleation, and perhaps allosteric nuclease activation. Thus, accumulated mutations could compromise DNA unwinding and activation so much that a defect cannot be overcome by an appended DBD.

In one embodiment, the present invention contemplates an SpCas9^{MT#}-DBD fusion protein comprising a truncated sgRNA (tru-gRNA). Although it may be not necessary to understand the mechanism of an invention, it is believed that tru-gRNAs (i.e., for example, TS1, TS2, TS3 & TS4) improve, but do not eliminate, off-target activity. tru-gRNA (TS1) tested in a GFP reporter assay was found to display similar, or even slightly improved, on-target activity when used with a Cas9-DBD fusion protein relative to Cas9 alone, FIG. 6. Similar improvements may be expected in precision with tru-gRNA/SpCas9^{MT}-DBD combinations on endogenous targets. Should residual off-target effects persist even when a SpCas9^{MT}-DBD fusion protein may be combined with a tru-gRNA, dual Cas9 nickases (nCas9) or dual FokI-dCas9 nucleases in the context of two different DBDs may help target each monomer to a neighboring target site. In one embodiment, a dual Cas9-DBD fusion protein may comprise orthogonal Cas9 systems (e.g. nSpCas9-DBD & nNmCas9-DBD).

In one embodiment, the present invention contemplates an SpCas9 comprising refined PAM specificity wherein genome editing may be improved. In one embodiment, the present invention contemplates a plurality of SpCas9^{MT} variants that can target essentially any sequence within the genome with maximal precision, and that may be capable of allele-specific targeting. Selection strategies that generate SpCas9 variants having altered PAM specificity (SpCas9-PAM^{MT}) have been discussed herein in the context of an altered SpCas9-DBD fusion protein. The precision of these SpCas9^{MT}-DBD variants may be characterized within a genome and tested for allele-specific targeting, using PAM SNPs as discriminators.

For example, SpCas9-PAM specificity may be refined through mutagenesis of PAM recognition residues. A GFP reporter assay testing PAM recognition mutants demonstrated attenuation of intrinsic nuclease activity (e.g., for example, R¹³³³ & R¹³³⁵). FIG. 9. To assess the potential breadth of PAM specificities that can be achieved with SpCas9, the impact of additional mutations may tested using amino acids including, but not limited to, K¹¹⁷, S¹¹³⁶, E¹²¹⁹, R¹³³³ & R¹³³⁵ that make direct or indirect contact with a PAM. Anders et al., Structural basis of PAM-dependent target DNA recognition by the Cas9 endonuclease. Nature. 2014 Sep. 25; 513(7519): 569-73; and FIG. 9.

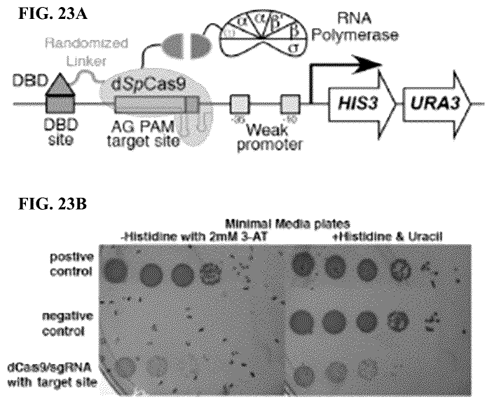

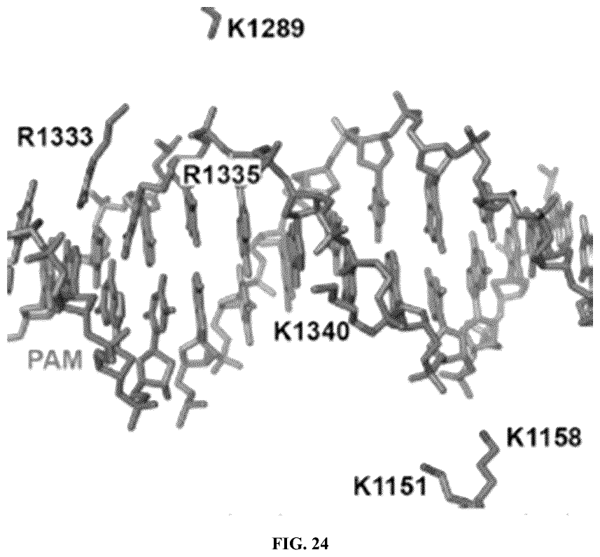

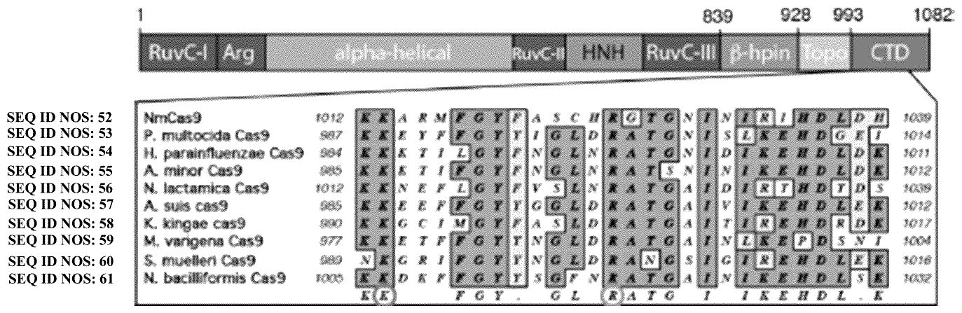

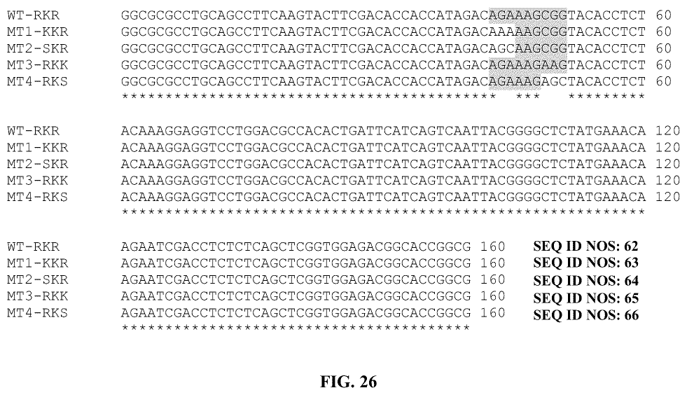

Using a B2H system, libraries may be searched of sufficient complexity (˜108) to cover all possible amino acid combinations for possible PAM recognition mutants (FIG. 23A&B). Meng et al., A bacterial one-hybrid system for determining the DNA-binding specificity of transcription factors. Nature Biotechnology. 2005 August; 23(8):988-94; and Noyes et al., A systematic characterization of factors that regulate Drosophila segmentation via a bacterial one-hybrid system. Nucleic Acids Research. 2008 May; 36(8):2547-60. Randomized libraries can be assembled by known PCR methods and cloned into a B2H dCas9-DBD/sgRNA expression plasmid. Although it may be not necessary to understand the mechanism of an invention, it is believed that these selections affect binding, not nuclease activity, and therefore may be performed in a nuclease-dead (dCas9) fusion protein. These libraries can then be screened as dCas9-DBD/sgRNA combinations on HIS3/URA3 reporter vectors that contain each of the 16 different PAMs (with NGG as a positive control) to select combinations that permit recognition of each PAM. A dCas9-DBD library with each PAM target site may be plated on various concentrations of 3-AT to define a selection stringency where only a few hundred clones survive. These clones may be pooled and deep-sequenced to identify a consensus at the randomized positions. Chu et al., Exploring the DNA-recognition potential of homeodomains. Genome Research. 2012 October; 22(10):1889-98. The specificity of SpCas9^{MT} clones, similar to the consensus sequence for each PAM selection, can be evaluated on all 16 PAMs in the GFP reporter assay and subsequently within a genome by T7EI assay. Cas9 mutant dependence on an attached DBD for nuclease function can be attenuated as necessary by mutation of residues that contact the DNA phosphates.

A negative selection protocol may be used to identify functional nucleases at alternate PAMs. For example, a bacterial 5-FOA/URA3 counter-selection system was reported that may be suitable for the identification of Cas9-DBD variants with mutated PAM sequences. Meng et al., Counter-selectable marker for bacterial-based interaction trap systems. Biotechniques. 2006 February; 40(2):179-84. For example, a low-copy, IPTG-inducible URA3 plasmid (pSC101 origin, kanR-marked) containing a Cas9-DBD target site may be introduced with a mutated PAM sequence into a uracil auxotroph strain (ApyrF). Meng et al., A bacterial one-hybrid system for determining the DNA-binding specificity of transcription factors. Nature Biotechnology. 2005 August; 23(8):988-94; and Lutz et al., Independent and tight regulation of transcriptional units in Escherichia coli via the LacR/O, the TetR/O and AraC/I1-I2 regulatory elements. Nucleic Acids Research. 1997 Mar. 15; 25(6):1203-10. After transformant selection (KanR), these cells may be electroporated with a Cas9-DBD/sgRNA plasmid library (marked with ampR), and plated on YM media with ampicillin, IPTG (to induce URA3) and 5-FOA. Functional Cas9-DBDs variants can cleave and eliminate the URA3 plasmid, permitting survival; cells with nonfunctional Cas9-DBDs retain the plasmid and die via 5-FOA counter-selection. Surviving clones may be pooled and deep-sequenced to identify a consensus at the randomized positions. Chu et al., Exploring the DNA-recognition potential of homeodomains. Genome Research. 2012 October; 22(10):1889-98. The specificity of individual SpCas9^{MT} clones similar to the consensus sequence for each PAM selection can then be evaluated as described above using, for example, a B2H selection approach.

Alternatively, a library depletion strategy may be employed that may be analogous to RNAi-based strategies in mammalian cells to identify essential genes in a particular pathway. Murugaesu et al., High-throughput RNA interference screening using pooled shRNA libraries and next generation sequencing. Genome Biol. 2011; 12(10):R104; Moffat et al., A lentiviral RNAi library for human and mouse genes applied to an arrayed viral high-content screen. Cell. 2006 Mar. 24; 124(6):1283-98; and Root et al., Genome-scale loss-of-function screening with a lentiviral RNAi library. Nature Methods. 2006 September; 3(9):715-9. In these screens, shRNA clones that target essential genes in a pathway of interest are depleted from the initial library because they are lost from the population.

Deep sequencing may be used to compare the distribution of clones in the initial library and in the survivors to identify shRNAs that are lost, which are then retested individually to assess their activity. Similarly, a depletion strategy may be used to identify barcoded clones of the above library that are active in bacteria at a desired PAM site within a kanR-marked plasmid. Based on a protocol for RNAi-based screens, an approximate ˜1000-fold oversampling of a library may observe reliable depletion of active Cas9-DBD clones. Thus, a smaller library (˜10⁵ clones) may be used to retain sufficient depth in a lane of HiSeq2000 sequencing (˜2×10⁸ reads/lane) to effectively employ this approach. Clones may be recovered that define a primary consensus sequence useful for bootstrapping through a second library construction (with fixed residues at positions of consensus from clones recovered from the first selection) and a deeper search of neighboring sequence space to identify the most active sequences. The specificity of each of these selected SpCas9-PAM^{MT} clones may then be evaluated using a B2H selection technique as described above.

In one embodiment, the present invention contemplates a method for determining precision of SpCas9^{MT} clones using a genome-wide survey. For example, precision of an SpCas9^{MT} clone at a specific genomic target site and predicted off-target genomic sites can be determined by comparing new target sites for each SpCas9^{MT} clone that have an appropriate PAM sequence (i.e., for example, a specific non-NGG PAM). An appropriate DBD can be constructed to target each sequence to create an SpCas9^{MT}-DBD fusion protein. The most favorable off-target sites can then be predicted for these sgRNAs using, for example, a CRISPRseek algorithm. Zhu et al., CRISPRseek: A Bioconductor Package to Identify Target-Specific Guide RNAs for CRISPR-Cas9 Genome-Editing Systems. PLoS ONE. 2014; 9(9):e108424. In addition, GUIDE-seq analysis can be performed (Tsai, S. Q. et al. GUIDE-seq enables genome-wide profiling of off-target cleavage by CRISPR-Cas nucleases. Nature biotechnology 33, 187-197 (2015).). Regions exhibiting significant GUIDE-seq oligonucleotide incorporation may be characterized for off-target cleavage rates in the nuclease-treated cells using PCR-based deep sequencing. Gupta et al., Zinc finger protein-dependent and -independent contributions to the in vivo off-target activity of zinc finger nucleases. Nucleic Acids Research. 2011 Jan. 1; 39(1):381-92.

In one embodiment, the present invention contemplates a SpCas9^{MT}-DBD fusion protein comprising mutated PAM sequences comprising unexpected and superior specific genomic target binding precision. Although it may be not necessary to understand the mechanism of an invention, it is believed that a Cas9^{MT}-DBD fusion protein allows a precise cleavage of nearly any sequence within the genome and can provide allele-specific targeting through the use of SNPs that distinguish between alleles. For example, the inactivation of specific dominant-negative alleles could have great utility for gene therapy. In one embodiment, the method contemplates an SNP for siRNA-mediated silencing of Huntington alleles that contain CAG repeat expansions. Pfister et al., Five siRNAs targeting three SNPs may provide therapy for three-quarters of Huntington's disease patients. Curr Biol. 2009 May 12; 19(9):774-8. In principle, Cas9s with allele-specific activity could provide an alternate therapeutic strategy to disable specific harmful alleles in patients.

In one embodiment, the present invention contemplates a method of stringently discriminating between single alleles by targeting a particular heterozygous SNP within a PAM. The data presented herein demonstrates that Cas9 and various PAM recognition mutants already generated could utilize a Cas9-DBD fusion protein to edit single alleles that are distinguished by functional vs. non-functional PAMs.

A database may be used to define cell lines with SNPs that could be used to test the allele-specific discrimination of a Cas9-DBD fusion protein. Forbes et al., COSMIC: mining complete cancer genomes in the Catalogue of Somatic Mutations in Cancer. Nucleic Acids Research. 2011 January; 39(Database issue):D945-50. The Forbes et al. database contains sequences from >100 cell lines, each with a searchable table of validated SNPs (e.g., 26 heterozygous SNPs in U2OS cells). Potentially distinguishable SNPs and sequence candidate loci in cell lines can be identified from this database to confirm heterozygosity. For validated SNPs, SpCas9^{MT}-DBD/sgRNA combinations may be designed to target a single allele. The allelic targeting ratios (relative to negative controls lacking the cognate sgRNA or the appended DBD) can be determined by deep-sequencing PCR amplicons from treated cells.

PAM mutations can also be defined that attenuate NmCas9 activity to achieve dependence on an attached DBD for nuclease activity (FIG. 49A&B). Similar to SpCas9 platforms, two different approaches may be taken to weaken intrinsic DNA binding by NmCas9: 1) attenuation of PAM recognition, and 2) neutralization of arginine and lysine contacts to DNA phosphates. Although the exact structure of NmCas9 has not been reported, a structure of a related Type II-C Cas9 from A. naeslundii may be available. Jinek et al., Structures of Cas9 endonucleases reveal RNA mediated conformational activation. Science. 2014 Mar. 14; 343(6176):1247997. The A. naeslundii Cas9 structure confirms that a core nuclease domain organization may be similar between the Type II-A (SpCas9) and Type II-C(NmCas9) families, though peripheral domains differ. Consequently, a C-terminal region (e.g., positions 928 to 1082) may provide the best candidates for PAM- and phosphate-contacting residues to optimize NmCas9 fusion proteins. As with SpCas9 fusion proteins two complementary approaches may identify these residues: 1) protein-DNA photo-crosslinking and 2) sequence conservation in highly related orthologs.

A preferred PAM of NmCas9 (i.e., for example, NNNNGATT (SEQ ID NO: 1)), wherein a T may be well-tolerated in place of the A, may be suited for protein-DNA photo-crosslinking using a commercially available, photoactivatible crosslinker 5-iododeoxyuridine (5IdU), which may be isosteric with T90. Each of the three individual T-to-5IdU substitutions within the NNNNGTTT PAM of an oligonucleotide duplex may be bound to a purified, nuclease-dead NmCas9 (i.e., for example, a D16A/H588A double mutant, already expressed and purified) in the presence of a complementary sgRNA.

A single T can also be substituted on an opposite strand of the same duplex that carries a NNNNGATT PAM. Photo-crosslinking efficiency for each radiolabeled, 5IdU-substituted target duplex (following irradiation at 308 nm) can also be determined by SDS-PAGE. Wolfe et al., Unusual Rel-like architecture in the DNA-binding domain of the transcription factor Fact. Nature. 1997 Jan. 9; 385(6612):172-6; and Liu et al., Evidence for a non-alpha-helical DNA-binding motif in the Rel homology region. Proc Natl Acad Sci USA. 1994 Feb. 1; 91(3):908-12. Mutant PAMs with inactivating mutations on the non-5IdU-substituted strand can serve as specificity controls. Photo-crosslinking reactions for 5IdU positions displaying efficient, specific crosslinking may be scaled up for mass-spectrometric analysis of trypsin- and S1 nuclease/phosphatase-digested peptide fragments.

DNA contact residues identified by photo-crosslinking, as well as nearby arginine, lysine and glutamine residues, may be mutated and activity of each NmCas9^{MT#} relative to wild-type NmCas9 evaluated in a GFP reporter assay. Luscombe et al., Amino acid-base interactions: a three-dimensional analysis of protein-DNA interactions at an atomic level. Nucleic Acids Research. Oxford University Press; 2001 Jul. 1; 29(13):2860-74. NmCas9^{MT#} clones with attenuated activity may then be fused to DBDs to test for recovery of nuclease activity. PAM specificities can be evaluated in a GFP reporter assay, where initially all PAM variants can be evaluated that have three of the four bases in the NNNNGATT consensus sequence preserved (e.g., 12 combinations).

The above discussed identification of SpCas9 PAM recognition residues, R¹³³³ & R¹³³⁵, was made before any reported structure of these interactions. This discovery was facilitated both by available SpCas9 structural models and sequence alignments of closely related Cas9 orthologs, with the expectation that Cas9-DNA contacts are likely to be conserved. In protein-DNA complexes, guanine contacts (GATT PAM) and DNA phosphate contacts are likely to be mediated by either arginine or lysine residues. Luscombe et al., Amino acid-base interactions: a three-dimensional analysis of protein-DNA interactions at an atomic level. Nucleic Acids Research. Oxford University Press; 2001 Jul. 1; 29(13):2860-74. Consequently, mutations of conserved NmCas9 arginine or lysine residues to an alanine are most likely to affect cleavage activity. FIG. 25. Attenuated clones can then be tested as NmCas9^{MT}-DBD fusions to confirm recovery of nuclease activity. Some of these mutations successfully attenuate NmCas9 activity (K1013A and R1025A), which can be restored by an attached DBD (FIG. 49A&B)

Based on the above data demonstrating attenuation of SpCas9^{MT#} nuclease activity, it can be expected that, as a result of Cas9 PAM amino acid conservation, NmCas9^{MT} would also demonstrate attenuated nuclease activity. The analysis of relevant residues may be aided by photo-crosslinking data, which should help to clarify DNA-proximal regions. Alterations in PAM specificity for these mutants can be evaluated in the GFP reporter assay. Genome editing activity of favorable NmCas9^{MT} clones can be evaluated on genomic targets in HEK293T cells fused to DBDs programmed to bind neighboring sites. Differences in activity between each NmCas9^{MT} versus NmCas9^{MT}-DBD can be examined by T7EI assay. As with SpCas9, further characterization may be performed using PCR amplification of the genomic targets and deep sequencing to quantify editing frequencies at each target site with and without the DBD. Improvements in precision can also be further validated using the above described genome-wide analysis.

For example, a genome-wide assay may be used to define optimal NmCas9^{MT#}-DBD fusion proteins for precise target cleavage in human cell lines. Precision of the most promising NmCas9^{MT#}-DBD clones can be evaluated at target sites and predicted off-target sites within the genome. Appropriate DBDs can be created to facilitate targeting of each genomic sequence with an NmCas9^{MT#}-DBD fusion protein. A set of the most favorable off-target sites can be predicted for these sgRNAs considering both the similarity of the sgRNA to genomic sequences and possible alternate PAMs that could be functional for each NmCas9^{MT}#clone based on an evaluation in a GFP reporter assay and predictions developed using the CRISPRseek algorithm. Zhu et al., CRISPRseek: A Bioconductor Package to Identify Target-Specific Guide RNAs for CRISPR-Cas9 Genome-Editing Systems. PLoS ONE. 2014; 9(9):e108424. In addition, GUIDE-seq analysis can be performed (Tsai, S. Q. et al. GUIDE-seq enables genome-wide profiling of off-target cleavage by CRISPR-Cas nucleases. Nature biotechnology 33, 187-197 (2015).). Regions exhibiting significant GUIDE-seq oligonucleotide incorporation may be characterized for off-target cleavage rates in the nuclease-treated cells using PCR-based deep sequencing.

### J. Improved Cas9 Linkers

In one embodiment, the present invention contemplates a Cas9-DBD construct comprising a linker. In one embodiment, the linker comprises approximately sixty (60) amino acids. Although it may be not necessary to understand the mechanism of an invention, it is believed that such a linker improves the precision of specific genomic target binding. It has been observed that if a DBD binding site is merely repositioned or reoriented relative to the specific genomic target little improvement in precision results. These data indicate that linker flexibility reduces precision via off-target binding due to a large number of sgRNA/DBD binding site permutations that can potentially be cleaved. GFP reporters may be constructed containing alternate spacing and orientation of a DBD binding site relative to a Cas9 target site with a suboptimal NAG PAM. This configuration may also include finer intervals around the most active positions, as well as positions further removed from the Cas9 target site, to better define the distance dependence.

Fusion proteins such as SpCas9-Zif268 or SpCas9-TAL268 may contain a series of shorter linkers to define a minimal length that retains maximum activity at one (or more) binding site positions, but may place further restrictions on activity at other binding site positions. In one embodiment, the present invention contemplates a Cas9-TALE fusion protein or a Cas9-ZFP fusion protein comprising an optimized linker that can recognize virtually any target site. Cermak et al., Efficient design and assembly of custom TALEN and other TAL effector-based constructs for DNA targeting. Nucleic Acids Research. 2011 July; 39(12):e82-2; Lamb et al., Directed evolution of the TALE N-terminal domain for recognition of all 5′ bases. Nucleic Acids Research. 2013 November; 41(21):9779-85; Kim et al., A library of TAL effector nucleases spanning the human genome. Nature Biotechnology. 2013 March; 31(3):251-8; and Briggs et al., Iterative capped assembly: rapid and scalable synthesis of repeat-module DNA such as TAL effectors from individual monomers. Nucleic Acids Research. 2012 Jun. 26.

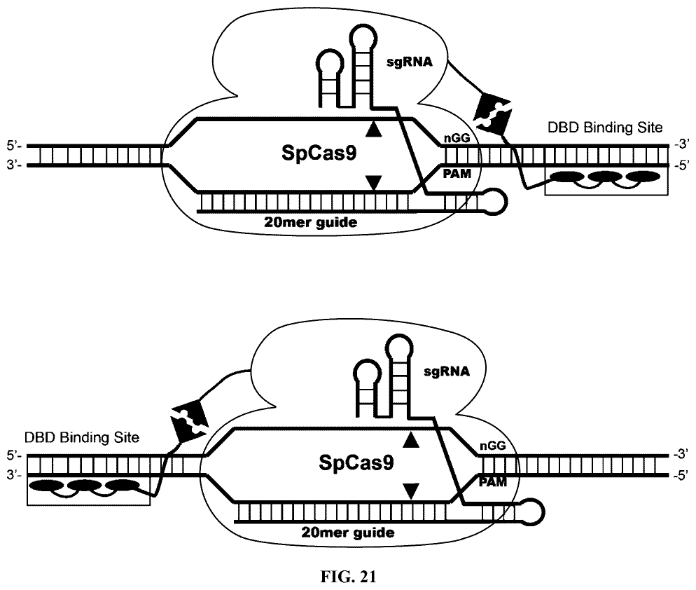

Using a GFP system SpCas9-DBD fusion proteins may be constructed with short linkers (e.g., less than sixty amino acids) that display both high activity and more selectivity in the particular arrangement of the Cas9 and DBD binding sites. Although it may be not necessary to understand the mechanism of an invention, it is believed that a maximum improvement in linker length and/or binding site position/orientation for a DBD relative to a Cas9 nuclease will differ between ZFPs and TALEs due to their respective structural folds and docking with the DNA. Mak et al., The crystal structure of TAL effector PthXol bound to its DNA target. Science. 2012 Feb. 10; 335(6069):716-9; and Deng et al., Structural basis for sequence-specific recognition of DNA by TAL effectors. Science. 2012 Feb. 10; 335(6069):720-3; and Pavletich et al., Zinc finger-DNA recognition: crystal structure of a Zif268-DNA complex at 2.1 A. Science. 1991 May 10; 252(5007):809-17. This linkage will also need to be optimized for any Cas9-nuclease fusion to an orthogonal Cas9 used as the DTU (FIG. 20). Likewise if dimerization domains are employed to associate the Cas9 nuclease with the DTU, it may be likely that a linker between a dimerization domain and a Cas9 nuclease or a dimerization domain and a DTU will need to be improved. (FIG. 20 & FIG. 21).

A functional B2H selection system was established that may be sensitive to the binding of nuclease-dead SpCas9 (dSpCas9) to a target site upstream of a pair of selectable reporter genes. FIG. 23A&B. This B2H selection strain may be a histidine and uracil auxotroph, so survival on minimal media lacking histidine and uracil requires expression of HIS3 and URA3 genes from a reporter vector containing a very weak core promoter. Meng et al., A bacterial one-hybrid system for determining the DNA-binding specificity of transcription factors. Nature Biotechnology. 2005 August; 23(8):988-94; Noyes et al., A systematic characterization of factors that regulate Drosophila segmentation via a bacterial one-hybrid system. Nucleic Acids Research. 2008 May; 36(8):2547-60; and Meng et al., Counter-selectable marker for bacterial-based interaction trap systems. Biotechniques. 2006 February; 40(2):179-84. Transcriptional activity of these reporter genes may be increased dramatically by recruiting RNA polymerase via a two-hybrid interaction. A dCas9/sgRNA complex has been established that can activate these reporter genes in the context of a two-hybrid interaction system. FIG. 23A&B. Improved linker lengths for either TALE or ZFP DBD domains may be defined by a GFP reporter analysis. Combinatorial randomization of a conventional linker library and/or randomization of amino acid positions that are most proximal to Cas9 and a DBD are likely to provide linkers that have improvements at junction points. For example, an improved ZFP-homeodomain linker selection was identified with specific residues (e.g., for example, proline) at positions neighboring each DBD. A dCas9 that targets a sub-optimal NAG PAM may be combined with an optimally positioned ZFP or TALE binding site to select dCas9-ZFP or dCas9-TALE constructs with improved activity in the selection system. Clones with improved binding activity (and thus improved linkers) may be recovered by plating the cells on increasing concentrations of 3-aminotriazole (3-AT, a competitive inhibitor of His3) until only a few clones survive. Selected linkers for Cas9-ZFPs or Cas9-TALEs can be validated as nucleases on genomic targets in HEK293T cells.

Linkages for NmCas9-DBD fusion proteins may also improve precision and activity using a similar procedure to that described above for SpCas9. In particular, an improvement protocol finds a fusion point (N- or C-terminal) and approximate linker length capable of creating a functional fusion between NmCas9 and a DBD (e.g., TALE or ZFP). PAM specificities have been interrogated for NmCas9 and may be believed to involve a consensus NNNNGATT sequence. To evaluate NmCas9-DBD fusion activities a suboptimal PAM (i.e., for example, NNNNGAAT) may be used to assess improvements in activity that are imparted by a fused DBD.

As discussed above in the context of SpCas9, experiments can be carried out in two steps to validate a functional NmCas9 fusion: 1) using a GFP reporter assay to define an optimal linker length; and 2); a bacterial (e.g., E. coli) two hybrid selection of the linker sequence. Esvelt et al., Orthogonal Cas9 proteins for RNA guided gene regulation and editing. Nature Methods. 2013 November; 10(11):1116-21. The ability to utilize a NmCas9 system with a bacterial selection system has been widely reported. Hou et al., Efficient genome engineering in human pluripotent stem cells using Cas9 from Neisseria meningitidis. Proceedings of the National Academy of Sciences. 2013 Sep. 24; 110(39):15644-9; Zhang et al., Processing-independent CRISPR RNAs limit natural transformation in Neisseria meningitidis. Molecular Cell. 2013 May 23; 50(4):488-503; Zhu et al., Using defined finger-finger interfaces as units of assembly for constructing zinc-finger nucleases. Nucleic Acids Research. 2013 Feb. 1; 41(4):2455-65; Gupta et al., An optimized two-finger archive for ZFN-mediated gene targeting. Nature Methods. 2012 Apr. 29; 9(6):588-90; Meng et al., A bacterial one-hybrid system for determining the DNA-binding specificity of transcription factors. Nature Biotechnology. 2005 August; 23(8):988-94; Noyes et al., A systematic characterization of factors that regulate Drosophila segmentation via a bacterial one-hybrid system. Nucleic Acids Research. 2008 May; 36(8):2547-60; Noyes et al., Analysis of homeodomain specificities allows the family-wide prediction of preferred recognition sites. Cell. 2008 Jun. 27; 133(7):1277-89; and Enuameh et al., Global analysis of Drosophila Cys₂-His₂ zinc finger proteins reveals a multitude of novel recognition motifs and binding determinants. Genome Research. 2013 June; 23(6):928-40.

Functionality of the NmCas9-DBDs may be verified through assays on genomic target sites with DBDs that are programmed to recognize neighboring sequences, where activity can be assessed by T7EI assay. In these genomic assessments, activity on properly spaced/oriented binding sites and the absence of activity on improperly spaced/oriented sites can be determined.

III. Improved Precision Using Mutant pDBDs

In some embodiments, the present invention contemplates a chimeric Cas9 system that dramatically improves the precision and targeting range of the Cas9 nuclease. In one embodiment, precision and targeting range is improved by augmention of its specificity with an attached pDBD. In one embodiment, the Cas9-pDBD precision is tunable. In one embodiment, the tunable precision includes, but is not limited to, specificity and/or affinity of the associated pDBD. Although it is not necessary to understand the mechanism of an invention, it is believed that therapeutic genome editing, where cleavage precision is of paramount importance, utilizing customized Cas9-pDBDs will play a role in clinical development process.

A. Improved Precision With Mutant Cas9 pDBD Fusions

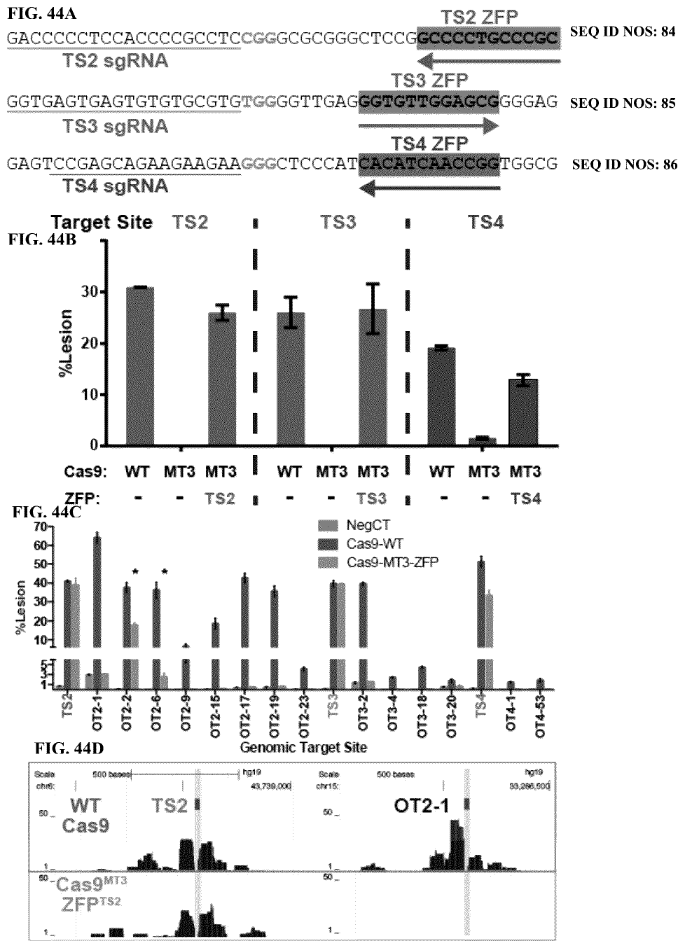

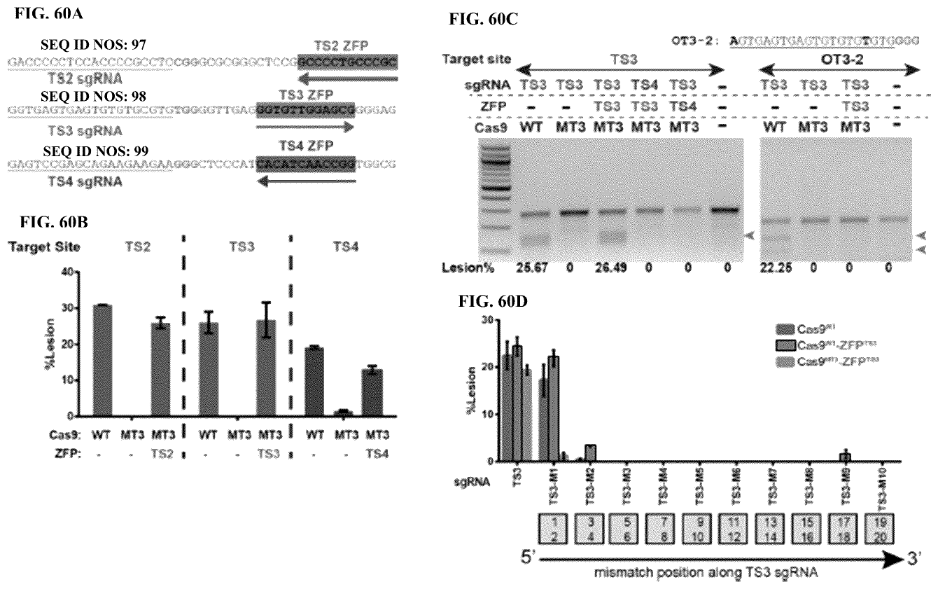

The data presented herein evaluates an improved precision of a SpCas9^{MT#}-pDBD framework at SpCas9 target sites (e.g., for example, TS2, TS3 & TS4; all with NGG PAMs). SgRNAs that recognize these sites have defined on- and off-target activities, which provide a known benchmark to assess improvements in precision. A ZFP was constructed to recognize a sequence near each target site and compared the editing activities of sgRNA programmed SpCas9, SpCas9^{MT3} and SpCas9^{WT3}-ZFP^{TS#}. FIG. 44A. It was confirmed that SpCas9^{MT3} was nearly inactive at all target sites, where this activity was restored by the presence of a cognate ZFP fusion. FIG. 44B. However, the activity was not rescued with a non-cognate sgRNA or ZFP (FIG. 13 & FIG. 60C). To assess improvements in precision at previously defined off-target sites, PCR products spanning these loci were deep sequenced for sgRNA^{TS2/TS3/TS4}. The nuclease activity of SpCas9, and SpCas9^{MT3}-ZFP^{TS2/TS3/TS4} was then compared at these target and off-target sites. It was found that SpCas9^{MT3}-ZFP^{TS2/TS3/TS4} dramatically increased the precision of target site cleavage, FIG. 44C. In most cases, utilizing SpCas9^{MT3}-ZFP^{TS2/TS3/TS4} reduced lesion rates at off-target sites to background levels resulting in improvements in specificity of up to 150-fold. Only one off-target site (OT2-2), which has a neighboring sequence that is similar to the expected ZFPTS2 recognition sequence (data not shown), still displays high lesion rates. One other site (OT2-6), displays some residual activity both for SpCas9^{MT3} and SpCas9^{MT3}-ZFP^{TS2} that is above the background error rate within the sequencing data. These data demonstrate a dramatic overall enhancement of the precision of SpCas9^{MT#}-ZFPs relative to standard SpCas9.

To discover new off-target sites of SpCas9^{MT3}-ZFPs, a GUIDE-seq analysis was performed on SpCas9 and SpCas9^{MT3}-ZFP^{TS#}. These data are consistent with the focused deep sequencing data of known off target sites: there is a dramatic improvement in precision for the SpCas9^{MT3}-ZFP^{TS#}. In addition, ESAT peak picking analysis (garberlab.umassmed.edu/software/esat) of the SpCas9^{MT3}-ZFP^{TS#} GUIDE-seq data reveal that there is a dramatic reduction in SpCas9^{MT3}-ZFP^{TS#} off-target activity genome-wide, FIG. 44D. A small number of weak peaks (less than 5 reads, likely noise) are unique to the SpCas9^{MT3}-ZFP^{TS#} (relative to SpCas9 programmed with the same sgRNA), but none appear to be valid off-target sites based on the absence of guide complementary sequence with 6 or fewer mismatches. Thus, SpCas9^{MT} nuclease activity is muzzled, but can be reactivated at specific genomic regions through fusion of a pDBD recognizing a neighboring sequence.

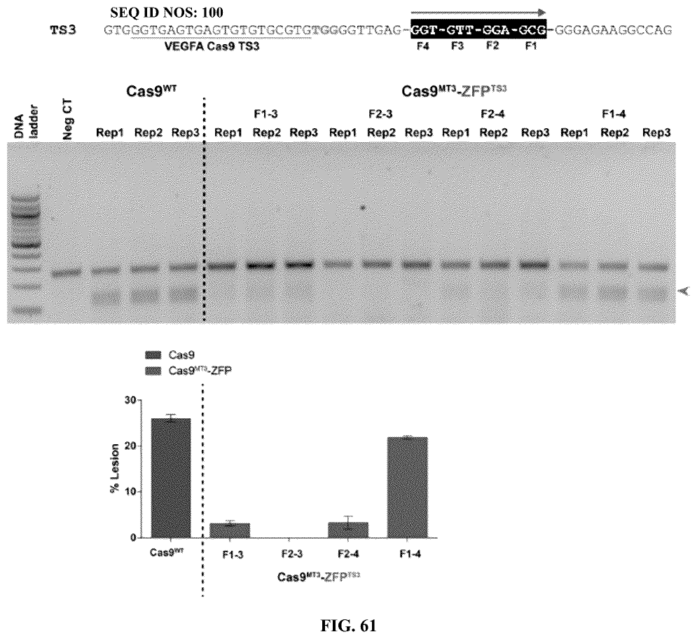

The precision of SpCas9-ZFPs to SpCas9 was compared using sgRNAs with previously defined off-target sites^{14,25}. Three different four-finger ZFPs were constructed to recognize 12 base pair sequences neighboring the TS2, TS3 or TS4 sgRNA target sites for use as SpCas9^{MT3}-ZFP fusions. FIG. 60A. The activity of SpCas9, SpCas9^{MT3} and SpCas9^{MT3}-ZFP^{TS2/TS3/TS4} with a corresponding sgRNA was compared at each target site. In all cases SpCas9^{MT3} dramatically decreased cleavage efficiencies, which were restored by a cognate ZFP fusion, FIG. 60B. The activity of SpCas9^{MT3}-ZFP was dependent on the presence of both a cognate sgRNA and ZFP, FIG. 60C. Consistent with the dependence on ZFP binding, truncation of one zinc finger from either end of ZFP^{TS3} reduced the activity of SpCas9^{MT3}-ZFP^{TS3} at the TS3 target site, and the removal of two zinc fingers abrogated activity, FIG. 61. The introduction of a third stage of target site licensing supplied by the pDBD dramatically increased the precision of SpCas9^{MT3}-ZFP^{TS3} relative to wild type SpCas9 (SpCas9^{WT}); lesion rates at the most active off-target site (OT3-2) for sgRNA^{TS3} were 22% by T7EI assay with wild type Cas9, but were undetectable with SpCas9^{MT3}-ZFP^{TS3}, FIG. 60C.

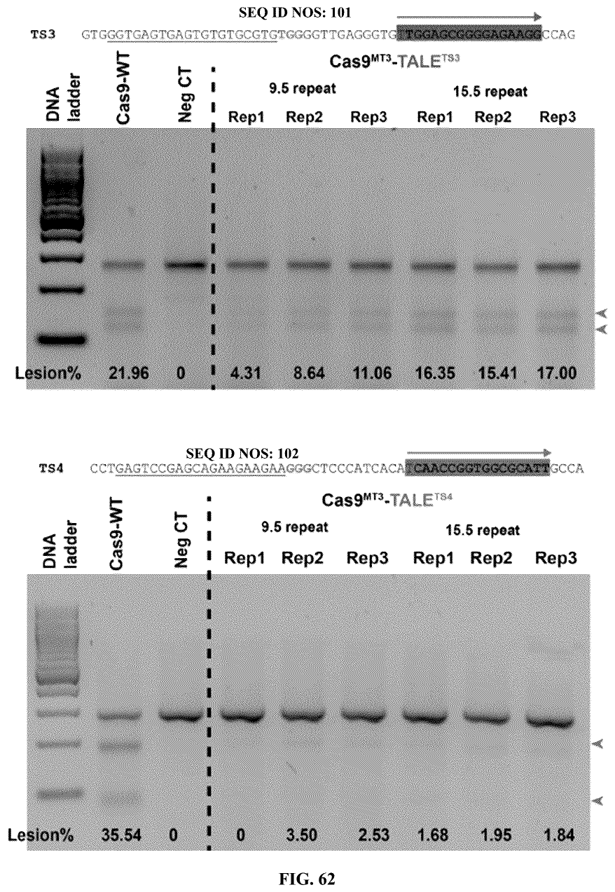

Two TALE arrays were also programmed to target SpCas9^{MT3} to TS3 and TS4 (TALE-TS3 and TALE-TS4). Nuclease activity at the TS3 site but not TS4 can be restored by the related SpCas9^{MT3}-TALE fusion, FIG. 62. To examine the catalytic tolerance of the SpCas9^{MT3}-ZFP^{TS3}/sgRNA complex to mismatches between the guide and a target sequence, a set of guides was used that progressively shift blocks of 2 base mismatches from the 5′ to the 3′ end of the guide sequence. SpCas9^{MT3}-ZFP^{TS3} has a lower tolerance for mismatches between the guide and target site relative to SpCas9^{WT}, whereas SpCas9^{WT}-ZFP^{TS3} appears to modestly increase the tolerance for mismatches, FIG. 60D and Table 3.

TABLE 3
Average nuclease activity (% Lesion) values of TS3 sgRNA mismatches

| SEQ ID | sgRNA | | | Cas9^{WT}- | Cas9^{MT3}- |
|---|---|---|---|---|---|
| NO: | Name | sgRNA sequence | Cas9^{WT} | ZFP^{TS3} | ZFP^{TS3} |
| 23 | TS3 | GGTGAGTGAGTGTGTGCGTG | 22.44 | 24.39 | 19.34 |
| 24 | TS3-M1 | gCCTGAGTGAGTGTGTGCGTG | 17.17 | 21.9 | 1.28 |
| 25 | TS3-M2 | GGCAAGTGAGTGTGTGCGTG | 0.41 | 3.24 | N.D |
| 26 | TS3-M3 | GGTGCTTGAGTGTGTGCGTG | N.D | N.D. | N.D |
| 27 | TS3-M4 | GGTGAGGAAGTGTGTGCGTG | N.D | N.D | N.D |
| 28 | TS3-M5 | GGTGAGTGCTTGTGTGCGTG | N.D | N.D | N.D |
| 29 | TS3-M6 | GGTGAGTGAGCATGTGCGTG | N.D | N.D | N.D |
| 30 | TS3-M7 | GGTGAGTGAGTGCATGCGTG | N.D | N.D | N.D |
| 31 | TS3-M8 | GGTGAGTGAGTGTGGTCGTG | N.D | N.D | N.D |
| 32 | TS3-M9 | GGTGAGTGAGTGTGTGTATG | N.D | 1.57 | N.D |
| 33 | TS3-M10 | GGTGAGTGAGTGTGTGCGCA | N.D | N.D | N.D |

N.D: Not Desecred

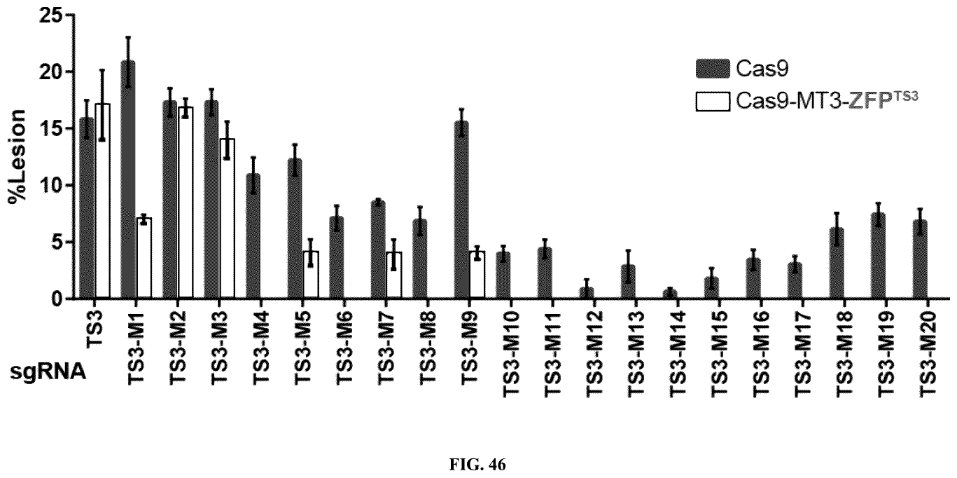

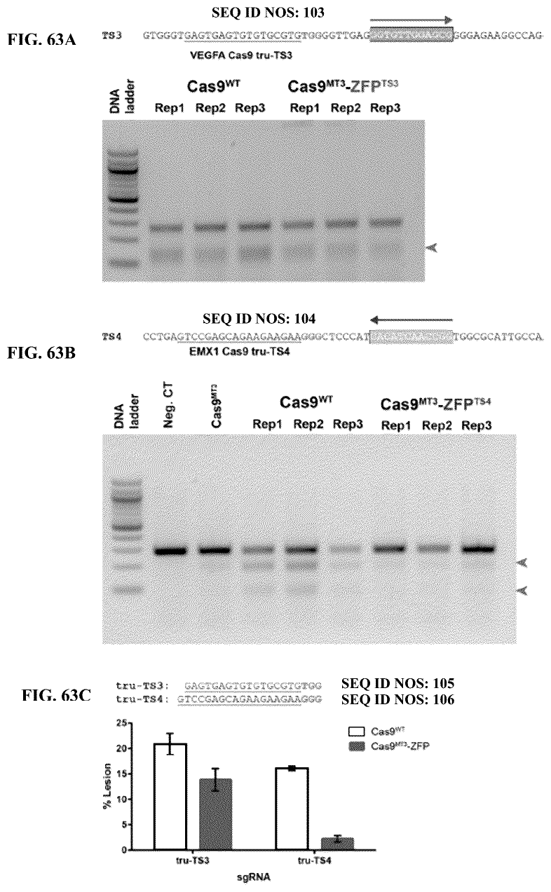

Consistent with an increased sensitivity to disruptions in sgRNA-target interactions, SpCas9^{MT3}-ZFPs exhibit reduced activity with truncated sgRNAs²⁵, confirming that a higher degree of guide-target site complementarity is required for efficient cleavage with our chimeras. FIG. 63A-C. In addition a series of single base mutations with the sgRNA shifted across the TS3 target site indicates that SpCas9^{MT3}-ZFP has superior discrimination to wild-type Cas9 (FIG. 46).

### B. Cas9-pDBD System Tunability

One advantage of a SpCas9-pDBD system over other Cas9 platforms is the ability to rapidly tune the affinity and specificity of the attached pDBD to improve its precision. In one embodiment, improved precision of SpCas9^{MT3}-ZFP^{TS2} was achieved by truncating the four zinc finger array to reduce its affinity for off-target site OT2-2. High activity at the TS2 target site was maintained despite removal of either of the terminal zinc fingers from SpCas9^{MT3}-ZFP^{TS2} However, these truncations reduced or eliminated activity at OT2-2, reflecting a profound improvement in the precision of SpCas9^{MT3}-ZFP^{TS2}. Similarly, utilization of a ZFP^{TS2}* that recognizes an alternate sequence neighboring the TS2 guide target site also abolishes off-target activity at OT2-2, FIG. 45.

Given the improvements in precision realized by these selective alterations in the composition of a ZFP, it should be possible to achieve even greater enhancements in precision via more focused modification of a ZFP composition and a linker connecting it to SpCas9. These data demonstrate the functionality of SpCas9-pDBD chimeras, their broader targeting range and improved precision when compared to standard SpCas9.

C. Increased SpCas9 Precision Through Direct and Drug-Dependent pDBD Fusions

In one embodiment, the present invention establishes a framework to facilitate use of the SpCas9-pDBD system to efficiently design, assay and permute this platform to achieve single-site precision for editing the human genome. There are a number of parameters that remain to be optimized in the SpCas9-pDBD system. For example, an initial four-finger SpCas9-ZFPs still retains a low level of off-target activity, FIG. 44A-D. Some of this is residual activity present in the SpCas9^{MT3} (R1335K) mutant that is independent of a pDBD. In addition, linker length/composition as well as improved pDBD affinity and specificity also contribute to improved precision and efficiency.

### 1. Improved Precision Using SpCas9-pDBD Frameworks

In some embodiments, the present invention contemplates a method utilizing different parameters regulating precision and activity of a SpCas9-pDBD framework to define a framework for highly active and extremely precise nucleases.

a. The Cas9-pDBD Linker

In one embodiment, a SpCas9^{MT3}-pDBD construct is connected by a 60-aa linker and displays improvements in precision, FIG. 2B. Although it is not necessary to understand the mechanism of an invention, it is believed that an improved linker length provides additional precision improvements by reducing the number of alternate (e.g., off-target) sgRNA/pDBD binding site permutations.

For example, a GFP reporter assay is used herein to identify improved linker lengths joining SpCas9 to either ZFPs or TALEs that increase their fidelity of target site cleavage. In one embodiment, the GFP reporter assay defines a minimal linker length for SpCas9-Zif268 and SpCas9-TAL268 constructs that retains maximum activity at one (or more) binding site positions, but places further restrictions on the activity at other positions. Improved linkers may be tested for both activity and precision in the context of SpCas9^{MT3}-pDBDs designed for TS2/TS3/TS4 genomic sites, FIG. 44A-D. Initial activity and precision may be assessed by T7EI assays, FIG. 44A-D and FIG. 45.

The most promising linkers can be further evaluated by GUIDE-seq to assess genome-wide off-target activity. GUIDE-seq results may be verified by targeted deep sequencing of PCR products spanning these loci, FIG. 44A-D. The GFP reporter assay and subsequent validation at the TS2/TS3/TS4 genomic targets via GUIDE-seq can identify linkers that display both high activity and more selectivity in the particular arrangement of SpCas9 and pDBD binding sites. The improved linker length and improved binding site position/orientation may differ between ZFPs and TALEs due to their differing mechanisms of DNA recognition.

b. Improved Precision Using DNA Recognition Modules (ZFP or TALE)

The data herein has shown that the precision of SpCas9-ZFPs is dependent on the number of ZFP recognition modules, where excessive affinity reduces precision, FIG. 45. Alternatively, in some embodiments binding site size and affinity of TALEs or ZFPs can be tuned by changing the number of incorporated recognition modules. For example, ZFPs may be modified by altering the number of fingers, the type inter-finger linkage and/or the number of DNA phosphate contacts. Alternatively TALEs may be modified by altering the number of modules and/or the use of non-canonical RVD recognition residues. As described above, the TS2/TS3/TS4 genomic sites can be utilized to assay activity and precision, first by T7EI assays, followed by evaluation of the most promising SpCas9^{MT3}-pDBDs by GUIDE-seq and targeted deep sequencing. Although it is not necessary to understand the mechanisms of an invention, it is believed that while an optimal number of fingers/modules within a pDBD may vary from site to site, a range of fingers/modules may be defined that is likely to be more favorable with regards to both target activity and precision.

c. SpCas9 Modifications for pDBD Functional Dependence

As shown above, PAM-attenuated SpCas9^{MT3} displays residual nuclease activity at TS2/TS3/TS4 in the absence of the pDBD. Further, attenuation of SpCas9 DNA-binding affinity increases absolute pDBD dependence and thus its precision. In one embodiment, the present invention contemplates at least one mutation in at least two regions of SpCas9 to reduce its intrinsic activity including, but not limited to; i) PAM recognition residues, and ii) phosphate-contacting residues near the PAM binding site.

In one embodiment, the present invention contemplates a Cas9 complex comprising PAM recognition residue mutations. In one embodiment, the mutations are located at arginine residues (e.g., for example, R1333 & R1335) that make base-specific PAM contacts. In one embodiment, the mutations are a combination mutations (e.g. combining R1333K & R1335K). Such combination mutations are believed to further attenuate independent SpCas9 activity but retain activity in the presence of a fused pDBD. The double-strand break (DSB) formation rate in the absence and presence of the pDBD may be estimated by qPCR-based quantification of the rate of capture of GUIDE-seq oligos at each target site (TS2/TS3/TS4) as a proxy for deep sequencing.

In one embodiment, the present invention contemplates arginine or lysine residue mutations that contact DNA phosphates. Although it is not necessary to understand the mechanism of an invention, it is believed that neutralization of phosphate contacts within pDBDs can modulate their binding affinities. In one embodiment, SpCas9 is mutated at lysine or arginine residues that are positioned to make non-specific contacts with the DNA downstream of the PAM-contacting residues, and so should not affect the efficiency of R-loop formation or the precision of DNA cleavage. The activity of these mutants may be assayed as described for the PAM recognition mutants.

Mutations can be identified that render SpCas9 completely dependent on an attached pDBD. Since the capture of GUIDE-seq oligos is not be a perfect surrogate for the rate of DSB formation, lesion rates may be assessed for the most promising mutants by deep sequencing. Alternatively, lysine or arginine mutants can be combined with PAM mutants for further attenuation of SpCas9 DNA-binding affinity. Although it is not necessary to understand the mechanism of an invention, it is believed that the improved precision of the presently disclosed SpCas9^{MT}-pDBDs for TS2/TS3/TS4 are vastly superior to those previously reported. To confirm that superiority, the precision should be shown to be cell line-independent via deep-sequencing and GUIDE-seq analysis.

### 2. Allele-Specific Targeting Using Single Nucleotide Polymorphisms

The ability to selectively inactivate specific dominant-negative alleles could have great utility. For example, single nucleotide polymorphisms (SNPs) have been proposed as discriminators for siRNA-mediated silencing of Huntingtin alleles that contain CAG repeat expansions. Cas9s with allele-specific activity could provide a therapeutic strategy to disable specific harmful alleles. SpCas9 has been used to achieve incomplete discrimination using a SNP within the guide recognition sequence. Analysis of the presently disclosed Cas9^{MT3}-ZFP framework has revealed dramatically improved discrimination for single-base changes within a target sequence. FIG. 46. This increased sensitivity is consistent with improved precision. The feasibility of using SNPs within a guide recognition sequence or a PAM as discriminators are examined herein.

For example, a COSMIC database may be used comprising a list of validated cell line SNPs to test the feasibility of this approach (e.g., identifying twenty-six heterozygous SNPs in U2OS cells). Candidate loci may then be sequenced to confirm the reported SNP heterozygosity and then design SpCas9^{MT}-pDBD/sgRNA combinations to target a single allele. Allelic targeting ratios (relative to negative controls lacking the cognate sgRNA or the appended pDBD) may be determined by a frequency that each allele captures GUIDE-seq oligos (via deep sequencing). If DSBs are restricted to a single allele, then only the targeted SNP should be found neighboring the GUIDE-seq oligo sequence. As SpCas9 mutants are identified that have improved attenuation, single base change discrimination can then be examined. Although it is not necessary to understand the mechanism of an invention, it is believed that SpCas9^{MT}-pDBDs have great potential for allele-specific targeting but should be subjected empirical verification. If necessary, discrimination can be tested using paralogous sequences that differ by a single base within the genome (e.g. CCR2 and CCR5, which contain many >30 bp regions that differ by a single nucleotide). Relative editing efficiencies on one paralog or the other can be assessed by the PCR/deep sequencing approach described above.

3. Drug- or Photo-Dependent spCas9-pDBD Nuclease Regulation

Small molecule- or photo-dependent dimerization systems have been developed that permit the control of activity of a two-component system. Since SpCas9/sgRNA off-target activity is dose dependent, these systems have been adapted to regulate the association of two fragments of Cas9 (e.g., Split-Cas9).

In one embodiment, the presently disclosed SpCas9-pDBD system comprises a drug- or photo-dependent dimerization system that regulates the association of SpCas9 and the pDBD. In one embodiment, the present invention contemplates a rapamycin-dependent Cas9 complex comprising a SpCas9-FRB/FKBP-ZFP and/or a SpCas9-FRB/FKBP-TALE and/or Split-SpCas9^{MT}-pDBD, FIG. 47, FIG. 99, FIG. 100, FIG. 101 and FIG. 102. The data show that the target activity (with drug) is similar to SpCas9^{WT} without sacrificing of the enhanced precision of the SpCas9-pDBD system. In addition, swapping the drug-dependent dimerization domains (e.g. SpCas9-FKBP/FRB-ZFP) and changing the relative order of these domains (e.g. SpCas9-FKBP/ZFP-FRB) can improve the activity and precision of these constructs (FIGS. 99 & 101). This type of improvement of components (fusion partners and their relative position) can be attained for any combination of dimerization domains in principle.

### 4. SpCas9-FKBP/pDBD-FRB System Improvements

a. SpCas9-FKBP/pDBD-FRB Linkers

In one embodiment, the present invention contemplates a GFP reporter system comprising genomic targets to identify a optimal linker length joining Cas9 to a dimerization domain and the pDBD to a dimerization domain that maximizes activity and restricts the relative spacing and orientation of the active binding sites. In one embodiment, the linker joins an SpCas9-FKBP domain and an pDBD-FRB domain.

b. ZFP or TALE DNA Recognition Modules

In one embodiment, the present invention contemplates DNA recognition modules that improve SpCas9-FKBP/pDBD-FRB precision at sites including, but not limited to, TS2, TS3 and TS4 sites. Although it is not necessary to understand the mechanism of an invention it is believed that the optimal number and composition of recognition modules in the pDBD may differ when compared to a Cas9-pDBD covalent system, since greater cooperativity in the binding is likely to occur in the covalent system.

c. Nm-dCas9 as pDBDs

In one embodiment, the present invention contemplates a nuclease-dead NmCas9 as a pDBD for an association through dimerization (FIG. 20). In one embodiment, a mutated nuclease (e.g., SpCas9^{MT}) and Nm-dCas9 are programmed through orthogonal sgRNAs to recognize neighboring sequences. Although it is not necessary to understand the mechanism of an invention, it is believed that for this type of dimerization system (e.g. SpCas9^{MT}-FKBP/Nm-dCas9-FRB) fusion partners for each dimerization domain and their position on the nuclease are empirically determined. In principle other Cas9 isoforms could be substituted for SpCas9 or Nm-dCas9.)

d. Nuclear Export Sequences

Photo-dependent TALE regulators or drug-dependent Split-SpCas9 fusions have been reported to decrease off-target activity by fusing a nuclear export sequence (NES) instead of a Nuclear Localization Sequence (NLS) to one component. It is believed that an Cas9-NES fusion protein is restricted to the cytoplasm until the inducer is present (light/drug), at which point an NLS-tagged partner can drive nuclear import. In one embodiment, an NES-SpCas9^{MT}-FRB fusion protein may be excluded from the nucleus in the absence of rapamycin. In one embodiment, a combination of an NLS with NES-SpCas9^{MT}-FRB fusion protein facilitates a transit between the nucleus and cytoplasm in the presence of rapamycin allowing more efficient import of the partner that is located in the cytoplasm (e.g. FIG. 99).

Assessments of activity and precision for constructs of particular interest may occur at an TS2/TS3/TS4 loci initially by T7EI assays such that dose and duration of rapamycin exposure on activity and precision can be examined. The precision of the most promising constructs may be evaluated by GUIDE-seq followed by targeted deep sequencing (e.g. FIG. 102).

e. The Abscisic Acid Regulatory System

A drug-based dimerization system has been previously described based on a plant hormone (i.e., for example, abscisic acid) and its protein partners (ABI & PYL; Liang, F.-S., Ho, W. Q. & Crabtree, G. R. Engineering the ABA plant stress pathway for regulation of induced proximity. Science Signaling 4, rs2-rs2 (2011).). Abscisic acid is believed to be bioavailable, and the plant-derived components should have minimal crosstalk with endogenous factors (unlike a rapamycin system). Consequently, a SpCas9^{MT}-ABI/PYL-pDBD system may be useful for drug-dependent regulation.

Photo-dependent (e.g., for example, visible light or non-visible light) regulation of TALE-effector and Split-SpCas9 nuclease function have been described. In one embodiment, the present invention contemplates a light-inducible dimerization domain comprising nMag/pMag or CRY2/CIB1 (Nihongaki, Y., Kawano, F., Nakajima, T. & Sato, M. Photoactivatable CRISPR-Cas9 for optogenetic genome editing. Nature biotechnology 33, 755-760 (2015)).

D. Improved Precision with NmCas9-pDBD and SaCas9-pDBD Frameworks

Development of a SpCas9-pDBD system (supra) has benefited from extensive data on the 1368-aa SpCas9 protein. However, full realization of genome editing goals involves the development of additional Cas9 orthologs to provide additional PAM specificities and simultaneous deployment of Cas9s with orthogonal guides. In addition, for clinical deployment, the physical size of SpCas9's limits in vivo deliverability to platforms such as AAV vectors and synthetic mRNAs. Alternatively, most Type II-C Cas9s (e.g. N. meningitidis; 1082 residues) and a few Type II-A Cas9s (e.g. S. aureus; 1053 residues) are considerably smaller than SpCas9 and may have clinical delivery advantages over SpCas9 platforms.

For example, a compact Cas9 (i.e., for example, NmCas9) was recently validated for genome editing. Alternatively, an SaCas9 platform was also characterized, and its utility for editing in an all-in-one (Cas9+sgRNA) AAV format was documented. Because Cas9s is believed to have some propensity for promiscuous cleavage, compact orthologs should be modified to provide an enhanced precision to tap their clinical potential. In one embodiment, the present invention contemplates NmCas9- and SaCas9-based editing platforms with single-genomic-site accuracy.

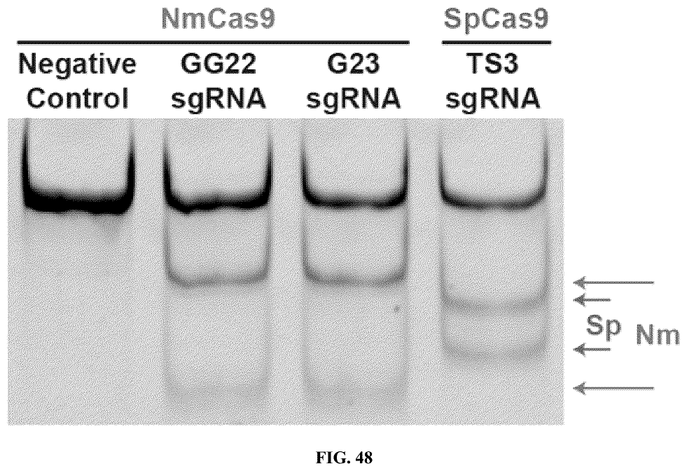

Preliminary data using NmCas9 demonstrate that a PAM consensus is 5′-N4GATT-3′, with considerable variation allowed during bacterial interference (data not shown). However, PAM requirements are more stringent in mammalian cells, and efficient editing has only been documented at N4G(A/C/T)TT, N4GAC(A/T), N4GATA, and N4GTCT PAMs, FIG. 48. It has been reported that mammalian genome editing by NmCas9 also requires strong sgRNA/target seed-sequence complementarity. NmCas9 guide sequences are naturally 24 nts long, though 22- to 26-nt lengths are functional (not shown). As shown above, when using a GFP assay in HEK293 cells, Zif268 fusion to NmCas9 (in conjunction with a Zif268 binding site downstream of the PAM) allows targets with otherwise non-functional PAMs to be cleaved. See, FIG. 49A. This demonstrates that an appended DBD can facilitate cleavage at non-canonical NmCas9/PAM interactions, as with SpCas9 (supra).

The structure of NmCas9 is not known, nor are associated PAM-recognition residues defined. Nonetheless, some information can be discerned from an A. naeslundii Type II-C Cas9 structure (AnCas9). For example, two positively charged NmCas9 residues (e.g., Lys1013 and Arg1025) are particularly well-conserved in Type II-C Cas9 alignments, and the corresponding AnCas9 residues map to a candidate PAM interaction region. The activity of the NmCas9 K1013A/R1025A double mutant (hereafter NmCas9^{DM1}) is severely attenuated in the GFP assay in HEK293 cells, but can be rescued by an appended Zif268 pDBD (with a Zif268 binding site downstream of the PAM). FIG. 49B. Although it is not necessary to understand the mechanism of an invention, it is believed that these observations, along with the mutant PAM rescue, strongly suggest that the PAM attenuation/pDBD fusion is a feasible strategy to create more precise compact Cas9 orthologs. Furthermore, recent reports have also provided information on SaCas9, including its functional PAM sequence (5′-NNGRR-3′) (SEQ ID NO: 34) and spacer lengths (21-23 nts), thereby facilitating this strategy's extension into a compact Type II-A system.

### 1. PAM Attenuation/pDBD Fusion Parameters for Enhanced-Precision NmCas9 and SaCas9

a. NmCas9^{MT}-pDBD and SaCas9^{MT}-pDBD Frameworks

The data presented herein demonstrates that a fused pDBD (either N- or C-terminal, with a 60-aa linker) allows editing of targets with nonfunctional PAMs having a pDBD binding site 5 bp from the PAM. FIG. 49A. Alternative embodiments include, but are not limited to, other NmCas9-pDBD spacings and orientations for SpCas9 and/or NmCas9 fusions to TAL268. For example, one SaCas9 embodiment comprises using a PAM variant (e.g., for example, NN(A/C/T)RR) (SEQ ID NO: 35) that is known to be non-functional. Alternatively, alanine and/or serine mutations, either individually or pair-wise, may be introduced that are within an ˜25-aa window around a putative PAM-interacting domain of SaCas9, based on Type II-A Cas9 sequence alignments. It can then be determined which of those mutations attenuate SaCas9 function and can be re-activated by Zif268 fusion, FIG. 49B. Initially, a GFP assay in HEK293 cells can be performed, and the most promising set of spacings, orientations, and Zif268-suppressible SaCas9-attenuating mutants may then be validated at corresponding genomic loci by T7EI assay. Several custom-designed ZFP and TALE modules can also be tested on other chromosomal targets with both Cas9^{MT} systems. Finally, NmCas9- and SaCas9-linker length improvements are determined as described above.

b. NmCas9 and SaCas9 Accuracy

In one embodiment, the present invention contemplates a GUIDE-seq assay to compare the editing precision of Cas9^{WT} orthologs and the Cas9^{MT}-pDBD variants. In one embodiment, the GUIDE-seq assay identifies Indel frequencies at off-target sites. In one embodiment, the Indel frequencies are quantified by deep-sequencing PCR-amplified loci. In one embodiment, mismatch tolerance at chromosomal editing sites measure the effects of PAM attenuation and pDBD fusion. In one embodiment, off-target propensities of the on-vs. off-target lesion rate ratios identify successful pDBD tunability by varying the number of ZFPs or TALE modules.

### 2. NmCas9 and SaCas9 Drug-Inducible Dimerization Systems

One disadvantage with AAV delivery of active Cas9/guideRNA combinations is that Cas9 activity (both on- and off-target) may persist indefinitely. Accordingly, by successfully implementing drug-inducible Cas9^{MT}-pDBD association in the context of one or both compact Cas9s, the system's accuracy enhancements are further improved, and by preventing on-going off-target lesions once the drug is withdrawn and after editing is complete. In one embodiment, the present invention contemplates a NmCas9 and/or a SaCas9 drug-inducible dimerization system.

For example, DNA-binding modules (e.g., ZFP and TALE) attached to NmCas9 or SaCas9 could both be RNA-guided. NmCas9 and its guideRNAs are orthogonal to all Type II-A Cas9s and sgRNAs tested to date, and SaCas9's expected orthogonality and its sgRNAs can be easily confirmed as well. Drug-inducible dimerization modules (e.g., for example, FRB/FKBP or ABI/PYL and all pair-wise combinations) can be fused to a PAM-attenuated but catalytically active version of a compact Cas9, and the nuclease-dead version of the other. Whether dCas9 can fulfill the same precision-enhancing function provided by the pDBD may then be tested. Initially, a GFP reporter system is used to improve PAM/target orientation and spacing, and then tested using actual chromosomal loci. If this framework can edit its chromosomal loci target sites efficiently, as judged by T7EI assay, an unbiased assay can define the precision of this system relative to the drug-induced pDBD dimerization system.

3. Functional AAV NmCas9^{MT}-pDBD and SaCas9^{MT}-pDBD Constructs

It is believed that native NmCas9 and SaCas9 ORFs are ˜3.25 and 3.16 kb, respectively, so even with added NLSs and minimal expression/processing signals, they are well under the ˜4.5 kb packaging limit of current AAV vectors. For example, a four-finger ZFP with a 60-aa linker would increase the ORF size by an additional 0.6 kb, still well within the AAV vector size limit. As explained herein, some embodiments of the present invention minimize linker length to further reduce an AAV Cas9-pDBD packaging size. In some embodiments, the present invention contemplates the delivery of NmCas9^{MT}-ZFPs and SaCas9^{MT}-ZFPs via AAV into cultured cells. In one embodiment, the AAV comprises a liver-specific promoter. In one embodiment, the AAV is an AAV8 serotype. In one embodiment, the AAV8 serotype is hepatocyte-tropic. In one embodiment, the cultured cells comprise HepG2 cells. In one embodiment, the genome of the HepG2 cells comprise Pcsk9 as an editing (NHEJ) target. In one embodiment, the AAV expression constructs is a transfection plasmid.

### E. Cas9-pDBD Mediated Gene Correction of Defective CYBB in CGD

Chronic granulomatous disease (CGD), a disorder of phagocytic function, generally presents early in life with severe recurrent infections. The estimated incidence per live birth is 1/200,000 in the US. Conventional clinical management allows many patients to reach adulthood, but CGD patients have only 50% cumulative survival at age 50, and the only curative therapy is hematopoietic stem and progenitor cell (HSPC) transplantation. The molecular defects causing CGD affect the phagocyte NADPH oxidase responsible for the generation of microbicidal reactive oxygen species. About 60% of cases are X-linked (X-CGD) due to mutations in CYBB, an Xp21.1 gene that encodes gp91phox, the glycoprotein subunit of the oxidase.

CGD has long been considered a prime target for gene therapy. Clinical improvement should occur with replacement of a low level of oxidase activity, as CGD patients with as little as 3% normal activity show a much milder phenotype. A normal phenotype could be achieved with high-level correction of only 5-10% of phagocytes, as occurs in asymptomatic XCGD carriers with a skewed Lyon distribution of X-inactivation. As all phagocytes are bone marrow-derived, gene therapy approaches have aimed to replace the defective gene ex vivo in blood or bone marrow HSPCs, and then engraft the autologous cells in the patient. For example, one such trial, using an SFFV-based retroviral vector, showed initial correction of the CGD phenotype in 2 of 3 subjects, but gene expression was eventually diminished or silenced. Further, peripheral blood myeloid cells showed expansion of clones containing insertions at loci associated with immortalization or leukemia. All patients eventually died or underwent HSPC transplantation.

Current CGD gene therapy approaches are focused on gene replacement in CD34+ HSPCs through insertion via self-inactivating lentivirus or knock-in at a safe-harbor locus (AAVS1) via ZFNs. A current trial employs a self-inactivating lentiviral vector encoding a chimeric myeloid promoter to drive CYBB expression. However, achieving near wild-type gp91phox expression requires 8 or more integrations per cell. Because lentivirus generates insertions throughout the genome there is also danger of viral integration causing disruption or dysregulation of nearby genes.

Targeted insertion in the AAVS1 locus limits random integration, but suffers from the challenge of finding a myeloid-specific promoter that can drive high level gp91phox expression with only one integration site. Ideally, gene repair at the defective locus would harness endogenous regulatory elements to drive appropriate gene expression. As inactivating mutations in CYBB are broadly distributed throughout the coding sequence, tailoring a gene correction cassette to each patient's specific mutation is impractical.

In one embodiment, the present invention contemplates a minigene cassette flanked by a splice acceptor and polyadenylation site, for insertion into an early intron to capture transcription from the locus and correct any downstream mutations. FIG. 50. This approach has been successfully utilized with ZFNs for factor IX gene correction. At the CYBB locus, a repair cassette introduced into intron 2 would correct 87% of previously described mutations.

To define neutral sites for repair cassette insertion, the CYBB regulatory landscape in three myeloid cell lines was analyzed using ENCODE H3K4Me1 ChIP-seq data and 3C analysis. These data revealed a complex regulatory landscape that extends to CYBB introns 1-3. In one embodiment, the present invention contemplates a gene correction strategy comprising high efficiency and precision, as well as a minimal impact of minigene insertion on gene expression levels, as some insertion sites may disrupt regulatory elements. For example, a Cas9^{MT}-pDBD nuclease may be used for correction of CYBB defects in CD34+ HSPCs from XCGD patients through a systematic optimization including, but not limited to: i) pilot experiments in XCGD-PLB-985 cells, a human myeloid cell line with a disruption in exon 3 of CYBB9; ii) optimization of gene correction in normal CD34+ HSPCs; and iii) assessment of efficacy in HSPCs from XCGD carriers. Although it is not necessary to understand the mechanism of an invention, it is believed that these preliminary data identifies improved nuclease precision and efficiency to provide a clinically effective platform for CGD gene therapy.

### 1. SpCas9^{MT}-pDBD Nuclease and Donor Constructs

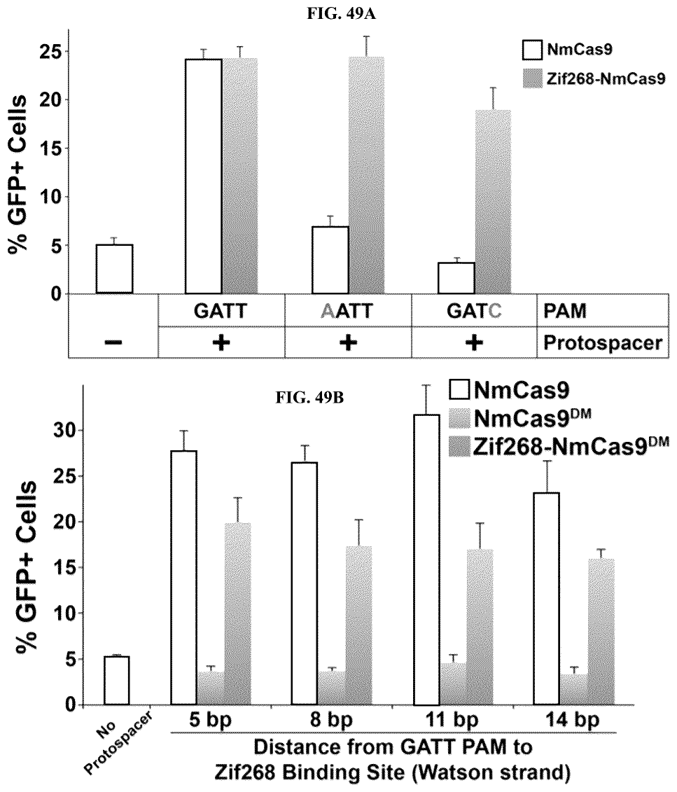

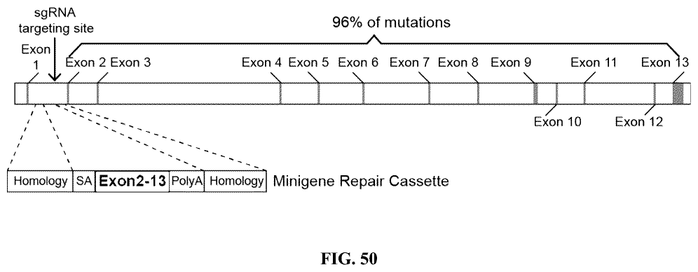

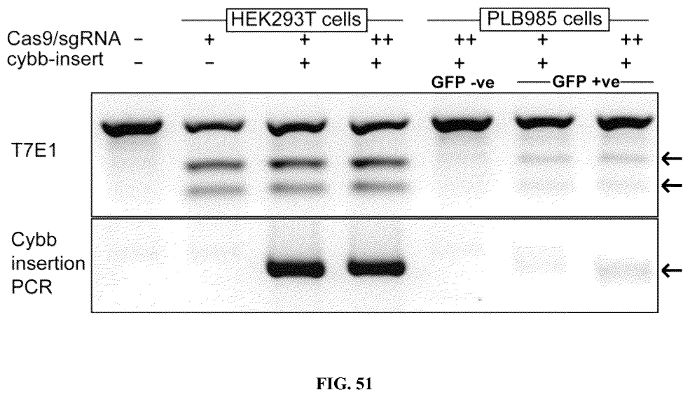

In one embodiment, the present invention contemplates assessing nuclease activity and precision in HEK293T cells. Preliminary data show that CYBB introns 1 & 2 are compatible with sgRNAs having NGG PAMs and are predicted to be highly active based on the latest genome-wide sgRNA analyses. These sgRNAs have few predicted off-target matches by CRISPRseek analysis and avoid potential regulatory regions identified in ENCODE data. SpCas9 nuclease activity mediated by sgRNAs of interest may be used to determine and identify active guides. FIG. 51.

In one embodiment, the present invention contemplates a construct comprising Cas9^{MT}-pDBDs for active sgRNAs. Nuclease activity may be confirmed by T7EI, and then GUIDE-seq followed by focused deep sequencing to determine off-target profiles. In one embodiment, active nuclease pDBDs can be tuned and precision improved to eliminate residual off-target activity. One advantage of the presently disclosed embodiments in contrast to conventional methods is the achievement of precise editing with off-target events that are undetectable by Illumina short-read sequencing. In one embodiment, the construct comprises single-stranded oligonucleotide (ssODN) donors with homology arms to the target site that encode a unique restriction enzyme (RE) site within the region. HDR efficiency may be assayed by PCR amplification and RE digestion.

### 2. Gene Correction Efficiency

XCGD-PLB-985 cells provide a model for gene correction of CYBB due to the presence of a single defective allele. In one embodiment, nucleofection conditions are improved for XCGD-PLB-985s to maximize the rate of nuclease-based HDR insertion of the validated ssODN compared to that of indel formation (e.g., using a T7EI assay). HDR efficiency and precision level obtained for each nuclease may then be confirmed using GUIDE-seq.

XCGD-PLB-985 cells were nucleofected with SpCas9-sgRNA, a Cybb-minigene cassette, and GFP (as a marker for nucleofection) and then flow-sorted for GFP expression. GFP(+) and (−) cells were assessed for SpCas9-induced lesions by T7EI assay, and for NHEJ-mediated minigene insertion by PCR amplification of a newly-formed junction. FIG. 51. GFP(+) cells demonstrated a functional correction of a CGD phenotype, with 5 cells per 1000 showing oxidase activity as NBT dye reduction at the higher dose of SpCas9/sgRNA, 2/1000 at the lower dose, and none in GFP(−) controls. FIG. 51. This data demonstrates a CYBB gene correction with the presently disclosed minigene cassette.

Although it is not necessary to understand the mechanism of an invention, it is believed that the present methods result in dramatic improvements in rescue frequency in comparison to conventionally available assays. Alternatively, the present invention contemplates a knock-in of a human codon-optimized minigene rescue construct comprising sequence features distinct from an endogenous locus. FIG. 50. For example, improvements in precision may include, but are not limited to, parameters comprising: i) homology arm length; ii) donor DNA source, including but not limited to plasmids, minicircles, or AAVs.

Donor DNA insertion efficiency can be evaluated by qPCR, and the integrity of donor integration assessed by PacBio SMRT sequencing to define the donor cassette insertion rate and fidelity. The rate of spurious donor integration can be determined by LAM-PCR sequencing. To increase rates of HDR, alternate DNA repair pathways can be inhibited. Differentiation of XCGD-PLB-985 cells containing targeted minigene insertions into neutrophils can assess the functional effects of gene correction. The rate of splice donor capture by an integrated minigene can be determined by qRT-PCR. XCGD-PLB-985-derived neutrophils can be determined by flow cytometric assays of mAb7D5 binding for gp91phox protein expression, dihydrorhodamine (DHR) fluorescence for NADPH oxidase activity, and/or loss of microbial propidium iodide staining for microbicidal activity. Although it is not necessary to understand the mechanism of an invention, it is believed that the above embodiments are able to define minigene insertion sites that permit an efficient correction of CYBB defects in XCGD-PLB-985 cells by optimizing a splice acceptor sequence of a repair cassette for efficient gene capture. Functional assays should allow correlation with correction of the CGD phenotype.

### F. Gene Correction Efficiency and Precision in CD34+ HSPCs

It is generally believed that achieving high levels of donor DNA integration via nuclease-mediated HDR is more challenging in primary HSPCs than in transformed cell lines. To overcome this disadvantage of conventional methods, due to a limited availability of XCGD patient-derived CD34+ cells, the presently disclosed nuclease-based knock-in strategy may be fine-tuned using CD34+ HSPCs from healthy male donors. It has been reported that SpCas9/sgRNA gene inactivation has been performed through the delivery of plasmid-encoded components, but efficient rates of donor DNA integration and cell viability in another study required delivery of nucleases as mRNAs.

In one embodiment, the present invention contemplates a method comparing the efficiency of gene editing and cell viability for SpCas9^{MT}-DBDs/sgRNA delivered by plasmid vs. mRNA/sgRNA nucleofection. For example, target site lesion rates can be assessed by T7EI assay¹⁹, and cell viability by Annexin V and 7-Aminoactinomycin D FACS analyses. Further, the efficiency of HDR can be examined using different donor DNAs encoding the required repair cassette. Due to potential plasmid toxicity in CD34+ cells, assays may be performed in both plasmid-based, minicircle-based and/or viral DNA donors (IDLV, Adenoviral and AAV, respectively), particularly AAV6, which efficiently transduces CD34+ HSPCs and has proven to be an efficient non-integrating donor for nuclease-mediated HDR. In some embodiments, the timing of the donor and nuclease delivery can be varied to maximize the efficiency of HDR. In other embodiments, small molecules that support progenitor maintenance during expansion may be used. The precision of the nucleases and the integrity and specificity of donor integration can be assessed as described above.

XCGD-like CD34+ HSPCs have recently been created by transducing normal CD34+ cells with a Cerulean-marked lentivirus encoding shRNAs targeting CYBB transcripts. This system can be utilized to assess the efficiency of CYBB gene correction mediated by the optimal nuclease and donor DNA, with a recoded minigene that is not targeted by the shRNAs, to determine the fraction of macrophages and neutrophils differentiated from marked CD34+ cells with restored NADPH oxidase activity and function. Although it is not necessary to understand the mechanism of an invention, it is believed that with the presently disclosed improved nucleases, alternate donor DNA platforms and supporting culture conditions, are able to achieve high levels of targeted gene correction in CD34+ HSPCs, that equal or exceed the 5-10% level needed for a functional CGD cure.

### G. Efficient Gene Correction in XCGD CD34+ HSPCs

In one embodiment, the present invention contemplates a nuclease-mediated CYBB correction in SCGD patient CD34+ HSPCs. In one embodiment, the nuclease comprises a minigene repair cassette having mutations. In one embodiment, improved targeted gene correction conditions (e.g., for example, nucleases, donors, cultures) that are shown to improve efficiency. In one embodiment, the method determines the fraction of functionally corrected macrophages and neutrophils differentiated from these cells.

RNA levels can also be assessed for a minigene donor cassette and the fraction of correctly spliced RNAs between the endogenous exon and the minigene cassette. In other embodiments, an in vivo engraftment potential and function of nuclease-manipulated HSPCs. Preferably, NSG-3GS mice can be evaluated, which unlike NSG mice, produce functional human phagocytes. Although it is not necessary to understand the mechanism of an invention, it is believed that the presently disclosed method achieves a frequency of appropriate RNA splicing with a repair cassette sufficient to generate gp91phox in patient-derived XCGD cells comprising endogenous locus regulatory elements.

### H. Excision or Inactivation of HIV Proviral DNA in Reservoir Cells.

Highly active antiretroviral therapy (HAART) has dramatically changed the prognosis for individuals infected with HIV-1. Yet, even when HIV-1 viremia has been well controlled by these drugs for years, termination of HAART results in viral rebound, most likely coming from latent provirus in long-lived memory CD4⁺ T cells. So long as latent HIV-1 provirus persists—probably for the life of the infected individual—HAART will be required. Most efforts to eradicate latent HIV-1 proviruses have focused on reactivation of proviral transcription to potentiate the elimination of cells bearing HIV-1 provirus. To date, though, such reactivation efforts have largely been unsuccessful. Alternative approaches for the effective elimination of latent HIV-1 provirus are therefore needed.

Recent advances in the development of targeted gene editing tools provide a potential method for direct inactivation or excision of latent HIV-1 provirus. Specifically, the Cas9/CRISPR programmable nuclease system, a versatile platform for the generation of targeted double-strand breaks within the genome, has been shown to excise HIV-1 provirus in cell lines. However, the activity and precision of the Cas9/CRISPR system is suboptimal for clinical application. SpCas9^{MT3}-ZFPs have been developed that specifically target the HIV LTR with higher precision than wild-type SpCas9.

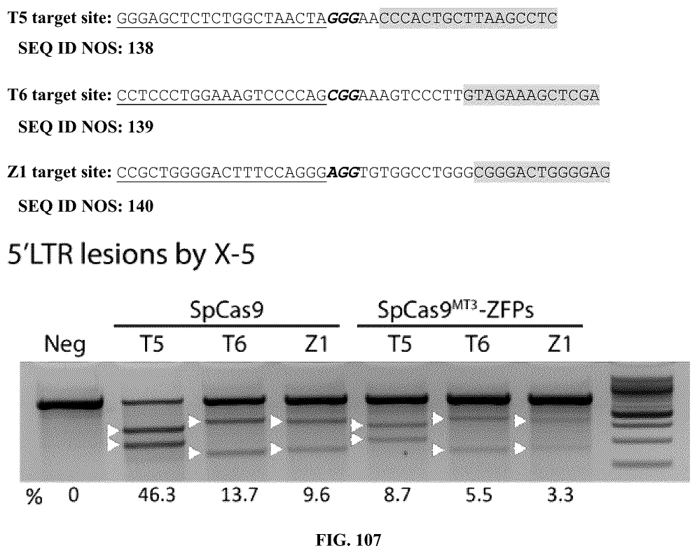

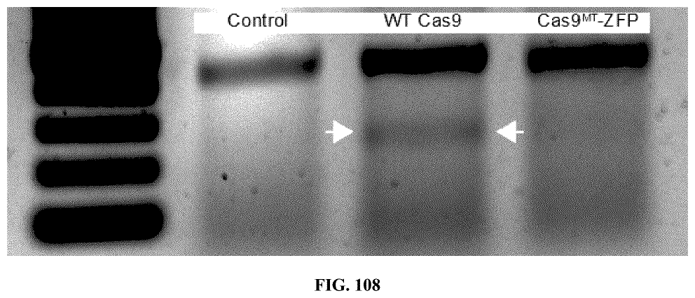

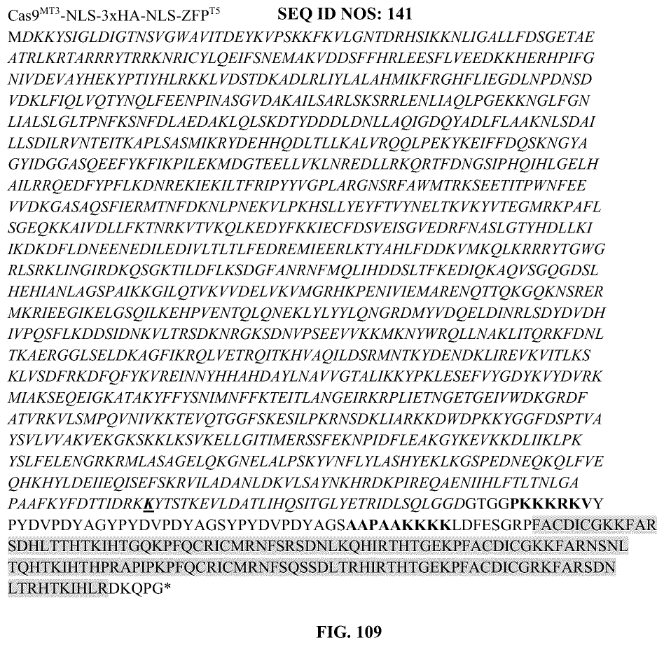

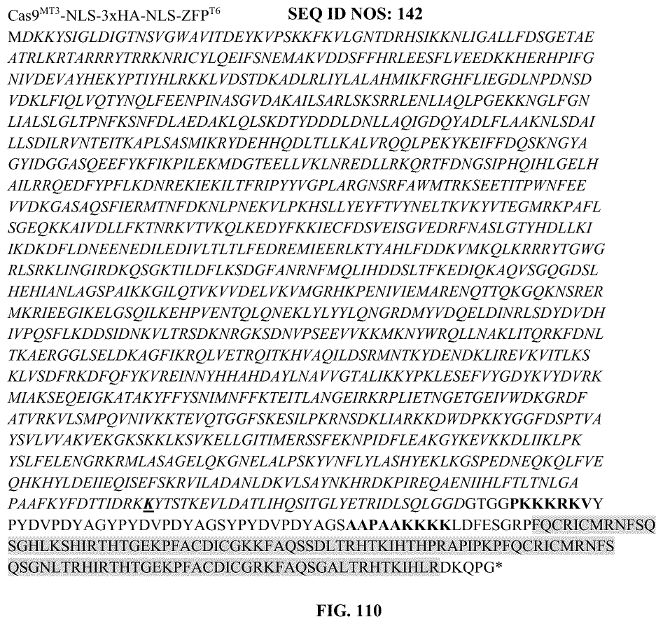

Three different SpCas9^{MT3}-ZFPs were generated that target different regions of the HIV LTR (T5, T6 and Z1; FIG. 107). Lesion rates of wild-type SpCas9 or SpCas9^{MT3}-ZFP were compared with T5, T6 and Z1 sgRNA and these nucleases have similar activity in the T7EI assay measuring nuclease-induced lesion rates (FIG. 107). The SpCas9^{MT3}-ZFPs have higher precision. Comparison of lesion rates at one computationally predicted off-target sites for the T5 sgRNAs with either wild-type SpCas9 or SpCas9^{MT3}-ZFP^{T5} reveals weak activity for the wild-type nuclease but no activity for SpCas9^{MT3}-ZFP^{T5} (FIG. 108). Further improvement of these SpCas9^{MT3}-ZFPs or the development of nucleases related to platforms described herein should allow the creation of an efficient, precise nuclease system for the inactivation/excision of HIV-1 provirus from reservoir cells of HIV infected individuals.

### IV. Deep Sequencing Analysis of Off-Target Activity

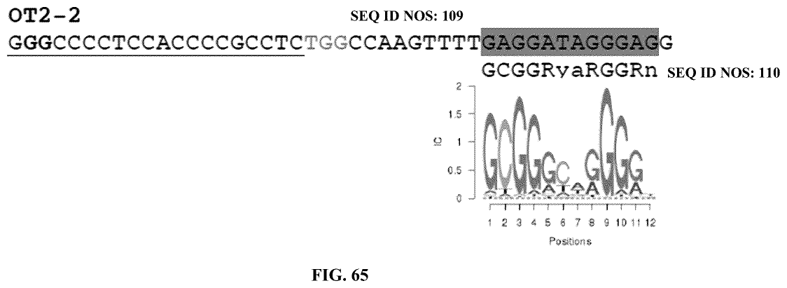

To more broadly assess improvements in Cas9-pDBD precision, PCR products were deep sequenced spanning previously defined off-target sites for sgRNA^{TS2/TS3/TS4; 14,25} as well as several additional genomic loci that have favorable ZFP^{TS2/TS3/TS4} recognition and were predicted using CRISPRseek^{21,22} to have some complementarity to the TS2/TS3/TS4 guide sequences. Nuclease activity was compared between SpCas9, SpCas9^{MT3} SpCaSg^{WT}-ZFP^{TS2/TS3/TS4} and SpCas9^{MT3}-ZFP^{TS2/TS3/TS4} at these target and off-target sites, and found that SpCas9^{MT3}-ZFP^{TS2/TS3/TS4} dramatically increased the precision of target site cleavage. FIG. 64A. In most cases, utilizing SpCas9^{MT3}-ZFP^{TS2/TS3/TS4} reduced lesion rates at off-target sites to background levels resulting in improvements in the Specificity Ratio of up to 150-fold, FIG. 64B. Only one off-target site (OT2-2), which has a neighboring sequence that is similar to the expected ZFP^{TS2} recognition sequence and still displays high lesion rates, FIG. 65. One other site (OT2-6), displays some residual activity both for SpCas9^{MT3} and SpCas9^{MT3}-ZFP^{TS2} that is above the background error rate within our sequencing data. Overall, these data demonstrate a dramatic enhancement in precision for SpCas9^{MT}-ZFPs relative to standard SpCas9 at previously defined active off-target sites.

### V. Clinical Applications And Insights

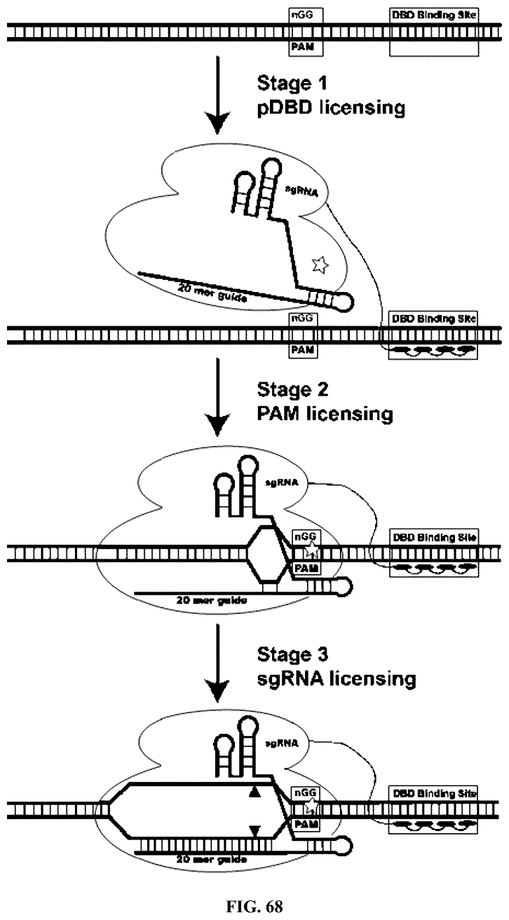

Some embodiments of the present invention encompass of the activity of SpCas9-pDBD chimeric activity that provide new insights into a mechanism of target site licensing by SpCas9 and the methods by which this mechanism can be exploited to improve precision. FIG. 68. Fusion of a pDBD to SpCas9 allows efficient utilization of a broader repertoire of PAM sequences by SpCas9, but even for SpCas9-pDBDs there remains a dichotomy between functional and inactive PAMs. The broader targeting range of SpCas9-pDBDs likely reflects the bypass of a kinetic barrier to R-loop formation that follows PAM recognition, as proposed by Seidel and colleagues⁶. pDBD tethering of SpCas9 may achieve activity at a target site containing a sub-optimal PAM by increasing the effective concentration of SpCas9 around the target site and hence, stabilizing the SpCas9-PAM interaction. For wild type SpCas9, only high affinity (nGG) PAM sites consistently have sufficient residence time to facilitate efficient progression to R-loop formation, but pDBD tethering increases the likelihood that SpCas9/sgRNA can overcome this barrier at sub-optimal PAMs. The data presented herein also support an allosteric licensing mechanism, as described by Doudna and colleagues⁵, which likely restricts Cas9 nuclease activity for the majority of sequence combinations in the PAM element even with the increased local concentration afforded by pDBD tethering. The enhanced sensitivity to guide-target site heteroduplex stability observed for the presently disclosed SpCas9^{MT3}-ZFP^{TS3} chimera further supports an interplay between PAM recognition and guide complementarity in the licensing of nuclease activity.

Mutations to the SpCas9 PAM interacting domain may introduce a third stage of licensing (pDBD site recognition) for efficient target site cleavage within the SpCas9^{MT}-pDBD system. The weakened interaction between mutant Cas9 and the PAM sequence now necessitates increased effective concentration for nuclease function that is achieved by the high affinity interaction of the tethered pDBD with its target site. This dramatically improves precision as assessed using targeted deep sequencing and GUIDE-seq analysis. Compared with previous GUIDE-seq analysis of TS2, TS3 and TS4 targets for SpCas9, five, three and three of the top 5 off-target sites, respectively, were found that were previously described¹⁷. The discrepancy between these studies could be due to our lower sequencing depth, the use of an alternate cell line, or different delivery methods. Nonetheless, the present analysis excludes the presence of a new class of highly active off-target sites that are generated by the fusion of the ZFP to Cas9. This system has advantages over other previously described Cas9 variant systems that improve precision^{10,25-30}. The presently disclosed SpCas9^{MT}-pDBD system increases the targeting range of the nuclease by expanding the repertoire of highly active PAM sequences. This is in contrast to dimeric systems (e.g., for example, dual nickases or FokI-dCas9 nucleases) that have a more restricted targeting range due to the requirement for a pair of compatible target sequences. Moreover, the presently disclosed chimeric system may be compatible with either of these dimeric nuclease variants, providing a further potential increase in precision while also expanding the number of compatible target sites for these platforms. In addition, the affinity and the specificity of the pDBD component can also be easily tuned to achieve the desired level of nuclease activity and precision for demanding gene therapy applications.

SpCas9-ZFPs targeting TS2/TS3/TS4 were programmed with four-finger ZFPs, as it was believed that these would have an optimal balance of specificity and affinity, for example, SpCas9^{MT3}-ZFP^{TS3}. However, SpCas9^{MT3}-ZFP^{TS2} resulted in improved precision by utilizing a three finger ZFP demonstrating pDBD flexibility. In addition to tuning a pDBD, further improvements by adjusting linker lengths and its composition should realize improvements in precision (and potentially activity) by further restricting the relative orientation and spacing of the SpCas9 and pDBD. Finally, it should be possible to generate Cas9-pDBD fusions for Cas9 orthologs from other species that have superior characteristics for gene therapy applications (e.g. more compact Type IIC Cas9 nucleases^{49,50} for viral delivery). Ultimately, for gene therapy applications where precision, activity and target site location are of paramount importance, the expanded targeting range and precision achieved by the Cas9-pDBD framework provides a potent platform for the optimization of nuclease-based reagents that cleave a single target site in the human genome.

### VI. Kits

In another embodiment, the present invention contemplates kits for the practice of the methods of this invention. The kits preferably include one or more containers containing a Cas9 nuclease-DNA targeting unit fusion protein to practice a method of this invention. The kit can optionally contain a Cas9 nuclease fused to a dimerization domain and a DNA-targeting unit fused to a complementary dimerization domain. The kit can optionally include a zinc finger protein. The kit can optionally include a transcription activator-like effector protein. The kit can optionally include a homeodomain protein. The kit can optionally include a orthogonal Cas9 protein serving as the DNA targeting unit. The kit can optionally include a Cas9 fusion protein comprising a mutated PAM recognition domain. The kit can optionally include a single guide RNA molecule or gene, complementary to a specific genomic target. The kit can optionally include a second single guide RNA molecule or gene, complementary to a specific genomic target for the orthogonal Cas9 protein serving as the DNA-targeting unit. The kit can optionally include a truncated single guide RNA molecule or gene, completely complementary to a desired specific genomic target. The kit can optionally include enzymes capable of performing PCR (i.e., for example, DNA polymerase, Taq polymerase and/or restriction enzymes). The kit can optionally include a pharmaceutically acceptable excipient and/or a delivery vehicle (e.g., a liposome). The reagents may be provided suspended in the excipient and/or delivery vehicle or may be provided as a separate component which can be later combined with the excipient and/or delivery vehicle. The kit may optionally contain additional therapeutics to be co-administered with the nuclease to drive the desired type of DNA repair (e.g. Non-homologous end joining or homology directed repair). The kit may include a small molecule to drive drug-dependent dimerization of the Cas9-nuclease and the DNA targeting unit. The kit may include an exogenous donor DNA (either single stranded or duplex) that can be used as a donor for introducing tailor-made changes to the DNA sequence. The kit may include a small molecule to drive a change in subcellular localization for the Cas9 nuclease or the DNA-targeting unit to control the kinetics of its activity. The kit may include a small molecule to stabilize the Cas9 nuclease-DTU by attenuating degradation due to an attached destabilization domain.

The kits may also optionally include appropriate systems (e.g. opaque containers) or stabilizers (e.g. antioxidants) to prevent degradation of the reagents by light or other adverse conditions.

The kits may optionally include instructional materials containing directions (i.e., protocols) providing for the use of the reagents in the editing and/or deletion of a specific genomic target. While the instructional materials typically comprise written or printed materials they are not limited to such. Any medium capable of storing such instructions and communicating them to an end user may be contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials or assistance in the design and implementation of the Cas9 nuclease—DTU for specific genomic targets.

### EXPERIMENTAL

### Example 1

### Plasmid Constructs

For Cas9-DBD experiments an sgRNA expression plasmid pLKO1-puro was used as described previously. Stewart et al., Lentivirus-delivered stable gene silencing by RNAi in primary cells. RNA. 2003 April; 9(4):493-501. SpCas9 and SpCas9-DBD fusions are expressed from pCS2-Dest gateway plasmid under chicken beta globin promoter. Villefranc et al., Gateway compatible vectors for analysis of gene function in the zebrafish. Dev Dyn. 2007 November; 236(11):3077-87. For SSA directed nuclease activity assay, an M427 plasmid was used as previously reported. Wilson et al., Design and Development of Artificial Zinc Finger Transcription Factors and Zinc Finger Nucleases to the hTERT Locus. Mol Ther Nucleic Acids. 2013 Apr. 23; 2:e87.

Cas9-DBD target sites are cloned into Sbf1 digested backbone in ligation-independently. The Sbf1 digested M427 vector backbone may be treated with T4 DNA polymerase to recess the ends. Small double stranded oligonucleotides with flanking ends compatible to the recessed ends of vector are hybridized with the vector backbone in a thermocycler and directly transformed into bacteria.

ZFPs were assembled as gBlocks (Integrated DNA Technologies) from finger modules based on previously described recognition preferences. ZFPs were cloned into a pCS2-Dest-SpCas9 plasmid backbone cloned thorough BspEI and XhoI sites.

TALEs were assembled via golden gate assembly⁵ into JDS TALE plasmids⁵⁶. Assembled TALEs were cloned into BbsI digested pCS2-Dest-SpCas9-TALEntry backbone through Acc65I and BamHI sites.

Sequences of the SpCas9-pDBDs are presented herein and these plasmids are deposited at addgene for distribution to the community. Plasmid reporter assays of nuclease activity utilized the restoration of GFP activity through SSA-mediated repair of an inactive GFP construct using the M427 plasmid⁴⁶. SpCas9 target sites were cloned into plasmid M427 via ligation independent methods following Sbf1 digestion. Mutations in the PAM interacting domain of SpCas9 were generated by cassette mutagenesis.

### Example II

### Cell Culture and Transfection

Human Embryonic Kidney (HEK293T) cells were cultured in high glucose DMEM with 10% FBS and 1% Penicillin/Streptomycin (Gibco) at 37° C. incubator with 5% CO2. For transient transfection, early to mid-passage cells (passage number 5-25) were used. Approximately 1.6×10⁵ cells were transfected with 50 ng SpCas9/DBD expressing plasmid, 50 ng sgRNA expressing plasmid, 100 ng mCherry plasmid via Polyfect transfection reagent (Qiagen) in 24-well format according to manufacturer suggested protocol. For SSA-reporter assay, 150 ng M427 SSA-reporter plasmid may be also supplemented to the co-transfection mix.

### Example III

### Western Blot Analysis

HEK293T cells are transfected with 500 ng Cas9 and 500 ng sgRNA expressing plasmid in a 6-well plate by Lipofectamine 3000 transfection reagent (Invitrogen) according to manufacturer's suggested protocol. 48 hours after transfection, cells are harvested and lysed with 100 ul RIPA buffer. 8 μl of cell lysate is used for electrophoresis and blotting. The blots are probed with anti-HA (Sigma #H9658) and anti alpha-tubilin (Sigma #T6074) primary antibodies; then HRP conjugated anti-mouse IgG (Abcam #ab6808) and anti-rabbit IgG secondary antibodies, respectively. Visualization employed Immobilon Western Chemiluminescent HRP substrate (EMD Millipore #WBKLS0100).

### Example IV

### Flow Cytometry Reporter Assay

48 hours post transfection; cells were trypsinized and harvested into a microcentrifuge tube. Cells were centrifuged at 500×g for 2 minutes, washed once with 1×PBS and resuspended in 1×PBS for flow cytometry (Becton Dickonson FACScan). For FACS analysis, 10000 events are counted from each sample. To minimize effect of transfection variations among samples, first cells were gated for mCherry expression, and the percentage of EGFP expressing cells were quantified within mCherry positive cells. All the experiment replicates were performed in triplicate on different days and mean values and standard error of the mean may be calculated.

### Example V

### Genomic Target Analysis (T7E1)

72 hours post transfection; cells were harvested and genomic DNA was extracted via DNeasy Blood and Tissue kit (Qiagen) according to manufacturer suggested protocol. 50 ng input DNA was PCR amplified with Phusion High Fidelity DNA Polymerase (New England Biolabs): 98° C., 15s; 67° C. 25s; 72° C. 18s)×30 cycles. 10 ul of a PCR product was hybridized and treated with 0.5 μl T7 Endonuclease I in 1×NEB Buffer2 for 45 minute⁵⁷. The samples were run on 2.5% agarose gel and quantified with ImageJ software (PMID 22930834). Indel percentages were calculated as previously described (PMID 23478401). All the experiment replicates were performed in triplicate on different days and mean values and standard error of the mean may be calculated.

### Example VI

### Targeted Deep-Sequencing

For each generation of each amplicon, a two-step PCR amplification approach was used to first amplify the genomic segments and then installed with barcodes and indexes.

In a first step, “locus-specific primers” were used bearing common overhangs with complementary tails to the TruSeq adaptor sequences. 50 ng input DNA was PCR amplified with Phusion High Fidelity DNA Polymerase (New England Biolabs): (98° C., 15s; 67° C. 25s; 72° C. 18s)×30 cycles. 5 μl of each PCR reaction was gel-quantified by ImageJ against a reference ladder and equal amounts from each genomic locus PCR were pooled for each treatment group (15 different treatment groups). The pooled PCR products from each group were run on a 2% agarose gel and the DNA from the expected product size (between 100 and 200 bp) was extracted and purified via QIAquick Gel Extraction Kit (Qiagen).

In a second step, the purified pool from each treatment group was amplified with a “universal forward primer and an indexed reverse primer” to reconstitute the TruSeq adaptors. 2 ng of input DNA was PCR amplified with Phusion High Fidelity DNA Polymerase (New England Biolabs): (98° C., 15s; 61° C., 25s; 72° C., 18s)×9 cycles. 5 μl of each PCR reaction was gel-quantified by ImageJ, and then equal amounts of the products from each treatment group were mixed and run on a 2% agarose gel. Full-size products (˜250 bp in length) were gel-extracted and purified via QIAquick Gel Extraction Kit (Qiagen). The purified library was deep sequenced using a paired-end 150 bp Miseq run. Sequences from each genomic locus within a specific index were identified based on a perfect match to the final 11bp of the proximal genomic primer used for locus amplification.

Insertions or deletions in a SpCas9 target region were defined based on the distance between a “prefix” sequence at the 5′ end of each off-target site (typically 10 bp) and a “suffix” sequence at the 3′ end of each off-target site (typically 10 bp)⁵⁹, where there were typically 33 bp between these elements in the unmodified locus.

Distances that were greater than expected were binned as “insertions (I)”, and distances that were shorter were binned as “deletions (D)”. Reads that did not contain the suffix sequence were marked as undefined (U). For some loci the background sequencing error rate was high. For example for OT2-1 a homopolymer sequence in the guide region leads to a high error rate. All statistical analyses were performed using R, a system for statistical computation and graphics⁶⁰.

Log odd ratios of lesion were calculated for the on-target and off-target sites of each individual Cas9 treatment group vs. the untreated control for each of the three independent experiments. T-test was applied to assess whether the log odd ratio was significantly different from 0, i.e., whether there was a significant difference in lesion odds between each individual Cas9 treatment group and the untreated control for the on-target and off-target sites. Odds ratios and their 99% confidence intervals were obtained by taking exponent of the estimated log odds ratios and their 99% confidence intervals. These analyses were also applied to the sum of the lesion rates across all three replicates (combined).

To adjust for multiple comparisons, p-values were adjusted using the Benjamini-Hochberg (BH) method⁶¹. Only loci that have significant BH-adjusted p-values in the combined data for the treatment group relative to the control were considered significant. GUIDE-Seq off-target analysis for SpCas9-pDBDs. GUIDE-Seq was performed with some modifications to the original protocol¹⁷. The following primer sets were used for the positive (+) and negative (−) strands to get successful library amplification:

Nuclease_off_+_GSP1
(SEQ ID NO: 36)
GGATCTCGACGCTCTCCCTGTTTAATTGAGTTGTCATATGTTAATAAC +
Nuclease_off_-_GSP1
(SEQ ID NO: 37)
GGATCTCGACGCTCTCCCTATACCGTTATTAACATATGACA-
Nuclease_off_+_GSP2
(SEQ ID NO: 38)
CTCTCTATGGGCAGTCGGTGATTTGAGTTGTCATATGTTAATAACGGTA +
Nuclease_off_-_GSP2
(SEQ ID NO: 39)
CCTCTCTATGGGCAGTCGGTGATACATATGACAACTCAATTAAAC -

In addition, this protocol differed from a previously published protocol¹⁷ in the following manner: In a 24-well format, HEK293T cells were transfected with 250 ng Cas9, 150 ng sgRNA, 50 ng GFP, and 10 pmol of annealed GUIDE-Seq oligonucleotide using Lipofectamine 3000 transfection reagent (Invitrogen) according to manufacturer's suggested protocol. 48 hours post-transfection, genomic DNA was extracted via DNeasy Blood and Tissue kit (Qiagen) according to the manufacturer's suggested protocol. Library preparations were done with original adaptors according to protocols described by the Joung laboratory¹⁷, where each library was barcoded for pooled sequencing. The barcoded, purified libraries were deep sequenced as a pool using two paired-end 150 bp MiSeq runs.

Reads containing the identical molecular index and identical starting 8 bp elements on the Read1 were pooled into one unique read. The initial 30 bp and the final 50 bp of the unique Read2 sequences were clipped for removal of the adapter sequence and low quality sequences and then mapped to the human genome (hg19) using Bowtie². Peaks containing mapped unique reads were identified using a pile-up program ESAT (garberlab. umassmed.edu/software/esat/) using a window of 25 bp with a 15 bp overlap. Neighboring windows that were on different strands of the genome and less than 50 bp apart were merged using Bioconductor package ChIPpeakAnno^{62,63}. Peaks that were present with multiple different guides (hotspots¹⁷) or do not contain unique reads for both sense and anti-sense libraries¹⁷ were discarded. The remaining peaks were searched for sequence elements that were complementary to the nuclease target site using CRISPRseek²¹. Only peaks that harbor a sequence with less than 7 mismatches to the target site were considered potential off-target sites. The number of reads from these regions of the sense and the anti-sense libraries were combined into the final read number.

### Example VII

### CRISPRseek Analysis

Human hg19 exon and promoter sequences were fetched using Bioconductor packages ChIPpeakAnno^{62,63} and TxDb.Hsapiens.UCSC.hg19.knownGene. A subset of 16500 exons and 192 promoter sequences of 2 kb each were selected for sgRNA searching and genome-wide off target analyis was using Bioconductor package CRISPRseek^{21,22} using the default settings (both nGG and nAG PAMs were allowed) except BSgenomeName=BSgenome.Hsapiens.UCSC.hgl9, annotateExon=FALSE, outputUniqueREs=FALSE, exportAllgRNAs=“fasta” and fetchSequence=FALSE.

After excluding sgRNAs with on-target or/and off-targets in the haplotype blocks, there were 124793 unique sgRNAs from exon sequences and 55687 unique gRNA from promoter sequences included in the analysis. Each guide was binned based on either the off-target site with the fewest number of mismatches to the guide sequence or the sum of the off-target scores for the top 10 off-target sites. The fraction of guides in each bin for exons or promoters was displayed as a pie chart.

### Example VIII

### Cas9-ZFP Fusions

In principle, Zinc Finger Protein (ZFPs) containing from three to six fingers can be designed for the construction of Cas9-ZFPs, which bind 9 bp to 18 bp target sites respectively (e.g., approximately 3 bp per finger). Based on the data presented herein with the Cas9-ZFP^{TS2/TS3/TS4} system, construction of a four-finger ZFP is preferable for initial testing of Cas9-ZFPs at a particular target site.

For Cas9-ZFPs containing a 58 aa linker the target site can be 5 to 14 bp downstream of the last base pair of the PAM triplet and can be on either the Watson or the Crick strand. If longer ZFPs are desired (5 or 6 fingers), one or more TGSQKP linkers are preferable to break an array into 2 or 3 finger module sets¹. Other modified linkers can be utilized to skip a base between pairs of zinc finger modules to achieve more favorable recognition by neighboring arrays if desired. For the commercial design of zinc fingers, Sangamo Biosciences' proprietary zinc finger module archive has a design density likely less than every 10 bp⁴, combined with the flexibility of the spacing and orientation, multiple ZFPs can be designed and tested around almost any Cas9 target site. These ZFPs can be purchased from Sigma Aldrich.

In addition, a number of open-source systems have been described for selecting or assembling ZFPs. Highly specific ZFPs can be selected from randomized finger libraries using phage or bacterial selections, but this process is labor intensive and may be accessible to only few laboratories. By contrast, modular assembly^{6,7,16-20} wherein pre-characterized single zinc finger modules that recognize 3-base-pair (bp) subsites are joined into arrays, rapidly yields ZFPs that bind desired target sites, and has proven to be an effective method for the creation of active Cas9-ZFPs. For modular assembly, a number of zinc finger archives have been described focusing on single-finger (1F)^{5,17,19,21} and two-finger (2F) modules^{6,7,16,18,22} Using phage-based selections, Barbas lab identified 1F-modules that target 49 of the 64 triplets^{1-14,17} The Kim lab has reported 1F-modules recognizing 38 of the 64 triplets¹⁹. A curated archive of 1F-modules that bind 27 of 64 triplets has been published²¹.

Recently, using bacterial-one-hybrid based selections Noyes lab defined zinc finger modules that can recognize each of the 64 DNA triplets allowing targeting virtually any DNA sequence⁵. In addition, two-finger archives have been published that take into account finger-finger interface and therefore can yield ZFPs with higher specificity but the targeting range of these 2F archives is more limited^{6,7,16,18}. The 1F and 2F archives described herein can be used to design a ZFP roughly every 10 bp, whereas some of the other finger archives can achieve even higher design densities. With the number of finger archives now available, it is possible to design a ZFPA targeting almost every DNA sequence.

Moreover, there are a number of tools available to help users to identify the best target site and design a ZFP. A web-based tool has been designed for the identification of Cas9-ZFP target sites for which ZFPs can be designed from our zinc finger archive. mccb.umassmed.edu/Cas9-pDBD_search. This site provides a simple scoring function for the evaluation of ZFPs with higher activity based on the number of arginine-guanine contacts that are present. Tools from other laboratories are available for the construction of ZFPAs. The “Zinc Finger Tools” published by Barbas lab can identify target sites for single ZFPs and design ZFPs using their archive of 49 1F-modules²³. scripps.edu/barbas/zfdesign/zfdesignhome.php. The Joung laboratory has developed a suite of tools “ZiFiT” that allows the design of ZFPAs for a particular target sequence²⁴. zifit.partners.org/ZiFiT/. In addition, a zinc finger tool developed by Noyes laboratory can be used to design zinc finger arrays one finger at a time for a desired target sitess. zf.princeton.edu/bIh/dna.html. This tool provides multiple zinc finger(s) for every DNA triplet but does not identify the best zinc finger site in a given target sequence.

### Example IX

### Cas9-TALE Fusions

When designing TALE-arrays for Cas9-TALE fusion, a minimum of a 10 bp target site is preferred (excluding the 5′ T) located approximately 10-14 bp downstream and on the Watson strand relative to the NGG PAM site. Alternatively, a target site may comprise a 5′ T²⁵. Multiple programs are available that allow design of single TAL-arrays including TALE-NT²⁶ (tale-nt.cac.cornell.edu/) and SAPTA TAL Targeter Tool²⁷. bao.rice.edu/Research/BioinformaticTools/TAL_targeter.html.

### REFERENCES

1. Doudna, J. A. & Charpentier, E. Genome editing. The new frontier of genomeengineering with CRISPR-Cas9. Science 346, 1258096-1258096 (2014).
2. Sander, J. D. & Joung, J. K. CRISPR-Cas systems for editing, regulating and targeting genomes. Nature biotechnology 32, 347-355 (2014).
3. Hsu, P. D., Lander, E. S. & Zhang, F. Development and Applications of CRISPRCas9 for Genome Engineering. Cell 157, 1262-1278 (2014).
4. Jinek, M. et al. A programmable dual-RNA-guided DNA endonuclease in adaptivebacterial immunity. Science 337, 816-821 (2012).
5. Sternberg, S. H., Redding, S., Jinek, M., Greene, E. C. & Doudna, J. A. DNAinterrogation by the CRISPR RNA-guided endonuclease Cas9. Nature 507, 62-67 (2014).
6. Szczelkun, M. D. et al. Direct observation of R-loop formation by single RNAguided Cas9 and Cascade effector complexes. Proceedings of the National Academy of Sciences 111, 9798-9803 (2014).
7. Anders, C., Niewoehner, O., Duerst, A. & Jinek, M. Structural basis of PAMdependen target DNA recognition by the Cas9 endonuclease. Nature 513, 569-573 (2014).
8. Jiang, F., Zhou, K., Ma, L., Gressel, S. & Doudna, J. A. STRUCTURAL BIOLOGY. A Cas9-guide RNA complex preorganized for target DNA recognition. Science 348, 1477-1481 (2015).
9. Hsu, P. D. et al. DNA targeting specificity of RNA-guided Cas9 nucleases. Nature biotechnology 31, 827-832 (2013).
10. Tsai, S. Q. et al. Dimeric CRISPR RNA-guided FokI nucleases for highly specific genome editing. Nature biotechnology 32, 569-576 (2014).
11. Zhang, Y. et al. Comparison of non-canonical PAMs for CRISPR/Cas9-mediated DNA cleavage in human cells. Sci Rep 4, 5405 (2014).
12. Gabriel, R., Kalle, von, C. & Schmidt, M. Mapping the precision of genome editing. Nature biotechnology 33, 150-152 (2015).
13. Ledford, H. CRISPR, the disruptor. Nature 522, 20-24 (2015).
14. Fu, Y. et al. High-frequency off-target mutagenesis induced by CRISPR-Cas nucleases in human cells. Nature biotechnology 31, 822-826 (2013).
15. Lin, Y. et al. CRISPR/Cas9 systems have off-target activity with insertions or deletions between target DNA and guide RNA sequences. Nucleic Acids Research 42, 7473-7485 (2014).
16. Pattanayak, V. et al. High-throughput profiling of off-target DNA cleavage reveals RNA-programmed Cas9 nuclease specificity. Nature biotechnology 31, 839-843 (2013).
17. Tsai, S. Q. et al. GUIDE-seq enables genome-wide profiling of off-target cleavage by CRISPR-Cas nucleases. Nature biotechnology 33, 187-197 (2015).
18. Frock, R. L. et al. Genome-wide detection of DNA double-stranded breaks induced by engineered nucleases. Nature biotechnology 33, 179-186 (2015).
19. Kim, D. et al. Digenome-seq: genome-wide profiling of CRISPR-Cas9 off-target effects in human cells. Nature Methods 12, 237-243 (2015).
20. Wang, X. et al. Unbiased detection of off-target cleavage by CRISPR-Cas9 and TALENs using integrase-defective lentiviral vectors. Nature biotechnology (2015).
21. Zhu, L. J., Holmes, B. R., Aronin, N. & Brodsky, M. H. CRISPRseek: A Bioconductor Package to Identify Target-Specific Guide RNAs for CRISPR-Cas9 Genome-Editing Systems.

PLoS ONE 9, e108424 (2014).
22. Zhu, L. J. Overview of guide RNA design tools for CRISPR-Cas9 genome editing technology. Frontiers in Biology (2015).
23. Brunet, E. et al. Chromosomal translocations induced at specified loci in human stem cells. Proceedings of the National Academy of Sciences 106, 10620-10625 (2009).
24. Lee, H. J., Kim, E. & Kim, J.-S. Targeted chromosomal deletions in human cells using zinc finger nucleases. Genome Research 20, 81-89 (2010).
25. Fu, Y., Sander, J. D., Reyon, D., Cascio, V. M. & Joung, J. K. Improving CRISPRCas nuclease specificity using truncated guide RNAs. Nature biotechnology 32, 279-284 (2014).
26. Cho, S. W. et al. Analysis of off-target effects of CRISPR/Cas-derived RNAguided endonucleases and nickases. Genome Research 24, 132-141 (2014).
27. Ran, F. A. et al. Double Nicking by RNA-Guided CRISPR Cas9 for Enhanced Genome Editing Specificity. Cell 154, 1380-1389 (2013).
28. Mali, P. et al. CAS9 transcriptional activators for target specificity screening and paired nickases for cooperative genome engineering. Nature biotechnology 31, 833-838 (2013).
29. Guilinger, J. P., Thompson, D. B. & Liu, D. R. Fusion of catalytically inactive Cas9 to FokI nuclease improves the specificity of genome modification. Nature biotechnology 32, 577-582 (2014).
30. Zetsche, B., Volz, S. E. & Zhang, F. A split-Cas9 architecture for inducible genome editing and transcription modulation. Nature biotechnology 33, 139-142 (2015).
31. Nihongaki, Y., Kawano, F., Nakajima, T. & Sato, M. Photoactivatable CRISPRCas9 for optogenetic genome editing. Nature biotechnology (2015).
32. Wright, A. V. et al. Rational design of a split-Cas9 enzyme complex. Proceedings of the National Academy of Sciences 112, 2984-2989 (2015).
33. Davis, K. M., Pattanayak, V., Thompson, D. B., Zuris, J. A. & Liu, D. R. Small molecule-triggered Cas9 protein with improved genome-editing specificity. Nat Chem Biol (2015). doi:10.1038/nchembio.1793
34. Kleinstiver, B. P. et al. Engineered CRISPR-Cas9 nucleases with altered PAM specificities. Nature (2015).
35. Kim, S., Kim, D., Cho, S. W., Kim, J. & Kim, J.-S. Highly efficient RNA-guided genome editing in human cells via delivery of purified Cas9 ribonucleoproteins. Genome Research 24, 1012-1019 (2014).
36. Ramakrishna, S. et al. Gene disruption by cell-penetrating peptide-mediated delivery of Cas9 protein and guide RNA. Genome Research 24, 1020-1027 (2014).
37. Zuris, J. A. et al. Cationic lipid-mediated delivery of proteins enables efficient protein-based genome editing in vitro and in vivo. Nature biotechnology 33, 73-80 (2015).
38. Tsai, S. Q. & Joung, J. K. What's changed with genome editing?Cell Stem Cell 15, 3-4 (2014).
39. Urnov, F. D., Rebar, E. J., Holmes, M. C., Zhang, H. S. & Gregory, P. D. Genome editing with engineered zinc finger nucleases. Nat Rev Genet 11, 636-646 (2010).
40. Joung, J. K. & Sander, J. D. TALENs: a widely applicable technology for targeted genome editing. Nat. Rev. Mol. Cell Biol. 14, 49-55 (2013).
41. Persikov, A. V. et al. A systematic survey of the Cys2His2 zinc finger DNA-binding landscape. Nucleic Acids Research 43, 1965-1984 (2015).
42. Lamb, B. M., Mercer, A. C. & Barbas, C. F. Directed evolution of the TALE Nterminal domain for recognition of all 5′ bases. Nucleic Acids Research 41, 9779-9785 (2013).
43. Boissel, S. et al. megaTALs: a rare-cleaving nuclease architecture for therapeutic genome engineering. Nucleic Acids Research 42, 2591-2601 (2014).
44. Khalil, A. S. et al. A synthetic biology framework for programming eukaryotic transcription functions. Cell 150, 647-658 (2012).
45. Meckler, J. F. et al. Quantitative analysis of TALE-DNA interactions suggests polarity effects. Nucleic Acids Research 41, 4118-4128 (2013).
46. Wilson, K. A., Chateau, M. L. & Porteus, M. H. Design and Development of Artificial Zinc Finger Transcription Factors and Zinc Finger Nucleases to the hTERT Locus. Mol Ther Nucleic Acids 2, e87 (2013).
47. Atkinson, H. & Chalmers, R. Delivering the goods: viral and non-viral gene therapy systems and the inherent limits on cargo DNA and internal sequences. Genetica 138, 485-498 (2010).
48. Klemm, J. D. & Pabo, C. O. Oct-1 POU domain-DNA interactions: cooperative binding of isolated subdomains and effects of covalent linkage. Genes & Development 10, 27-36 (1996).
49. Chylinski, K., Makarova, K. S., Charpentier, E. & Koonin, E. V. Classification and evolution of type II CRISPR-Cas systems. Nucleic Acids Research 42, 6091-6105 (2014).
50. Hou, Z. et al. Efficient genome engineering in human pluripotent stem cells using Cas9 from Neisseria meningitidis. Proceedings of the National Academy of Sciences 110, 15644-15649 (2013).
51. Kearns, N. A. et al. Cas9 effector-mediated regulation of transcription and differentiation in human pluripotent stem cells. Development 141, 219-223 (2014).
52. Villefranc, J. A., Amigo, J. & Lawson, N. D. Gateway compatible vectors for analysis of gene function in the zebrafish. Dev Dyn 236, 3077-3087 (2007).
53. Gupta, A. et al. An optimized two-finger archive for ZFN-mediated gene targeting. Nature Methods 9, 588-590 (2012).
54. Zhu, C. et al. Using defined finger-finger interfaces as units of assembly for constructing zinc-finger nucleases. Nucleic Acids Research 41, 2455-2465 (2013).
55. Cermak, T. et al. Efficient design and assembly of custom TALEN and other TAL effector-based constructs for DNA targeting. Nucleic Acids Research 39, e82-e82 (2011).
56. Kok, F. O., Gupta, A., Lawson, N. D. & Wolfe, S. A. Construction and application of site-specific artificial nucleases for targeted gene editing. Methods Mol Biol 1101, 267-303 (2014).
57. Gupta, A. et al. Targeted chromosomal deletions and inversions in zebrafish. Genome Research 23, 1008-1017 (2013).
58. Schneider, C. A., Rasband, W. S. & Eliceiri, K. W. NIH Image to ImageJ: 25 years of image analysis. Nature Methods 9, 671-675 (2012).
59. Gupta, A., Meng, X., Zhu, L. J., Lawson, N. D. & Wolfe, S. A. Zinc finger proteindependent and -independent contributions to the in vivo off-target activity of zinc finger nucleases. Nucleic Acids Research 39, 381-392 (2011).
60. Ihaka, R. & Gentleman, R. R: A Language for Data Analysis and Graphics. Journal of Computational and Graphical Statistics 5, 299-314 (1996).
61. Benjamini, Y. & Hochberg, Y. Controlling the false discovery rate: a practical and powerful approach to multiple testing. Journal of the Royal Statistical Society Series B 57, 289-300 (1995).
62. Zhu, L. J. et al. ChIPpeakAnno: a Bioconductor package to annotate ChIP-seq and ChIP-chip data. BMC Bioinformatics 11, 237 (2010).
63. Zhu, L. J. in Methods in Molecular Biology (eds. Lee, T.-L. & Shui Luk, A. C.) 1067, 105-124 (Humana Press, 2013).

### SUPPLEMENTARY REFERENCES

Li, H. et al. In vivo genome editing restores haemostasis in a mouse model of haemophilia. Nature 475, 217-221 (2011).
Yusa, K. et al. Targeted gene correction of al-antitrypsin deficiency in induced pluripotent stem cells. Nature 478, 391-394 (2011).
Mahiny, A. J. et al. In vivo genome editing using nuclease-encoding mRNA corrects SP-B deficiency. Nature biotechnology (2015).
Gupta, R. M. & Musunuru, K. Expanding the genetic editing tool kit: ZFNs, TALENs, and CRISPR-Cas9. J Clin Invest 124, 4154-4161 (2014).
Persikov, A. V. et al. A systematic survey of the Cys2His2 zinc finger DNAbinding landscape. Nucleic Acids Research 43, 1965-1984 (2015).
Zhu, C. et al. Using defined finger-finger interfaces as units of assembly for constructing zinc-finger nucleases. Nucleic Acids Research 41, 2455-2465 (2013).
Gupta, A. et al. An optimized two-finger archive for ZFN-mediated gene targeting. Nature Methods 9, 588-590 (2012).
Maeder, M. L., Thibodeau-Beganny, S., Sander, J. D., Voytas, D. F. & Joung, J. K. Oligomerized pool engineering (OPEN): an ‘open-source’ protocol for making customized zinc-finger arrays. Nat Protoc 4, 1471-1501 (2009).
Maeder, M. et al. Rapid “Open-Source” Engineering of Customized Zinc-Finger Nucleases for Highly Efficient Gene Modification. Molecular Cell 31, 294-301 (2008).
Meng, X., Noyes, M. B., Zhu, L. J., Lawson, N. D. & Wolfe, S. A. Targeted gene inactivation in zebrafish using engineered zinc-finger nucleases. Nature biotechnology 26, 695-701 (2008).
Dreier, B. et al. Development of zinc finger domains for recognition of the 5′-CNN-3′ family DNA sequences and their use in the construction of artificial transcription factors. J Biol Chem 280, 35588-35597(2005).
Dreier, B., Beerli, R., Segal, D., Flippin, J. & Barbas, C. Development of zinc finger domains for recognition of the 5′-ANN-3′ family of DNA sequences and their use in the construction of artificial transcription factors. Journal of Biological Chemistry 276, 29466 (2001).
Dreier, B., Segal, D. J. & Barbas, C. F. Insights into the molecular recognition of the 5′-GNN-3′ family of DNA sequences by zinc finger domains. J Mol Biol 303, 489-502 (2000).
Segal, D. J., Dreier, B., Beerli, R. R. & Barbas, C. F. Toward controlling gene expression at will: selection and design of zinc finger domains recognizing each of the 5′-GNN-3′ DNA target sequences. Proc Natl Acad Sci USA 96, 2758-2763 (1999).
Greisman, H. A. & Pabo, C. O. A general strategy for selecting high-affinity zinc finger proteins for diverse DNA target sites. Science 275, 657-661 (1997).
Sander, J. D. et al. Selection-free zinc-finger-nuclease engineering by context-dependent assembly (CoDA). Nature Methods 8, 67-69 (2011).
Carroll, D., Morton, J. J., Beumer, K. J. & Segal, D. J. Design, construction and in vitro testing of zinc finger nucleases. Nat Protoc 1, 1329-1341 (2006).
Kim, S., Lee, M. J., Kim, H., Kang, M. & Kim, J.-S. Preassembled zincfinger arrays for rapid construction of ZFNs. Nature Methods 8, 7 (2011).
Kim, H. J., Lee, H. J., Kim, H., Cho, S. W. & Kim, J.-S. Targeted genome editing in human cells with zinc finger nucleases constructed via modular assembly. Genome Research 19, 1279-1288 (2009).
Bhakta, M. S. et al. Highly active zinc-finger nucleases by extended modular assembly. Genome Research 23, 530-538 (2013).
Zhu, C. et al. Evaluation and application of modularly assembled zincfinger nucleases in zebrafish. Development 138, 4555-4564 (2011).
Doyon, Y. et al. Heritable targeted gene disruption in zebrafish using designed zinc-finger nucleases. Nature biotechnology 26, 702-708 (2008).
Mandell, J. G. & Barbas, C. F. Zinc Finger Tools: custom DNA-binding domains for transcription factors and nucleases. Nucleic Acids Research 34, W516-W523 (2006).
Sander, J. D. et al. ZiFiT (Zinc Finger Targeter): an updated zinc finger engineering tool. Nucleic Acids Research 38, W462-W468 (2010).
Miller, J. C. et al. Improved specificity of TALE-based genome editing using an expanded RVD repertoire. Nature Methods (2015).
Doyle, E. L. et al. TAL Effector-Nucleotide Targeter (TALE-NT) 2.0: tools for TAL effector design and target prediction. Nucleic Acids Research 40, W 117-22 (2012).
Lin, Y. et al. SAPTA: a new design tool for improving TALE nuclease activity. Nucleic Acids Research gkt1363 (2014).
Zhu, L. J., Holmes, B. R., Aronin, N. & Brodsky, M. H. CRISPRseek: A Bioconductor Package to Identify Target-Specific Guide RNAs for CRISPRCas9 Genome-Editing Systems. PLoS ONE 9, e108424 (2014).
Lin, Y. et al. CRISPR/Cas9 systems have off-target activity with insertions or deletions between target DNA and guide RNA sequences. Nucleic Acids Research 42, 7473-7485 (2014).
Anders, C., Niewoehner, O., Duerst, A. & Jinek, M. Structural basis of PAM-dependent target DNA recognition by the Cas9 endonuclease. Nature 513, 569-573 (2014).
Elrod-Erickson, M., Rould, M. A., Nekludova, L. & Pabo, C. O. Zif268 protein-DNA complex refined at 1.6 A: a model system for understanding zinc finger-DNA interactions. Structure 4, 1171-1180 (1996).
Lu, X.-J. & Olson, W. K. 3DNA: a versatile, integrated software system for the analysis, rebuilding and visualization of three-dimensional nucleic-acid structures. Nat Protoc 3, 1213-1227 (2008).
Wilson, K. A., Chateau, M. L. & Porteus, M. H. Design and Development of Artificial Zinc Finger Transcription Factors and Zinc Finger Nucleases to the hTERT Locus. Mol Ther Nucleic Acids 2, e87 (2013).
Fu, Y., Sander, J. D., Reyon, D., Cascio, V. M. & Joung, J. K. Improving CRISPR-Cas nuclease specificity using truncated guide RNAs. Nature biotechnology 32, 279-284 (2014).
Gupta, A. et al. An improved predictive recognition model for Cys(2)-His(2)zinc finger proteins. Nucleic Acids Research 42, 4800-4812 (2014).

1-42. (canceled)

43. A method for genome editing of DNA within a cell, comprising:
contacting the DNA in the cell with:
a Cas9 fusion protein comprising (i) a first Cas9 nuclease comprising a mutated protospacer adjacent motif (PAM) recognition domain, wherein the mutated PAM recognition domain comprises a mutation at any one of positions R1333, R1335, R1025, or K1013 and (ii) a second Cas9 nuclease;
a first guide RNA for the first Cas9 nuclease, wherein the first guide RNA is complementary to a first target site; and
a second guide RNA for the second Cas9 nuclease, wherein the second guide RNA is complementary to a second target site;
such that the first Cas9 nuclease cleaves the first target site.

44. The method of claim 43, wherein the second Cas9 nuclease is an orthogonal Cas9 isoform.

45. The method of claim 43, wherein the first target site comprises a protospacer adjacent motif sequence for the first Cas9 nuclease, and the second target site comprises a protospacer adjacent motif sequence for the second Cas9 nuclease.

46. The method of claim 43, further comprising cleaving the second target site with the second Cas9 nuclease.

47. The method of claim 46, wherein a genomic deletion is generated.

48. The method of claim 43, wherein the first and second Cas9 nucleases are selected from the group consisting of Streptococcus pyogenes Cas9 (SpCas9), Staphylococcus aureus Cas9 (SaCas9), Neisseria meningitidis Cas9 (NmCas9) and Actinomyces naeslundii Cas9 (AnCas9).

49. The method of claim 43, wherein the second Cas9 nuclease is selected from the group consisting of nuclease-dead NmCas9 (NmdCas9), NmCas9 nuclease, NmCas9 HNH nickase, and NmCas9 RuvC nickase.

50. The method of claim 43, wherein said second Cas9 nuclease is selected from the group consisting of a Cas9 nickase and a nuclease-dead Cas9 (dCas9).

51. The method of claim 43, wherein the first Cas9 nuclease has a single point mutation.

52. The method of claim 43, wherein the first Cas9 nuclease has a double mutation.

53. The method of claim 43, wherein the mutated PAM recognition domain of the first Cas9 nuclease comprises a lysine, serine, or alanine substitution at position R1333.

54. The method of claim 43, wherein the mutated PAM recognition domain of the first Cas9 nuclease comprises a lysine, serine, or alanine substitution at position R1335.

55. The method of claim 43, wherein the mutated PAM recognition domain of the first Cas9 nuclease comprises a lysine, serine, or alanine substitution at position R1025.

56. The method of claim 43, wherein the mutated PAM recognition domain of the first Cas9 nuclease comprises a lysine, serine, or alanine substitution at position K1013.

57. The method of claim 43, wherein said first and second Cas9 nucleases are bound to a guide RNA comprising a guide sequence element.

58. The method of claim 57, wherein said guide sequence element is truncated.

59. The method of claim 43, wherein the fusion protein comprises a linker.

60. The method of claim 44, wherein the fusion protein comprises a linker that ranges between 20 and sixty amino acids.

61. The method of claim 43, wherein the first guide RNA is a single guide RNA or a combination of crRNA and tracrRNA, and wherein the second guide RNA is a single guide RNA or a combination of crRNA and tracrRNA.


SEQUENCE LISTING (153 sequences)

SEQ ID NO: 1-3 - skipped (3 entries)

SEQ ID NO: 4 - DNA, 12 bp, synthetic construct
  misc_feature 1..12: note = Synthetic
  source 1..12: mol_type = other DNA
gcgggcaggggc

SEQ ID NO: 5 - DNA, 12 bp, synthetic construct
  misc_feature 1..12: note = Synthetic
  source 1..12: mol_type = other DNA
gcaggggccgga

SEQ ID NO: 6 - DNA, 12 bp, synthetic construct
  misc_feature 1..12: note = Synthetic
  source 1..12: mol_type = other DNA
ggcgttggagcg

SEQ ID NO: 7 - DNA, 12 bp, synthetic construct
  misc_feature 1..12: note = Synthetic
  source 1..12: mol_type = other DNA
ccggttgatgtg

SEQ ID NO: 8 - skipped

SEQ ID NO: 9 - DNA, 12 bp, synthetic construct
  misc_feature 1..12: note = Synthetic
  source 1..12: mol_type = other DNA
gaaacgggatcg

SEQ ID NO: 10 - DNA, 15 bp, synthetic construct
  misc_feature 1..15: note = Synthetic
  source 1..15: mol_type = other DNA
acacagtacctggca

SEQ ID NO: 11 - DNA, 12 bp, synthetic construct
  misc_feature 1..12: note = Synthetic
  source 1..12: mol_type = other DNA
gaaaagtatcaa

SEQ ID NO: 12 - DNA, 10 bp, synthetic construct
  misc_feature 1..10: note = Synthetic
  source 1..10: mol_type = other DNA
tgcgtgggcg

SEQ ID NO: 13 - DNA, 11 bp, synthetic construct
  misc_feature 1..11: note = Synthetic
  source 1..11: mol_type = other DNA
ttggagcgggg

SEQ ID NO: 14 - DNA, 17 bp, synthetic construct
  misc_feature 1..17: note = Synthetic
  source 1..17: mol_type = other DNA
ttggagcggggagaagg

SEQ ID NO: 15 - DNA, 11 bp, synthetic construct
  misc_feature 1..11: note = Synthetic
  source 1..11: mol_type = other DNA
tcaaccggtgg

SEQ ID NO: 16 - DNA, 17 bp, synthetic construct
  misc_feature 1..17: note = Synthetic
  source 1..17: mol_type = other DNA
tcaaccggtggcgcatt

SEQ ID NO: 17-22 - skipped (6 entries)

SEQ ID NO: 23 - DNA, 20 bp, synthetic construct
  misc_feature 1..20: note = Synthetic
  source 1..20: mol_type = other DNA
ggtgagtgagtgtgtgcgtg

SEQ ID NO: 24 - DNA, 21 bp, synthetic construct
  misc_feature 1..21: note = Synthetic
  source 1..21: mol_type = other DNA
gcctgagtgagtgtgtgcgtg

SEQ ID NO: 25 - DNA, 20 bp, synthetic construct
  misc_feature 1..20: note = Synthetic
  source 1..20: mol_type = other DNA
ggcaagtgagtgtgtgcgtg

SEQ ID NO: 26 - DNA, 20 bp, synthetic construct
  misc_feature 1..20: note = Synthetic
  source 1..20: mol_type = other DNA
ggtgcttgagtgtgtgcgtg

SEQ ID NO: 27 - DNA, 20 bp, synthetic construct
  misc_feature 1..20: note = Synthetic
  source 1..20: mol_type = other DNA
ggtgaggaagtgtgtgcgtg

SEQ ID NO: 28 - DNA, 20 bp, synthetic construct
  misc_feature 1..20: note = Synthetic
  source 1..20: mol_type = other DNA
ggtgagtgcttgtgtgcgtg

SEQ ID NO: 29 - DNA, 20 bp, synthetic construct
  misc_feature 1..20: note = Synthetic
  source 1..20: mol_type = other DNA
ggtgagtgagcatgtgcgtg

SEQ ID NO: 30 - DNA, 20 bp, synthetic construct
  misc_feature 1..20: note = Synthetic
  source 1..20: mol_type = other DNA
ggtgagtgagtgcatgcgtg

SEQ ID NO: 31 - DNA, 20 bp, synthetic construct
  misc_feature 1..20: note = Synthetic
  source 1..20: mol_type = other DNA
ggtgagtgagtgtggtcgtg

SEQ ID NO: 32 - DNA, 20 bp, synthetic construct
  misc_feature 1..20: note = Synthetic
  source 1..20: mol_type = other DNA
ggtgagtgagtgtgtgtatg

SEQ ID NO: 33 - DNA, 20 bp, synthetic construct
  misc_feature 1..20: note = Synthetic
  source 1..20: mol_type = other DNA
ggtgagtgagtgtgtgcgca

SEQ ID NO: 34-35 - skipped (2 entries)

SEQ ID NO: 36 - DNA, 48 bp, synthetic construct
  misc_feature 1..48: note = Synthetic
  source 1..48: mol_type = other DNA
ggatctcgacgctctccctgtttaattgagttgtcatatgttaataac

SEQ ID NO: 37 - DNA, 41 bp, synthetic construct
  misc_feature 1..41: note = Synthetic
  source 1..41: mol_type = other DNA
ggatctcgacgctctccctataccgttattaacatatgaca

SEQ ID NO: 38 - DNA, 49 bp, synthetic construct
  misc_feature 1..49: note = Synthetic
  source 1..49: mol_type = other DNA
ctctctatgggcagtcggtgatttgagttgtcatatgttaataacggta

SEQ ID NO: 39 - DNA, 45 bp, synthetic construct
  misc_feature 1..45: note = Synthetic
  source 1..45: mol_type = other DNA
cctctctatgggcagtcggtgatacatatgacaactcaattaaac

SEQ ID NO: 40 - DNA, 43 bp, synthetic construct
  misc_feature 1..43: note = Synthetic
  source 1..43: mol_type = other DNA
gggcccaacctagggcatggaggcggctgctggtgcgtgggcg

SEQ ID NO: 41 - DNA, 43 bp, synthetic construct
  misc_feature 1..43: note = Synthetic
  source 1..43: mol_type = other DNA
gcatgggtgatgtcaatgccaaggccagtcaggtgcgtgggcg

SEQ ID NO: 42 - DNA, 43 bp, synthetic construct
  misc_feature 1..43: note = Synthetic
  source 1..43: mol_type = other DNA
cctggacccaacgccccaggagaagagcgaaggtgcgtgggcg

SEQ ID NO: 43 - DNA, 43 bp, synthetic construct
  misc_feature 1..43: note = Synthetic
  source 1..43: mol_type = other DNA
gctggcggaagacagagtgctgctattcacctctgcgtgggcg

SEQ ID NO: 44 - DNA, 36 bp, synthetic construct
  misc_feature 1..36: note = Synthetic
  source 1..36: mol_type = other DNA
ttcgacaccaccatagacagaaagcggtacacctct

SEQ ID NO: 45 - DNA, 36 bp, synthetic construct
  misc_feature 1..36: note = Synthetic
  source 1..36: mol_type = other DNA
ttcgacaccaccatagacaaaaagcggtacacctct

SEQ ID NO: 46 - DNA, 36 bp, synthetic construct
  misc_feature 1..36: note = Synthetic
  source 1..36: mol_type = other DNA
ttcgacaccaccatagacagcaagcggtacacctct

SEQ ID NO: 47 - DNA, 36 bp, synthetic construct
  misc_feature 1..36: note = Synthetic
  source 1..36: mol_type = other DNA
ttcgacaccaccatagacagaaagaagtacacctct

SEQ ID NO: 48 - DNA, 36 bp, synthetic construct
  misc_feature 1..36: note = Synthetic
  source 1..36: mol_type = other DNA
ttcgacaccaccatagacagaaagagctacacctct

SEQ ID NO: 49 - DNA, 48 bp, synthetic construct
  misc_feature 1..48: note = Synthetic
  source 1..48: mol_type = other DNA
gtgggtgagtgagtgtgtgcgtgtggggttgagggcgttggagcgggg

SEQ ID NO: 50 - DNA, 53 bp, synthetic construct
  misc_feature 1..53: note = Synthetic
  source 1..53: mol_type = other DNA
cccgaccccctccaccccgcctccgggcgcgggctccggcccctgcccgcggc

SEQ ID NO: 51 - DNA, 48 bp, synthetic construct
  misc_feature 1..48: note = Synthetic
  source 1..48: mol_type = other DNA
cctgagtccgagcagaagaagaagggctcccatcacatcaaccggtgg

SEQ ID NO: 52 - protein, 28 aa, Neisseria meningitidis
KKARMFGYFASCHRGTGNINIKINDLDN

SEQ ID NO: 53 - protein, 28 aa, Pasteurella multocida
KKEYFFGYYIGLDRATGNISLKEHDGEI

SEQ ID NO: 54 - protein, 28 aa, Haemophilus parainfluenzae
KKKTILGYFNGLNRATGNIDIKEHDLDK

SEQ ID NO: 55 - protein, 28 aa, Actinobacillus minor
KKKTIFGYFNGLNRATSNINIKEHDLDK

SEQ ID NO: 56 - protein, 28 aa, Neisseria lactamica
KKNEFLGYGVSLNRATGAIDIRTHDTDS

SEQ ID NO: 57 - protein, 28 aa, Actinobacillus suis
KKEEFFGYYGGLDRATGAIVIKEHDLEK

SEQ ID NO: 58 - protein, 28 aa, Kingella kingae
KKGCIMGYFASLDRATGAITIREHDRDK

SEQ ID NO: 59 - protein, 28 aa, Mannheimia varigena
KKETFFGYYNGLDRATGAINLKEPDSNI

SEQ ID NO: 60 - protein, 28 aa, Simonsiella muelleri
NKGRIFGYYNGLDRANGSIGIREHDLEK

SEQ ID NO: 61 - protein, 28 aa, Neisseria bacilliformis
KKDKFFGYYSGFNRATGAINIKEHDLSK

SEQ ID NO: 62 - DNA, 160 bp, Streptococcus pyogenes
  source 1..160: mol_type = genomic DNA
ggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacggcaccggcg

SEQ ID NO: 63 - DNA, 160 bp, synthetic construct
  misc_feature 1..160: note = Synthetic
  source 1..160: mol_type = other DNA
ggcgcgcctgcagccttcaagtacttcgacaccaccatagacaaaaagcggtacacctctacaaaggaggtcctggacgccacactgattcatcagtcaattacggggctctatgaaacaagaatcgacctctctcagctcggtggagacggcaccggcg

SEQ ID NO: 64 - DNA, 160 bp, synthetic construct
  misc_feature 1..160: note = Synthetic
  source 1..160: mol_type = other DNA
ggcgcgcctgcagccttcaagtacttcgacaccaccatagacagcaagcggtacacctctacaaaggaggtcctggacgccacactgattcatcagtcaattacggggctctatgaaacaagaatcgacctctctcagctcggtggagacggcaccggcg

SEQ ID NO: 65 - DNA, 160 bp, synthetic construct
  misc_feature 1..160: note = Synthetic
  source 1..160: mol_type = other DNA
ggcgcgcctgcagccttcaagtacttcgacaccaccatagacagaaagaagtacacctctacaaaggaggtcctggacgccacactgattcatcagtcaattacggggctctatgaaacaagaatcgacctctctcagctcggtggagacggcaccggcg

SEQ ID NO: 66 - DNA, 160 bp, synthetic construct
  misc_feature 1..160: note = Synthetic
  source 1..160: mol_type = other DNA
ggcgcgcctgcagccttcaagtacttcgacaccaccatagacagaaagagctacacctctacaaaggaggtcctggacgccacactgattcatcagtcaattacggggctctatgaaacaagaatcgacctctctcagctcggtggagacggcaccggcg

SEQ ID NO: 67 - DNA, 8729 bp, synthetic construct
  misc_feature 1..8729: note = Synthetic
  source 1..8729: mol_type = other DNA
accgccattctgcctggggacgtcggagcaagcttgatttaggtgacactatagaatacaagctacttgttctttttgcaggatcccatcgattcgaattcaaggatcaacaagtttgtacaaaaaagcaggctggcgccaccatggacaagaagtactccattgggctcgatatcggtaccaacagcgtcggctgggccgtcattacggacgagtacaaggtgccgagcaaaaaattcaaagttctgggcaataccgatcgccacagcataaagaagaacctcattggagccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagctggtagacagtactgataaggctgacttgcggttgatctatctcgcgctggcgcacatgatcaaatttcggggacacttcctcatcgagggggacctgaacccagacaacagcgatgtcgacaaactctttatccaactggttcagacttacaatcagcttttcgaggagaacccgatcaacgcatccggcgttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagctgcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagctgaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgctcggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagagacgccgatatacaggatgggggcggctgtcaagaaaactgatcaatggcatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaaccggttgtccgactacgacgtggatcatatcgtgccccaaagctttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagctggaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccgcaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatccagcttcgagaaaaaccccatcgactttctcgaagcgaaaggatataaagaggtcaaaaaagacctcatcattaagctgcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgagttctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacggcaccggcgggcccaagaagaagaggaaggtatacccatacgatgttcctgactatgcgggctatccctatgacgtcccggactatgcaggatcgtatccttatgacgttccagattacgctggatccgccgctccggcagctaagaaaaagaaactggatttcgaatccggacgcccatatgcttgccctgtcgagtcctgcgatcgccgcttttctcgctcggatgagcttacccgccatatccgcatccataccggtcagaagcccttccagtgtcgaatctgcatgcgtaacttcagtcgtagtgaccaccttaccacccacatccgcacccacacaggcgagaagccttttgcctgtgacatttgtgggaggaagtttgccaggagtgatgaacgcaagaggcataccaaaatccatttaagacagaaggactaagcggccgcctcgagatatctagacccagctttcttgtacaaagtggttgatcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttaggga

SEQ ID NO: 68 - DNA, 8831 bp, synthetic construct
  misc_feature 1..8831: note = Synthetic
  source 1..8831: mol_type = other DNA
accgccattctgcctggggacgtcggagcaagcttgatttaggtgacactatagaatacaagctacttgttctttttgcaggatcccatcgattcgaattcaaggatcaacaagtttgtacaaaaaagcaggctggcgccaccatgggtagagccgcgccggcagctaagaaaaagaaactggatcaattgcgcccatatgcttgccctgtcgagtcctgcgatcgccgcttttctcgctcggatgagcttacccgccatatccgcatccataccggtcagaagcccttccagtgtcgaatctgcatgcgtaacttcagtcgtagtgaccaccttaccacccacatccgcacccacacaggcgagaagccttttgcctgtgacatttgtgggaggaagtttgccaggagtgatgaacgcaagaggcataccaaaatccatttaagacagaaggaccccgggtctggaggtagcggctcaagtggccgtacggcagctcctgcggcaaaaaagaaaaagttggactctgaattcggaagcgacaagaagtactccattgggctcgatatcggtaccaacagcgtcggctgggccgtcattacggacgagtacaaggtgccgagcaaaaaattcaaagttctgggcaataccgatcgccacagcataaagaagaacctcattggagccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagctggtagacagtactgataaggctgacttgcggttgatctatctcgcgctggcgcacatgatcaaatttcggggacacttcctcatcgagggggacctgaacccagacaacagcgatgtcgacaaactctttatccaactggttcagacttacaatcagcttttcgaggagaacccgatcaacgcatccggcgttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagctgcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagctgaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgctcggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagagacgccgatatacaggatgggggcggctgtcaagaaaactgatcaatggcatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaaccggttgtccgactacgacgtggatcatatcgtgccccaaagctttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagctggaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccgcaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatccagcttcgagaaaaaccccatcgactttctcgaagcgaaaggatataaagaggtcaaaaaagacctcatcattaagctgcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgagttctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacggcaccggcgggcccaagaagaagaggaaggtatacccatacgatgttcctgactatgcgggctatccctatgacgtcccggactatgcaggatcgtatccttatgacgttccagattacgctggatcctaatgattcgaatccggataagcggccgcctcgagatatctagacccagctttcttgtacaaagtggttgatcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttaggga

SEQ ID NO: 69 - DNA, 10061 bp, synthetic construct
  misc_feature 1..10061: note = Synthetic
  source 1..10061: mol_type = other DNA
accgccattctgcctggggacgtcggagcaagcttgatttaggtgacactatagaatacaagctacttgttctttttgcaggatcccatcgattcgaattcaaggatcaacaagtttgtacaaaaaagcaggctggcgccaccatggacaagaagtactccattgggctcgatatcggtaccaacagcgtcggctgggccgtcattacggacgagtacaaggtgccgagcaaaaaattcaaagttctgggcaataccgatcgccacagcataaagaagaacctcattggagccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagctggtagacagtactgataaggctgacttgcggttgatctatctcgcgctggcgcacatgatcaaatttcggggacacttcctcatcgagggggacctgaacccagacaacagcgatgtcgacaaactctttatccaactggttcagacttacaatcagcttttcgaggagaacccgatcaacgcatccggcgttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagctgcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagctgaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgctcggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagagacgccgatatacaggatgggggcggctgtcaagaaaactgatcaatggcatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaaccggttgtccgactacgacgtggatcatatcgtgccccaaagctttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagctggaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccgcaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatccagcttcgagaaaaaccccatcgactttctcgaagcgaaaggatataaagaggtcaaaaaagacctcatcattaagctgcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgagttctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacggcaccggcgggcccaagaagaagaggaaggtatacccatacgatgttcctgactatgcgggctatccctatgacgtcccggactatgcaggatcgtatccttatgacgttccagattacgctggatccgccgctccggcagctaagaaaaagaaactggatttcgaatccggagtacctatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgcccctgaacctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgctaataataacggaggacggccagccttggagtccatcgtagcccaattgtccaggcccgatcccgcgttggctgcgttaacgaatgaccatctggtggcgttggcatgtcttggtggacgacccgcgctcgatgcagtcaaaaagggtctgcctcatgctcccgcattgatcaaaagaaccaaccggcggattcccgagagaacttcccatcgagtcgcgggatcttaagcggccgcctcgagatatctagacccagctttcttgtacaaagtggttgatcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttaggga

SEQ ID NO: 70 - DNA, 10163 bp, synthetic construct
  misc_feature 1..10163: note = Synthetic
  source 1..10163: mol_type = other DNA
accgccattctgcctggggacgtcggagcaagcttgatttaggtgacactatagaatacaagctacttgttctttttgcaggatcccatcgattcgaattcaaggatcaacaagtttgtacaaaaaagcaggctggcgccaccatgggtagagccgcgccggcagctaagaaaaagaaactggatcaattggtacctatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgcccctgaacctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgctaataataacggaggacggccagccttggagtccatcgtagcccaattgtccaggcccgatcccgcgttggctgcgttaacgaatgaccatctggtggcgttggcatgtcttggtggacgacccgcgctcgatgcagtcaaaaagggtctgcctcatgctcccgcattgatcaaaagaaccaaccggcggattcccgagagaacttcccatcgagtcgcgggatctcccgggtctggaggtagcggctcaagtggccgtacggcagctcctgcggcaaaaaagaaaaagttggactctgaattcggaagcgacaagaagtactccattgggctcgatatcggtaccaacagcgtcggctgggccgtcattacggacgagtacaaggtgccgagcaaaaaattcaaagttctgggcaataccgatcgccacagcataaagaagaacctcattggagccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagctggtagacagtactgataaggctgacttgcggttgatctatctcgcgctggcgcacatgatcaaatttcggggacacttcctcatcgagggggacctgaacccagacaacagcgatgtcgacaaactctttatccaactggttcagacttacaatcagcttttcgaggagaacccgatcaacgcatccggcgttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagctgcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagctgaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgctcggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagagacgccgatatacaggatgggggcggctgtcaagaaaactgatcaatggcatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaaccggttgtccgactacgacgtggatcatatcgtgccccaaagctttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagctggaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccgcaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatccagcttcgagaaaaaccccatcgactttctcgaagcgaaaggatataaagaggtcaaaaaagacctcatcattaagctgcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgagttctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacggcaccggcgggcccaagaagaagaggaaggtatacccatacgatgttcctgactatgcgggctatccctatgacgtcccggactatgcaggatcgtatccttatgacgttccagattacgctggatcctaatgattcgaatccggataagcggccgcctcgagatatctagacccagctttcttgtacaaagtggttgatcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttaggga

SEQ ID NO: 71 - DNA, 8768 bp, synthetic construct
  misc_feature 1..8768: note = Synthetic
  source 1..8768: mol_type = other DNA
accgccattctgcctggggacgtcggagcaagcttgatttaggtgacactatagaatacaagctacttgttctttttgcaggatcccatcgattcgaattcaaggatcaacaagtttgtacaaaaaagcaggctggcgccaccatggacaagaagtactccattgggctcgatatcggtaccaacagcgtcggctgggccgtcattacggacgagtacaaggtgccgagcaaaaaattcaaagttctgggcaataccgatcgccacagcataaagaagaacctcattggagccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagctggtagacagtactgataaggctgacttgcggttgatctatctcgcgctggcgcacatgatcaaatttcggggacacttcctcatcgagggggacctgaacccagacaacagcgatgtcgacaaactctttatccaactggttcagacttacaatcagcttttcgaggagaacccgatcaacgcatccggcgttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagctgcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagctgaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgctcggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagagacgccgatatacaggatgggggcggctgtcaagaaaactgatcaatggcatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaaccggttgtccgactacgacgtggatcatatcgtgccccaaagctttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagctggaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccgcaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatccagcttcgagaaaaaccccatcgactttctcgaagcgaaaggatataaagaggtcaaaaaagacctcatcattaagctgcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgagttctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacggcaccggcgggcccaagaagaagaggaaggtatacccatacgatgttcctgactatgcgggctatccctatgacgtcccggactatgcaggatcgtatccttatgacgttccagattacgctggatccgccgctccggcagctaagaaaaagaaactggatttcgaatccggaaagccctataaatgtcctgaatgtggcaagtccttctcggagaagagccacctgacacggcaccaacgcacgcacactggtgagaagccatacgcgtgtcctgtcgagtcctgtgaccgccgcttcagtcggagcgaccacctgacacagcacatccgcattcacacagggcaaaaaccgtttcaatgccgcatctgcatgaggaacttcagcgacaagggccacctgacccggcacatccgcacccacacaggagaaaagcccttcgcctgtgacatctgcggcaggaagttcgcgcggagcgacgacctgacacggcacaccaagatccacctccgtcagaaagaccccgggtaatgactcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttaggga

SEQ ID NO: 72 - DNA, 8768 bp, synthetic construct
  misc_feature 1..8768: note = Synthetic
  source 1..8768: mol_type = other DNA
accgccattctgcctggggacgtcggagcaagcttgatttaggtgacactatagaatacaagctacttgttctttttgcaggatcccatcgattcgaattcaaggatcaacaagtttgtacaaaaaagcaggctggcgccaccatggacaagaagtactccattgggctcgatatcggtaccaacagcgtcggctgggccgtcattacggacgagtacaaggtgccgagcaaaaaattcaaagttctgggcaataccgatcgccacagcataaagaagaacctcattggagccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagctggtagacagtactgataaggctgacttgcggttgatctatctcgcgctggcgcacatgatcaaatttcggggacacttcctcatcgagggggacctgaacccagacaacagcgatgtcgacaaactctttatccaactggttcagacttacaatcagcttttcgaggagaacccgatcaacgcatccggcgttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagctgcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagctgaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgctcggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagagacgccgatatacaggatgggggcggctgtcaagaaaactgatcaatggcatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaaccggttgtccgactacgacgtggatcatatcgtgccccaaagctttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagctggaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccgcaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatccagcttcgagaaaaaccccatcgactttctcgaagcgaaaggatataaagaggtcaaaaaagacctcatcattaagctgcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgagttctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagaagtacacctctacaaaggaggtcctggacgccacactgattcatcagtcaattacggggctctatgaaacaagaatcgacctctctcagctcggtggagacggcaccggcgggcccaagaagaagaggaaggtatacccatacgatgttcctgactatgcgggctatccctatgacgtcccggactatgcaggatcgtatccttatgacgttccagattacgctggatccgccgctccggcagctaagaaaaagaaactggatttcgaatccggaaagccctataaatgtcctgaatgtggcaagtccttctcggagaagagccacctgacacggcaccaacgcacgcacactggtgagaagccatacgcgtgtcctgtcgagtcctgtgaccgccgcttcagtcggagcgaccacctgacacagcacatccgcattcacacagggcaaaaaccgtttcaatgccgcatctgcatgaggaacttcagcgacaagggccacctgacccggcacatccgcacccacacaggagaaaagcccttcgcctgtgacatctgcggcaggaagttcgcgcggagcgacgacctgacacggcacaccaagatccacctccgtcagaaagaccccgggtaatgactcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttaggga

SEQ ID NO: 73 - DNA, 8768 bp, synthetic construct
  misc_feature 1..8768: note = Synthetic
  source 1..8768: mol_type = other DNA
accgccattctgcctggggacgtcggagcaagcttgatttaggtgacactatagaatacaagctacttgttctttttgcaggatcccatcgattcgaattcaaggatcaacaagtttgtacaaaaaagcaggctggcgccaccatggacaagaagtactccattgggctcgatatcggtaccaacagcgtcggctgggccgtcattacggacgagtacaaggtgccgagcaaaaaattcaaagttctgggcaataccgatcgccacagcataaagaagaacctcattggagccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagctggtagacagtactgataaggctgacttgcggttgatctatctcgcgctggcgcacatgatcaaatttcggggacacttcctcatcgagggggacctgaacccagacaacagcgatgtcgacaaactctttatccaactggttcagacttacaatcagcttttcgaggagaacccgatcaacgcatccggcgttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagctgcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagctgaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgctcggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagagacgccgatatacaggatgggggcggctgtcaagaaaactgatcaatggcatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaaccggttgtccgactacgacgtggatcatatcgtgccccaaagctttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagctggaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccgcaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatccagcttcgagaaaaaccccatcgactttctcgaagcgaaaggatataaagaggtcaaaaaagacctcatcattaagctgcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgagttctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacggcaccggcgggcccaagaagaagaggaaggtatacccatacgatgttcctgactatgcgggctatccctatgacgtcccggactatgcaggatcgtatccttatgacgttccagattacgctggatccgccgctccggcagctaagaaaaagaaactggatttcgaatccggaaagccctataaatgtcctgaatgtggcaagtccttctcgcggagcgacgacctgacacggcaccaacgtacgcacactggtgagaagccatacgcgtgtcctgtcgagtcctgtgaccgccgcttcagtcagaagggacacctgacacggcacatccgcattcacacagggcaaaaaccgtttcaatgccgcatctgcatgaggaacttcagcatccgtagcagcctgacacggcacatccgcacccacacaggagaaaagcccttcgcctgtgacatctgcggcaggaagttcgcgctgagccaccacctgacacggcacaccaagatccacctccgtcagaaagaccccgggtaatgactcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttaggga

SEQ ID NO: 74 - DNA, 8768 bp, synthetic construct
  misc_feature 1..8768: note = Synthetic
  source 1..8768: mol_type = other DNA
accgccattctgcctggggacgtcggagcaagcttgatttaggtgacactatagaatacaagctacttgttctttttgcaggatcccatcgattcgaattcaaggatcaacaagtttgtacaaaaaagcaggctggcgccaccatggacaagaagtactccattgggctcgatatcggtaccaacagcgtcggctgggccgtcattacggacgagtacaaggtgccgagcaaaaaattcaaagttctgggcaataccgatcgccacagcataaagaagaacctcattggagccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagctggtagacagtactgataaggctgacttgcggttgatctatctcgcgctggcgcacatgatcaaatttcggggacacttcctcatcgagggggacctgaacccagacaacagcgatgtcgacaaactctttatccaactggttcagacttacaatcagcttttcgaggagaacccgatcaacgcatccggcgttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagctgcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagctgaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgctcggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagagacgccgatatacaggatgggggcggctgtcaagaaaactgatcaatggcatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaaccggttgtccgactacgacgtggatcatatcgtgccccaaagctttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagctggaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccgcaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatccagcttcgagaaaaaccccatcgactttctcgaagcgaaaggatataaagaggtcaaaaaagacctcatcattaagctgcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgagttctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagaagtacacctctacaaaggaggtcctggacgccacactgattcatcagtcaattacggggctctatgaaacaagaatcgacctctctcagctcggtggagacggcaccggcgggcccaagaagaagaggaaggtatacccatacgatgttcctgactatgcgggctatccctatgacgtcccggactatgcaggatcgtatccttatgacgttccagattacgctggatccgccgctccggcagctaagaaaaagaaactggatttcgaatccggaaagccctataaatgtcctgaatgtggcaagtccttctcgcggagcgacgacctgacacggcaccaacgtacgcacactggtgagaagccatacgcgtgtcctgtcgagtcctgtgaccgccgcttcagtcagaagggacacctgacacggcacatccgcattcacacagggcaaaaaccgtttcaatgccgcatctgcatgaggaacttcagcatccgtagcagcctgacacggcacatccgcacccacacaggagaaaagcccttcgcctgtgacatctgcggcaggaagttcgcgctgagccaccacctgacacggcacaccaagatccacctccgtcagaaagaccccgggtaatgactcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttaggga

SEQ ID NO: 75 - DNA, 8768 bp, synthetic construct
  misc_feature 1..8768: note = Synthetic
  source 1..8768: mol_type = other DNA
accgccattctgcctggggacgtcggagcaagcttgatttaggtgacactatagaatacaagctacttgttctttttgcaggatcccatcgattcgaattcaaggatcaacaagtttgtacaaaaaagcaggctggcgccaccatggacaagaagtactccattgggctcgatatcggtaccaacagcgtcggctgggccgtcattacggacgagtacaaggtgccgagcaaaaaattcaaagttctgggcaataccgatcgccacagcataaagaagaacctcattggagccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagctggtagacagtactgataaggctgacttgcggttgatctatctcgcgctggcgcacatgatcaaatttcggggacacttcctcatcgagggggacctgaacccagacaacagcgatgtcgacaaactctttatccaactggttcagacttacaatcagcttttcgaggagaacccgatcaacgcatccggcgttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagctgcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagctgaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgctcggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagagacgccgatatacaggatgggggcggctgtcaagaaaactgatcaatggcatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaaccggttgtccgactacgacgtggatcatatcgtgccccaaagctttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagctggaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccgcaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatccagcttcgagaaaaaccccatcgactttctcgaagcgaaaggatataaagaggtcaaaaaagacctcatcattaagctgcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgagttctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacggcaccggcgggcccaagaagaagaggaaggtatacccatacgatgttcctgactatgcgggctatccctatgacgtcccggactatgcaggatcgtatccttatgacgttccagattacgctggatccgccgctccggcagctaagaaaaagaaactggatttcgaatccggaaagccctataaatgtcctgaatgtggcaagtccttctcgcagagcggagacctgacacggcaccaacgtacgcacactggtgagaagccatacgcgtgtcctgtcgagtcctgtgaccgccgcttcagtgagaagagccacctgacacggcacatccgcattcacacagggcaaaaaccgtttcaatgccgcatctgcatgaggaacttcagctgtgcccaccacctgacacggcacatccgcacccacacaggagaaaagcccttcgcctgtgacatctgcggcaggaagttcgcggaccggagcaccctgacacagcacaccaagatccacctccgtcagaaagaccccgggtaatgactcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttaggga

SEQ ID NO: 76 - DNA, 8768 bp, synthetic construct
  misc_feature 1..8768: note = Synthetic
  source 1..8768: mol_type = other DNA
accgccattctgcctggggacgtcggagcaagcttgatttaggtgacactatagaatacaagctacttgttctttttgcaggatcccatcgattcgaattcaaggatcaacaagtttgtacaaaaaagcaggctggcgccaccatggacaagaagtactccattgggctcgatatcggtaccaacagcgtcggctgggccgtcattacggacgagtacaaggtgccgagcaaaaaattcaaagttctgggcaataccgatcgccacagcataaagaagaacctcattggagccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagctggtagacagtactgataaggctgacttgcggttgatctatctcgcgctggcgcacatgatcaaatttcggggacacttcctcatcgagggggacctgaacccagacaacagcgatgtcgacaaactctttatccaactggttcagacttacaatcagcttttcgaggagaacccgatcaacgcatccggcgttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagctgcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagctgaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgctcggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagagacgccgatatacaggatgggggcggctgtcaagaaaactgatcaatggcatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaaccggttgtccgactacgacgtggatcatatcgtgccccaaagctttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagctggaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccgcaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatccagcttcgagaaaaaccccatcgactttctcgaagcgaaaggatataaagaggtcaaaaaagacctcatcattaagctgcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgagttctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagaagtacacctctacaaaggaggtcctggacgccacactgattcatcagtcaattacggggctctatgaaacaagaatcgacctctctcagctcggtggagacggcaccggcgggcccaagaagaagaggaaggtatacccatacgatgttcctgactatgcgggctatccctatgacgtcccggactatgcaggatcgtatccttatgacgttccagattacgctggatccgccgctccggcagctaagaaaaagaaactggatttcgaatccggaaagccctataaatgtcctgaatgtggcaagtccttctcgcagagcggagacctgacacggcaccaacgtacgcacactggtgagaagccatacgcgtgtcctgtcgagtcctgtgaccgccgcttcagtgagaagagccacctgacacggcacatccgcattcacacagggcaaaaaccgtttcaatgccgcatctgcatgaggaacttcagctgtgcccaccacctgacacggcacatccgcacccacacaggagaaaagcccttcgcctgtgacatctgcggcaggaagttcgcggaccggagcaccctgacacagcacaccaagatccacctccgtcagaaagaccccgggtaatgactcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttaggga

SEQ ID NO: 77 - protein, 1510 aa, synthetic construct
  REGION 1..1510: note = Synthetic
MDKKYSIGLDIGTNSVGWAVITDEYKVPSKKFKVLGNTDRHSIKKNLIGALLFDSGETEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDFNASLGTYHDLLKIIKDKDFLDNEENEDILEDIVLTLTLFEDREMIEERLKTYAHLFDDKVMKQLKRRRYTGWGRLSRKLINGTRDKQSGKTILDFLKSDGFANRNFMQLIHDDSLTFKEDIQKAQVSGQGDSLHEHIANLAGSPAIKKGILQTVKVVDELVKVMGRHKPENIVIEMARNQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDAFTVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLTIHQSITGLYETRIDLSQLGGDGTGGPKKKRKVYPYDVPDYAGYPYDVPDYAGSYPYDVPDYAGSAAPAAKKKKLDFESGRPYACPVESCDRRFSRSDELTRHIRIHTGQKPFQCRICMRNFSRSDHLTTHIRTHTGEKPFACDICGRKFARSDERKRHTKIHLRQKD

SEQ ID NO: 78 - protein, 1542 aa, synthetic construct
  REGION 1..1542: note = Synthetic
MGRAAPAAKKKKLDQLRPYACPVESCDRRFSRSDELTRHIRIHTGQKPFQCRICMRNFSRSDHLTTHIRTHTGEKPFACDICGRKFARSDERKRHTKIHLRQKDPGSGGSGSSGRTAAPAAKKKKLDSEFGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGTGGPKKKRKVYPYDVPDYAGYPYDVPDYAGSYPYDVPDYAGS

SEQ ID NO: 79 - protein, 2008 aa, synthetic construct
  REGION 1..2008: note = Synthetic
MGRAAPAAKKKKLDQLGASGSPGSGGSGSSGRTAAPAAKKKKLDSEFGSGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGTGGPKKKRKVYPYDVPDYAGYPYDVPDYAGSYPYDVPDYAGSAAPAAKKKKLDFESGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLPLNLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIANNNGGRPALESIVAQLSRPDPALAALTNDHLVALACLGGRPALDAVKKGLPHAPALIKRTNRRIPERTSHRVAGS

SEQ ID NO: 80 - protein, 1986 aa, synthetic construct
  REGION 1..1986: note = Synthetic
MGRAAPAAKKKKLDQLVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLPLNLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIANNNGGRPALESIVAQLSRPDPALAALTNDHLVALACLGGRPALDAVKKGLPHAPALIKRTNRRIPERTSHRVAGSPGSGGSGSSGRTAAPAAKKKKLDSEFGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGTGGPKKKRKVYPYDVPDYAGYPYDVPDYAGSYPYDVPDYAGS

SEQ ID NO: 81 - protein, 1544 aa, synthetic construct
  REGION 1..1544: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGTGGPKKKRKVYPYDVPDYAGYPYDVPDYAGSYPYDVPDYAGSAAPAAKKKKLDFESGKPYKCPECGKSFSQSGDLTRHQRTHTGEKPYACPVESCDRRFSEKSHLTRHIRIHTGQKPFQCRICMRNFSCAHHLTRHIRTHTGEKPFACDICGRKFADRSTLTQHTKIHLRQKDPG

SEQ ID NO: 82 - protein, 1544 aa, synthetic construct
  REGION 1..1544: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGTGGPKKKRKVYPYDVPDYAGYPYDVPDYAGSYPYDVPDYAGSAAPAAKKKKLDFESGKPYKCPECGKSFSQKGHLTRHQRTHTGEKPYACPVESCDRRFSDRSDLTRHIRIHTGQKPFQCRICMRNFSRSDHLTRHIRTHTGEKPFACDICGRKFAQSGDLTRHTKIHLRQKDPG

SEQ ID NO: 83 - protein, 1544 aa, synthetic construct
  REGION 1..1544: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGTGGPKKKRKVYPYDVPDYAGYPYDVPDYAGSYPYDVPDYAGSAAPAAKKKKLDFESGKPYKCPECGKSFSRSDDLTRHQRTHTGEKPYACPVESCDRRFSQKGHLTRHIRIHTGQKPFQCRICMRNFSIRSSLTRHIRTHTGEKPFACDICGRKFALSHHLTRHTKIHLRQKDPG

SEQ ID NO: 84 - DNA, 47 bp, synthetic construct
  misc_feature 1..47: note = Synthetic
  source 1..47: mol_type = other DNA
gaccccctccaccccgcctccgggcgcgggctccggcccctgcccgc

SEQ ID NO: 85 - DNA, 47 bp, synthetic construct
  misc_feature 1..47: note = Synthetic
  source 1..47: mol_type = other DNA
ggtgagtgagtgtgtgcgtgtggggttgagggtgttggagcggggag

SEQ ID NO: 86 - DNA, 47 bp, synthetic construct
  misc_feature 1..47: note = Synthetic
  source 1..47: mol_type = other DNA
gagtccgagcagaagaagaagggctcccatcacatcaaccggtggcg

SEQ ID NO: 87 - DNA, 58 bp, synthetic construct
  misc_feature 1..58: note = Synthetic
  source 1..58: mol_type = other DNA
gccgaccccctccaccccgcctccgggcgcgggctccggcccctgcccgcggctcgcc

SEQ ID NO: 88 - DNA, 58 bp, synthetic construct; identical to SEQ ID NO: 87
  misc_feature 1..58: note = Synthetic
  source 1..58: mol_type = other DNA

SEQ ID NO: 89 - DNA, 43 bp, synthetic construct; identical to SEQ ID NO: 40
  misc_feature 1..43: note = Synthetic
  source 1..43: mol_type = other DNA

SEQ ID NO: 90 - DNA, 43 bp, synthetic construct; identical to SEQ ID NO: 41
  misc_feature 1..43: note = Synthetic
  source 1..43: mol_type = other DNA

SEQ ID NO: 91 - DNA, 43 bp, synthetic construct; identical to SEQ ID NO: 42
  misc_feature 1..43: note = Synthetic
  source 1..43: mol_type = other DNA

SEQ ID NO: 92 - DNA, 43 bp, synthetic construct; identical to SEQ ID NO: 43
  misc_feature 1..43: note = Synthetic
  source 1..43: mol_type = other DNA

SEQ ID NO: 93 - protein, 43 aa, synthetic construct
  REGION 1..43: note = Synthetic
GGGCCCAACCTAGGGCATGGAGGCGGCTGCTGGTGCGTGGGCG

SEQ ID NO: 94 - DNA, 43 bp, synthetic construct; identical to SEQ ID NO: 40
  misc_feature 1..43: note = Synthetic
  source 1..43: mol_type = other DNA

SEQ ID NO: 95 - DNA, 49 bp, synthetic construct
  misc_feature 1..49: note = Synthetic
  source 1..49: mol_type = other DNA
atggacccctaggtctccggaggctacacgtgttccccgatcccgtttc

SEQ ID NO: 96 - DNA, 47 bp, synthetic construct
  misc_feature 1..47: note = Synthetic
  source 1..47: mol_type = other DNA
tgtttggtgggtactcacccagggctaagagctgccaggtactgtgt

SEQ ID NO: 97 - DNA, 47 bp, synthetic construct; identical to SEQ ID NO: 84
  misc_feature 1..47: note = Synthetic
  source 1..47: mol_type = other DNA

SEQ ID NO: 98 - DNA, 47 bp, synthetic construct; identical to SEQ ID NO: 85
  misc_feature 1..47: note = Synthetic
  source 1..47: mol_type = other DNA

SEQ ID NO: 99 - DNA, 47 bp, synthetic construct; identical to SEQ ID NO: 86
  misc_feature 1..47: note = Synthetic
  source 1..47: mol_type = other DNA

SEQ ID NO: 100 - DNA, 58 bp, synthetic construct
  misc_feature 1..58: note = Synthetic
  source 1..58: mol_type = other DNA
gtgggtgagtgagtgtgtgcgtgtggggttgagggtgttggagccgggagaaggccag

SEQ ID NO: 101 - DNA, 58 bp, synthetic construct
  misc_feature 1..58: note = Synthetic
  source 1..58: mol_type = other DNA
gtgggtgagtgagtgtgtgcgtgtggggttgagggtgttggagcggggagaaggccag

SEQ ID NO: 102 - DNA, 58 bp, synthetic construct
  misc_feature 1..58: note = Synthetic
  source 1..58: mol_type = other DNA
cctgagtccgagcagaagaagaagggctcccatcacatcaaccggtggcgcattgcca

SEQ ID NO: 103 - DNA, 58 bp, synthetic construct; identical to SEQ ID NO: 101
  misc_feature 1..58: note = Synthetic
  source 1..58: mol_type = other DNA

SEQ ID NO: 104 - DNA, 58 bp, synthetic construct; identical to SEQ ID NO: 102
  misc_feature 1..58: note = Synthetic
  source 1..58: mol_type = other DNA

SEQ ID NO: 105 - DNA, 20 bp, synthetic construct
  misc_feature 1..20: note = Synthetic
  source 1..20: mol_type = other DNA
gagtgagtgtgtgcgtgtgg

SEQ ID NO: 106 - DNA, 21 bp, synthetic construct
  misc_feature 1..21: note = Synthetic
  source 1..21: mol_type = other DNA
gtccgagcagaagaagaaggg

SEQ ID NO: 107 - DNA, 58 bp, synthetic construct; identical to SEQ ID NO: 87
  misc_feature 1..58: note = Synthetic
  source 1..58: mol_type = other DNA

SEQ ID NO: 108 - DNA, 58 bp, synthetic construct; identical to SEQ ID NO: 87
  misc_feature 1..58: note = Synthetic
  source 1..58: mol_type = other DNA

SEQ ID NO: 109 - DNA, 45 bp, synthetic construct
  misc_feature 1..45: note = Synthetic
  source 1..45: mol_type = other DNA
gggcccctccaccccgcctctggccaagttttgaggatagggagg

SEQ ID NO: 110 - DNA, 12 bp, synthetic construct
  misc_feature 1..12: note = Synthetic
  misc_feature 12: note = n is a, c, g, t or u
  source 1..12: mol_type = other DNA
gcggryarggrn

SEQ ID NO: 111 - DNA, 58 bp, synthetic construct; identical to SEQ ID NO: 87
  misc_feature 1..58: note = Synthetic
  source 1..58: mol_type = other DNA

SEQ ID NO: 112 - DNA, 58 bp, synthetic construct; identical to SEQ ID NO: 87
  misc_feature 1..58: note = Synthetic
  source 1..58: mol_type = other DNA

SEQ ID NO: 113 - DNA, 58 bp, synthetic construct
  misc_feature 1..58: note = Synthetic
  source 1..58: mol_type = other DNA
taggggcccctccaccccgcctctggccaagttttgaggatagggaggtgggtagccc

SEQ ID NO: 114 - protein, 1110 aa, Neisseria meningitidis
MVPKKKRKVAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRYPYDVPDYAAAPAAKKKKLD

SEQ ID NO: 115 - protein, 1110 aa, synthetic construct
  REGION 1..1110: note = Synthetic
MVPKKKRKVAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHAGTGNINIRIHDLDHKIGKNGILEGIGVKTALSFQKYQIDELGKEIRPCRLKKRPPVRYPYDVPDYAAAPAAKKKKLD

SEQ ID NO: 116 - protein, 1110 aa, synthetic construct
  REGION 1..1110: note = Synthetic
MVPKKKRKVAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKAARMFGYFASCHAGTGNINIRIHDLDHKIGKNGILEGIGVKTALSFQKYQIDELGKEIRPCRLKKRPPVRYPYDVPDYAAAPAAKKKKLD

SEQ ID NO: 117 - protein, 1240 aa, synthetic construct
  REGION 1..1240: note = Synthetic
MGRAAPAAKKKKLDQLRPYACPVESCDRRFSRSDELTRHIRIHTGQKPFQCRICMRNFSRSDHLTTHIRTHTGEKPFACDICGRKFARSDERKRHTKIHLRQKDPGSGGSGSSGRTAAPAAKKKKLDIRKLVPKKKRKVAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRYPYDVPDYAAAPAAKKKKLD

SEQ ID NO: 118 - protein, 1246 aa, synthetic construct
  REGION 1..1246: note = Synthetic
MVPKKKRKVAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRYPYDVPDYAAGTGGPKKKRKVYPYDVPDYAGYPYDVPDYAGSYPYDVPDYAGSAAPAAKKKKLDFESGRPYACPVESCDRRFSRSDELTRHIRIHTGQKPFQCRICMRNFSRSDHLTTHIRTHTGEKPFACDICGRKFARSDERKRHTKIHLRQKD

SEQ ID NO: 119 - protein, 1240 aa, synthetic construct
  REGION 1..1240: note = Synthetic
MGRAAPAAKKKKLDQLRPYACPVESCDRRFSRSDELTRHIRIHTGQKPFQCRICMRNFSRSDHLTTHIRTHTGEKPFACDICGRKFARSDERKRHTKIHLRQKDPGSGGSGSSGRTAAPAAKKKKLDIRKLVPKKKRKVAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHAGTGNINIRIHDLDHKIGKNGILEGIGVKTALSFQKYQIDELGKEIRPCRLKKRPPVRYPYDVPDYAAAPAAKKKKLD

SEQ ID NO: 120 - protein, 1246 aa, synthetic construct
  REGION 1..1246: note = Synthetic
MVPKKKRKVAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHAGTGNINIRIHDLDHKIGKNGILEGIGVKTALSFQKYQIDELGKEIRPCRLKKRPPVRYPYDVPDYAAGTGGPKKKRKVYPYDVPDYAGYPYDVPDYAGSYPYDVPDYAGSAAPAAKKKKLDFESGRPYACPVESCDRRFSRSDELTRHIRIHTGQKPFQCRICMRNFSRSDHLTTHIRTHTGEKPFACDICGRKFARSDERKRHTKIHLRQKD

SEQ ID NO: 121 - protein, 1240 aa, synthetic construct
  REGION 1..1240: note = Synthetic
MGRAAPAAKKKKLDQLRPYACPVESCDRRFSRSDELTRHIRIHTGQKPFQCRICMRNFSRSDHLTTHIRTHTGEKPFACDICGRKFARSDERKRHTKIHLRQKDPGSGGSGSSGRTAAPAAKKKKLDIRKLVPKKKRKVAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKAARMFGYFASCHAGTGNINIRIHDLDHKIGKNGILEGIGVKTALSFQKYQIDELGKEIRPCRLKKRPPVRYPYDVPDYAAAPAAKKKKLD

SEQ ID NO: 122 - protein, 1246 aa, synthetic construct
  REGION 1..1246: note = Synthetic
MVPKKKRKVAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKAARMFGYFASCHAGTGNINIRIHDLDHKIGKNGILEGIGVKTALSFQKYQIDELGKEIRPCRLKKRPPVRYPYDVPDYAAGTGGPKKKRKVYPYDVPDYAGYPYDVPDYAGSYPYDVPDYAGSAAPAAKKKKLDFESGRPYACPVESCDRRFSRSDELTRHIRIHTGQKPFQCRICMRNFSRSDHLTTHIRTHTGEKPFACDICGRKFARSDERKRHTKIHLRQKD

SEQ ID NO: 123 - DNA, 59 bp, synthetic construct
  misc_feature 1..59: note = Synthetic
  source 1..59: mol_type = other DNA
tccccggcatcctagcgcgctgggctagcaatcgcctccgcgtcccttccaacagtacc

SEQ ID NO: 124 - DNA, 59 bp, synthetic construct
  misc_feature 1..59: note = Synthetic
  source 1..59: mol_type = other DNA
tccccggcatcctagcgcgctgggctagcggtactgttggaagggacgcggaggcgatt

SEQ ID NO: 125 - DNA, 59 bp, synthetic construct
  misc_feature 1..59: note = Synthetic
  source 1..59: mol_type = other DNA
ggtactgttggaagggacgcggaggcgattgctagctccccggcatcctagcgcgctgg

SEQ ID NO: 126 - DNA, 59 bp, synthetic construct
  misc_feature 1..59: note = Synthetic
  source 1..59: mol_type = other DNA
aatcgcctccgcgtcccttccaacagtaccgctagctccccggcatcctagcgcgctgg

SEQ ID NO: 127 - DNA, 59 bp, synthetic construct; identical to SEQ ID NO: 123
  misc_feature 1..59: note = Synthetic
  source 1..59: mol_type = other DNA

SEQ ID NO: 128 - DNA, 59 bp, synthetic construct; identical to SEQ ID NO: 124
  misc_feature 1..59: note = Synthetic
  source 1..59: mol_type = other DNA

SEQ ID NO: 129 - DNA, 59 bp, synthetic construct; identical to SEQ ID NO: 125
  misc_feature 1..59: note = Synthetic
  source 1..59: mol_type = other DNA

SEQ ID NO: 130 - DNA, 59 bp, synthetic construct; identical to SEQ ID NO: 126
  misc_feature 1..59: note = Synthetic
  source 1..59: mol_type = other DNA

SEQ ID NO: 131 - DNA, 71 bp, synthetic construct
  misc_feature 1..71: note = Synthetic
  source 1..71: mol_type = other DNA
ggtgagtgagtgtgtgcgtgtggggttgagggcgttggagcggggagaaggccaggggtcactccaggatt

SEQ ID NO: 132 - DNA, 23 bp, synthetic construct
  misc_feature 1..23: note = Synthetic
  source 1..23: mol_type = other DNA
gaccccctccaccccgcctccgg

SEQ ID NO: 133 - DNA, 23 bp, synthetic construct
  misc_feature 1..23: note = Synthetic
  source 1..23: mol_type = other DNA
ctgcccccccaccccgccactgg

SEQ ID NO: 134 - protein, 1544 aa, synthetic construct
  REGION 1..1544: note = Synthetic
MGRAAPAAKKKKLDQLGASGSPGSGGSGSSGRTAAPAAKKKKLDSEFGSGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKKYTSTKEVLDATLIHQSITGLYETRIDLSQLGGDGTGGPKKKRKVIGSSGMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLELE

SEQ ID NO: 135 - protein, 341 aa, synthetic construct
  REGION 1..341: note = Synthetic
MGRGSLQAAPAAKKKKLDQLASGGGGSGAAPKKKRKVSELGGGGSDYKDHDGDYKDHDIDYKDDDDKLEGGGGSGLDLASLILGNPGSLDLASLILPRGGGGSGAGGGVPSGKPYKCPECGKSFSEKSHLTRHQRTHTGEKPYACPVESCDRRFSRSDHLTQHIRIHTGQKPFQCRICMRNFSDKGHLTRHIRTHTGEKPFACDICGRKFARSDDLTRHTKIHLRQKDPGGSGSGGGGSGGGGSRTILWHEMWHEGLEEASRLYFGERNVKGMFEVLEPLHAMMERGPQTLKETSFNQAYGRDLMEAQEWCRKYMKSGNVPDLLQAFDLYYHVFRRISKEF

SEQ ID NO: 136 - protein, 707 aa, synthetic construct
  REGION 1..707: note = Synthetic
MGRAAPAAKKKKLDQLGASGSPGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIEGTGGPKKKRKVIGSSGILWHEMWHEGLEEASRLYFGERNVKGMFEVLEPLHAMMERGPQTLKETSFNQAYGRDLMEAQEWCRKYMKSGNVKDLTQAWDLYYHVFRRISKLE

SEQ ID NO: 137 - protein, 1157 aa, synthetic construct
  REGION 1..1157: note = Synthetic
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGAGGCFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKKYTSTKEVLDATLIHQSITGLYETRIDLSQLGGDGTGGPKKKRKVYPYDVPDYAGYPYDVPDYAGSYPYDVPDYAGSAAPAAKKKKLDFESGKPYKCPECGKSFSRSDDLTRHQRTHTGEKPYACPVESCDRRFSQKGHLTRHIRIHTGQKPFQCRICMRNFSIRSSLTRHIRTHTGEKPFACDICGRKFALSHHLTRHTKIHLRQKDPGGSGSLEGDYKDHDGDYKDHDIDYKDDDDKQGSELGSGRAAPAAKKKKLDQLGASGSSGGSSGAAPKKKRKVSEF

SEQ ID NO: 138 - DNA, 42 bp, synthetic construct
  misc_feature 1..42: note = Synthetic
  source 1..42: mol_type = other DNA
gggagctctctggctaactagggaacccactgcttaagcctc

SEQ ID NO: 139 - DNA, 46 bp, synthetic construct
  misc_feature 1..46: note = Synthetic
  source 1..46: mol_type = other DNA
cctccctggaaagtccccagcggaaagtcccttgtagaaagctcga

SEQ ID NO: 140 - DNA, 47 bp, synthetic construct
  misc_feature 1..47: note = Synthetic
  source 1..47: mol_type = other DNA
ccgctggggactttccagggaggtgtggcctgggcgggactggggag

SEQ ID NO: 141 - protein, 1575 aa, synthetic construct
  REGION 1..1575: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKKYTSTKEVLDATLIHQSITGLYETRIDLSQLGGDGTGGPKKKRKVYPYDVPDYAGYPYDVPDYAGSYPYDVPDYAGSAAPAAKKKKLDFESGRPFACDICGKKFARSDHLTTHTKIHTGQKPFQCRICMRNFSRSDNLKQHIRTHTGEKPFACDICGKKFARNSNLTQHTKIHTHPRAPIPKPFQCRICMRNFSQSSDLTRHIRTHTGEKPFACDICGRKFARSDNLTRHTKIHLRDKQPG

SEQ ID NO: 142 - protein, 1547 aa, synthetic construct
  REGION 1..1547: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKKYTSTKEVLDATLIHQSITGLYETRIDLSQLGGDGTGGPKKKRKVYPYDVPDYAGYPYDVPDYAGSYPYDVPDYAGSAAPAAKKKKLDFESGRPFQCRICMRNFSQSGHLKSHIRTHTGEKPFACDICGKKFAQSSDLTRHTKIHTHPRAPIPKPFQCRICMRNFSQSGNLTRHIRTHTGEKPFACDICGRKFAQSGALTRHTKIHLRDKQPG

SEQ ID NO: 143 - protein, 1547 aa, synthetic construct
  REGION 1..1547: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKKYTSTKEVLDATLIHQSITGLYETRIDLSQLGGDGTGGPKKKRKVYPYDVPDYAGYPYDVPDYAGSYPYDVPDYAGSAAPAAKKKKLDFESGRPFQCRICMRNFSRSDNLTRHIRTHTGEKPFACDICGKKFARSDHLTRHTKIHTHPRAPIPKPFQCRICMRNFSLKGNLTRHIRTHTGEKPFACDICGRKFARSDHLSDHTKIHLRDKQPG

SEQ ID NO: 144 - DNA, 103 bp, synthetic construct
  misc_feature 1..103: note = Synthetic
  source 1..103: mol_type = other DNA
gaccccctccaccccgcctcgttttagagctagaaatagcaagttaaaataaggctagtccgttatcaacttgaaaaagtggcaccgagtcggtgcttttttt

SEQ ID NO: 145 - DNA, 103 bp, synthetic construct
  misc_feature 1..103: note = Synthetic
  source 1..103: mol_type = other DNA
ggtgagtgagtgtgtgcgtggttttagagctagaaatagcaagttaaaataaggctagtccgttatcaacttgaaaaagtggcaccgagtcggtgcttttttt

SEQ ID NO: 146 - DNA, 103 bp, synthetic construct
  misc_feature 1..103: note = Synthetic
  source 1..103: mol_type = other DNA
gagtccgagcagaagaagaagttttagagctagaaatagcaagttaaaataaggctagtccgttatcaacttgaaaaagtggcaccgagtcggtgcttttttt

SEQ ID NO: 147 - DNA, 103 bp, synthetic construct
  misc_feature 1..103: note = Synthetic
  source 1..103: mol_type = other DNA
tacttccgggcacagtccacgttttagagctagaaatagcaagttaaaataaggctagtccgttatcaacttgaaaaagtggcaccgagtcggtgcttttttt

SEQ ID NO: 148 - DNA, 150 bp, synthetic construct
  misc_feature 1..150: note = Synthetic
  source 1..150: mol_type = other DNA
ggggagaaggccaggggtcactgttgtagctccctttctcatttcggaaacgaaatgagaaccgttgctacaataaggccgtctgaaaagatgtgccgcaacgctctgccccttaaagcttctgctttaaggggcatcgtttattttttt

SEQ ID NO: 149 - protein, 2546 aa, synthetic construct
  REGION 1..2546: note = Synthetic
MPKKKRKVAAFKPNPINYILGLAIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIAAALPFSRTWDDSFNNKVLVLGSEAQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRSRADPKKKRKVEASDYKDHDGDYKDHDIDYKDDDDKTSGGGSGGGSRTAAPAAKKKKLDSEFGSGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKKYTSTKEVLDATLIHQSITGLYETRIDLSQLGGDGTGGPKKKRKVIGSSGSGGSGSLE

SEQ ID NO: 150 - protein, 2551 aa, synthetic construct
  REGION 1..2551: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKKYTSTKEVLDATLIHQSITGLYETRIDLSQLGGDGTGGPKKKRKVYPYDVPDYAGYPYDVPDYAGSYPYDVPDYAGSAAPAAKKKKLDFESGMPKKKRKVAAFKPNPINYILGLAIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIAAALPFSRTWDDSFNNKVLVLGSEAQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRSRADPKKKRKVEASDYKDHDGDYKDHDIDYKDDDDK

SEQ ID NO: 151 - protein, 2551 aa, synthetic construct
  REGION 1..2551: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKKYTSTKEVLDATLIHQSITGLYETRIDLSQLGGDGTGGPKKKRKVYPYDVPDYAGYPYDVPDYAGSYPYDVPDYAGSAAPAAKKKKLDFESGMPKKKRKVAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIAAALPFSRTWDDSFNNKVLVLGSEAQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRSRADPKKKRKVEASDYKDHDGDYKDHDIDYKDDDDK

SEQ ID NO: 152 - protein, 2551 aa, synthetic construct
  REGION 1..2551: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKKYTSTKEVLDATLIHQSITGLYETRIDLSQLGGDGTGGPKKKRKVYPYDVPDYAGYPYDVPDYAGSYPYDVPDYAGSAAPAAKKKKLDFESGMPKKKRKVAAFKPNPINYILGLAIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRSRADPKKKRKVEASDYKDHDGDYKDHDIDYKDDDDK

SEQ ID NO: 153 - protein, 2551 aa, synthetic construct
  REGION 1..2551: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKKYTSTKEVLDATLIHQSITGLYETRIDLSQLGGDGTGGPKKKRKVYPYDVPDYAGYPYDVPDYAGSYPYDVPDYAGSAAPAAKKKKLDFESGMPKKKRKVAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRSRADPKKKRKVEASDYKDHDGDYKDHDIDYKDDDDK